SEQUENCE LISTING (4 sequences)

SEQ ID NO: 1 - DNA, 600 bp, Clostridium difficile 630
ttacagctttctgattttgataaatttaaaacttaccatctaatactaataacaggttaattttatctaattattatagattctcatactgtgccttattctatctataaatacaatttaagtgtccatattgaaatatttgtattgtaatacagctggatattacttaaatccaattgtttccattataattttatgttaaaataatattacaaaatacatctgtttttcttcataaacgggtgaaattccctatcggcggtaaaagcccgcgagccttatggcataatttggtcatattccaaagccaacagtaaaatctggatggtagaagaaaatagtatatgagtacctttatgtaattttacatgagtaatctatacaaatccttcaactaccgtatttattcatgaaattagacacattcaaggtacctaatatacaggtgctttttttgttgtttattttacaattatatcgtacttataaaatctattaagattggagtgttatcatgaaacaaaaatggatagtattgattatcatctgtattggtgtatttatgtctactcttgatggaagtatactaaatatcgcaaa

SEQ ID NO: 2 - DNA, 116 bp, Clostridium difficile 630
gtttttcttcataaacgggtgaaattccctatcggcggtaaaagcccgcgagccttatggcataatttggtcatattccaaagccaacagtaaaatctggatggtagaagaaaata

SEQ ID NO: 3 - DNA, 10 bp, Clostridium difficile 630
atgaaacaaa

SEQ ID NO: 4 - DNA, 19 bp, Clostridium difficile 630
gtacctaatatacaggtgc

(19) United States
(12) Patent Application Publication
(10) Pub. No.: US 2012/0295903 A1
(43) Pub. Date: Nov. 22, 2012

(54) FLAVIN DERIVATIVES

(76) Inventors: Kenneth F. Blount, Guilford, CT (US); Philip D.G. Coish, North Haven, CT (US); Brian R. Dixon, Woodbridge, CT (US); Jayhyuk Myung, Woodbridge, CT (US); David Osterman, Glastonbury, CT (US); Phil Wickens, Richmond Hill (CA); Stephanie Avola, Woodbridge (CA); Nick Baboulas, Scarborough (CA); Angelica Bello, North York (CA); Judd Berman, Toronto (CA); Harpreet Kaur, Brampton (CA); David Moon, Brampton (CA); Vinh Pham, Markham (CA); Andrew Roughton, Port Hope (CA); Jeffrey Wilson, Toronto (CA); Jeffrey A. Leiby, Kalamazoo, MI (US); Dennis Underwood, Jamica Plain, MA (US); Paul Adrian Aristoff, Dexter, MI (US); Heinrich J. Schostarez, Portage, MI (US); Robert A. Chrusciel, Portage, MI (US); Thomas R. Belliotti, Portage, MI (US); Bruce R. Evans, Kalamazoo, MI (US); Frank C. Sciavolino, Waterford, CT (US); Manuel A. Navia, Boston, MA (US)

(21) Appl. No.: 13/381,809

(22) PCT Filed: Jun. 30, 2010
(86) PCT No.: PCT/US10/01876

§ 371 Date: Jul. 19, 2012

Related U.S. Application Data
(60) Provisional application No. 61/221,937, filed on Jun. 30, 2009.
(60) Provisional application No. 61/303,237, filed on Feb. 10, 2010.

(51) Int. Cl. C07D 487/04; A01P 1/00; A61K 31/5377; A61P 31/04; A61K 31/525; A01N 43/90

(52) U.S. Cl. 514/232.8; 544/251; 514/251; 544/115

(57) ABSTRACT

The present invention relates novel flavin derivatives and other flavin derivatives, their use and compositions for use as riboswitch ligands and/or anti-infectives. The invention also provides method of making novel flavin derivatives.

This application claims priority from provisional application No. 61/221,937 filed Jun. 30, 2009, and provisional application No. 61/303,237, filed Feb. 10, 2010, the contents of each of which are incorporated by reference in their entirety.

### TECHNICAL FIELD

The present invention relates to flavin derivatives and their use and compositions for use as riboswitch ligands and/or anti-infectives. The invention also provides methods of making novel flavin derivatives.

### BACKGROUND OF THE INVENTION

The fast growing rate of antibiotic resistance over the past decades has raised serious concerns that the antibiotic treatment options currently available will soon be ineffective. With the widespread usage of antibiotics in combination with the rapid growing rate of bacterial resistance in stark contrast with the decade-old chemical scaffolds available for their treatment, it is imperative that new drugs are developed in the battle against bacterial pathogens.

In many bacteria and fungi, RNA structures termed riboswitches regulate the expression of various genes crucial for survival or virulence. Typically located within the 5′-untranslated region (5′-UTR) of certain mRNAs, members of each known class of riboswitch can fold into a distinct, three-dimensionally structured receptor that recognizes a specific organic metabolite. When the cognate metabolite is present at sufficiently high concentrations during transcription of the mRNA, the riboswitch receptor binds to the metabolite and induces a structural change in the nascent mRNA that prevents expression of the open reading frame (ORF), thereby altering gene expression. In the absence of the cognate metabolite, the riboswitch folds into a structure that does not interfere with the expression of the ORF.

Sixteen different classes of riboswitches have been reported. Members of each class of riboswitch bind to the same metabolite and share a highly conserved sequence and secondary structure. Riboswitch motifs have been identified that bind to thiamine pyrophosphate (TPP), flavin mononucleotide (FMN), glycine, guanine, 3′-5′-cyclic diguanylic acid (c-di-GMP), molybdenum cofactor, glucosamine-6-phosphate (GlcN6P), lysine, adenine, and adocobalamin (AdoCbl) riboswitches. Additionally, four dinstinct riboswitch motifs have been identified that recognize S-adenosylmethionine (SAM) I, II and III, IV and two distinct motifs that recognize pre-queosine-1 (PreQ1). Several antimetabolite ligands have also been identified that bind to known riboswitch classes, including pyrithiamine pyrophosphate (PTPP) which binds TPP riboswitches, L-aminoethylcysteine (AEC) and DL-4-oxalysine which bind to lysine riboswitches and roseoflavin and FMN which bind to FMN riboswitches. The riboswitch-receptors bind to their respective ligands in an interface that approaches the level of complexity and selectivity of proteins. This highly specific interaction allows riboswitches to discriminate against most intimately related analogs of ligands. For instance, the receptor of a guanine-binding riboswitch from Bacillus subtilis forms a three-dimensional structure such that the ligand is almost completely enveloped. The guanine is positioned between two aromatic bases and each polar functional group of the guanine hydrogen bonds with four additional riboswitch nucleotides surrounding it. This level of specificity allows the riboswitch to discriminate against most closely related purine analogs. Similarly, studies of the SAM-binding riboswitches reveal that nearly every functional group of SAM is critical in binding the ligands, allowing it to discriminate highly similar compounds such as S-adenosylhomocysteine (SAH) and S-adenosylmethionine (SAM), which only differ by a single methyl group. Likewise, TPP riboswitches comprise one subdomain that recognizes every polar functional group of the 4-amino-5-hydroxymethyl-2-methylpyrimidine (HMP) moiety, albeit not the thiazole moiety, and another subdomain that coordinates two metal ions and several water molecules to bind the negatively charged pyrophosphate moiety of the ligand. Similar to TPP, guanine and SAM riboswitches, FMN riboswitches form receptor structures that are highly specific for the natural metabolite FMN. It is by this highly specific interaction that allows for the design of small molecules for the regulation of specific genes.

FMN riboswitches are of particular interest of this invention because it is believed that the riboswitch binds to flavin mono-nucleotide (FMN) and represses the expression of enzymes responsible for riboflavin and FMN biosynthesis. Riboflavin is a water-soluble vitamin that is converted by flavokinases and FAD synthases to co-factors FMN and FAD, which are indispensable cofactors involved in energy metabolism and metabolism of fats, ketones, carbohydrates and proteins crucial for all living organisms. Although vertebrates rely on uptake of vitamin from their gut for riboflavin sources, most prokaryotes, fungi and plants synthesize the necessary riboflavin for survival. It is therefore suggested that compounds that are selective for FMN riboswitches may be useful targets against bacterial pathogens by shutting down biosynthesis of riboflavin crucial for survival or virulence. In addition, no examples of the FMN, TPP, nor any other riboswitch class have presently been identified in humans. Therefore, riboswitches appear to offer the potential for the discovery of selective antipathogenic drugs. Additionally, CD3299 riboswitches, which are found in C. difficile bacteria, are of particular interest of this invention. It is therefore the objective of this invention to provide novel flavin derivatives for targeting FMN and/or the CD3299 riboswitches and/or are active against various bacterial strains, along with methods of treating infections comprising administering flavin derivatives.

### SUMMARY OF THE INVENTION

In the first aspect, the invention provides to a Compound of Formula Q:

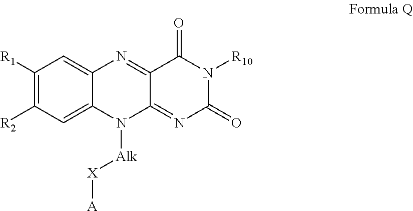

wherein:
(i) Alk is C₁₋₆alkylene (e.g., methylene, ethylene, n-propylene, n-butylene or n-pentylene);
(ii) X is —N(R₆) and A is:
   —C₁₋₄alkyl-N(R₁₁)(R₁₂),
   —C₀₋₄alkyl-aryl¹ (e.g., phenyl, naphthyl, benzyl), or —C₀₋₄alkyl-heteroaryl¹ (e.g., isoxazolyl, (isoxazol-5-yl)methyl, tetrazolyl, pyridyl, for example pyrid-3-yl, (pyrid-5-yl)methyl, indolyl, 1,2,5-oxadiazolyl, pyrrolyl), wherein the alkyl group of said -alkylaryl¹ and -alkylheteroaryl¹ is optionally substituted with hydroxy or another aryl¹ (e.g., phenyl), and the aryl¹ and heteroaryl¹ group of said -alkylaryl¹ and -alkylheteroaryl¹ are independently substituted with one or more:
      —N(Rₐ)—C(O)—C₁₋₄alkyl (e.g., —NHC(O)CH₃), wherein Rₐ is H or C₁₋₄alkyl,
      —OH,
      heteroaryl¹ (e.g., imidazolyl),
      heteroC₃₋₈cycloalkyl (e.g., morpholinyl),
      aryl¹ (e.g., phenyl),
      —O-halo-C₁₋₄alkyl (e.g., —OCF₃),
      —NO₂,
      —N(Rₐ)(R_{b}), wherein Rₐ is H or C₁₋₄alkyl and R_{b} is C₁₋₄alkyl,
      —SO₂—C₁₋₄alkyl (e.g., —SO₂—CH₃);
   —C₀₋₄alkyl-pyridyl substituted with one or more hydroxy (e.g., 2-hydroxypyrid-4-ylmethyl or 2-hydroxypyrid-3-yl);
   —C₀₋₄alkyl-benzotriazolyl (e.g., 1H-benzotriazol-5-yl); —C₀₋₄alkyl-indolyl (e.g., -indol-5-ylmethyl, indol-2-ylmethyl, indol-3-ylethyl);
   —C₀₋₄alkyl-tetrazolyl (e.g., 1,2,3,5-tetrazol-4-ylethyl);
   —C₀₋₄alkyl-oxadiazolyl (e.g., 1,2,5-oxadiazol-3-yl);
   —C₀₋₄alkyl-benzodioxolyl (e.g., 1,3-benzodioxol-5-ylmethyl);
   —C₀₋₄alkyl-benzimidazolyl optionally substituted with —C₀₋₄alkyl (e.g., 1-methylbenzimidazol-2-ylmethyl, benzimidazol-5-ylmethyl);
   —C₀₋₄alkyl-imidazolyl optionally substituted with C₁₋₄alkyl (e.g., 1-methyl-imidazol-5-ylmethyl);
   —C₀₋₄alkyl-pyrrolyl optionally substituted with —C₀₋₄alkyl (e.g., 1-methylpyrrolidin-2-ylmethyl); or
   para-phenylbenzyl;
or
X is a single bond, and A is a monocyclic heteroaryl² (e.g., pyrrolyl, for example pyrrol-1-yl; pyridyl, for example pyrid-2-yl, pyrid-4-yl or pyrid-3-yl; tetrazolyl, for example 1,2,3,4-tetrazol-1-yl; imidazolyl, for example imidazol-1-yl; or isoxazolyl, for example isoxazol-5-yl) wherein said monocyclic heteroaryl² is optionally substituted with C₁₋₄alkyl (e.g., methyl);
or
X is a single bond, —N(R₆)—, —N(R₆)—CH₂—, —N(R₆)—CH₂CH₂—, —N(R₆)—C(H)(CH₃)—, or —C(O)—; and:
   A is a C₃₋₈cycloalkyl² (e.g., C₄cycloalkyl² or C₅₋₆cycloalkyl²) wherein one or more carbon atoms of said cycloalkyl² are optionally and independently replaced with N, O, S, S(O)₂ or —C(O)—, for example:
      cyclobutyl,
      cyclopentyl,
      cyclohexyl,
      1-methylcyclohex-1-yl,
      piperidinyl (e.g., piperidin-1-yl),
      pyrrolidinyl (e.g., pyrrolidin-1-yl),
      morpholinyl (e.g., morpholin-4-yl),
      azapanyl (e.g., azapan-1-yl),
      piperazinyl
      2,5-dioxopiperazin-1-yl,
      tetrahydropyranyl (e.g., tetrahydropyran-4-yl),
      isoxazolidinyl (isoxazolidin-5-yl),
      1,1,4-trioxo-1,2,5-thiadiazolidin-2-yl,
      1,1,3-trioxo-1,2,5-thiazolidin-2-yl,
      2-oxocyclopentylidenyl,
      2-oxooxazolidin-5-yl,
      2-oxopyrimidin-1-yl, or
      2,4-dioxo-imidazolidin-3-yl);
   wherein said cycloalkyl² is optionally substituted with one or more
      C₁₋₄alkyl (e.g., methyl),
      —C(O)OR₇,
      —CH₂C(O)OR₇,
      —N(R₆)C(O)OR₇,
      —OH,
      hydroxy-C₁₋₄alkyl (e.g., hydroxymethyl),
      C₁₋₄alkoxy (e.g., methoxy),
      —CH₂N(R₆)—C(O)OR₇,
      aryl² (e.g., phenyl) or aryl²-C₁₋₄alkyl (e.g., benzyl) wherein said aryl² group of said aryl² or aryl²-alkyl is optionally substituted with C₁₋₄alkyl (e.g., methyl), for example, 4-methylphenyl, 2-methylphenyl,
      heteroaryl² (e.g., 2H-tetrazol-5-yl),
      heteroaryl²-C₁₋₄alkyl (e.g., 2H-tetrazol-5-yl-methyl),
      —C₁₋₄alkyl-N(R₈)(R₉) (e.g., -methyl-NH₂— or -ethyl-NH₂),
      C₁₋₄alkoxy (e.g., methoxy),
      —C(O)N(R₅)—S(O)₂—C₁₋₄alkyl (e.g., —C(O)N(H)S(O)₂—CH₃),
      —N(H)—S(O)₂—C₁₋₄alkyl (e.g., —N(H)—S(O)₂-methyl),
      —S(O)₂—N(R₈)(R₉) (e.g., —S(O)₂—NH₂),
      —C(O)N(H)CN,
      —C(O)N(R₈)(R₉), or
      —N(R₈)(R₉);
   or
   A is a 7-11 membered fused cycloalkyl-aryl or spiral compound wherein one or more carbon atoms may be a hetero atom selected from N, O or S and wherein said fused cycloalkyl-aryl or spiral group is optionally substituted with one or more hydroxy, C₁₋₄alkyl (e.g., methyl) or oxo (i.e., ═O), for example
      3,9-diazaspiro[5.5]undecan-3-yl,
      3,9-diazaspiro[5.5]undecan-9-yl,
      (6-oxo-7-oxa-2-azaspiro[4.4]nonan-2-yl),
      (9-oxo-8-oxa-3-azaspiro[4.4]nonan-3-yl),
      (1-oxo-2,8-diazaspiro[4.5]decan-8-yl),
      (2,4-dioxo-3,8-diazaspiro[4,5]decan-8-yl),
      Indolinyl (e.g., indolin-1-yl),
      Indanyl (e.g., indan-1-yl, indan-2-yl or 2-hydroxyindan-1-yl),
      tetralinyl (e.g., tetralin-2-yl, tetralin-1-yl),
      isoindolinyl (e.g., isoindolin-2-yl),
      adamantyl,
      3,4-dihydro-1H-isoquinolin-2-yl or 3,4-dihydro-2H-quinolin-1-yl,
      1,3,4,5-tetrahydro-2-benzazepin-2-yl,
      2,3,4,5-tetrahydro-1-benzazepin-1-yl,
      1,2,4,5-tetrahydro-3-benzazepin-3-yl,
(iii) R₁ is H or C₁₋₈ alkyl (e.g., methyl);
(iv) R₂ is H, halo (e.g., chloro), C₁₋₄alkyl (e.g., methyl), —N(R₄)(R₅) or —O—C₃₋₈cycloalkyl (e.g., —O-cyclopentyl);
(v) R₄ and R₅ are independently selected from
   H,
   C₃₋₇cycloalkyl² (e.g., cyclopropyl or cyclopentyl),
   —C₁₋₄alkyl (e.g., methyl or ethyl), wherein said alkyl is optionally substituted with one or more groups selected from —OH, —C(O)OR₇,
   aryl² optionally substituted with halo (e.g., 4-fluorophenyl),
   aryl²-C₁₋₄alkyl wherein said aryl² group is optionally substituted with halo (e.g., fluoro), for example, 4-fluorophenylethyl;
(vi) R₆ is H or C₁₋₄alkyl (e.g., methyl);
(vii) R₇ is H, C₁₋₄alkyl (e.g., methyl, ethyl or tert-butyl), —CH₂OC(O)CH₃;
(viii) R₈ and R₉ are independently H or C₁₋₄alkyl;
(ix) R₁₀ is H or —C₁₋₄alkyl-OC(O)CH₃ (e.g., —CH₂OC(O)CH₃);
(x) R₁₁ and R₁₂ are independently H or C₁₋₄alkyl,
   in free or salt form.

The invention further relates to a Compound of Formula Q-I:

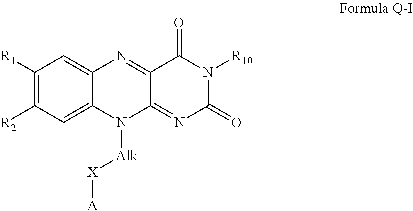

wherein:
(i) Alk is C₁₋₆alkylene (e.g., methylene, ethylene, n-propylene, n-butylene or n-pentylene);
(ii) X is a single bond, —N(R₆)—, —N(R₆)—CH₂—, —N(R₆)—CH₂CH₂—, —N(R₆)—C(H)(CH₃)—, or —C(O)— and
   A is a —C₃₋₈cycloalkyl² (e.g., C₄cycloalkyl² or C₅₋₆cycloalkyl²) wherein one or more carbon atoms of said cycloalkyl² are optionally and independently replaced with N, O, S, S(O)₂ or —C(O)—, for example:
      cyclobutyl,
      cyclopentyl,
      cyclohexyl,
      1-methylcyclohex-1-yl,
      piperidinyl (e.g., piperidin-1-yl),
      pyrrolidinyl (e.g., pyrrolidin-1-yl),
      morpholinyl (e.g., morpholin-4-yl),
      azapanyl (e.g., azapan-1-yl),
      piperazinyl,
      2,5-dioxopiperazin-1-yl,
      tetrahydropyranyl (e.g., tetrahydropyran-4-yl),
      isoxazolidinyl (isoxazolidin-5-yl),
      1,1,4-trioxo-1,2,5-thiadiazolidin-2-yl,
      1,1,3-trioxo-1,2,5-thiazolidin-2-yl,
      2-oxocyclopentylidenyl,
      2-oxooxazolidin-5-yl,
      2-oxopyrimidin-1-yl, or
      2,4-dioxo-imidazolidin-3-yl);
wherein said cycloalkyl² is optionally substituted with one or more C₁₋₄alkyl (e.g., methyl),
   —C(O)OR₇,
   —CH₂C(O)OR₇,
   —N(R₆)C(O)OR₇,
   —OH,
   hydroxy-C₁₋₄alkyl (e.g., hydroxymethyl),
   C₁₋₄alkoxy (e.g., methoxy),
   —CH₂N(R₆)—C(O)OR₇,
   aryl² (e.g., phenyl) or aryl²-C₁₋₄alkyl (e.g., benzyl) wherein said aryl² group of said aryl² or aryl²-alkyl is optionally substituted with C₁₋₄alkyl (e.g., methyl), for example, 4-methylphenyl, 2-methylphenyl,
   heteroaryl² (e.g., 2H-tetrazol-5-yl),
   heteroaryl²-C₁₋₄alkyl (e.g., 2H-tetrazol-5-yl-methyl),
   —C₁₋₄alkyl-N(R₈)(R₉) (e.g., -methyl-NH₂— or -ethyl-NH₂),
   C₁₋₄alkoxy (e.g., methoxy),
   —C(O)N(R₆)—S(O)₂—C₁₋₄alkyl (e.g., —C(O)N(H)S(O)₂—CH₃),
   —N(H)—S(O)₂—C₁₋₄alkyl (e.g., —N(H)—S(O)₂-methyl),
   —S(O)₂—N(R₈)(R₉) (e.g., —S(O)₂—NH₂),
   —C(O)N(H)CN,
   —C(O)N(R₈)(R₉), or
   —N(R₈)(R₉);
or
A is a 7-11 membered fused cycloalkyl-aryl or spiral compound wherein one or more carbon atoms may be a hetero atom selected from N, O or S and wherein said fused cycloalkyl-aryl or spiral group is optionally substituted with one or more hydroxy, C₁₋₄alkyl (e.g., methyl) or oxo (i.e., ═O), for example
   3,9-diazaspiro[5.5]undecan-3-yl,
   3,9-diazaspiro[5.5]undecan-9-yl,
   (6-oxo-7-oxa-2-azaspiro[4.4]nonan-2-yl),
   (9-oxo-8-oxa-3-azaspiro[4.4]nonan-3-yl),
   (1-oxo-2,8-diazaspiro[4.5]decan-8-yl),
   (2,4-dioxo-3,8-diazaspiro[4.5]decan-8-yl),
   Indolinyl (e.g., indolin-1-yl),
   Indanyl (e.g., indan-1-yl, indan-2-yl or 2-hydroxyindan-1-yl),
   tetralinyl (e.g., tetralin-2-yl, tetralin-1-yl),
   isoindolinyl (e.g., isoindolin-2-yl),
   adamantyl,
   3,4-dihydro-1H-isoquinolin-2-yl or 3,4-dihydro-2H-quinolin-1-yl,
   1,3,4,5-tetrahydro-2-benzazepin-2-yl,
   2,3,4,5-tetrahydro-1-benzazepin-1-yl,
   1,2,4,5-tetrahydro-3-benzazepin-3-yl,
(iii) R₁ is H or C₁₋₈alkyl (e.g., methyl);
(iv) R₂ is H, halo (e.g., chloro), C₁₋₄alkyl (e.g., methyl), —N(R₄)(R₅) or —O—C₃₋₈cycloalkyl (e.g., —O-cyclopentyl);
(v) R₄ and R₅ are independently selected from H,
   C₃₋₇cycloalkyl² (e.g., cyclopropyl or cyclopentyl),
   —C₁₋₄alkyl (e.g., methyl or ethyl), wherein said alkyl is optionally substituted with one or more groups selected from —OH, —C(O)OR₇,
   aryl² optionally substituted with halo (e.g., 4-fluorophenyl),
   aryl²-C₁₋₄alkyl wherein said aryl² group is optionally substituted with halo (e.g., fluoro), for example, 4-fluorophenylethyl;
(vi) R₆ is H or C₁₋₄alkyl (e.g., methyl);
(vii) R₇ is H, C₁₋₄alkyl (e.g., methyl, ethyl or tert-butyl), —CH₂OC(O)CH₃;
(viii) R₈ and R₉ are independently H or C₁₋₄alkyl;
(ix) R₁₀ is H or —C₁₋₄alkyl-OC(O)CH₃ (e.g., —CH₂OC(O)CH₃), in free or salt form.

The invention further relates to a Compound of Formula Q-II

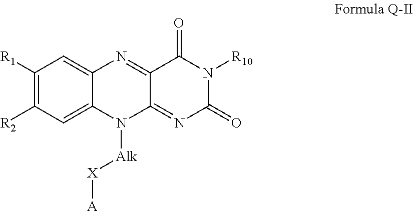

wherein:
(i) Alk is C₁₋₆alkylene (e.g., methylene, ethylene, n-propylene, n-butylene or n-pentylene);
(ii) X is a single bond and A is:
   a monocyclic heteroaryl² (e.g., pyrrolyl, for example pyrrol-1-yl; pyridyl, for example pyrid-2-yl, pyrid-4-yl or pyrid-3-yl; tetrazolyl, for example 1,2,3,4-tetrazol-1-yl; imidazolyl, for example imidazol-1-yl; or isoxazolyl, for example isoxazol-5-yl); or
   wherein said heteroaryl² is optionally substituted with one or more C₁₋₄alkyl (e.g., methyl),
(iii) R₁ is H or C₁₋₈ alkyl (e.g., methyl);
(iv) R₂ is H, halo (e.g., chloro), C₁₋₄alkyl (e.g., methyl), —N(R₄)(R₅) or —O—C₃₋₈cycloalkyl² (e.g., —O-cyclopentyl);
(v) R₄ and R₅ are independently selected from H,
   C₃₋₇cycloalkyl² (e.g., cyclopropyl or cyclopentyl),
   —C₁₋₄alkyl (e.g., methyl or ethyl), wherein said alkyl is optionally substituted with one or more groups selected from —OH, —C(O)OR₇,
   aryl² optionally substituted with halo (e.g., 4-fluorophenyl),
   aryl²-C₁₋₄alkyl wherein said aryl group is optionally substituted with halo (e.g., fluoro), for example, 4-fluorophenylethyl;
(vi) R₇ is H, C₁₋₄alkyl (e.g., methyl, ethyl or tert-butyl), —CH₂OC(O)CH₃;
(vii) R₁₀ is H or —C₁₋₄alkyl-OC(O)CH₃ (e.g., —CH₂OC(O)CH₃), in free or salt form.

The invention further relates to a Compound of Formula Q-III:

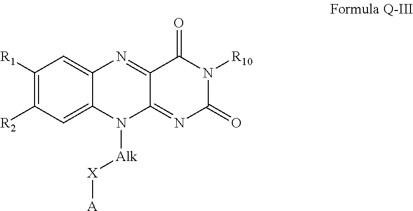

wherein:
(i) Alk is C₁₋₆alkylene (e.g., methylene, ethylene, n-propylene, n-butylene or n-pentylene);
(ii) X is —N(R₆) and A is:
   —C₁₋₄alkyl-N(R₁₁)(R₁₂),
   —C₀₋₄alkyl-aryl¹ (e.g., phenyl, naphthyl, benzyl), or —C₀₋₄alkyl-heteroaryl¹ (e.g., isoxazolyl, (isoxazol-5-yl)methyl, tetrazolyl, pyridyl, for example pyrid-3-yl, (pyrid-5-yl)methyl, indolyl, 1,2,5-oxadiazolyl, pyrrolyl), wherein the alkyl group of said -alkylaryl¹ and -alkylheteroaryl¹ is optionally substituted with hydroxy or another aryl¹ (e.g., phenyl), and the aryl¹ and heteroaryl¹ group of said -alkylaryl¹ and -alkylheteroaryl¹ are independently substituted with one or more:
      —N(Rₐ)—C(O)—C₁₋₄alkyl (e.g., —NHC(O)CH₃), wherein Rₐ is H or C₁₋₄alkyl,
      —OH,
      heteroaryl¹ (e.g., imidazolyl),
      heteroC₃₋₈cycloalkyl¹ (e.g., morpholinyl),
      aryl¹ (e.g., phenyl),
      —O-halo-C₁₋₄alkyl (e.g., —OCF₃),
      —N(Rₐ)(R_{b}), wherein Rₐ is H or C₁₋₄alkyl and R_{b} is C₁₋₄alkyl,
      —SO₂—C₁₋₄alkyl (e.g., —SO₂—CH₃);
   —C₀₋₄alkyl-pyridyl substituted with one or more hydroxy (e.g., 2-hydroxypyrid-4-ylmethyl or 2-hydroxypyrid-3-yl);
   —C₀₋₄alkyl-benzotriazolyl (e.g., 1H-benzotriazol-5-yl);
   —C₀₋₄alkyl-indolyl (e.g., -indol-5-ylmethyl, indol-2-ylmethyl, indol-3-ylethyl);
   —C₀₋₄alkyl-tetrazolyl (e.g., 1,2,3,5-tetrazol-4-ylethyl);
   —C₀₋₄alkyl-oxadiazolyl (e.g., 1,2,5-oxadiazol-3-yl);
   —C₀₋₄alkyl-benzodioxolyl (e.g., 1,3-benzodioxol-5-ylmethyl);
   —C₀₋₄alkyl-benzimidazolyl optionally substituted with —C₀₋₄alkyl (e.g., 1-methylbenzimidazol-2-ylmethyl, benzimidazol-5-ylmethyl);
   —C₀₋₄alkyl-imidazolyl optionally substituted with C₁₋₄alkyl (e.g., 1-methyl-imidazol-5-ylmethyl);
   —C₀₋₄alkyl-pyrrolyl optionally substituted with —C₀₋₄alkyl (e.g., 1-methylpyrrolidin-2-ylmethyl);
   para-phenylbenzyl;
(iii) R₁ is H or C₁₋₈ alkyl (e.g., methyl);
(iv) R₂ is H, halo (e.g., chloro), C₁₋₄alkyl (e.g., methyl), —N(R₄)(R₅) or —O—C₃₋₈cycloalkyl² (e.g., —O-cyclopentyl);
(v) R₄ and R₅ are independently selected from H,
   C₃₋₇cycloalkyl² (e.g., cyclopropyl or cyclopentyl),
   —C₁₋₄alkyl (e.g., methyl or ethyl), wherein said alkyl is optionally substituted with one or more groups selected from —OH, —C(O)OR₇,
   aryl² optionally substituted with halo (e.g., 4-fluorophenyl),
   aryl²-C₁₋₄alkyl wherein said aryl group is optionally substituted with halo (e.g., fluoro), for example, 4-fluorophenylethyl;
(vi) R₆ is H or C₁₋₄alkyl (e.g., methyl);
(vii) R₇ is H, C₁₋₄alkyl (e.g., methyl, ethyl or tert-butyl), —CH₂OC(O)CH₃;
(viii) R₁₀ is H or —C₁₋₄alkyl-OC(O)CH₃ (e.g., —CH₂OC(O)CH₃);
(ix) R₁₁ and R₁₂ are independently H or C₁₋₄alkyl, in free or salt form.

The invention further relates to a Compound of Formula Q-IV

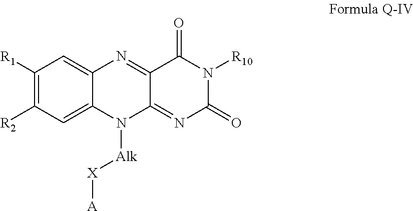

wherein
(i) Alk is C₁₋₄alkylene (e.g., methylene, ethylene, n-propylene);
(ii) X is a single bond and A is pyrrolyl, for example pyrrol-1-yl or imidazolyl, for example imidazol-1-yl);
   or
   X is a single bond and A is a pyrrolidinyl (e.g., pyrrolidin-1-yl) or piperidinyl (e.g., piperidin-1-yl) optionally substituted with another aryl (e.g., phenyl) or aryl-C₁₋₄alkyl (e.g., benzyl);
   or
   X is —N(R₆)— and A is tetralinyl (e.g., tetralin-2-yl);
(iii) R₁ is H or C₁₋₈alkyl (e.g., methyl);
(iv) R₂ is H, halo (e.g., chloro), C₁₋₄alkyl (e.g., methyl);
(v) R₆ is H or C₁₋₄alkyl (e.g., methyl);
(vi) R₁₀ is H,
   in free or salt form.

The invention further relates to a Compound of Formula Q-V:

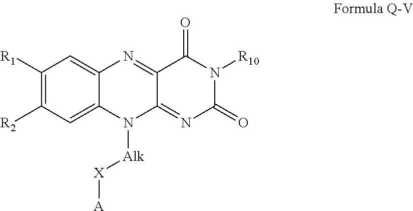

wherein:
(i) Alk is C₁₋₆alkylene (e.g., methylene, ethylene or n-propylene);
(ii) X is a single bond and A is pyrrolyl, for example pyrrol-1-yl, pyrrolidinyl (e.g., pyrrolidin-1-yl) or piperidinyl (e.g., piperidin-1-yl) optionally substituted with another aryl (e.g., phenyl) or aryl-C₁₋₄alkyl (e.g., benzyl);
(iii) R₁ is C₁₋₈ alkyl (e.g., methyl);
(iv) R₂ is C₁₋₄alkyl (e.g., methyl);
(v) R₁₀ is H,
   in free or salt form.

In a further embodiment of the first aspect, the invention provides a Compound of Formula Q, or any of Q-I to Q-V, wherein said compound is as described in the following formulae:
Q.1. the Compound of Formula Q, or any of Q-I to Q-V, wherein Alk is C₁₋₆alkylene (e.g., methylene, ethylene or n-propylene);
Q.2. the Compound of Formula Q, or any of Q-I to Q-V, Q.1 wherein Alk is ethylene;
Q.3. the Compound of Formula Q, or any of Q-I to Q-V, Q.1 or Q.2, wherein
   X is —N(R) and A is:
      —C₁₋₄alkyl-N(R₁₁)(R₁₂),
      —C₀₋₄alkyl-aryl¹ (e.g., phenyl, naphthyl, benzyl), or —C₀₋₄alkyl-heteroaryl¹ (e.g., isoxazolyl, (isoxazol-5-yl)methyl, tetrazolyl, pyridyl, for example pyrid-3-yl, (pyrid-5-yl)methyl, indolyl, 1,2,5-oxadiazolyl, pyrrolyl), wherein the alkyl group of said -alkylaryl¹ and -alkylheteroaryl¹ is optionally substituted with hydroxy or another aryl¹ (e.g., phenyl), and the aryl¹ and heteroaryl¹ group of said -alkylaryl¹ and -alkylheteroaryl¹ are independently substituted with one or more:
         —N(Rₐ)—C(O)—C₁₋₄alkyl (e.g., —NHC(O)CH₃), wherein Rₐ is H or C₁₋₄alkyl,
         —OH,
         heteroaryl¹ (e.g., imidazolyl),
         heteroC₃₋₈cycloalkyl (e.g., morpholinyl),
         aryl¹ (e.g., phenyl),
         —O-halo-C₁₋₄alkyl (e.g., —OCF₃),
         —NO₂,
         —N(Rₐ)(R_{b}), wherein Rₐ is H or C₁₋₄alkyl and R_{b} is C₁₋₄alkyl,
         —SO₂—C₁₋₄alkyl (e.g., —SO₂—CH₃);
      —C₀₋₄alkyl-pyridyl substituted with one or more hydroxy (e.g., 2-hydroxypyrid-4-ylmethyl or 2-hydroxypyrid-3-yl);
      —C₀₋₄alkyl-benzotriazolyl (e.g., 1H-benzotriazol-5-yl);
      —C₀₋₄alkyl-indolyl (e.g., -indol-5-ylmethyl, indol-2-ylmethyl, indol-3-ylethyl);
      —C₀₋₄alkyl-tetrazolyl (e.g., 1,2,3,5-tetrazol-4-ylethyl);
      —C₀₋₄alkyl-oxadiazolyl (e.g., 1,2,5-oxadiazol-3-yl);
      —C₀₋₄alkyl-benzodioxolyl (e.g., 1,3-benzodioxol-5-ylmethyl);
      —C₀₋₄alkyl-benzimidazolyl optionally substituted with —C₀₋₄alkyl (e.g., 1-methylbenzimidazol-2-ylmethyl, benzimidazol-5-ylmethyl);
      —C₀₋₄alkyl-imidazolyl optionally substituted with C₁₋₄alkyl (e.g., 1-methyl-imidazol-5-ylmethyl);
      —C₀₋₄alkyl-pyrrolyl optionally substituted with —C₀₋₄alkyl (e.g., 1-methylpyrrolidin-2-ylmethyl); or
      para-phenylbenzyl;
   or
   X is a single bond, and A is a monocyclic heteroaryl² (e.g., pyrrolyl, for example pyrrol-1-yl; pyridyl, for example pyrid-2-yl, pyrid-4-yl or pyrid-3-yl; tetrazolyl, for example 1,2,3,4-tetrazol-1-yl; imidazolyl, for example imidazol-1-yl; or isoxazolyl, for example isoxazol-5-yl) wherein said monocyclic heteroaryl² is optionally substituted with C₁₋₄alkyl (e.g., methyl);
   or
   X is a single bond, —N(R₆)—, —N(R₆)—CH₂—, —N(R₆)—CH₂CH₂—, —N(R₆)—C(H)(CH₃)—, or —C(O)—; and:
      A is a C₃₋₈cycloalkyl² (e.g., C₄cycloalkyl² or C₅₋₆cycloalkyl²) wherein one or more carbon atoms of said cycloalkyl² are optionally and independently replaced with N, O, S, S(O)₂ or —C(O)—, for example:
         cyclobutyl,
         cyclopentyl,
         cyclohexyl,
         1-methylcyclohex-1-yl,
         piperidinyl (e.g., piperidin-1-yl),
         pyrrolidinyl (e.g., pyrrolidin-1-yl),
         morpholinyl (e.g., morpholin-4-yl),
         azapanyl (e.g., azapan-1-yl),
         piperazinyl
         2,5-dioxopiperazin-1-yl,
         tetrahydropyranyl (e.g., tetrahydropyran-4-yl),
         isoxazolidinyl (isoxazolidin-5-yl),
         1,1,4-trioxo-1,2,5-thiadiazolidin-2-yl,
         1,1,3-trioxo-1,2,5-thiazolidin-2-yl,
         2-oxocyclopentylidenyl,
         2-oxooxazolidin-5-yl,
         2-oxopyrimidin-1-yl, or
         2,4-dioxo-imidazolidin-3-yl);
      wherein said cycloalkyl² is optionally substituted with one or more
         C₁₋₄alkyl (e.g., methyl),
         —C(O)OR₇,
         —CH₂C(O)OR₇,
         —N(R₆)C(O)OR₇,
         —OH,
         hydroxy-C₁₋₄alkyl (e.g., hydroxymethyl),
         C₁₋₄alkoxy (e.g., methoxy),
         —CH₂N(R₆)—C(O)OR₇,
         aryl² (e.g., phenyl) or aryl²-C₁₋₄alkyl (e.g., benzyl) wherein said aryl² group of said aryl² or aryl²-alkyl is optionally substituted with C₁₋₄alkyl (e.g., methyl), for example, 4-methylphenyl, 2-methylphenyl,
         heteroaryl² (e.g., 2H-tetrazol-5-yl),
         heteroaryl²-C₁₋₄alkyl (e.g., 2H-tetrazol-5-yl-methyl),
         —C₁₋₄alkyl-N(R₈)(R₉) (e.g., -methyl-NH₂— or -ethyl-NH₂)
         C₁₋₄alkoxy (e.g., methoxy),
         —C(O)N(R₆)—S(O)₂—C₁₋₄alkyl (e.g., —C(O)N(H)S(O)₂—CH₃);
         —N(H)—S(O)₂—C₁₋₄alkyl (e.g., —N(H)—S(O)₂-methyl),
         —S(O)₂—N(R₈)(R₉) (e.g., —S(O)₂—NH₂),
         —C(O)N(H)CN,
         —C(O)N(R₈)(R₉), or
         —N(R₈)(R₉);
      or
      A is a 7-11 membered fused cycloalkyl-aryl or spiral compound wherein one or more carbon atoms may be a hetero atom selected from N, O or S and wherein said fused cycloalkyl-aryl or spiral group is optionally substituted with one or more hydroxy, C₁₋₄alkyl (e.g., methyl) or oxo (i.e., ═O), for example
         3,9-diazaspiro[5.5]undecan-3-yl,
         3,9-diazaspiro[5.5]undecan-9-yl,
         (6-oxo-7-oxa-2-azaspiro[4.4]nonan-2-yl),
         (9-oxo-8-oxa-3-azaspiro[4.4]nonan-3-yl),
         (1-oxo-2,8-diazaspiro[4.5]decan-8-yl),
         (2,4-dioxo-3,8-diazaspiro[4.5]decan-8-yl),
         Indolinyl (e.g., indolin-1-yl),
         Indanyl (e.g., indan-1-yl, indan-2-yl or 2-hydroxyindan-1-yl),
         tetralinyl (e.g., tetralin-2-yl, tetralin-1-yl),
         isoindolinyl (e.g., isoindolin-2-yl),
         adamantyl,
         3,4-dihydro-1H-isoquinolin-2-yl or 3,4-dihydro-2H-quinolin-1-yl,
         1,3,4,5-tetrahydro-2-benzazepin-2-yl,
         2,3,4,5-tetrahydro-1-benzazepin-1-yl,
         1,2,4,5-tetrahydro-3-benzazepin-3-yl;
Q.4. the compound of Formula Q, or any of Q-I to Q-V, Q.1 or Q.2, wherein X is a single bond, —N(R₆)—, —N(R₆)—CH₂—, —N(R₆)—CH₂CH₂—, —N(R₆)—C(H)(CH₃)—, or —C(O)— and:
   A is a —C₃₋₈cycloalkyl² (e.g., C₄cycloalkyl² or C₅₋₆cycloalkyl²) wherein one or more carbon atoms of said cycloalkyl² are optionally and independently replaced with N, O, S, S(O)₂ or —C(O)—, for example:
      cyclobutyl,
      cyclopentyl,
      cyclohexyl,
      1-methylcyclohex-1-yl,
      piperidinyl (e.g., piperidin-1-yl),
      pyrrolidinyl (e.g., pyrrolidin-1-yl),
      morpholinyl (e.g., morpholin-4-yl),
      azapanyl (e.g., azapan-1-yl),
      piperazinyl,
      2,5-dioxopiperazin-1-yl,
      tetrahydropyranyl (e.g., tetrahydropyran-4-yl),
      isoxazolidinyl (isoxazolidin-5-yl),
      1,1,4-trioxo-1,2,5-thiadiazolidin-2-yl,
      1,1,3-trioxo-1,2,5-thiazolidin-2-yl,
      2-oxocyclopentylidenyl,
      2-oxooxazolidin-5-yl,
      2-oxopyrimidin-1-yl, or
      2,4-dioxo-imidazolidin-3-yl);
   wherein said cycloalkyl² is optionally substituted with one or more C₁₋₄alkyl (e.g., methyl),
      —C(O)OR₇,
      —CH₂C(O)OR₇,
      —N(R₆)C(O)OR₇,
      —OH,
      hydroxy-C₁₋₄alkyl (e.g., hydroxymethyl),
      C₁₋₄alkoxy (e.g., methoxy),
      —CH₂N(R₆)—C(O)OR₇,
      aryl² (e.g., phenyl) or aryl²-C₁₋₄alkyl (e.g., benzyl) wherein said aryl² group of said aryl² or aryl²-alkyl is optionally substituted with C₁₋₄alkyl (e.g., methyl), for example, 4-methylphenyl, 2-methylphenyl,
      heteroaryl² (e.g., 2H-tetrazol-5-yl),
      heteroaryl²-C₁₋₄alkyl (e.g., 2H-tetrazol-5-yl-methyl),
         —C₁₋₄alkyl-N(R₈)(R₉) (e.g., -methyl-NH₂— or -ethyl-NH₂),
         C₁₋₄alkoxy (e.g., methoxy),
         —C(O)N(R₆)—S(O)₂—C₁₋₄alkyl (e.g., —C(O)N(H)S(O)₂—CH₃),
         —N(H)—S(O)₂—C₁₋₄alkyl (e.g., —N(H)—S(O)₂-methyl),
         —S(O)₂—N(R₈)(R₉) (e.g., —S(O)₂—NH₂), —C(O)N(H)CN,
         —C(O)N(R₈)(R₉), or
         —N(R₈)(R₉);
      or
      A is a 7-11 membered fused cycloalkyl-aryl or spiral compound wherein one or more carbon atoms may be a hetero atom selected from N, O or S and wherein said fused cycloalkyl-aryl or spiral group is optionally substituted with one or more hydroxy, C₁₋₄alkyl (e.g., methyl) or oxo (i.e., ═O), for example
         3,9-diazaspiro[5.5]undecan-3-yl,
         3,9-diazaspiro[5.5]undecan-9-yl,
         (6-oxo-7-oxa-2-azaspiro[4.4]nonan-2-yl),
         (9-oxo-8-oxa-3-azaspiro[4.4]nonan-3-yl),
         (1-oxo-2,8-diazaspiro[4.5]decan-8-yl),
         (2,4-dioxo-3,8-diazaspiro[4.5]decan-8-yl),
         Indolinyl (e.g., indolin-1-yl),
         Indanyl (e.g., indan-1-yl, indan-2-yl or 2-hydroxyindan-1-yl),
         tetralinyl (e.g., tetralin-2-yl, tetralin-1-yl),
         isoindolinyl (e.g., isoindolin-2-yl),
         adamantyl,
         3,4-dihydro-1H-isoquinolin-2-yl or 3,4-dihydro-2H-quinolin-1-yl,
         1,3,4,5-tetrahydro-2-benzazepin-2-yl,
         2,3,4,5-tetrahydro-1-benzazepin-1-yl,
         1,2,4,5-tetrahydro-3-benzazepin-3-yl;
Q.5. the Compound of Formula Q, or any of Q-I to Q-V, Q.1 or Q.2, wherein X is a single bond and A is:
   a monocyclic heteroaryl² (e.g., pyrrolyl, for example pyrrol-1-yl; pyridyl, for example pyrid-2-yl, pyrid-4-yl or pyrid-3-yl; tetrazolyl, for example 1,2,3,4-tetrazol-1-yl; imidazolyl, for example imidazol-1-yl; or isoxazolyl, for example isoxazol-5-yl); or
   wherein said heteroaryl² is optionally substituted with one or more C₁₋₄alkyl (e.g., methyl);
Q.6. the Compound of Formula Q, or any of Q-I to Q-V, Q.1 or Q.2, wherein X is —N(R₆) and A is
   —C₁₋₄alkyl-N(R₁₁)(R₁₂),
   —C₀₋₄alkyl-aryl¹ (e.g., phenyl, naphthyl, benzyl), or —C₀₋₄alkyl-heteroaryl¹ (e.g., isoxazolyl, (isoxazol-5-yl)methyl, tetrazolyl, pyridyl, for example pyrid-3-yl, (pyrid-5-yl)methyl, indolyl, 1,2,5-oxadiazolyl, pyrrolyl), wherein the alkyl group of said -alkylaryl¹ and -alkylheteroaryl¹ is optionally substituted with hydroxy or another aryl¹ (e.g., phenyl), and the aryl¹ and heteroaryl¹ group of said -alkylaryl¹ and -alkylheteroaryl¹ are independently substituted with one or more:
      —N(Rₐ)—C(O)—C₁₋₄alkyl (e.g., —NHC(O)CH₃), wherein Rₐ is H or C₁₋₄alkyl,
      —OH,
      heteroaryl¹ (e.g., imidazolyl),
      heteroC₃₋₈cycloalkyl¹ (e.g., morpholinyl),
      aryl¹ (e.g., phenyl),
      —O-halo-C₁₋₄alkyl (e.g., —OCF₃),
      —NO₂,
      —N(Rₐ)(R_{b}), wherein Rₐ is H or C₁₋₄alkyl and R_{b} is C₁₋₄alkyl,
      —SO₂—C₁₋₄alkyl (e.g., —SO₂—CH₃);
   —C₀₋₄alkyl-pyridyl substituted with one or more hydroxy (e.g., 2-hydroxypyrid-4-ylmethyl or 2-hydroxypyrid-3-yl);
   —C₀₋₄alkyl-benzotriazolyl (e.g., 1H-benzotriazol-5-yl);
   —C₀₋₄alkyl-indolyl (e.g., -indol-5-ylmethyl, indol-2-ylmethyl, indol-3-ylethyl);
   —C₀₋₄alkyl-tetrazolyl (e.g., 1,2,3,5-tetrazol-4-ylethyl);
   —C₀₋₄alkyl-oxadiazolyl (e.g., 1,2,5-oxadiazol-3-yl);
   —C₀₋₄alkyl-benzodioxolyl (e.g., 1,3-benzodioxol-5-ylmethyl);
   —C₀₋₄alkyl-benzimidazolyl optionally substituted with —C₀₋₄alkyl (e.g., 1-methylbenzimidazol-2-ylmethyl, benzimidazol-5-ylmethyl);
   —C₀₋₄alkyl-imidazolyl optionally substituted with C₁₋₄alkyl (e.g., 1-methyl-imidazol-5-ylmethyl);
   —C₀₋₄alkyl-pyrrolyl optionally substituted with —C₀₋₄alkyl (e.g., 1-methylpyrrolidin-2-ylmethyl);
   para-phenylbenzyl;
Q.7. the Compound of Formula Q, or any of Q-I to Q-V, Q.1 or Q.2, wherein X is —N(R₆) and A is
   —C₁₋₄alkyl-aryl¹ (e.g., benzyl), or —C₁₋₄alkyl-heteroaryl¹ (e.g., isoxazol-5-yl)methyl, (pyrid-5-yl)methyl), wherein the alkyl group of said -alkylaryl¹ and -alkylheteroaryl¹ is optionally substituted with hydroxy or another aryl¹ (e.g., phenyl), and the aryl¹ and heteroaryl¹ group of said -alkylaryl¹ and -alkylheteroaryl¹ are independently substituted with one or more:
      —N(Rₐ)—C(O)—C₁₋₄alkyl (e.g., —NHC(O)CH₃), wherein Rₐ is H or
      —OH,
      heteroaryl¹ (e.g., imidazolyl),
      heteroC₃₋₈cycloalkyl¹ (e.g., morpholinyl),
      aryl¹ (e.g., phenyl),
      —O-halo-C₁₋₄alkyl (e.g., —OCF₃),
      —NO₂,
      —N(Rₐ(R_{b}), wherein Rₐ is H or C₁₋₄alkyl and R_{b} is C₁₋₄alkyl,
      —SO₂—C₁₋₄alkyl (e.g., —SO₂—CH₃);
Q.8. the Compound of Formula Q, or any of Q-I to Q-V, Q.1 or Q.2, wherein X is a single bond and A is pyrrolyl, for example pyrrol-1-yl or imidazolyl, for example imidazol-1-yl);
Q.9. the Compound of Formula Q, or any of Q-I to Q-V, Q.1 or Q.2, wherein X is a single bond and A is a pyrrolidinyl (e.g., pyrrolidin-1-yl) or piperidinyl (e.g., piperidin-1-yl) optionally substituted with another aryl (e.g., phenyl) or aryl-C₁₋₄alkyl (e.g., benzyl);
Q.10. the Compound of Formula Q, or any of Q-I to Q-V, Q.1 or Q.2, wherein X is —N(R₆)— and A is tetralinyl (e.g., tetralin-2-yl);
Q.11. the Compound of Formula Q, or any of Q-I to Q-V or any of Q.1-Q.10, wherein R₁ is C₁₋₈ alkyl (e.g., methyl);
Q.12. the Compound of Formula Q, or any of Q-I to Q-V or any of Q.1-Q.10, wherein R₁ is methyl;
Q.13. the Compound of Formula Q, or any of Q-I to Q-V or any of Q.1-Q.12, wherein R₂ is H, halo (e.g., chloro), C₁₋₄alkyl (e.g., methyl), —N(R₄)(R₅) or —O—C₃₋₈cycloalkyl (e.g., —O-cyclopentyl);
Q.14. the Compound of Formula Q, or any of Q-I to Q-V or any of Q.1-Q.13, wherein R₂ is C₁₋₄alkyl (e.g., methyl);
Q.15. the Compound of Formula Q, or any of Q-I to Q-V or any of Q.1-Q.14, wherein R₂ is methyl;
Q.16. the Compound of Formula Q, or any of Q-I to Q-V or any of Q.1-Q.13, wherein R₂ is —N(R₄)(R₅);
Q.17. formula Q.16, wherein R₄ and R₅ are independently selected from H,
   C₃₋₇cycloalkyl² (e.g., cyclopropyl or cyclopentyl),
   —C₁₋₄alkyl (e.g., methyl or ethyl), wherein said alkyl is optionally substituted with one or more groups selected from —OH, —C(O)OR₇,
   aryl² optionally substituted with halo (e.g., 4-fluorophenyl),
   aryl²-C₁₋₄alkyl wherein said aryl² group is optionally substituted with halo (e.g., fluoro), for example, 4-fluorophenylethyl;
Q.18. the Compound of Formula Q, or any of Q-I to Q-V or any of Q.1-Q.17, wherein R₆ is H or C₁₋₄alkyl (e.g., methyl);
Q.19. the Compound of Formula Q, or any of Q-I to Q-V or any of Q.1-Q.17, wherein R₆ is H;
Q.20. the Compound of Formula Q, or any of Q-I to Q-V or any of Q.1-Q.19, wherein R₇ is H, C₁₋₄alkyl (e.g., methyl, ethyl or tert-butyl), —CH₂OC(O)CH₃;
Q.21. the Compound of Formula Q, or any of Q-I to Q-V or any of Q.1-Q.20, wherein R₇ is H,;
Q.22. the Compound of Formula Q, or any of Q-I to Q-V or any of Q.1-Q.20, wherein R₇ is C₁₋₄alkyl (e.g., methyl, ethyl or tert-butyl);
Q.23. the Compound of Formula Q, or any of Q-I to Q-V or any of Q.1-Q.20, wherein R₇ is —CH₂OC(O)CH₃;
Q.24. the Compound of Formula Q, or any of Q-I to Q-V or any of Q.1-Q.23, wherein R₈ and R₉ are independently H or C₁₋₄alkyl;
Q.25. the Compound of Formula Q, or any of Q-I to Q-V or any of Q.1-Q.24, wherein R₈ and R₉ are H;
Q.26. the Compound of Formula Q, or any of Q-I to Q-V or any of Q.1-Q.24, wherein R₈ and R₉ are C₁₋₄alkyl;
Q.27. the Compound of Formula Q, or any of Q-I to Q-V or any of Q.1-Q.26, wherein R₁₀ is H or —C₁₋₄alkyl-OC(O)CH₃ (e.g., —CH₂OC(O)CH₃);
Q.28. the Compound of Formula Q, or any of Q-I to Q-V or any of Q.1-Q.27, wherein R₁₀ is —C₁₋₄alkyl-OC(O)CH₃ (e.g., —CH₂OC(O)CH₃);
Q.29. the Compound of Formula Q, or any of Q-I to Q-V or any of Q.1-Q.27, wherein R₁₀ is H;
Q.30. the Compound of Formula Q, or any of Q-I to Q-V or any of Q.1-Q.29, wherein R₁₁ and R₁₂ are independently H or C₁₋₄alkyl;
Q.31. the Compound of Formula Q, or any of Q-I to Q-V or any of Q.1-Q.30, wherein R₁₁ is H;
Q.32. the Compound of Formula Q, or any of Q-I to Q-V or any of Q.1-Q.30, wherein R₁₁ is C₁₋₄alkyl;
Q.33. the Compound of Formula Q, or any of Q-I to Q-V or any of Q.1-Q.32, wherein R₁₂ is H;
Q.34. the Compound of Formula Q, or any of Q-I to Q-V or any of Q.1-Q.32, wherein R₁₂ is C₁₋₄alkyl;
Q.35. any of the preceeding formulae wherein said compound is selected from the following:

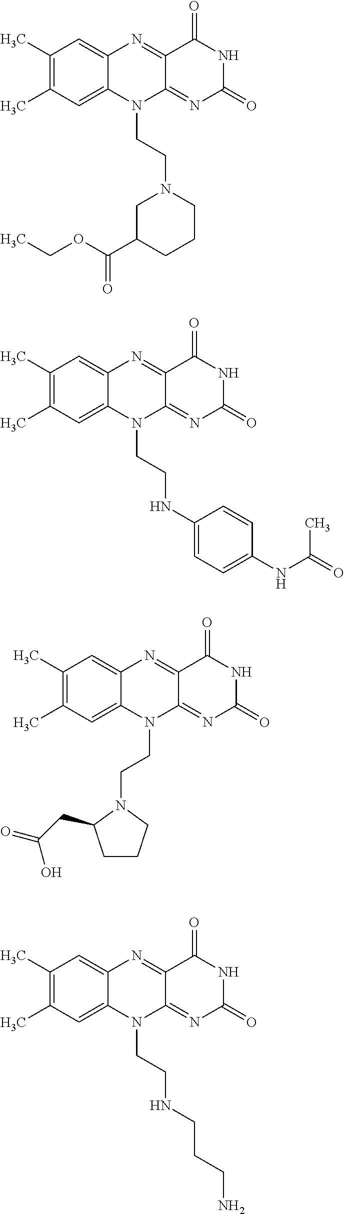

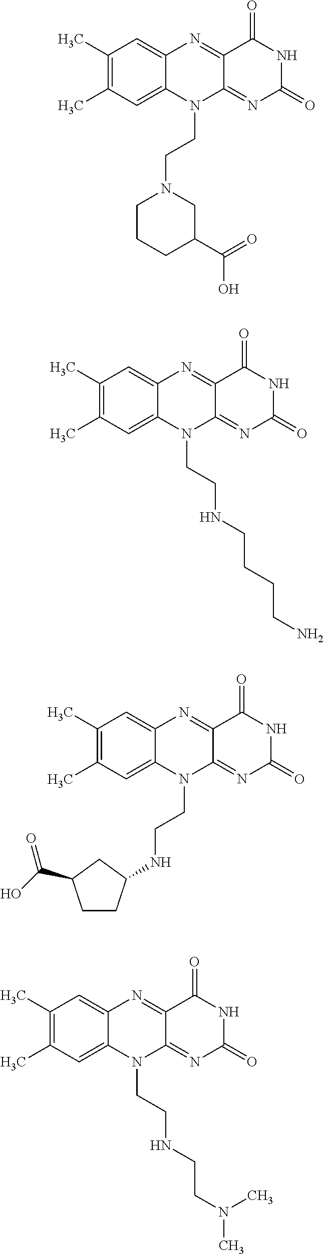

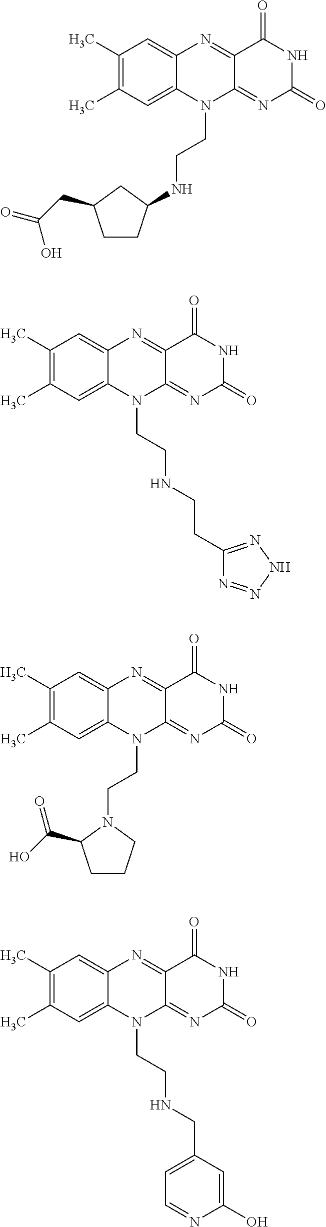

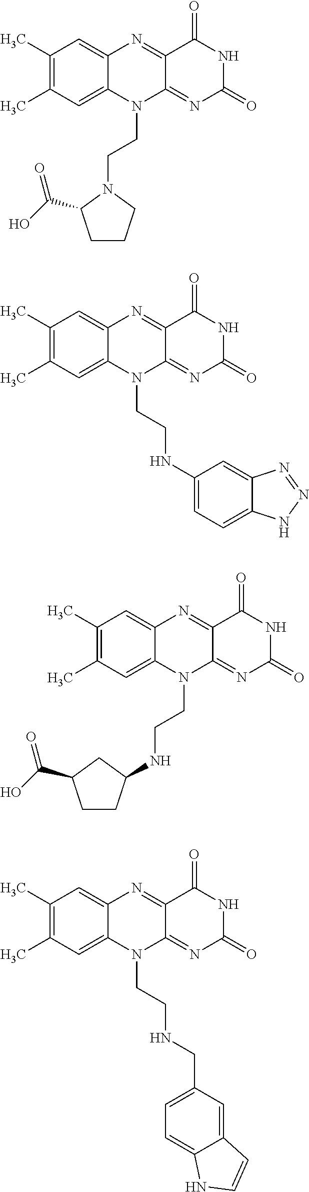

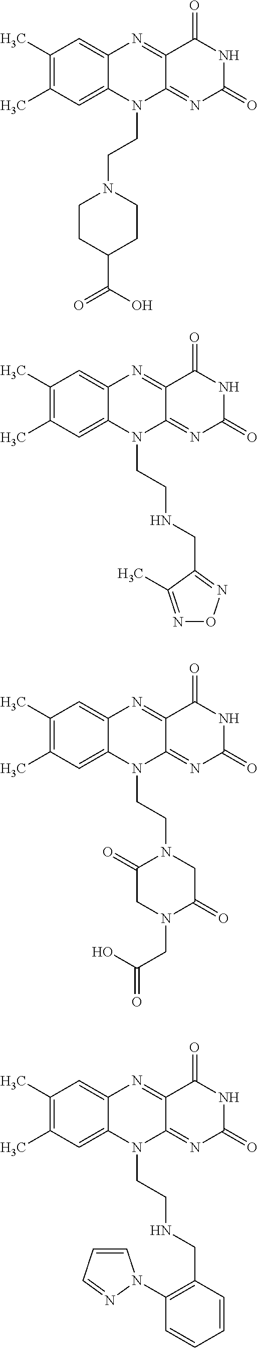

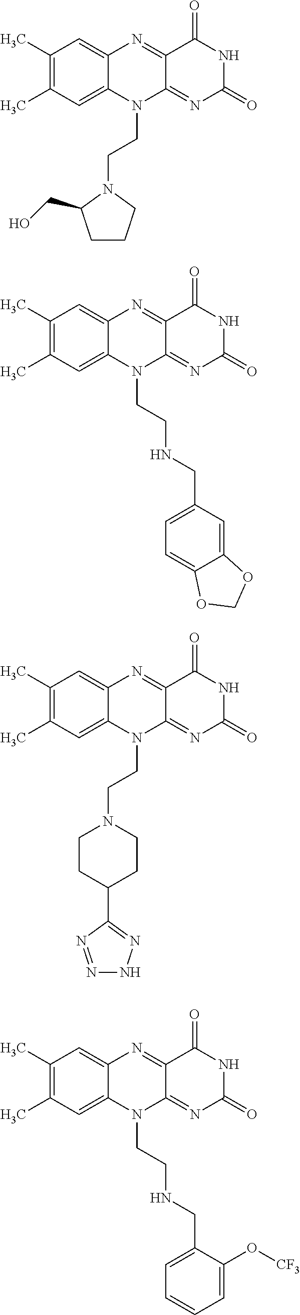

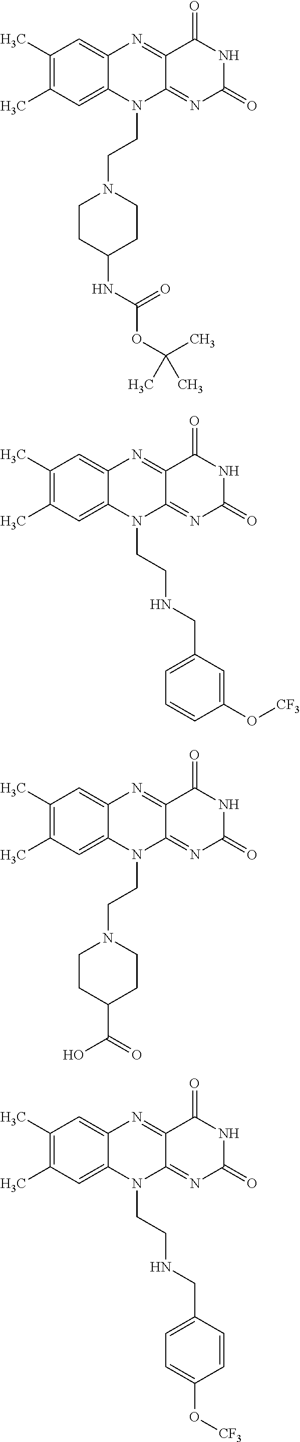

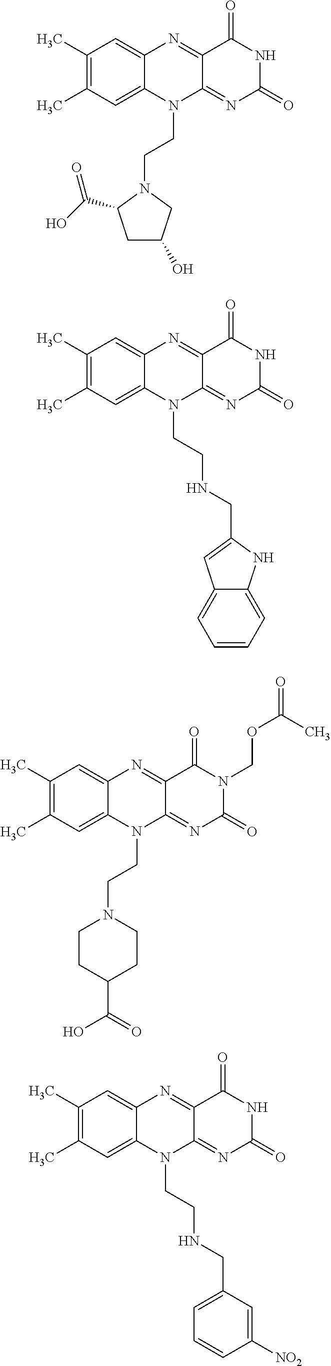

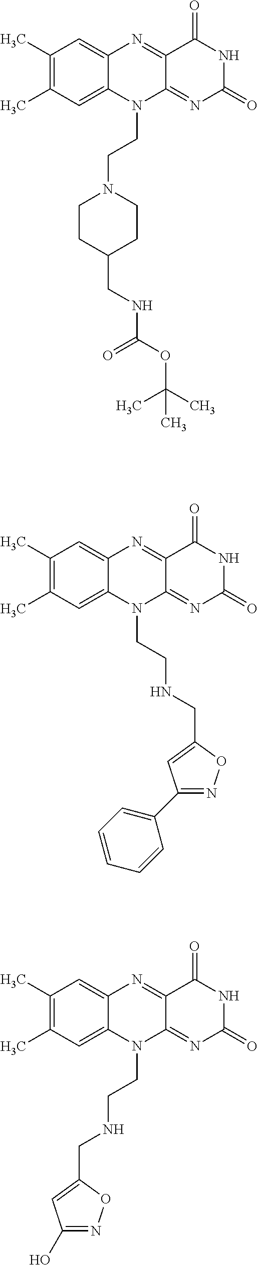

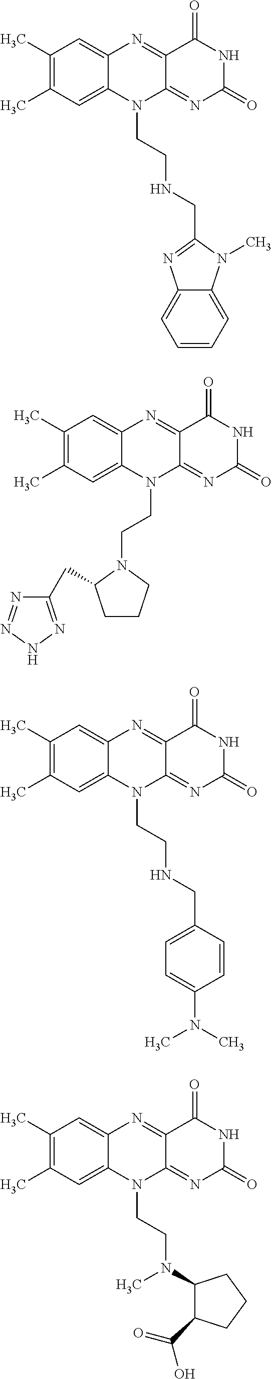

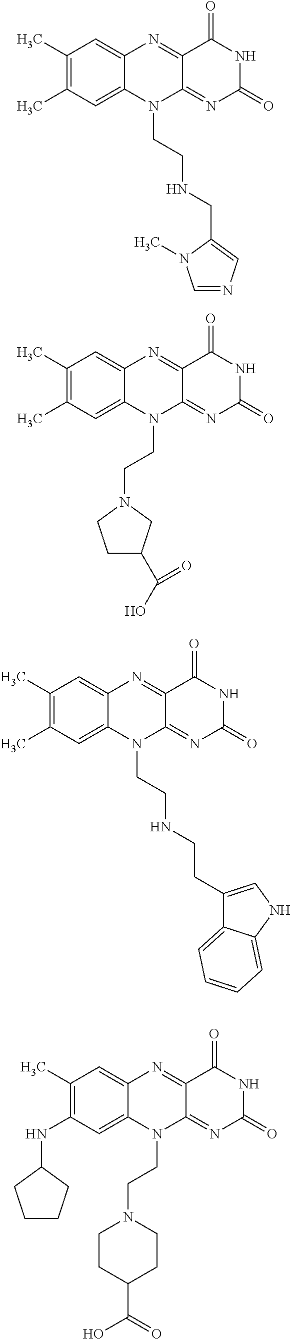

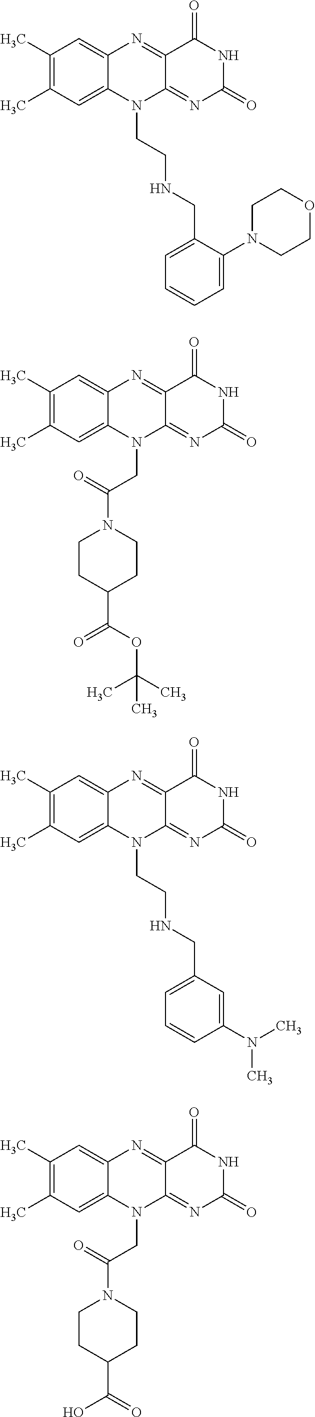

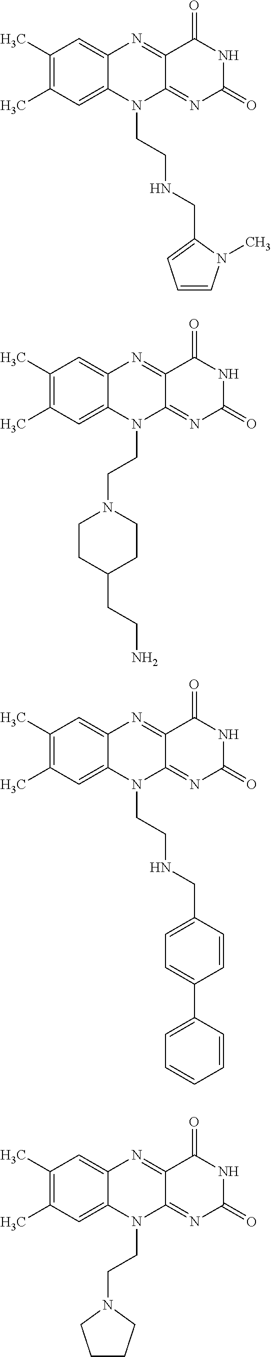

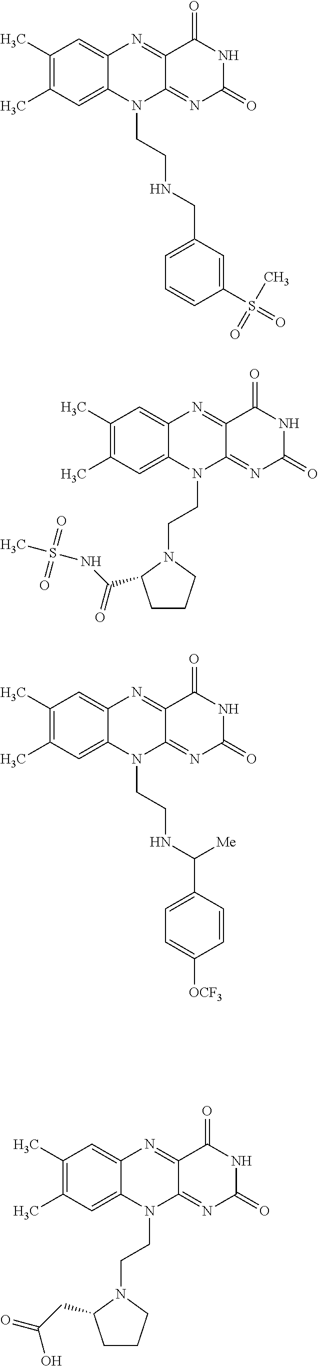

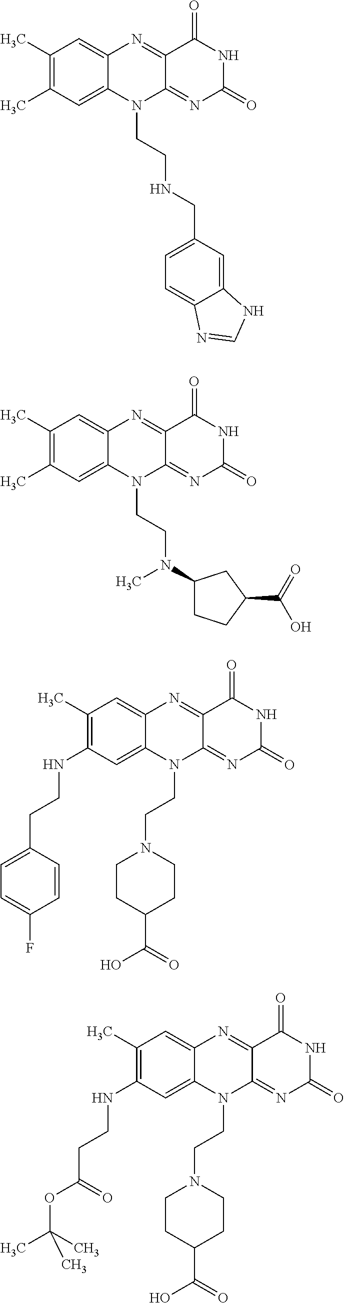

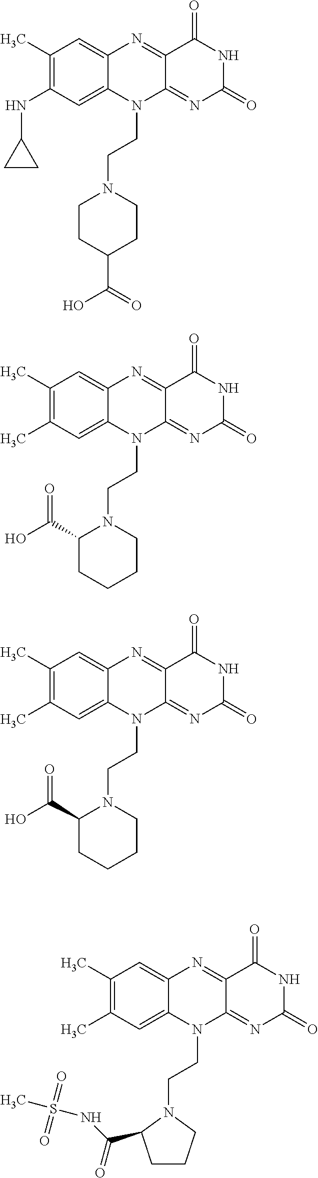

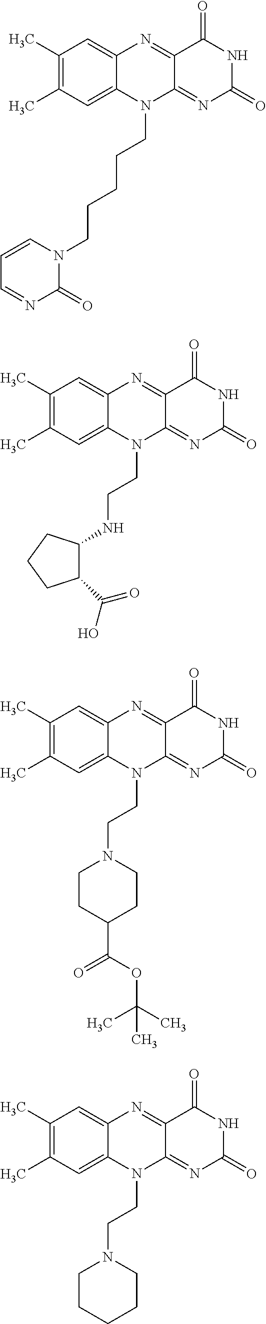

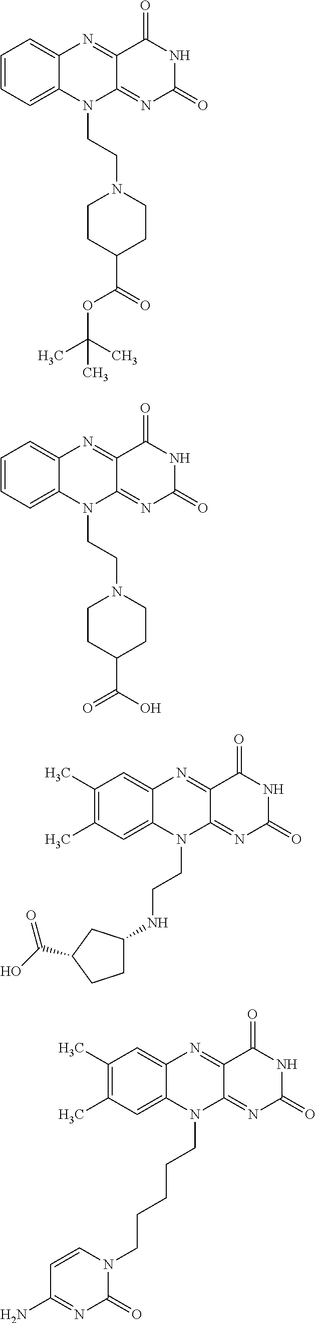

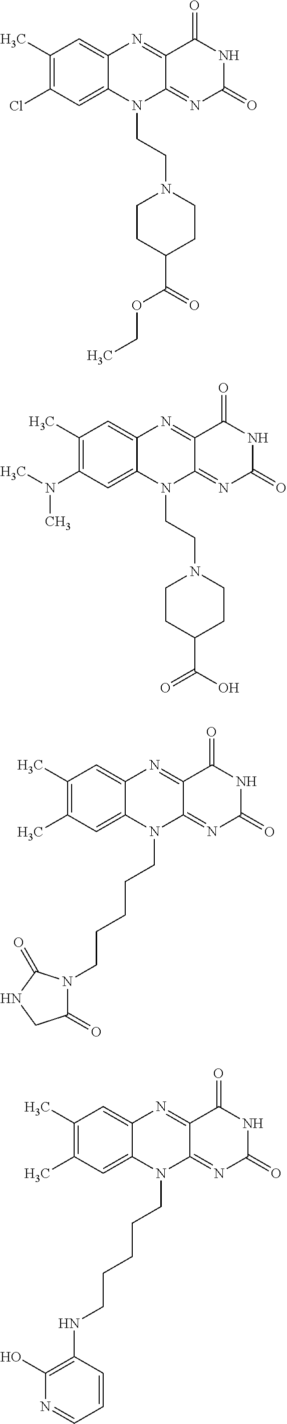

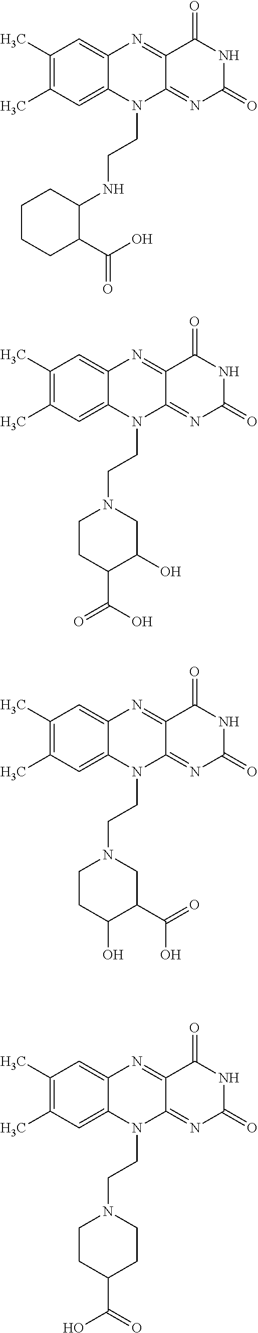

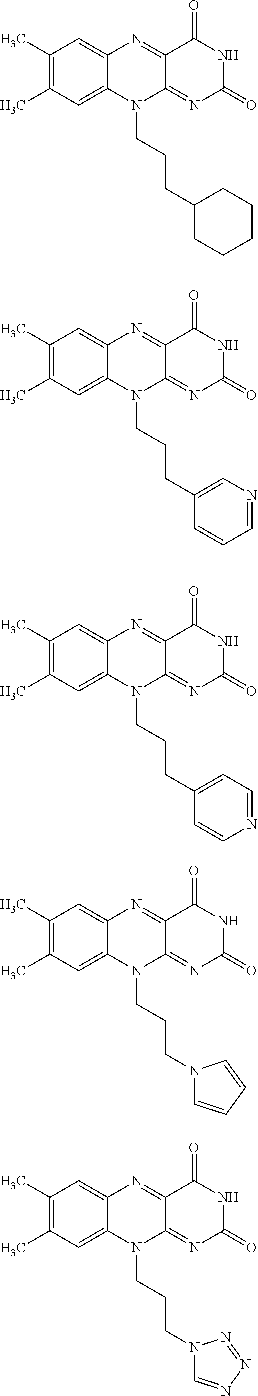

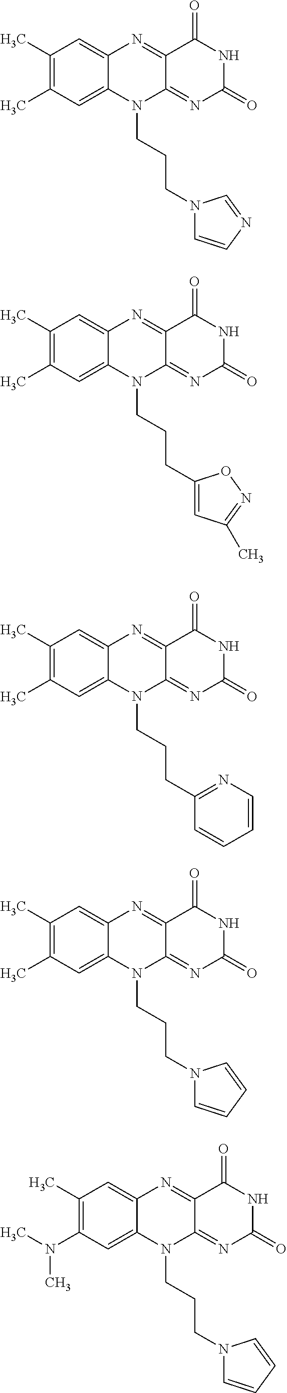

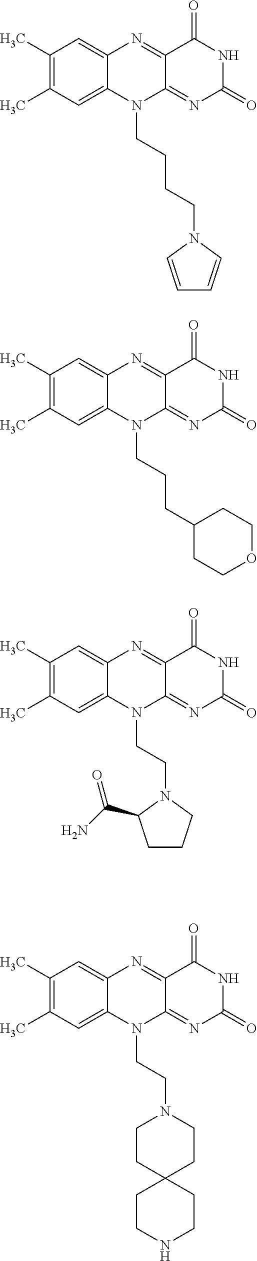

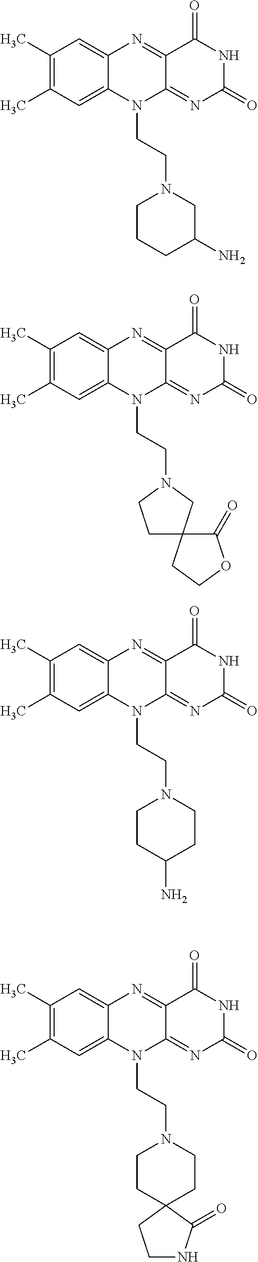

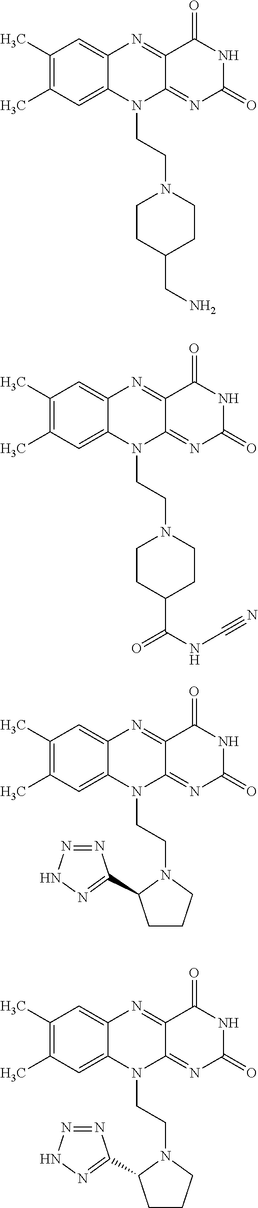

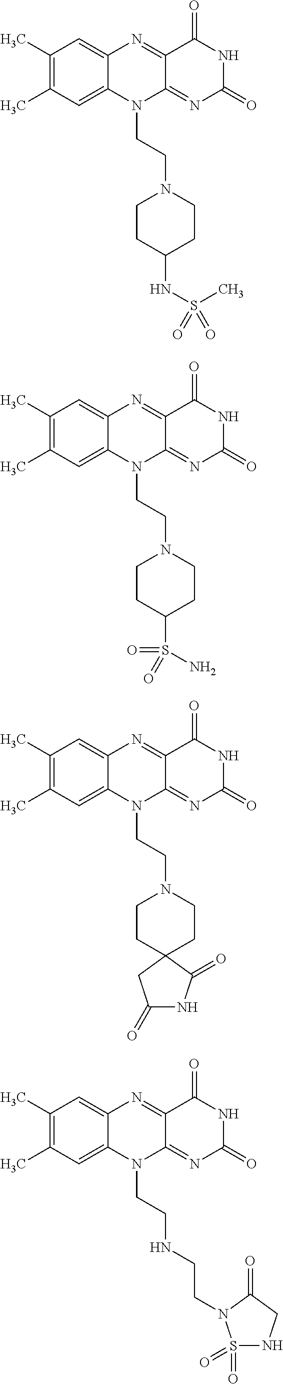

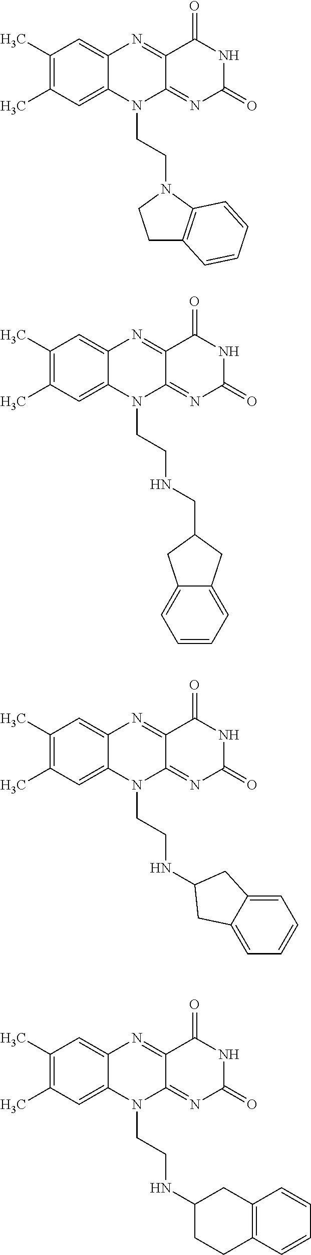

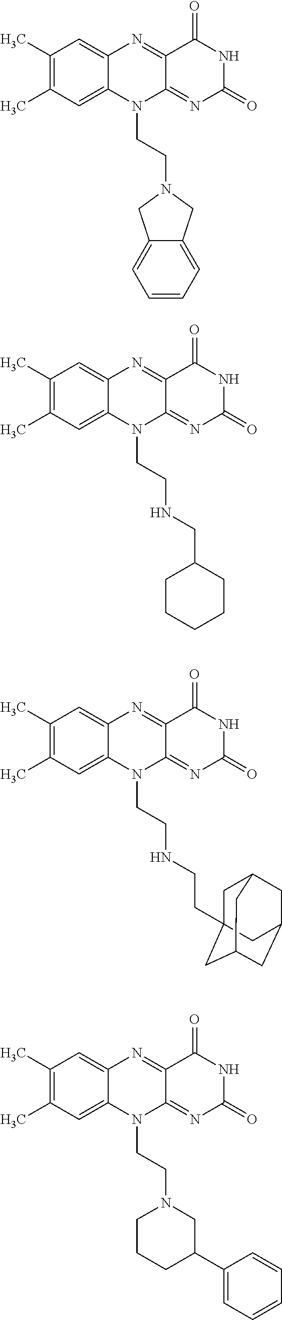

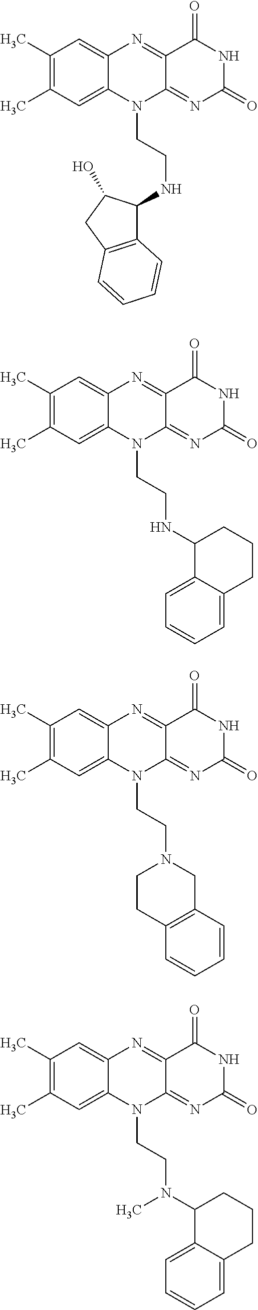

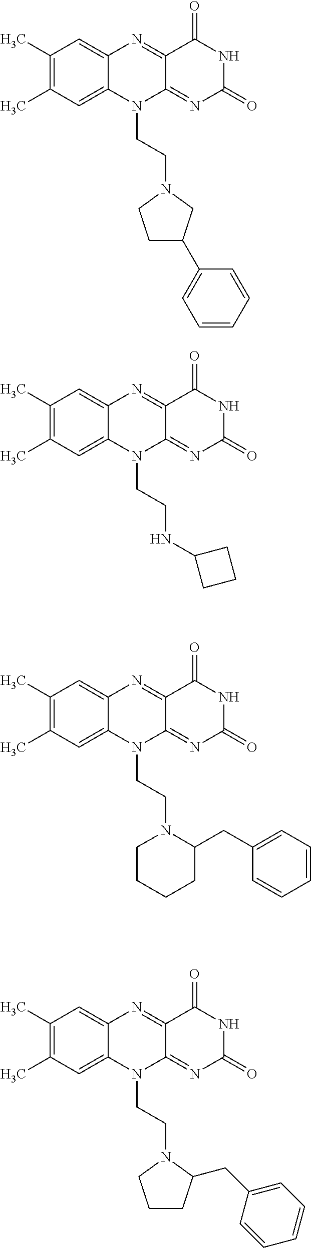

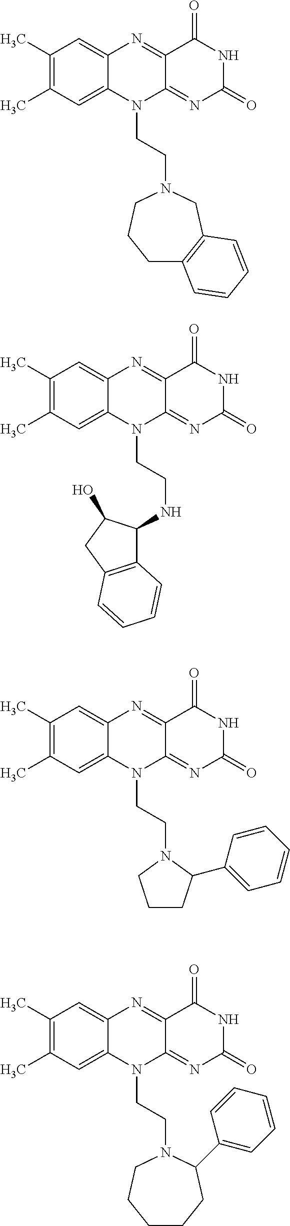

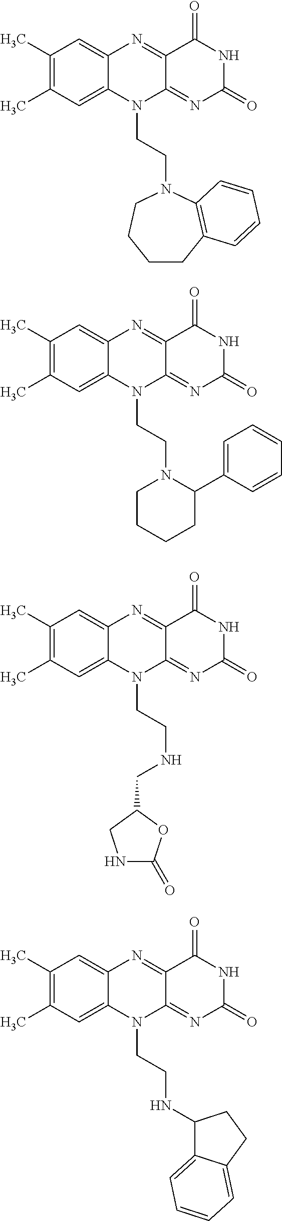

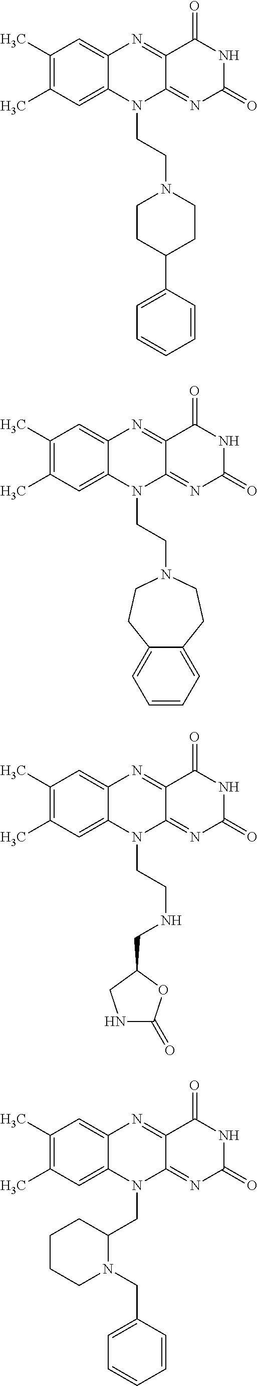

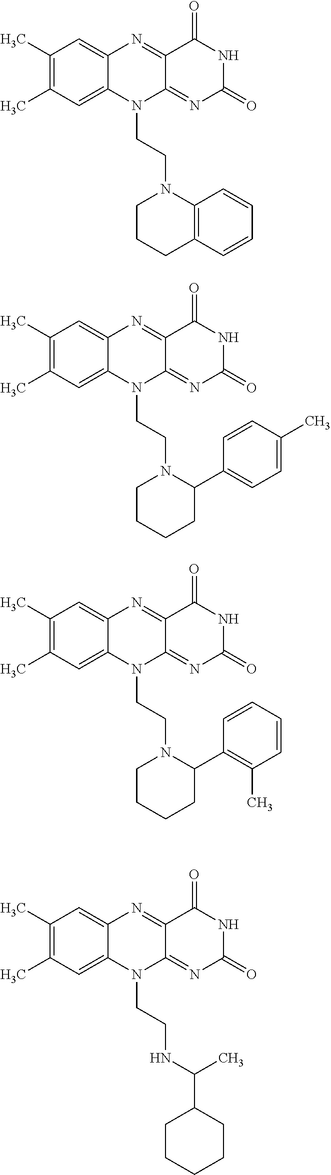

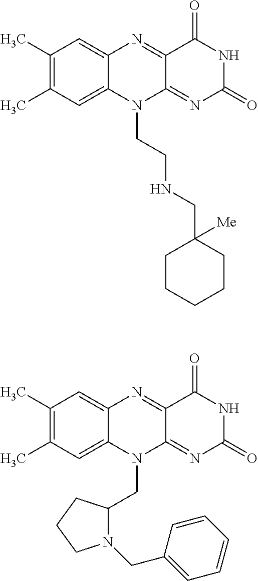

Q.36. any of the preceeding formulae wherein said compound is selected from the following:

Structures

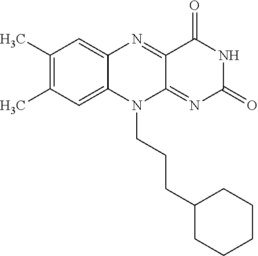

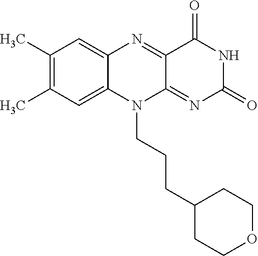

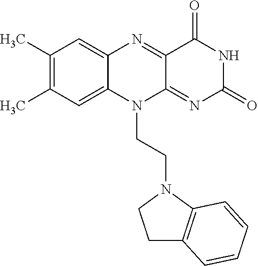

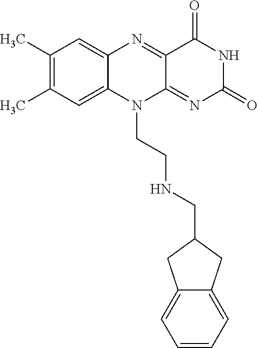

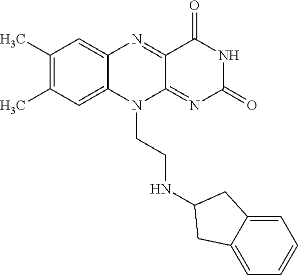

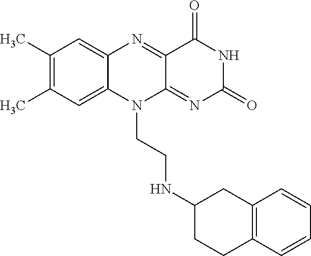

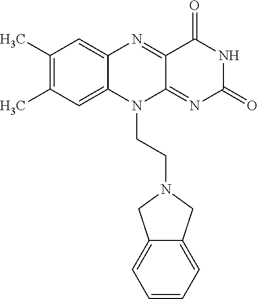

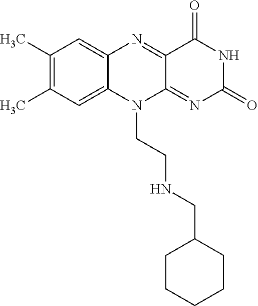

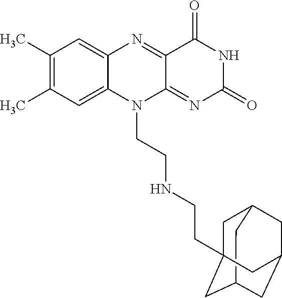

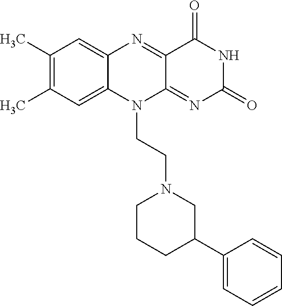

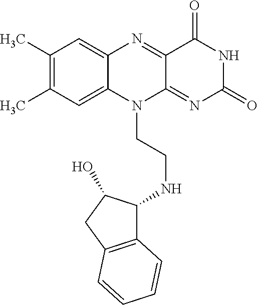

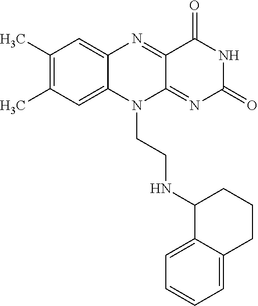

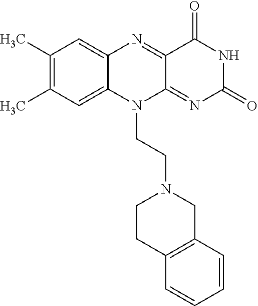

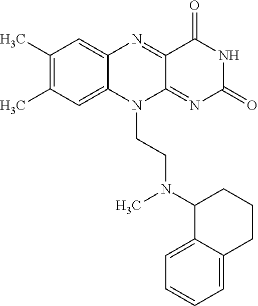

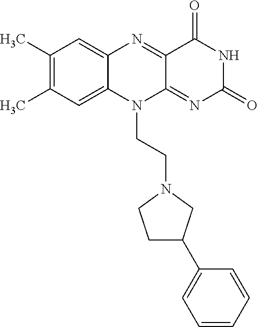

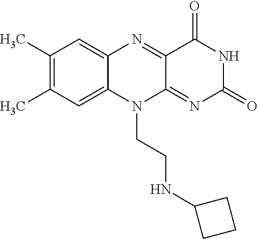

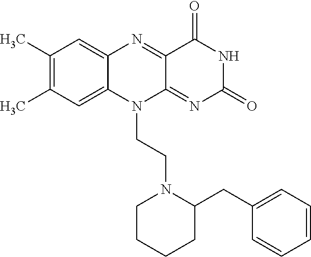

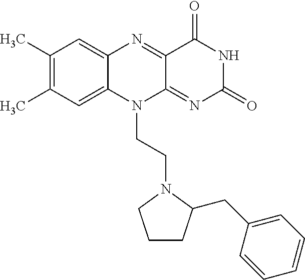

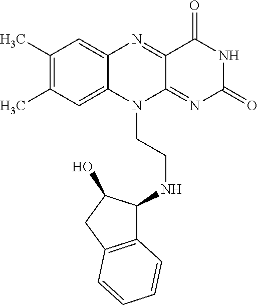

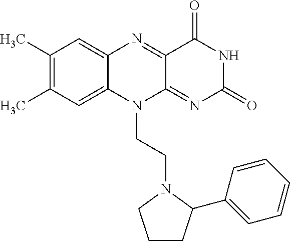

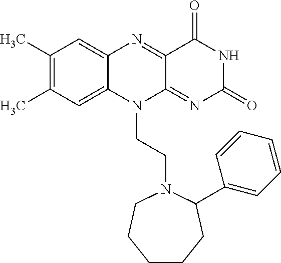

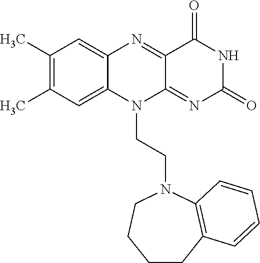

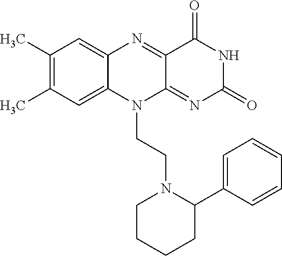

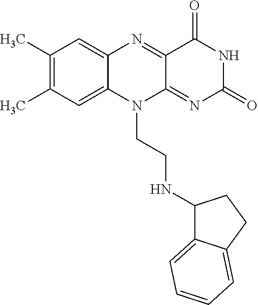

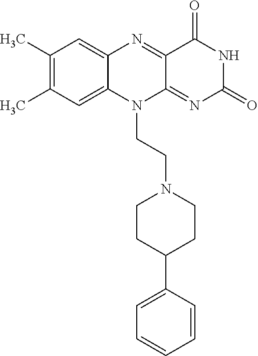

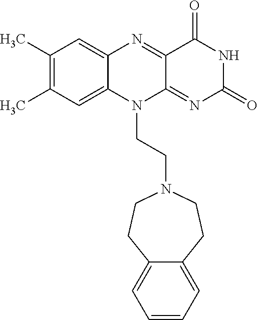

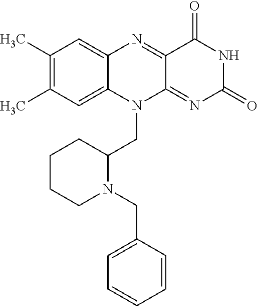

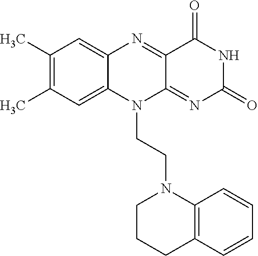

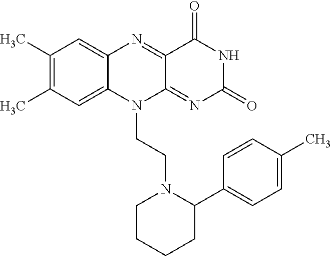

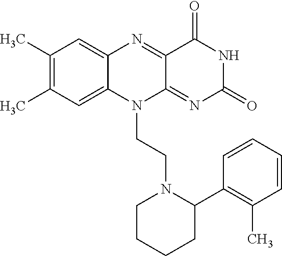

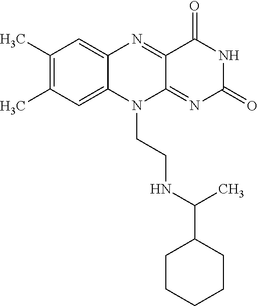

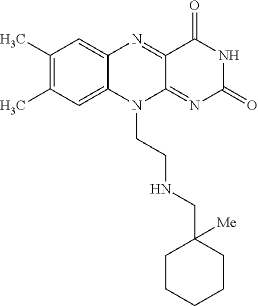

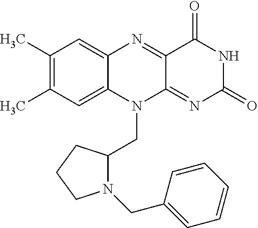

Q.37. any of the preceeding formulae wherein said compound is selected from the following:

Structures

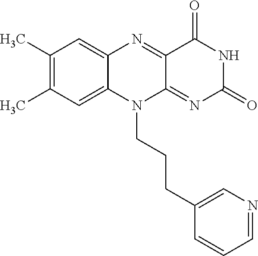

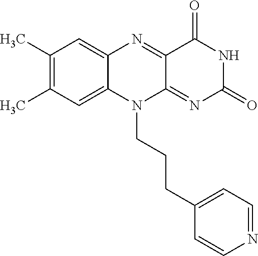

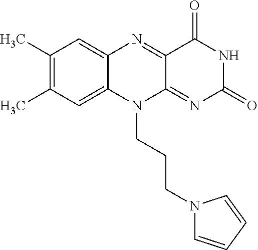

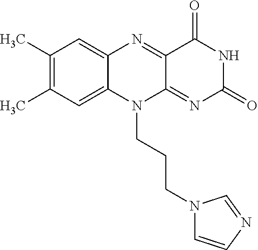

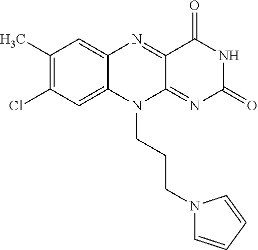

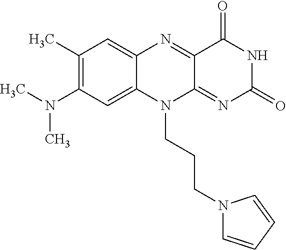

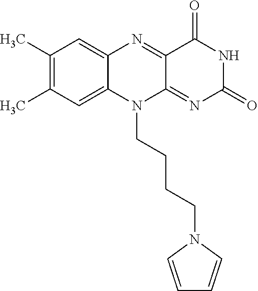

Q.38. any of the preceeding formulae wherein said compound is selected from the following:

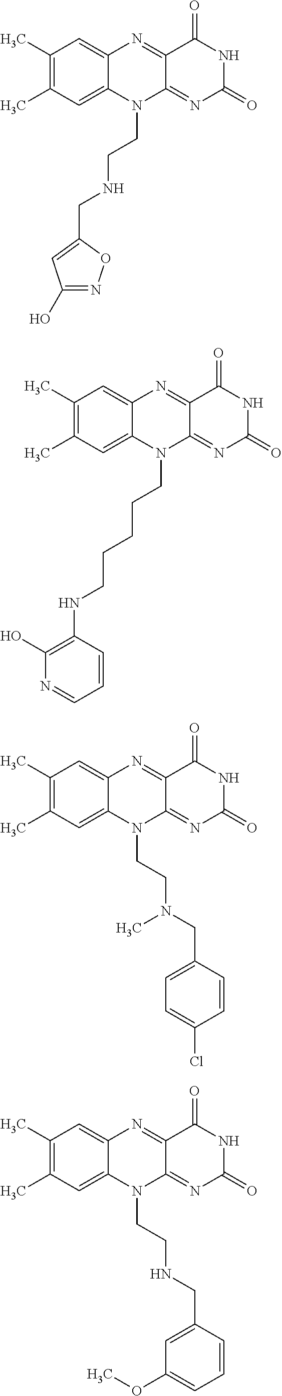

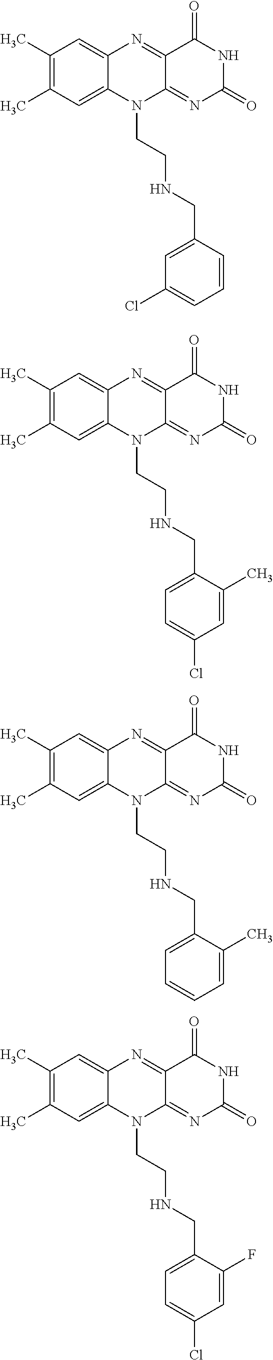

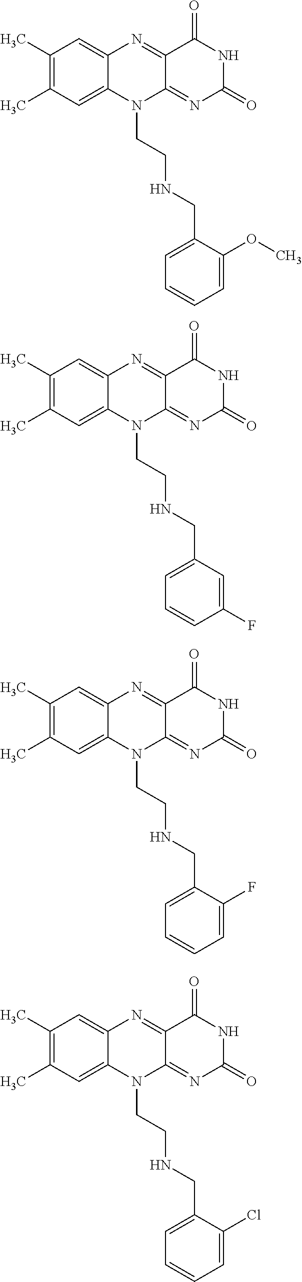

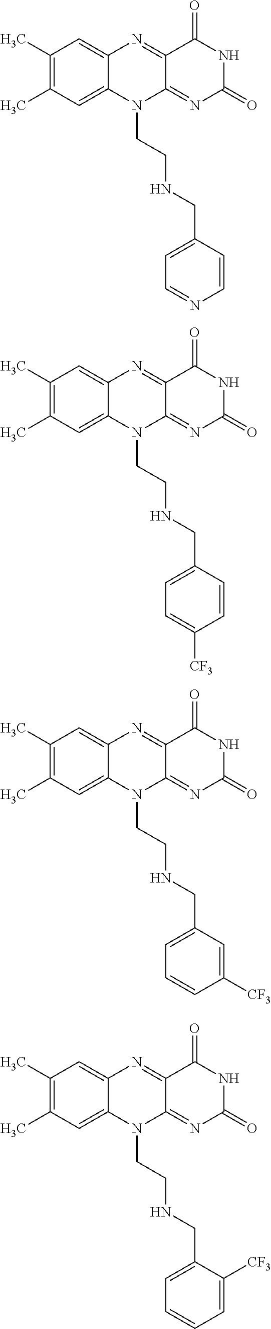

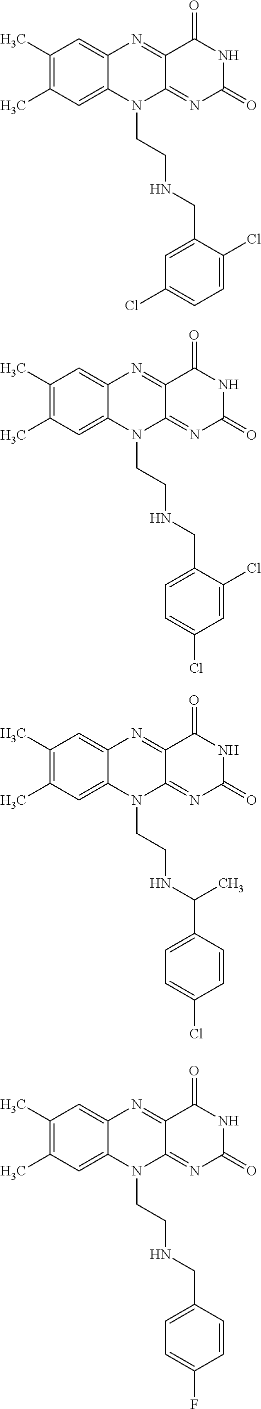

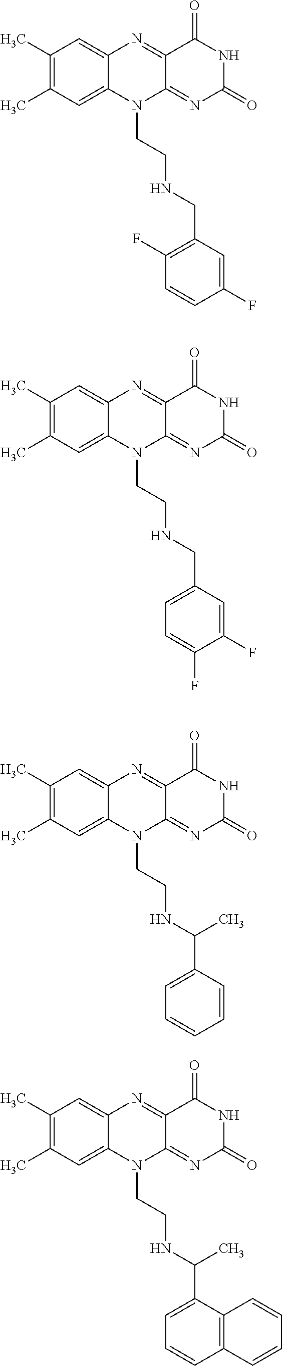

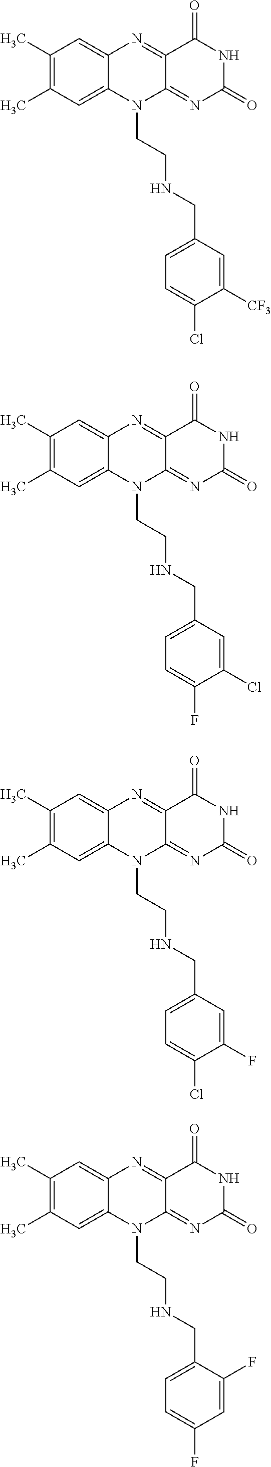

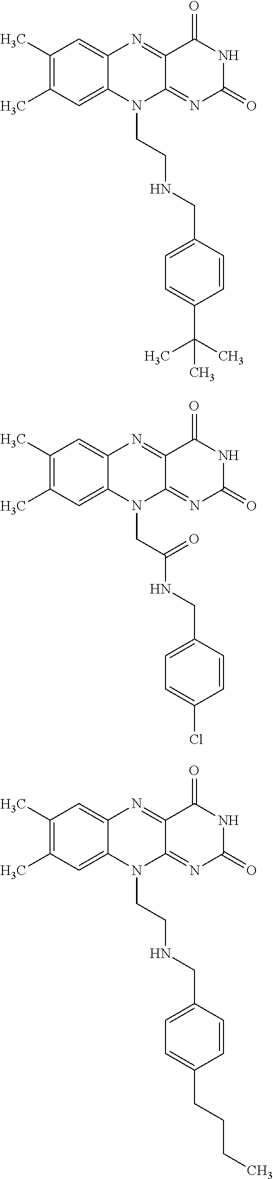

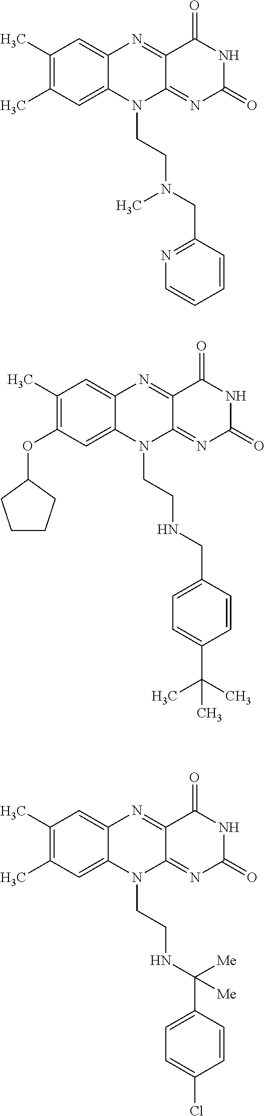

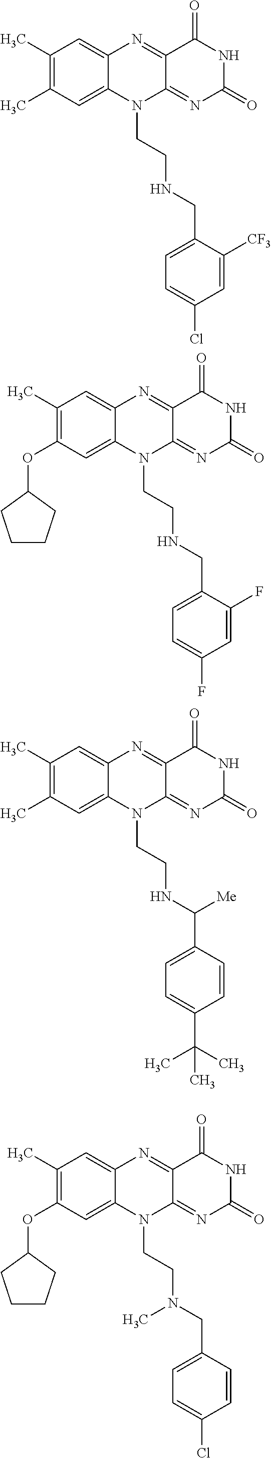

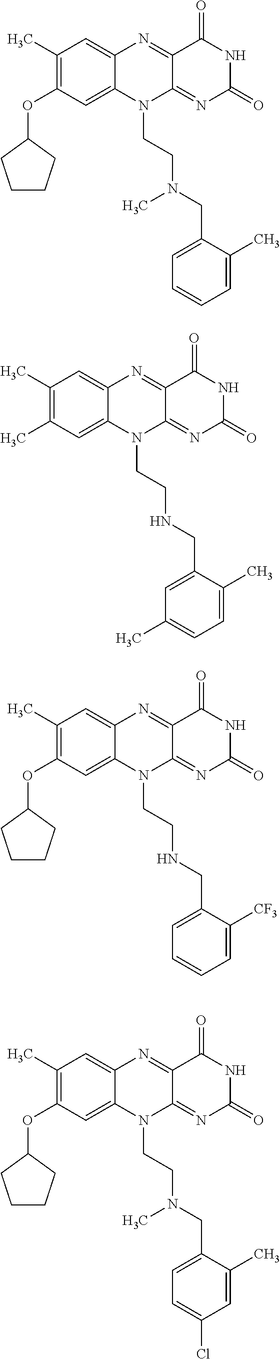

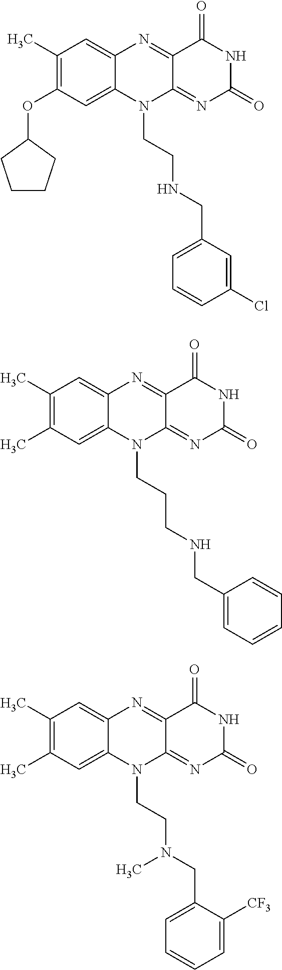

Q.39. any of the preceeding formulae wherein said compound is selected from the following

Structures

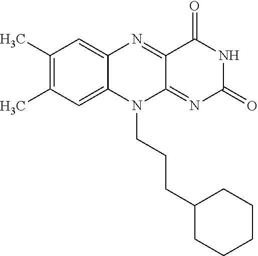

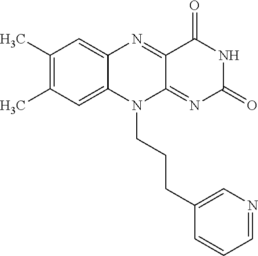

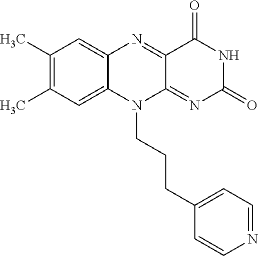

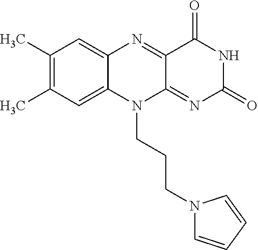

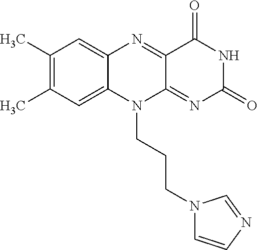

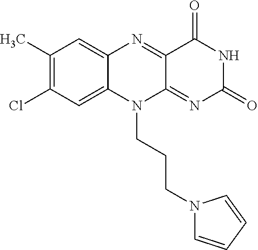

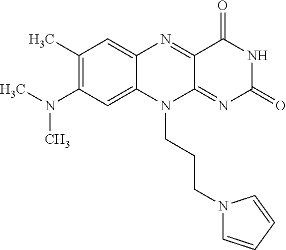

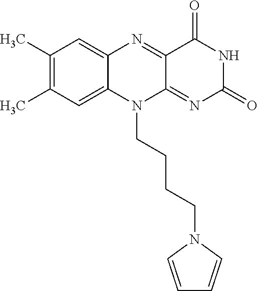

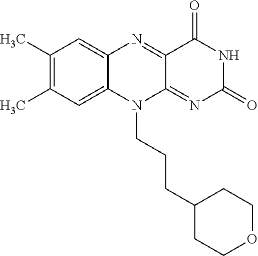

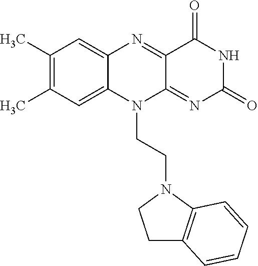

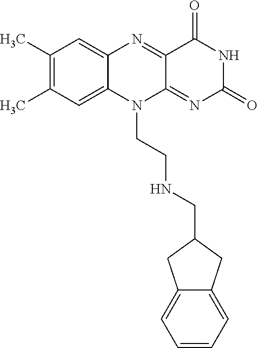

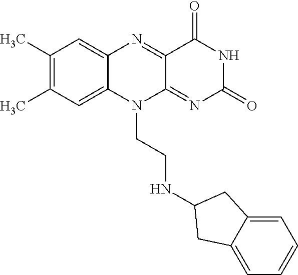

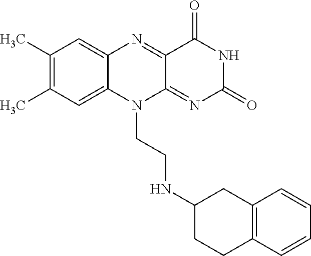

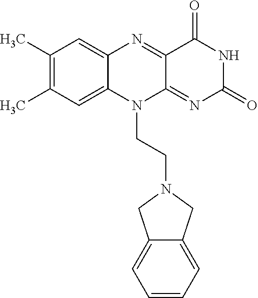

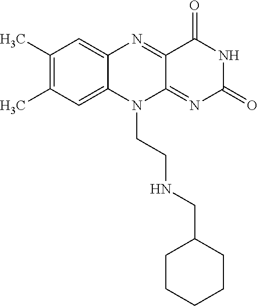

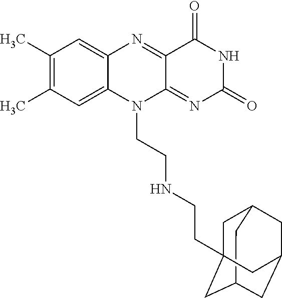

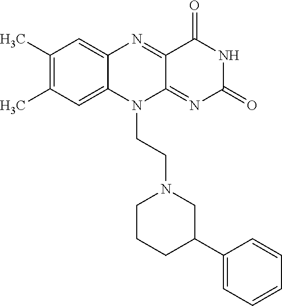

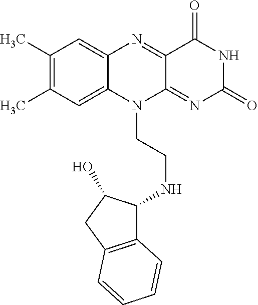

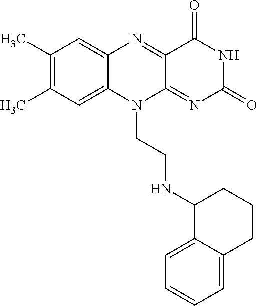

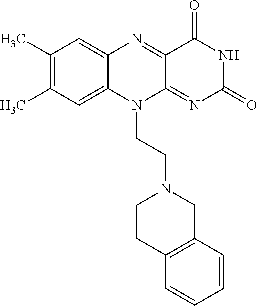

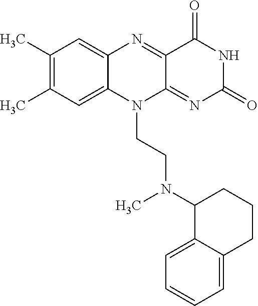

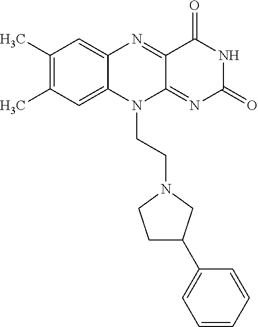

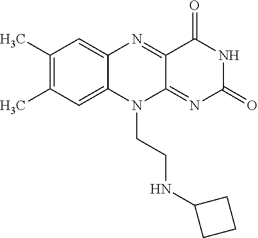

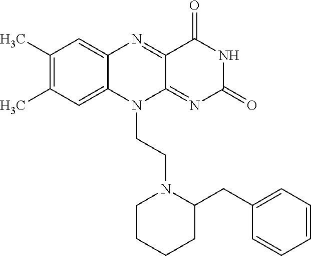

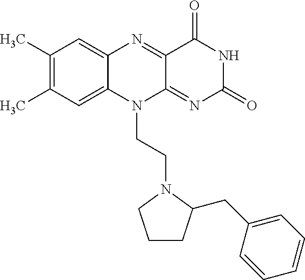

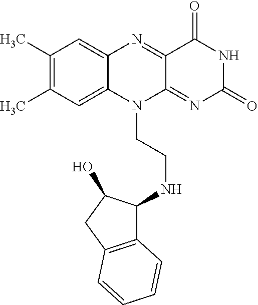

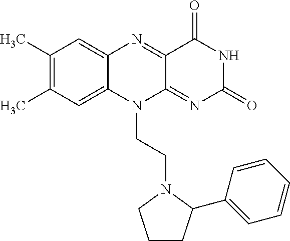

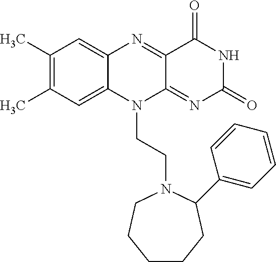

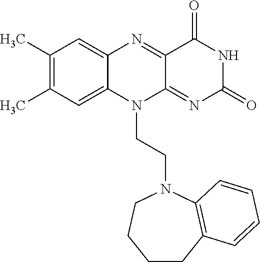

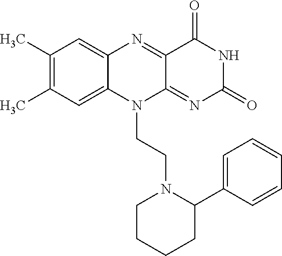

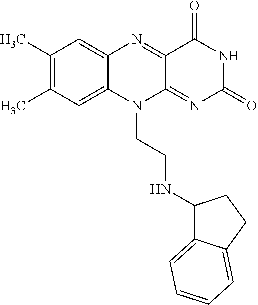

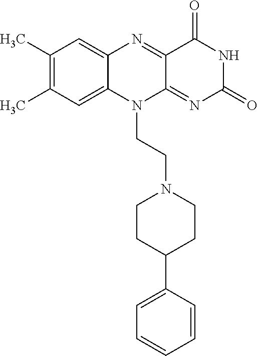

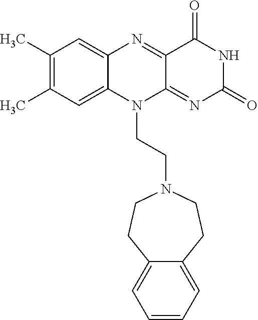

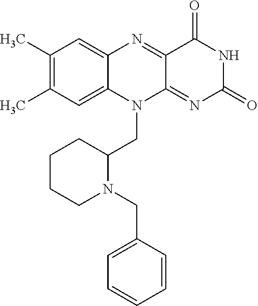

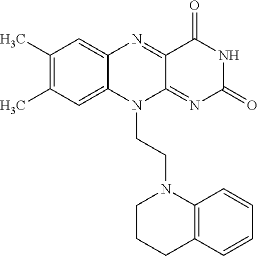

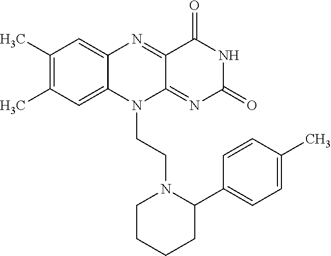

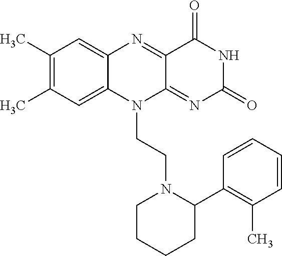

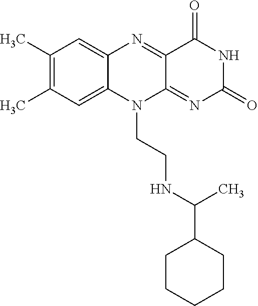

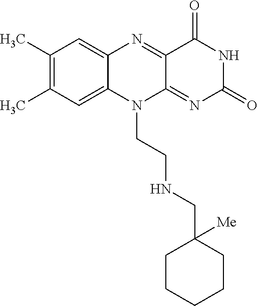

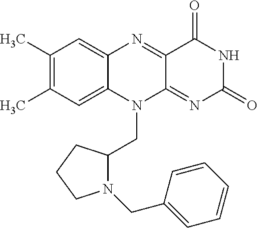

Q.40. any of the preceeding formulae wherein said compound is selected from the following:

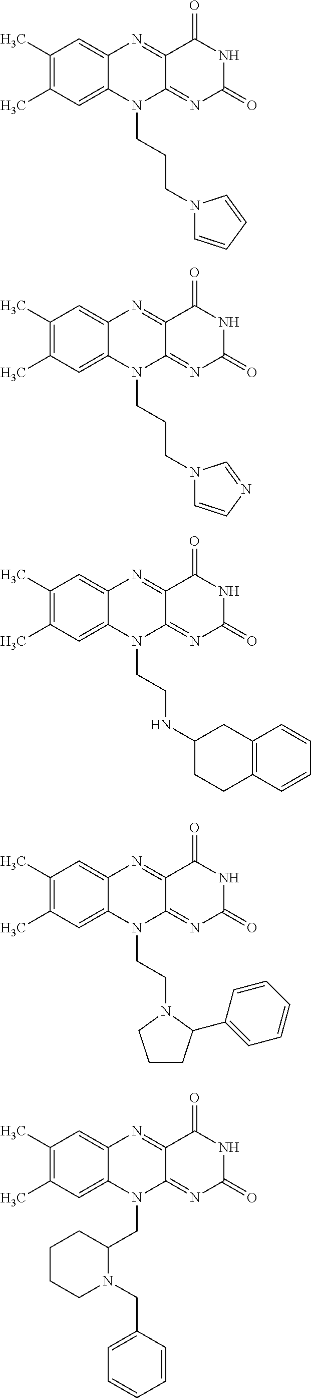

Q.41. any of the preceeding formulae wherein said compound is selected from the following:

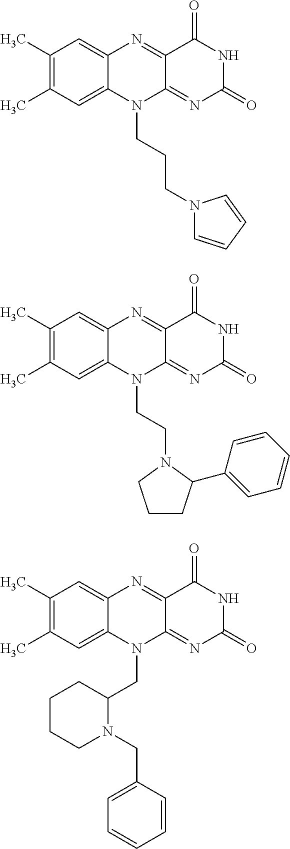

Q.42. any of the preceding formulae wherein the compound of Formula Q, or any of Q-I to Q-V, binds to FMN and/or CD3299 riboswitch, e.g., with an Imax of greater than 20% relative to the standard compound at 100 μM, in an assay, for example, as described in Example 1, or has an IC₅₀ value of less than or equal to 10 μM against the FMN riboswitch in an assay as described in Example 1, and/or has a Minimum Inhibitory Concentration (MIC) of less than or equal to 128 μg/mL, preferably less than or equal to 64 μg/mL, more preferably less than or equal to 32 μg/mL, for example, in an assay as described in Example 2,
   in free or salt form.

In a particular embodiment of the first aspect, the compound of Formula Q, or any of Q-I to Q-V, e.g., any of Q.1-Q.42, as hereinbefore described, contains the proviso that
(a) when R₂ is chloro, Alk is propylene, X is a single bond and A is pyrrolidin-1-yl, then R₁ is C₁₋₈ alkyl (e.g., methyl) or R₁₀ is —C₁₋₄alkyl-OC(O)CH₃ (e.g., —CH₂OC(O)CH₃), i.e., the compound is not 8-chloro-10-(3-pyrrolidin-1-ylpropyl)benzo[g]pteridine-2,4-dione;
(b) the compound is not 10-[3-(3,6-dioxo-1,4-cyclohexadien-1-yl)propyl)-3,7,8-trimethyl-benzo[g]pteridine-2,4-(3H,10H)-dione;
(c) A is not purinyl, e.g., the compound is not optionally substituted 1042-(9H-purin-9-yl)ethyl]-, 10-[3-(9H-purin-9-yl)propyl]- or 1046-(9H-purin-9-yl)hexyl]-7,8-dimethyl-benzo[g]pteridine-2,4-(3H,10H)-dione;
(d) A is not indol-3-yl, e.g., the compound is not 10-[3-(1H-indol-3-yl]ethyl]- or 10-[3-(1H-indol-3-yl)propyl]-7,8-dimethyl-benzo[g]pteridine-2,4-(3H,10H)-dione;
(e) -Alk-X-A is not 2-(2-oxocylopentylidene)ethyl,
   which compound Q, Q-I, Q-II, Q-III, Q-IV and Q-V having such proviso are referred to as Compound of Formula Q(i), Q-I(i), Q-II(i), Q-III(i), Q-IV(i), Q-V(i) respectively.

In the second aspect, the invention provides to a Compound of Formula I(A):

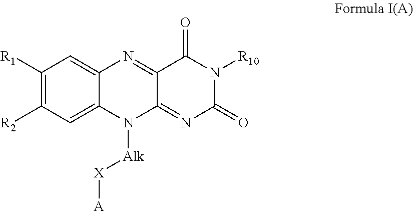

wherein:
(i) Alk is C₁₋₆alkylene (e.g., methylene or ethylene);
(ii) X is a single bond, —N(R₆)—, —N(R₆)—CH₂— or —C(O)—;
(iii) A is a monocyclic heteroaryl² (e.g., pyrid-4-yl or pyrid-3-yl) or C₅₋₆cycloalkyl² wherein one or more carbon atoms of said cycloalkyl² are optionally and independently replaced with N, O, S, S(O)₂ or —C(O)—, (for example, piperidinyl (e.g., piperidin-1-yl), pyrrolidinyl (e.g., pyrrolidin-1-yl), piperazinyl (e.g., 2,5-dioxopiperazin-1-yl), isoxazolidinyl (isoxazolidin-5-yl), 1,1-dioxo-1,4-thiazinan-4-yl, C₃₋₈cycloalkyl² (e.g., cyclopentyl, cyclohexyl or 2-oxocyclopentylidene), 2-oxopyrimidin-1-yl or 2,4-dioxo-imidazol-3-yl) wherein said heteroaryl² and cycloalkyl² are independently optionally substituted with one or more —C(O)OR₇, —CH₂C(O)OR₇, —N(R₆)C(O)OR₇, —OH, hydroxy-C₁₋₄alkyl (e.g., hydroxymethyl), —CH₂N(R₆)—C(O)OR₇, heteroaryl² (e.g., 2H-tetrazol-5-yl), heteroaryl²-C₁₋₄alkyl (e.g., 2H-tetrazol-5-yl-methyl), amineC₁₋₄alkyl (e.g., amine-ethyl), C₁₋₄alkoxy (e.g., methoxy), —C(O)N(R₆)—S(O)₂—C₁₋₄alkyl (e.g., —C(O)N(H)S(O)₂—CH₃) or —N(R₈)(R₉);
(iv) R₁ is H or C₁₋₈ alkyl (e.g., methyl);
(v) R₂ is H, halo (e.g., chloro), C₁₋₄alkyl (e.g., methyl), —N(R₄)(R₅);
(vi) R₄ and R₅ are independently selected from H, C₃₋₇ cycloalkyl² (e.g., cyclopropyl or cyclopentyl), —C₁₋₄alkyl (e.g., methyl or ethyl), wherein said alkyl is optionally substituted with one or more groups selected from —OH, —C(O)OR₇, aryl² optionally substituted with halo (e.g., 4-fluorophenyl);
(vii) R₆ is H or C₁₋₄alkyl (e.g., methyl);
(viii) R₇ is H, C₁₋₄alkyl (e.g., methyl, ethyl or tert-butyl), —CH₂OC(O)CH₃;
(ix) R₈ and R₉ are independently H or C₁₋₄alkyl;
(x) R₁₀ is H or —C₁₋₄alkyl-OC(O)CH₃ (e.g., —CH₂OC(O)CH₃),
   in free, salt or prodrug form.

The invention further relates to a Compound of Formula I(A) as follows:
1.1 a Compound of formula I(A), wherein Alk is C₁₋₆alkylene (e.g., ethylene);
1.2 a Compound of Formula I(A) or 1.1, wherein Alk is C₁₋₂alkylene (e.g., ethylene);
1.3 a Compound of Formula I(A) or 1.1, wherein Alk is ethylene;
1.4 a Compound of Formula I(A) or 1.1, wherein Alk is C₃₋₆alkylene (e.g., pentylene);
1.5 a Compound of Formula I(A) or any of 1.1-1.4, wherein X is a single bond, —N(R₆)—, —N(R₆)—CH₂— or —C(O)—;
1.6 a Compound of Formula I(A) or any of 1.1-1.5, X is —N(R₆)—;
1.7 a Compound of Formula I(A) or any of 1.1-1.5, X is —N(R₆)—CH₂—;
1.8 a Compound of Formula I(A) or any of 1.1-1.5, X is —C(O)—;
1.9 a Compound of Formula I(A) or any of 1.1-1.5, X is a single bond;
1.10 a Compound of Formula I(A) or any of 1.1-1.9, wherein A is a monocyclic heteroaryl² (e.g., pyrid-4-yl or pyrid-3-yl) or C₅₋₆cycloalkyl² wherein one or more carbon atoms of said cycloalkyl² are optionally replaced with N, O, S, S(O)₂ or —C(O)—, (for example, piperidinyl (e.g., piperidin-1-yl), pyrrolidinyl (e.g., pyrrolidin-1-yl), piperazinyl (e.g., 2,5-dioxopiperazin-1-yl), isoxazolidinyl (isoxazolidin-5-yl), 1,1-dioxo-1,4-thiazinan-4-yl, C₃₋₈acycloalkyl² (e.g., cyclopentyl, cyclohexyl or 2-oxocyclopentylidene), 2-oxopyrimidin-1-yl or 2,4-dioxo-imidazol-3-yl) wherein said heteroaryl² and cycloalkyl² are independently optionally substituted with one or more —C(O)OR₇, —CH₂C(O)OR₇, —N(R₆)C(O)OR₇, —OH, hydroxy-C₁₋₄alkyl (e.g., hydroxymethyl), —CH₂N(R₆)—C(O)OR₇, heteroaryl² (e.g., 2H-tetrazol-5-yl), heteroaryl²-C₁₋₄alkyl (e.g., 2H-tetrazol-5-yl-methyl), amineC₁₋₄ alkyl (e.g., amine-ethyl), C₁₋₄alkoxy (e.g., methoxy), —C(O)N(R₆)—S(O)₂—C₁₋₄alkyl (e.g., —C(O)N(H)S(O)₂—CH₃) or —N(R₈)(R₉);
1.11 a Compound of Formula I(A) or any of 1.1-1.10, wherein A is a monocyclic heteroaryl² (e.g., pyrid-4-yl or pyrid-3-yl);
1.12 a Compound of Formula I(A) or any of 1.1-1.10, wherein A is a C₅₋₆cycloalkyl² wherein one or more carbon atoms of said cycloalkyl² are optionally replaced with N, O, S, S(O)₂ or —C(O)— (for example, piperidinyl (e.g., piperidin-1-yl), pyrrolidinyl (e.g., pyrrolidin-1-yl), piperazinyl (e.g., 2,5-dioxopiperazin-1-yl), isoxazolidinyl (isoxazolidin-5-yl), 1,1-dioxo-1,4-thiazinan-4-yl, C₃₋₈acycloalkyl² (e.g., cyclopentyl or cyclohexyl), 2-oxopyrimidin-1-yl or 2,4-dioxo-imidazol-3-yl) wherein said heteroaryl² and cycloalkyl² are independently optionally substituted with one or more —C(O)OR₇, —CH₂C(O)OR₇, —N(R₆)C(O)OR₇, —OH, hydroxy-C₁₋₄alkyl (e.g., hydroxymethyl), —CH₂N(R₆)—C(O)OR₇, heteroaryl² (e.g., 2H-tetrazol-5-yl), heteroaryl²-C₁₋₄alkyl (e.g., 2H-tetrazol-5-yl-methyl), amineC₁₋₄alkyl (e.g., amine-ethyl), C₁₋₄alkoxy (e.g., methoxy), —C(O)N(R₆)—S(O)₂—C₁₋₄alkyl (e.g., —C(O)N(H)S(O)₂—CH₃) or —N(R_{g})(R₉);
1.13 a Compound of Formula I(A) or any of 1.1-1.10, wherein A is selected from any of the following: piperidinyl (e.g., piperidin-1-yl), pyrrolidinyl (e.g., pyrrolidin-1-yl), piperazinyl (e.g., 2,5-dioxopiperazin-1-yl), isoxazolidinyl (isoxazolidin-5-yl), 1,1-dioxo-1,4-thiazinan-4-yl, C₃₋₈cycloalkyl² (e.g., cyclopentyl or cyclohexyl), 2-oxopyrimidin-1-yl or 2,4-dioxo-imidazol-3-yl) wherein said cycloalkyl² is optionally substituted with one or more —C(O)OR₇, —CH₂C(O)OR₇, —N(R₆)C(O)OR₇, —OH, hydroxy-C₁₋₄alkyl (e.g., hydroxymethyl), —CH₂N(R₆)—C(O)OR₇, heteroaryl² (e.g., 2H-tetrazol-5-yl), heteroaryl²-C₁₋₄alkyl (e.g., 2H-tetrazol-5-yl-methyl), amineC₁₋₄alkyl (e.g., amine-ethyl), C₁₋₄alkoxy (e.g., methoxy), —C(O)N(R₆)—S(O)₂—C₁₋₄alkyl (e.g., —C(O)N(H)S(O)₂—CH₃) or —N(R₈)(R₉);
1.14 a Compound of Formula I(A) or any of 1.1-1.3 or 1.5-1.13, wherein -Alk-X-A is selected from any of the following:

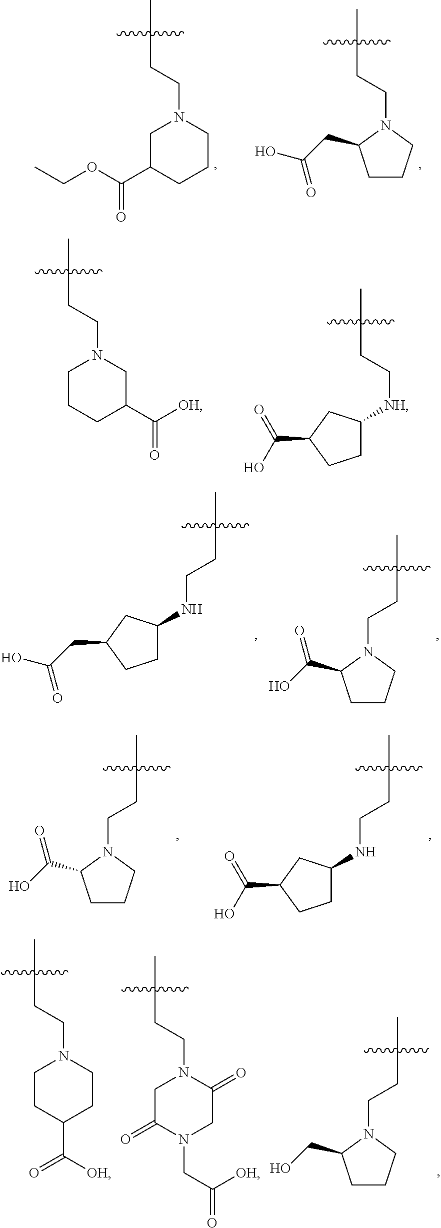

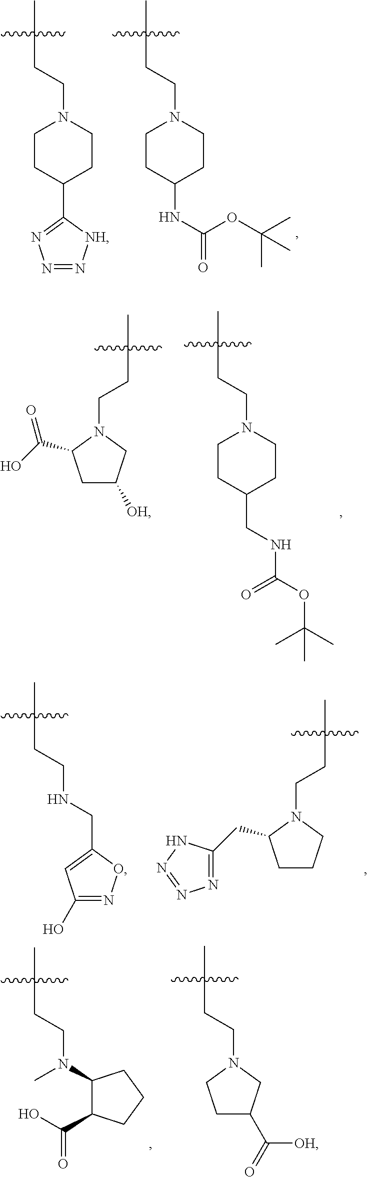

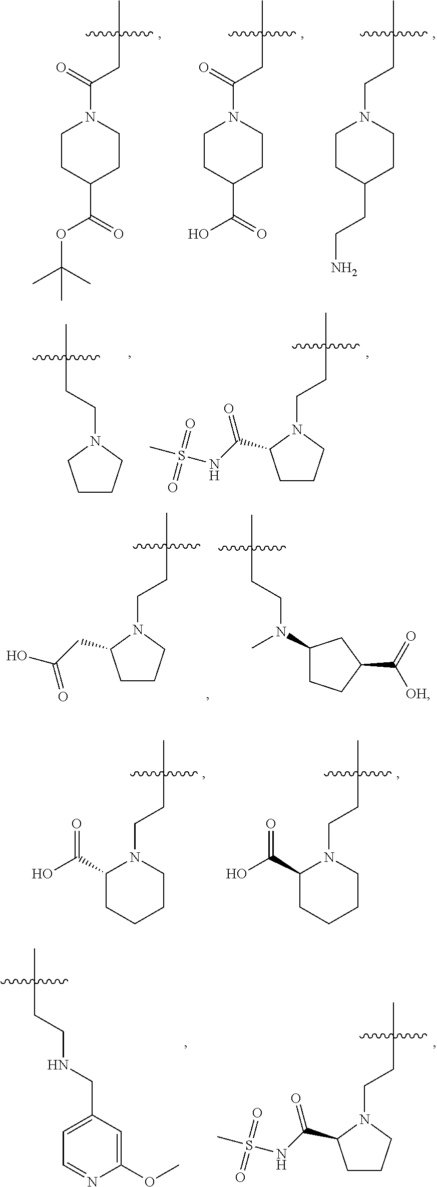

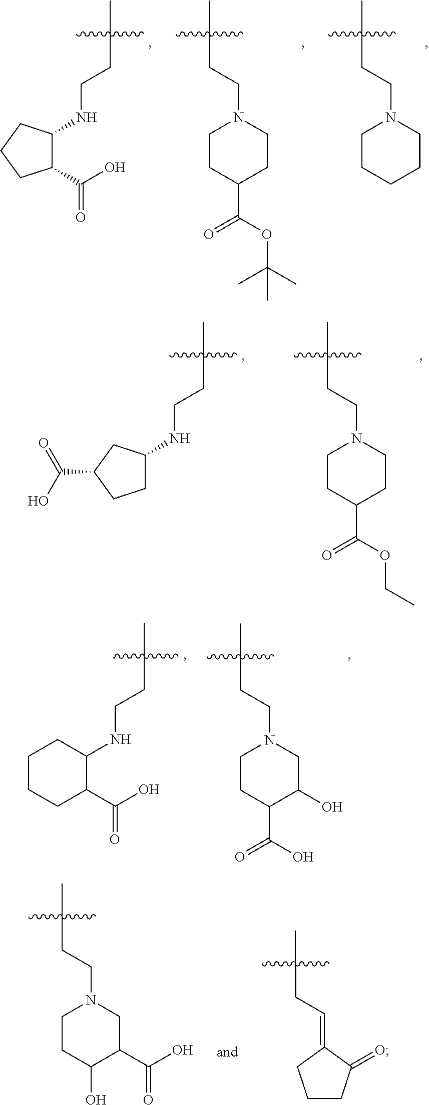

1.15 a Compound of Formula I(A) or any of 1.1-1.3 or 1.5-1.13, wherein -Alk-X-A is selected from any of the following:

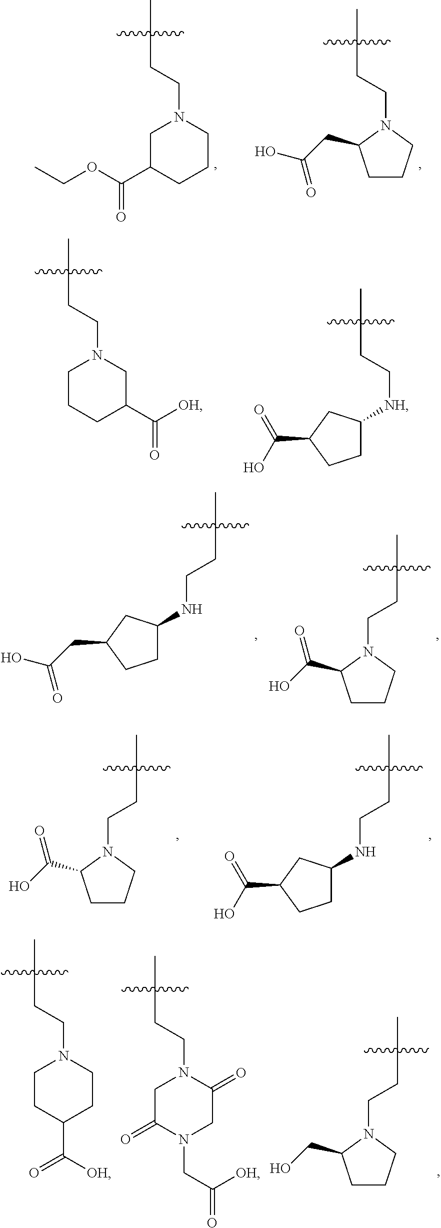

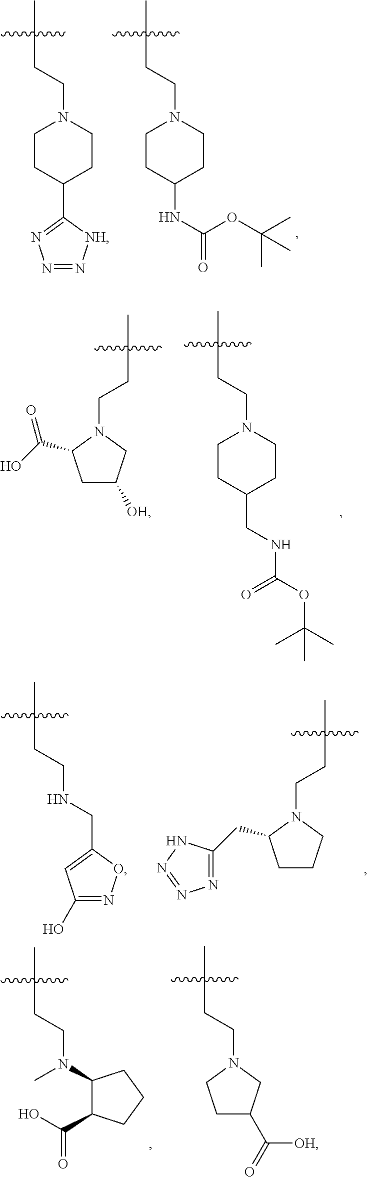

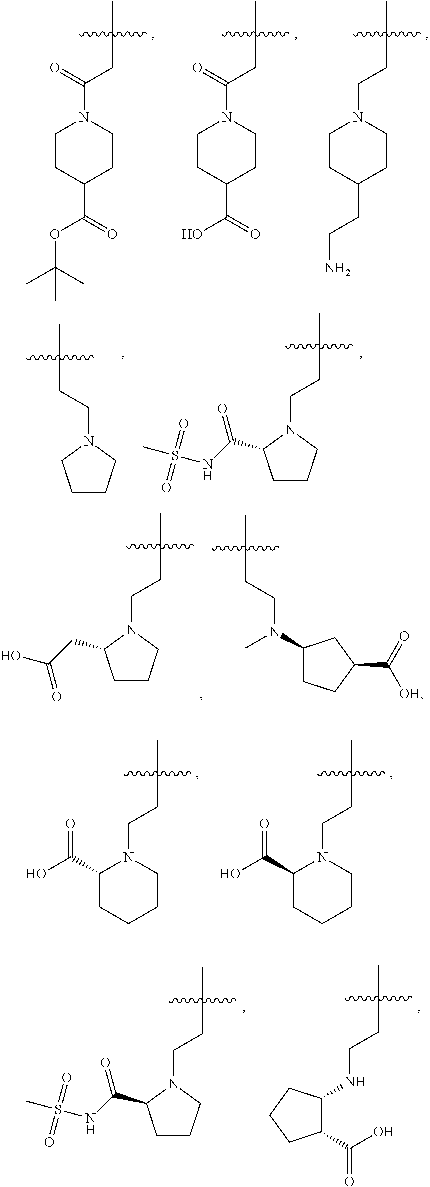

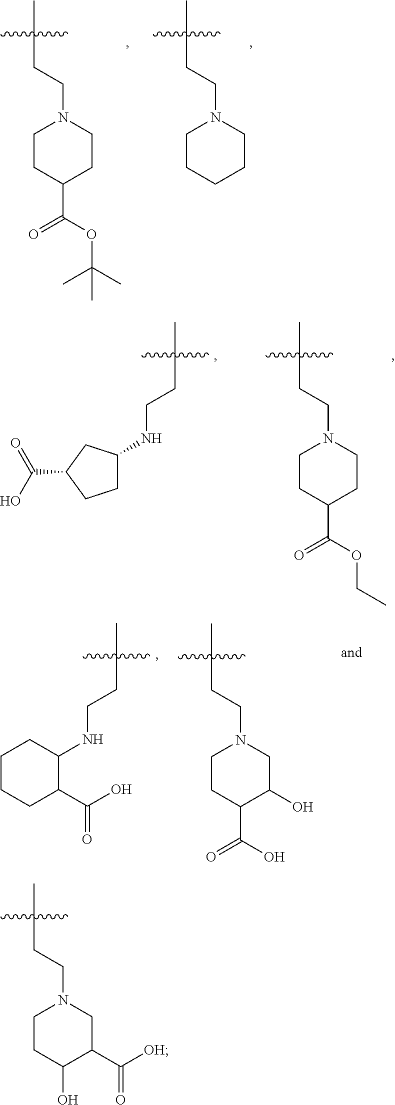

1.16 a Compound of Formula I(A), 1.1 or any of 1.4-1.6, 1.9-1.13 wherein -Alk-X-A are selected from any of the following:

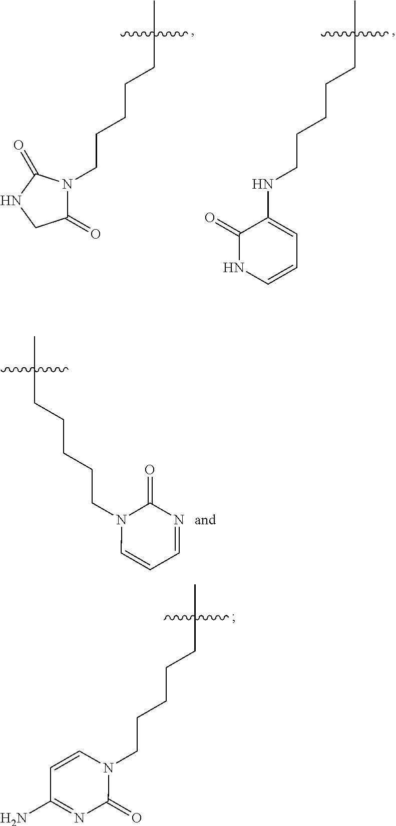

1.17 a Compound of Formula I(A) or any of 1.1-1.16, wherein R₁ is H or C₁₋₈ alkyl (e.g., methyl);
1.18 a Compound of Formula I(A) or any of 1.1-1.16, wherein R₁ is H,
1.19 a Compound of Formula I(A) or any of 1.1-1.16, wherein R₁ is C₁₋₈ alkyl (e.g., methyl);
1.20 a Compound of Formula I(A) or any of 1.1-1.19, wherein R₂ is H, halo (e.g., chloro), C₁₋₄alkyl (e.g., methyl), —N(R₄)(R₅);
1.21 a Compound of Formula I(A) or any of 1.1-1.19, wherein R₂ is H,
1.22 a Compound of Formula I(A) or any of 1.1-1.19, wherein R₂ is halo (e.g., chloro);
1.23 a Compound of Formula I(A) or any of 1.1-1.19, wherein R₂ is C₁₋₄alkyl (e.g., methyl);
1.24 a Compound of Formula I(A) or any of 1.1-1.19, wherein R₂ is —N(R₄)(R₅) wherein R₄ and R₅ are independently selected from H, C₃₋₇ cycloalkyl² (e.g., cyclopropyl or cyclopentyl), —C₁₋₄alkyl (e.g., methyl or ethyl), wherein said alkyl is optionally substituted with one or more groups selected from —OH, —C(O)OR₇, aryl optionally substituted with halo (e.g., 4-fluorophenyl);
1.25 Formula 1.24, wherein either R₄ or R₅ is H,
1.26 Formula 1.24 or 1.25, wherein either R₄ or R₅ is C₃₋₇cycloalkyl² (e.g., cyclopropyl or cyclopentyl);
1.27 Formula 1.24 or 1.25, wherein either R₄ or R₅ is —C₁₋₄alkyl (e.g., methyl or ethyl), wherein said alkyl is optionally substituted with one or more groups selected from —OH, —C(O)OR₇, aryl optionally substituted with halo (e.g., 4-fluorophenyl);
1.28 Formula 1.24 or 1.25, wherein either R₄ or R₅ is selected from any of the following: H, (e.g., methyl or ethyl), cyclopentylamine, —CH₂CH₂—C(O)OC(CH₃)₃ or hydroxyethyl;
1.29 a Compound of Formula I(A) or any of 1.1-1.28, wherein R₆ is H or C₁₋₄alkyl (e.g., methyl);
1.30 a Compound of Formula I(A) or any of 1.1-1.28, wherein R₆ is H,
1.31 a Compound of Formula I or any of 1.1-1.28, wherein R₆ is C₁₋₄alkyl (e.g., methyl);
1.32 a Compound of Formula I(A) or any of 1.1-1.31, wherein R₇ is H, C₁₋₄alkyl (e.g., methyl, ethyl or tert-butyl), —CH₂OC(O)CH₃;
1.33 a Compound of Formula I(A) or any of 1.1-1.31, wherein R₇ is H,
1.34 a Compound of Formula I(A) or any of 1.1-1.31, wherein R₇ is C₁₋₄alkyl (e.g., methyl, ethyl or tert-butyl);
1.35 a Compound of Formula I(A) or any of 1.1-1.31, wherein R₇ is —CH₂OC(O)CH₃;
1.36 a Compound of Formula I(A) or any of 1.1-1.35, wherein R₁₀ is H or —C₁₋₄alkyl-OC(O)CH₃ (e.g., CH₂OC(O)CH₃);
1.37 a Compound of Formula I(A) or any of 1.1-1.35, wherein R₁₀ is H,
1.38 a Compound of Formula I(A) or any of 1.1-1.35, wherein R₁₀ is —C₁₋₄alkyl-OC(O)CH₃ (e.g., CH₂OC(O)CH₃);
1.39 any of the preceding formulae wherein the Compound of Formula I(A) is selected from any of the following:

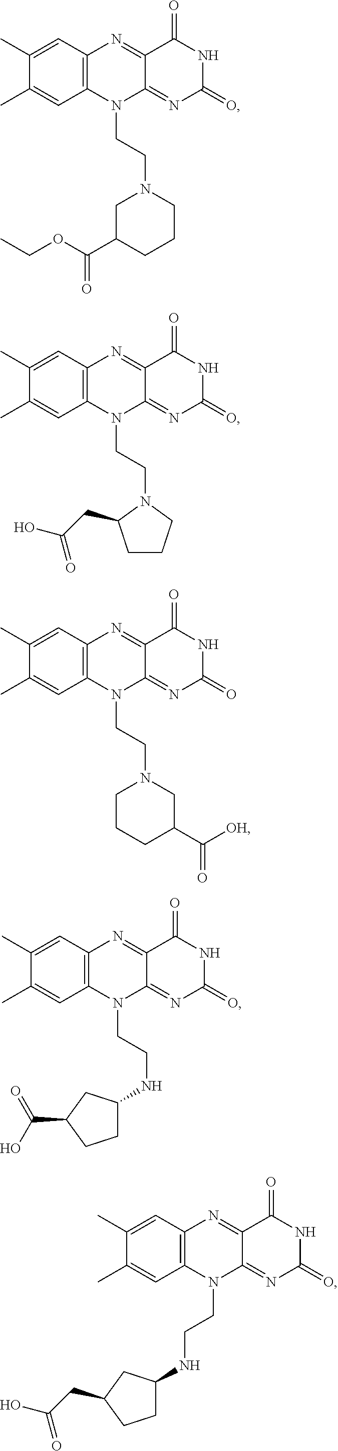

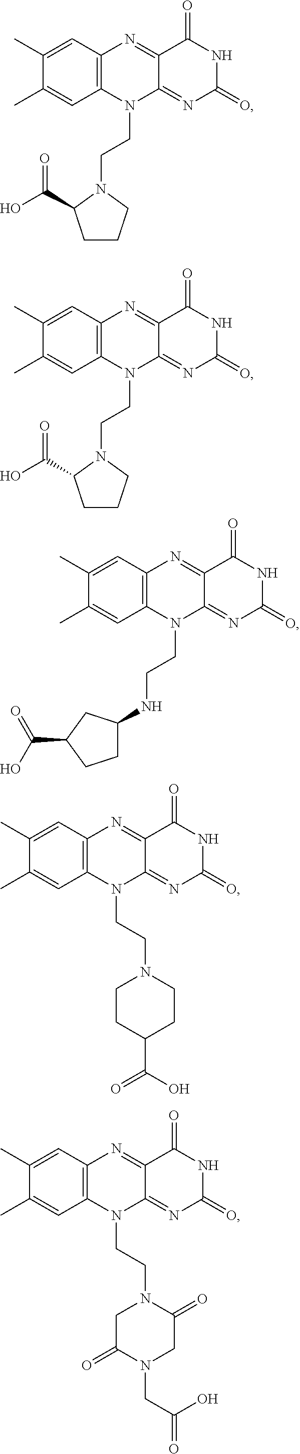

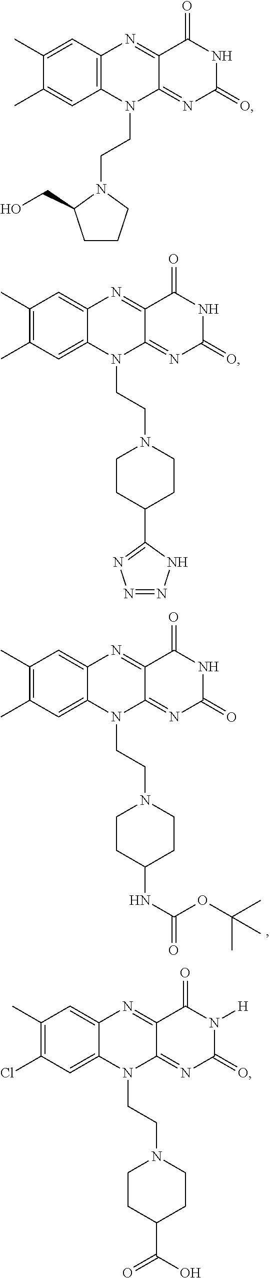

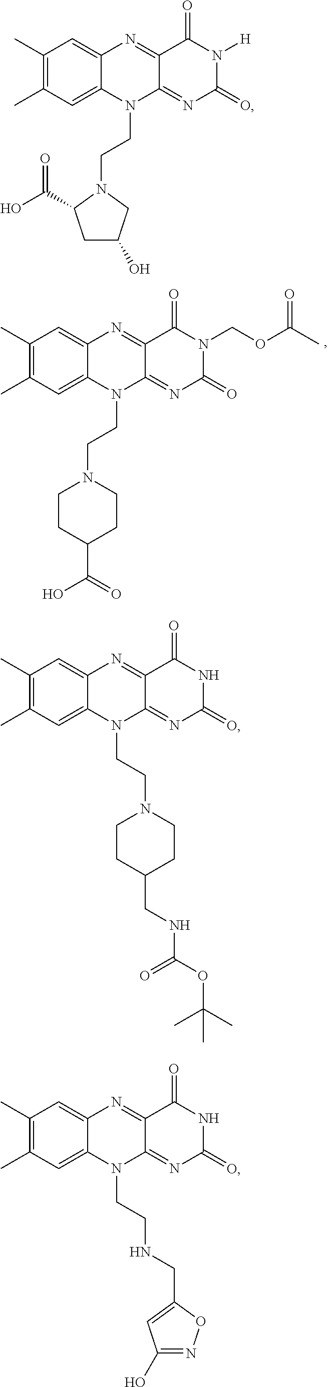

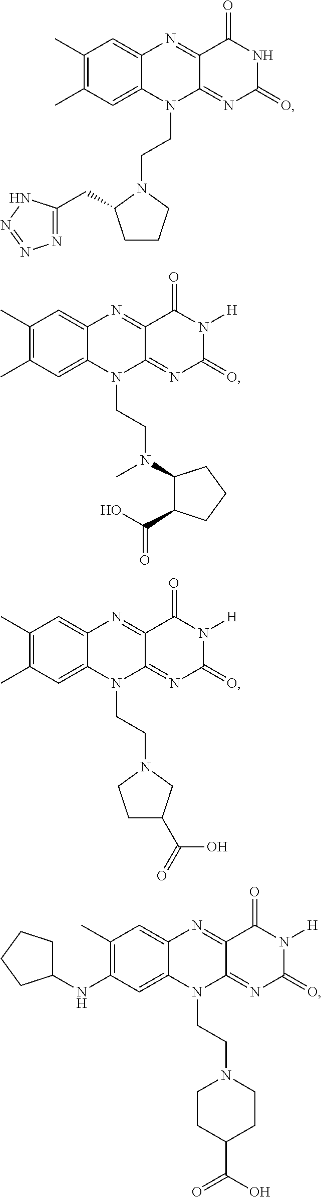

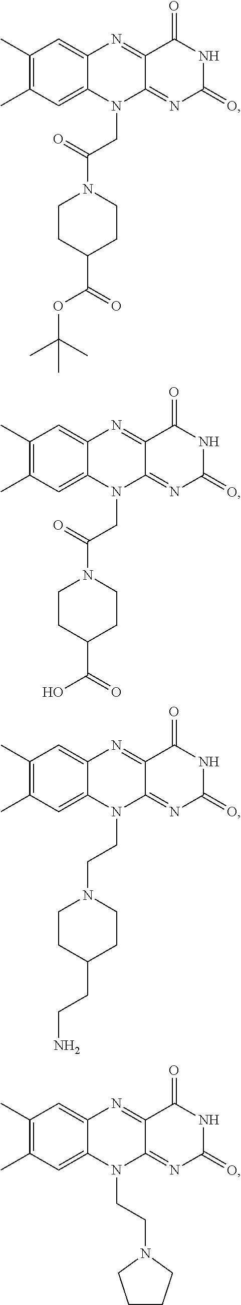

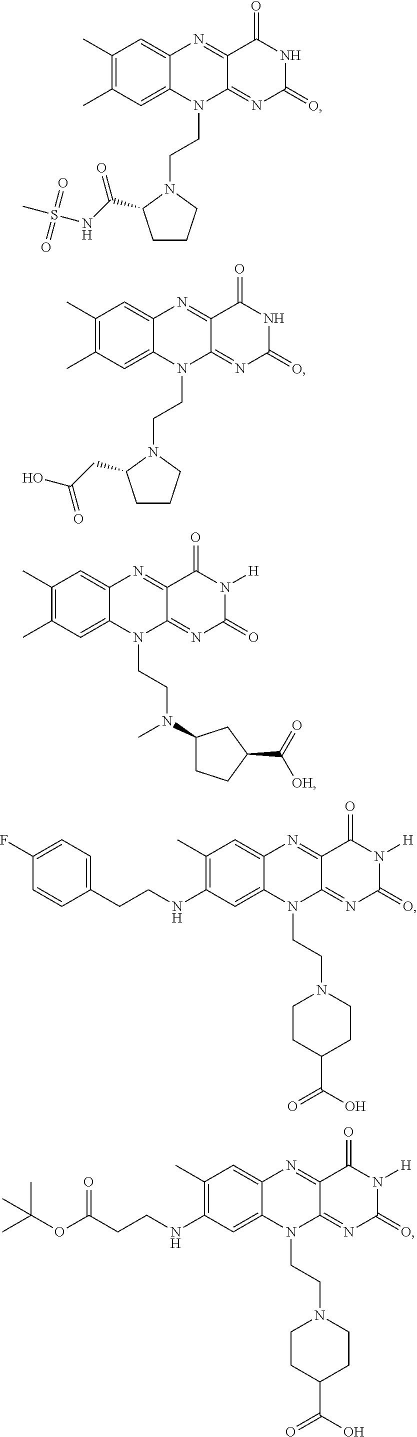

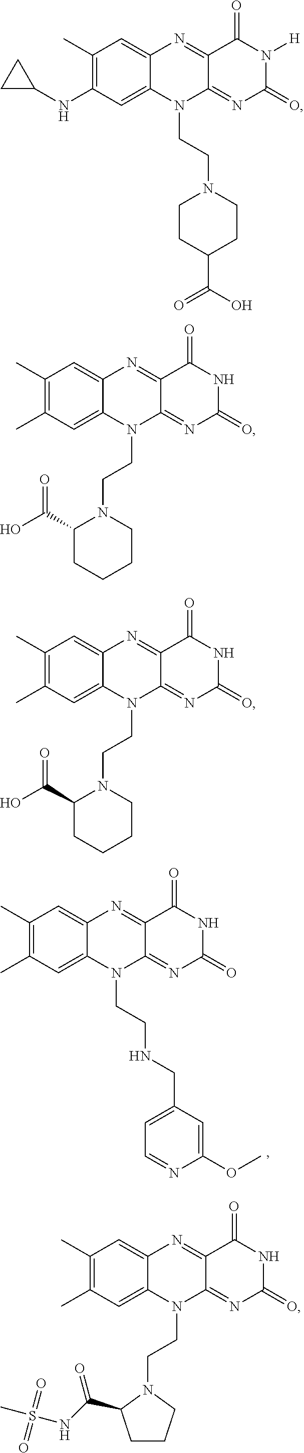

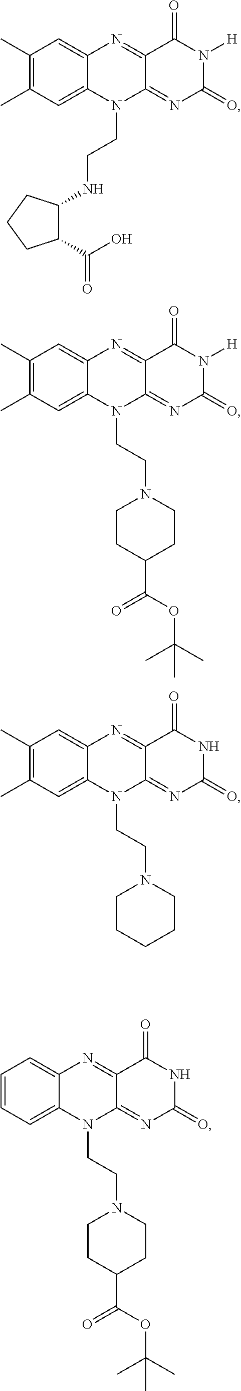

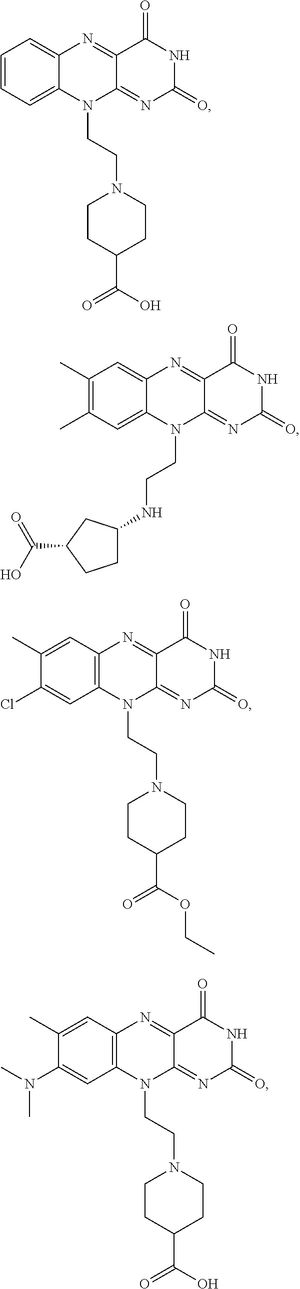

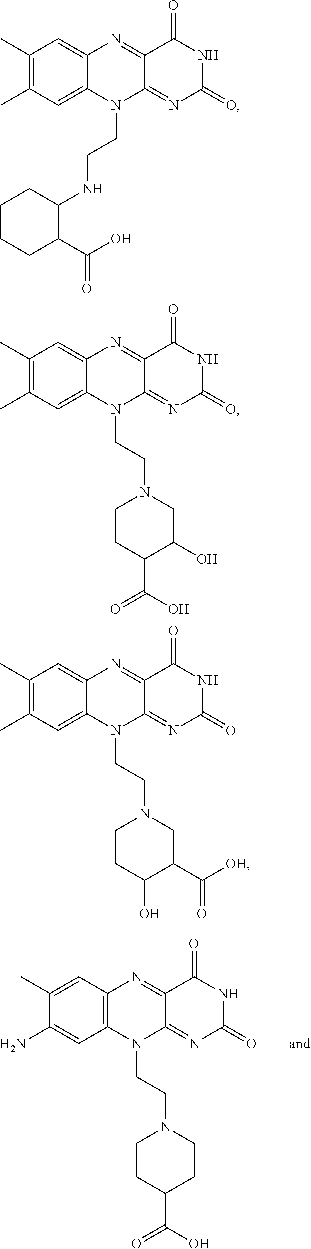

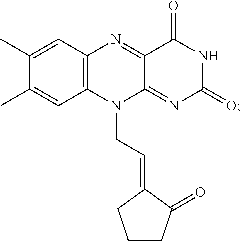

1.40 any of the preceding formulae wherein the Compound of Formula I(A) is selected from any of the following:

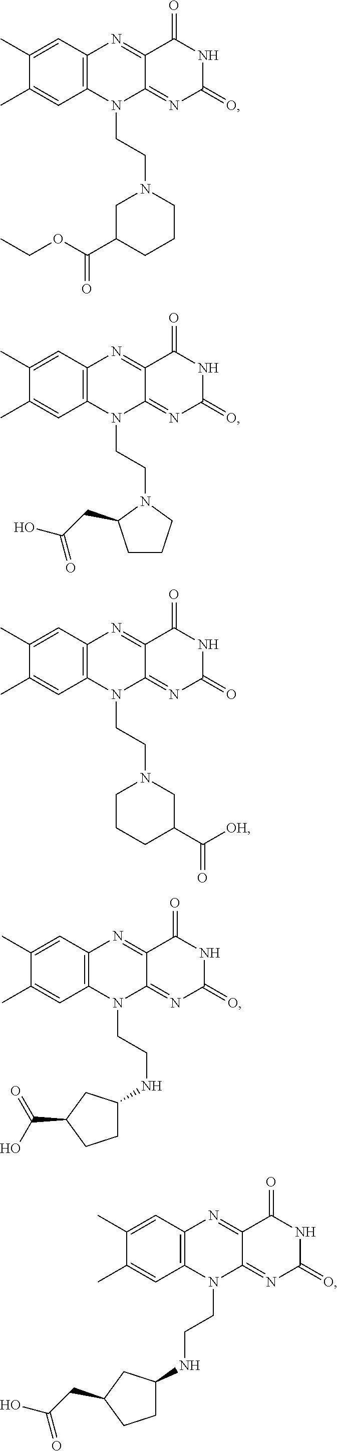

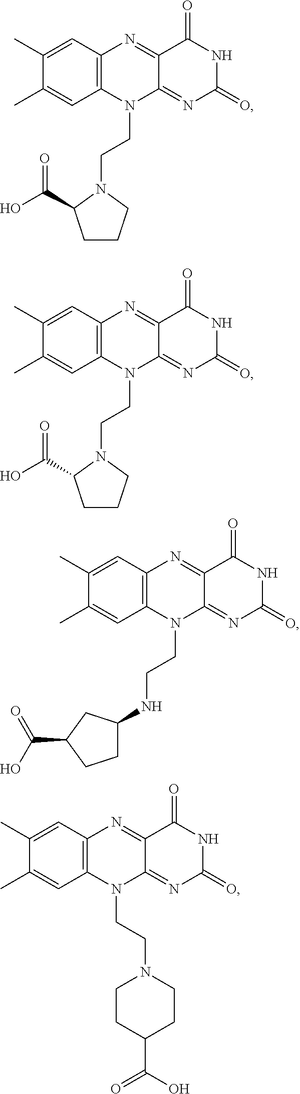

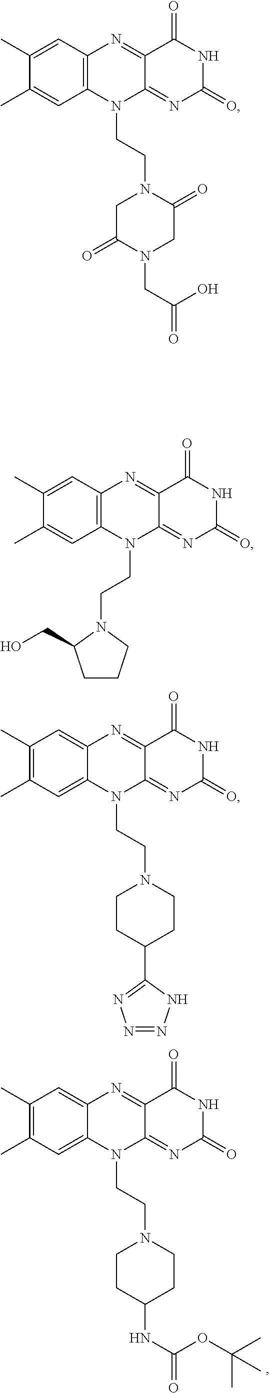

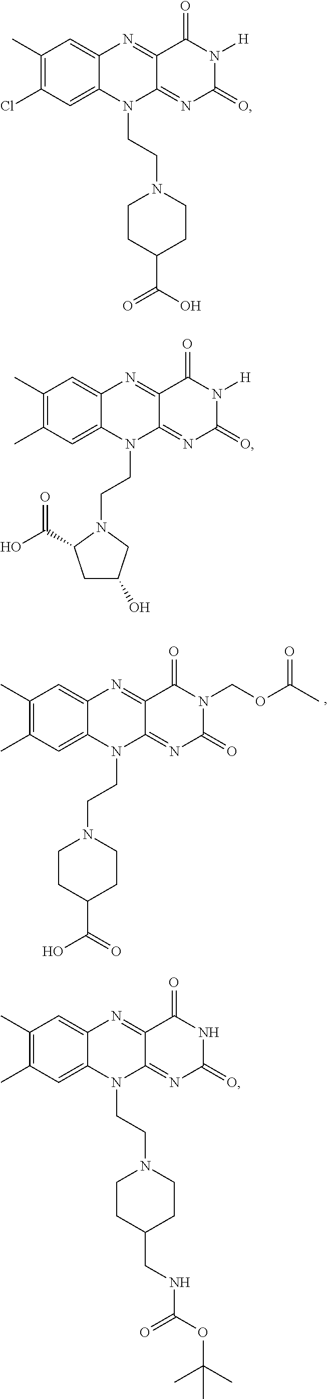

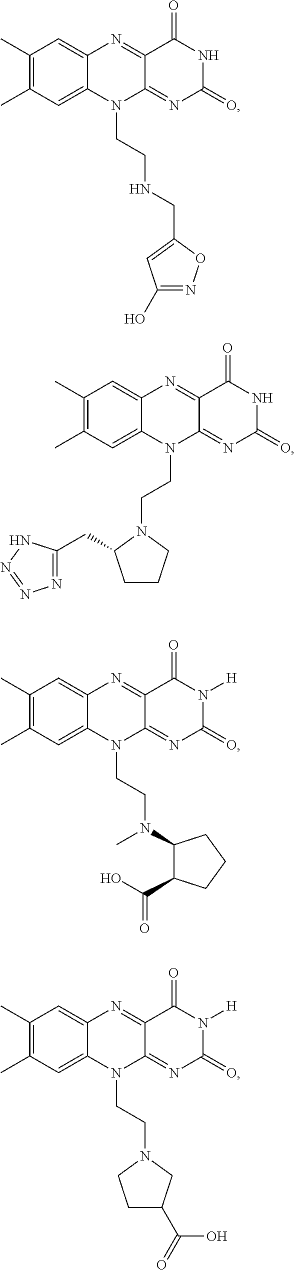

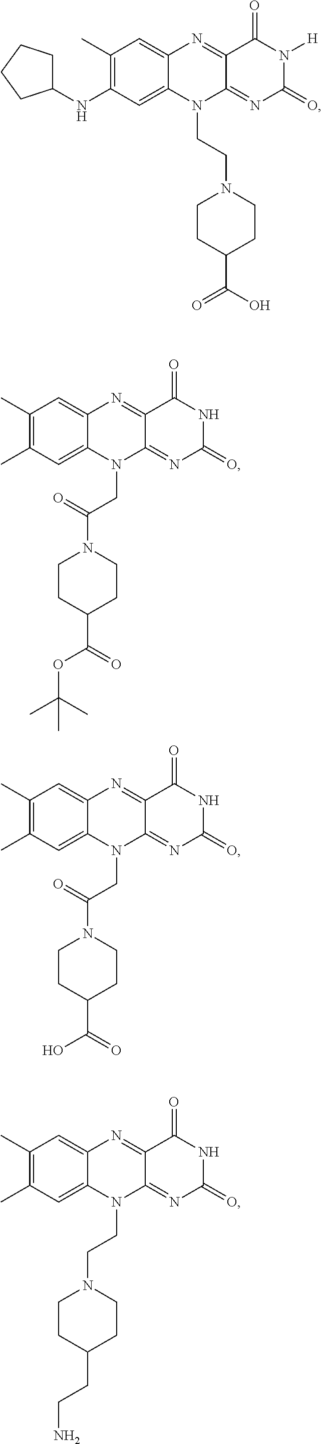

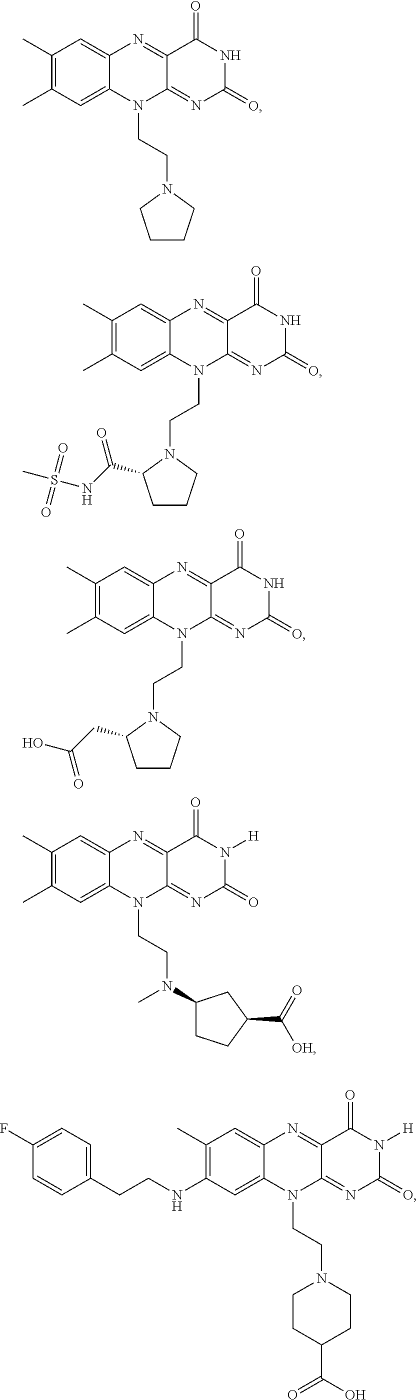

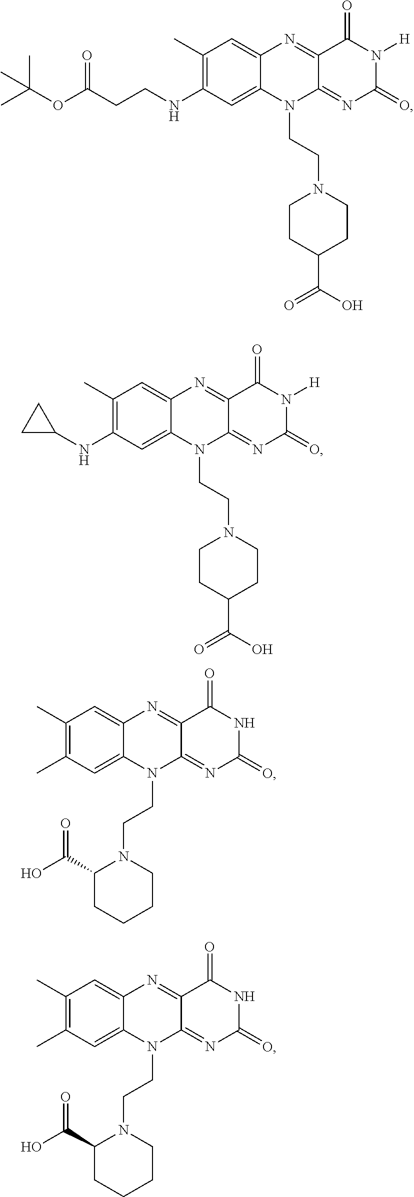

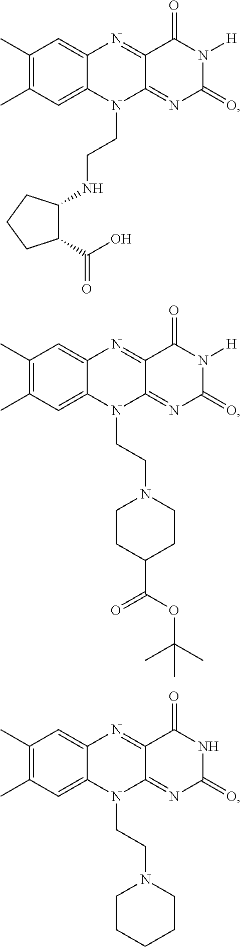

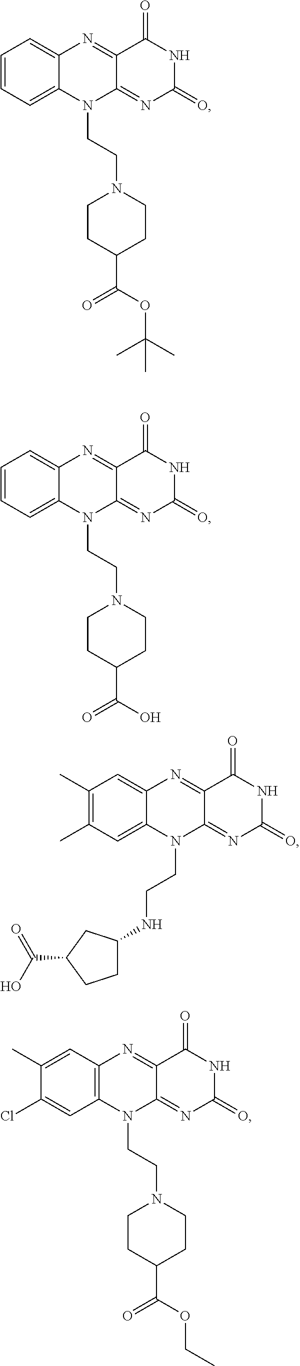

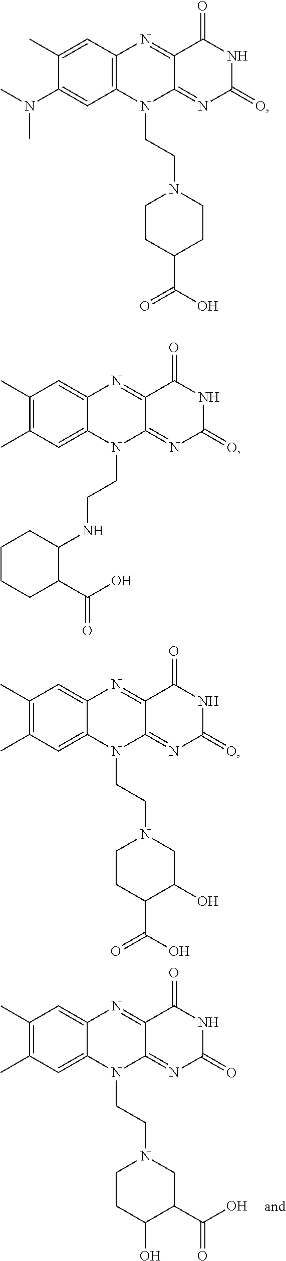

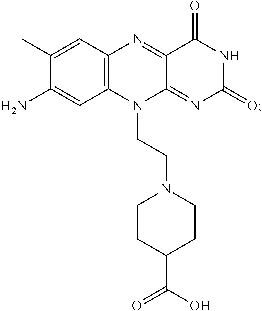

1.41 any of the preceding formulae wherein the Compound of Formula I(A) is selected from any of the following:

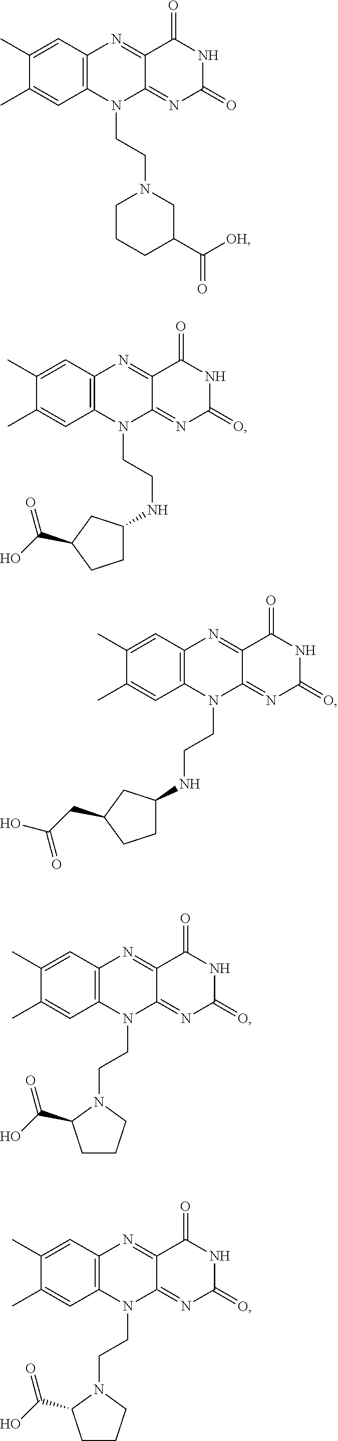

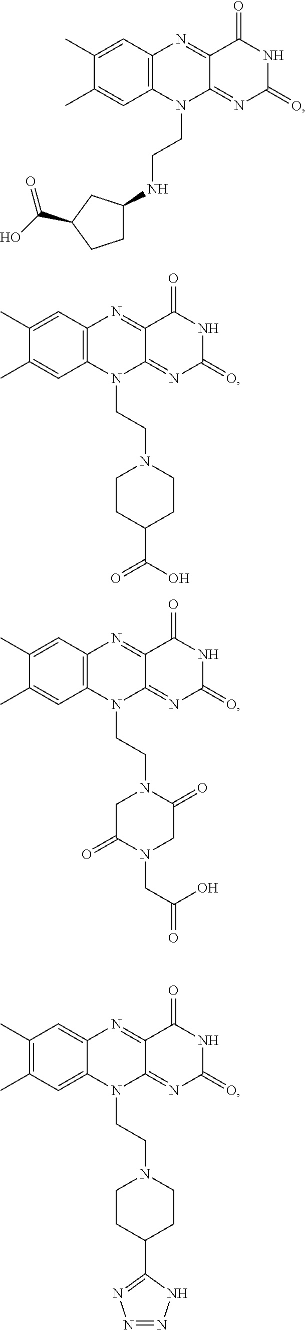

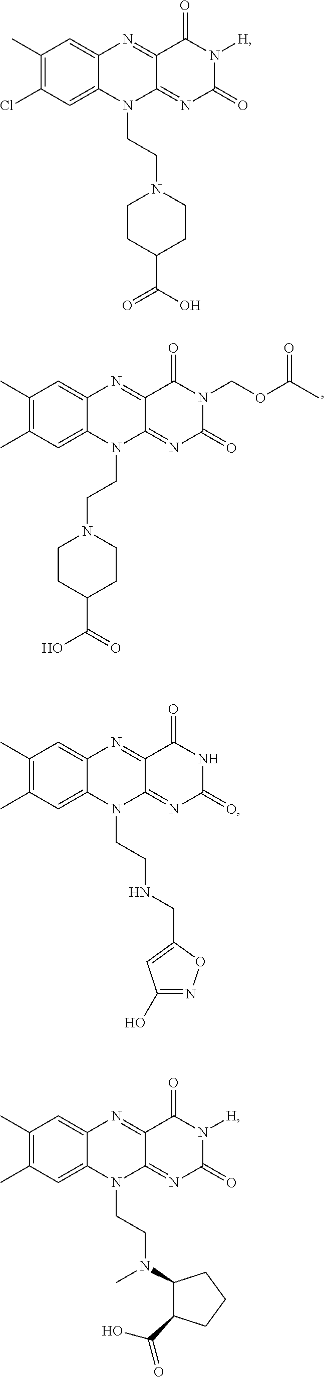

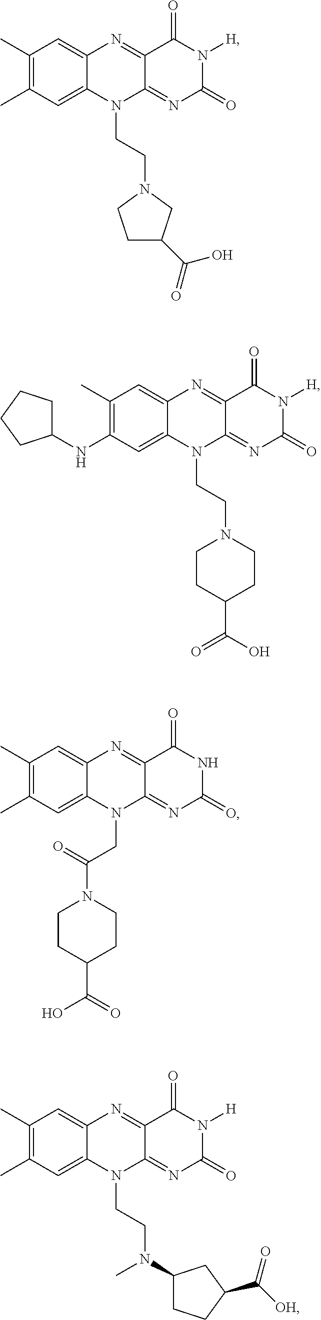

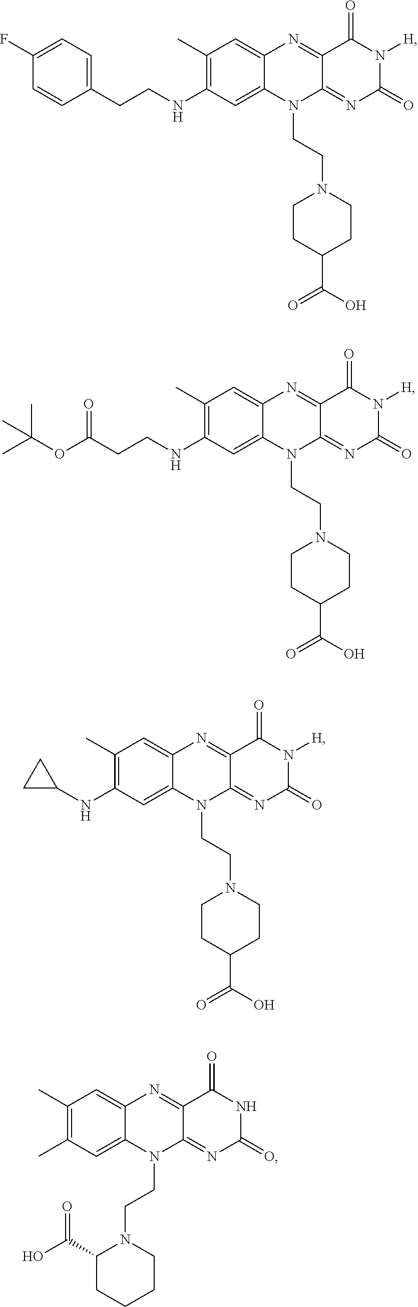

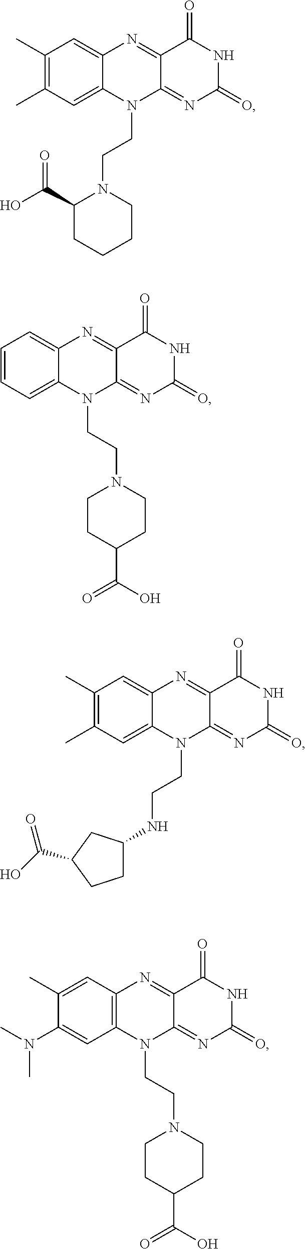

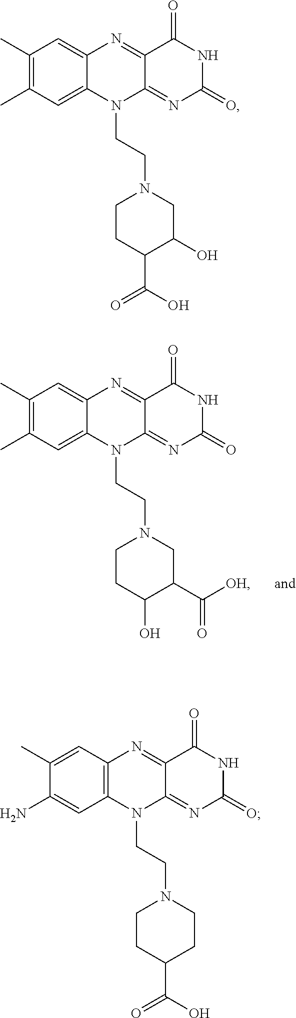

1.42 any of the preceding formulae wherein the Compound of Formula I(A) is selected from any of the following:

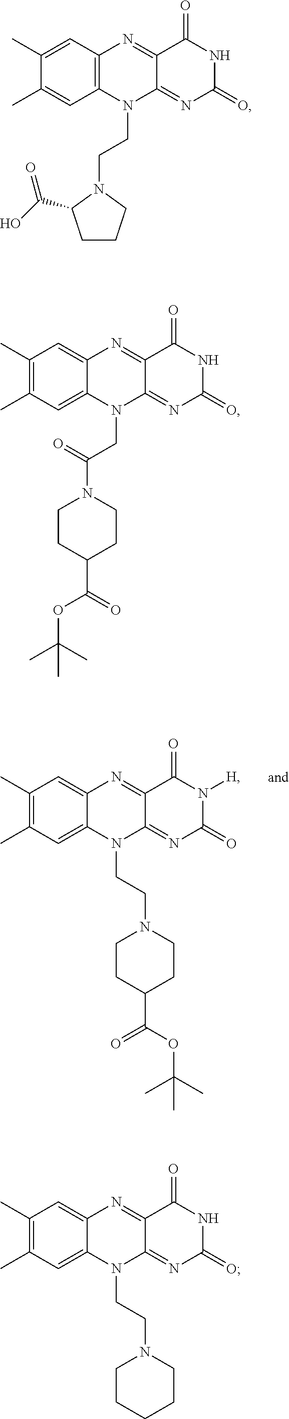

1.43 a Compound of Formula I(A) or any of 1.1-1.38 selected from any of the following:

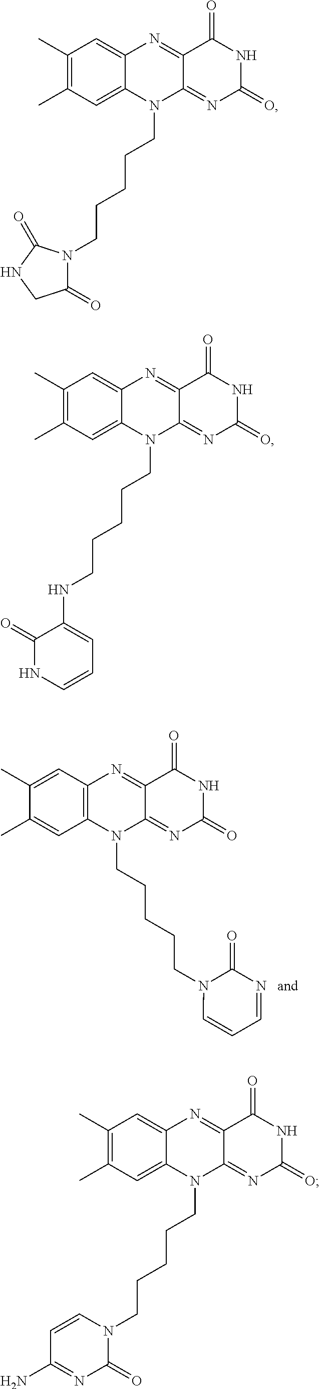

1.44 any of the preceding formulae wherein the Compound of Formula I(A) binds to FMN riboswitch, e.g., with an IC₅₀ of less than or equal to 100 μM, preferably less than 75 μM, more preferably less than 50 μM, still more preferably less than 25 μM, most preferably less than 10 μM in a binding assay, for example, as described in Example 1, and/or the Compound has a Minimum Inhibitory Concentration (MIC) of less than or equal to 128 μg/mL, preferably less than 32 μg/mL, for example, in an assay as described in Example 2,

in free, salt or prodrug form.

In a particular embodiment, the compound of Formula I(A), e.g., any of 1.1-1.44, as hereinbefore described, contains the proviso that when R₂ is chloro, Alk is propylene, X is a single bond and A is pyrrolidin-1-yl, then R₁ is C₁₋₈ alkyl (e.g., methyl) or R₁₀ is —C₁₋₄alkyl-OC(O)CH₃ (e.g., —CH₂OC(O)CH₃), i.e., the compound of Formula I(A) is not 8-chloro-10-(3-pyrrolidin-1-ylpropyl)benzo[g]pteridine-2,4-dione (which compound having such proviso is a Compound of Formula I(A)(i)).

In the third aspect, the invention provides a compound of Formula II(A):

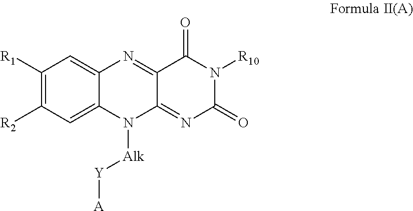

wherein
(i) Alk is C₁₋₆alkylene (e.g., methylene, ethylene, pentylene);
(ii) Y is —N(R₆)—C(O)— or —C(O)—N(R₆)—;
(iii) A is heteroaryl² (e.g., pyrid-3-yl) optionally substituted with one or more —C(O)OR₇, —CH₂C(O)OR₇, —N(R₆)C(O)OR₇, —OH, hydroxy-C₁₋₄alkyl (e.g., hydroxymethyl), —CH₂N(R₆)—C(O)OR₇, heteroaryl² (e.g., 2H-tetrazol-5-yl), heteroaryl²-C₁₋₄alkyl (e.g., 2H-tetrazol-5-yl-methyl), amineC₁₋₄alkyl (e.g., amine-ethyl), C₁₋₄alkoxy (e.g., methoxy), —C(O)N(R₆)—S(O)₂—C₁₋₄alkyl (e.g., —C(O)N(H)S(O)₂—CH₃) or —N(R₈)(R₉);
(iv) R₁ is H or C₁₋₈ alkyl (e.g., methyl);
(v) R₂ is H, halo (e.g., chloro), C₁₋₄alkyl (e.g., methyl), —N(R₄)(R₅);
(vi) R₄ and R₅ are independently selected from H, C₃₋₇ cycloalkyl² (e.g., cyclopropyl or cyclopentyl), —C₁₋₄alkyl (e.g., methyl or ethyl), wherein said alkyl is optionally substituted with one or more groups selected from —OH, —C(O)OR₇, aryl optionally substituted with halo (e.g., 4-fluorophenyl);
(vii) R₆ is H or C₁₋₄alkyl (e.g., methyl);
(viii) R₇ is H, C₁₋₄alkyl (e.g., methyl, ethyl or tert-butyl), —CH₂OC(O)CH₃;
(ix) R₈ and R₉ are independently H, C₁₋₄alkyl;
(x) R₁₀ is H or —C₁₋₄alkyl-OC(O)CH₃ (e.g., —CH₂OC(O)CH₃),
   in free, salt or prodrug form.

In a particular embodiment of the third aspect, the invention provides a compound of Formula II(A) as follows:
2.1 a compound of Formula II(A), wherein Y is —N(R₆)—C(O)—;
2.2 a compound of Formula II(A), wherein Y is —C(O)—N(R₆)—;
2.3 a compound of Formula II(A), 2.1 or 2.2, wherein A is heteroaryl² (e.g., pyrid-3-yl) optionally substituted with one or more —C(O)OR₇, —CH₂C(O)OR₇, —N(R₆)C(O)OR₇, —OH, hydroxy-C₁₋₄alkyl (e.g., hydroxymethyl), —CH₂N(R₆)—C(O)OR₇, heteroaryl² (e.g., 2H-tetrazol-5-yl), heteroaryl²-C₁₋₄alkyl (e.g., 2H-tetrazol-5-yl-methyl), amineC₁₋₄alkyl (e.g., amine-ethyl), C₁₋₄alkoxy (e.g., methoxy), —C(O)N(R₆)—S(O)₂—C₁₋₄alkyl (e.g., —C(O)N(H)S(O)₂—CH₃) or —N(R₈)(R₉);
2.4 a compound of Formula II(A) or any of 2.1-2.3, wherein A is heteroaryl² (e.g., pyrid-3-yl) substituted with one or more —C(O)OR₇, —CH₂C(O)OR₇, —N(R₆)C(O)OR₇, —OH, hydroxy-C₁₋₄alkyl (e.g., hydroxymethyl), —CH₂N(R₆)—C(O)OR₇, heteroaryl² (e.g., 2H-tetrazol-5-yl), heteroaryl²-C₁₋₄alkyl (e.g., 2H-tetrazol-5-yl-methyl), amineC₁₋₄alkyl (e.g., amine-ethyl), C₁₋₄alkoxy (e.g., methoxy), —C(O)N(R₆)—S(O)₂—C₁₋₄alkyl (e.g., —C(O)N(H)S(O)₂—CH₃) or —N(R₈)(R₉);
2.5 a compound of Formula II(A) or any of 2.1-2.3, wherein A is heteroaryl² (e.g., pyrid-3-yl) optionally substituted with one or more C₁₋₄alkoxy (e.g., methoxy);
2.6 a compound of Formula II(A) or any of 2.1-2.3, wherein A is 2-methoxy-pyrid-3-yl;
2.7 a compound of Formula II(A) or any of 2.1-2.6, wherein the substituents Alk, R₁, R₂, R₄, R₅, R₆, R₇, R₈, R₉ and R₁₀ are independently described in 1.1-1.4 and 1.16-1.38;
2.8 a compound of Formula II(A) wherein said compound is:

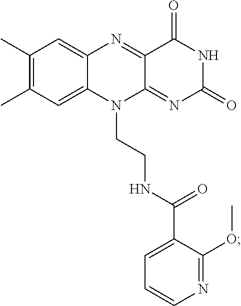

2.9 any of the preceding formulae wherein the Compound of Formula II(A) binds to FMN riboswitch, e.g., with an IC₅₀ of less than or equal to 100 μM, preferably less than 75 μM, more preferably less than 50 μM, still more preferably less than 25 μM, most preferably less than 10 μM in a binding assay, for example, as described in Example 1, and/or the Compound of Formula II has a Minimum Inhibitory Concentration (MIC) of less than or equal to 128 μg/mL, preferably less than 100 μg/mL, more preferably less than 50 μg/mL, still more preferably most preferably less than 35 μg/mL, for example, in an assay as described in Example 2,

in free, salt or prodrug form.

In a fourth aspect, the invention provides a Compound of Formula I(B):

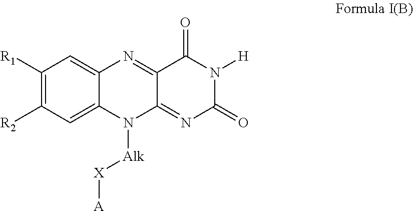

wherein:
(i) Alk is C₁₋₂alkylene (e.g., methylene or ethylene);
(ii) X is —N(R₆)—,
(iii) A is selected from a group consisting of:
   —C₁₋₄alkyl-N(R₁₁)(R₁₂),
   —C₀₋₄alkyl-aryl¹ (e.g., phenyl, naphthyl, benzyl), or —C₀₋₄alkyl-heteroaryl¹ (e.g., isoxazolyl, tetrazolyl, pyridyl, indolyl, 1,2,5-oxadiazolyl, pyrrolyl), wherein the alkyl group of said -alkylaryl¹ and -alkylheteroaryl¹ is optionally substituted with hydroxy or another aryl (e.g., phenyl), and the aryl¹ and heteroaryl¹ group of said -alkylaryl¹ and -alkylheteroaryl¹ are independently substituted with one or more:
      —N(Rₐ)—C(O)—C₁₋₄alkyl (e.g., —NHC(O)CH₃), wherein Rₐ is H or C₁₋₄alkyl,
      —OH,
      Heteroaryl¹ (e.g., imidazolyl),
      heteroC₃₋₈cycloalkyl¹ (e.g., morpholinyl),
      aryl¹ (e.g., phenyl),
      —O-halo-C₁₋₄alkyl (e.g., —OCF₃),
      —NO₂,
      —N(Rₐ)(R_{b}), wherein Rₐ is H or C₁₋₄alkyl and R_{b} is C₁₋₄alkyl,
      —SO₂—C₁₋₄alkyl (e.g., —SO₂—CH₃);
   —C₀₋₄alkyl-pyridyl substituted with one or more hydroxy (e.g., 2-hydroxypyrid-4-ylmethyl or 2-hydroxypyrid-3-yl);
   —C₀₋₄alkyl-benzotriazolyl (e.g., 1H-benzotriazol-5-yl);
   —C₀₋₄alkyl-indolyl (e.g., -indol-5-ylmethyl, indol-2-ylmethyl, indol-3-ylethyl);
   —C₀₋₄alkyl-tetrazolyl (e.g., 1,2,3,5-tetrazol-4-ylethyl);
   —C₀₋₄alkyl-oxadiazolyl (e.g., 1,2,5-oxadiazol-3-yl);
   —C₀₋₄alkyl-benzodioxolyl (e.g., 1,3-benzodioxol-5-ylmethyl);
   —C₀₋₄alkyl-benzimidazolyl optionally substituted with —C₀₋₄alkyl (e.g., 1-methylbenzimidazol-2-ylmethyl, benzimidazol-5-ylmethyl);
   —C₀₋₄alkyl-imidazolyl optionally substituted with C₁₋₄alkyl (e.g., 1-methyl-imidazol-5-ylmethyl);
   —C₀₋₄alkyl-pyrrolyl optionally substituted with —C₀₋₄alkyl (e.g., 1-methylpyrrolidin-2-ylmethyl);
   para-phenylbenzyl;
(iv) R₁ is H or C₁₋₄alkyl (e.g., methyl);
(v) R₂ is selected from a group consisting of H, C₁₋₄alkyl (e.g., methyl) and —O—C₃₋₈cycloalkyl¹ (e.g., —O-cyclopentyl);
(vi) R₆ is H or C₁₋₄alkyl (e.g., methyl);
(vii) R₁₁ and R₁₂ are independently H or C₁₋₄alkyl (e.g., methyl); in free or salt form.

The invention further relates to a Compound of Formula I(B) as described in the following formulae:
3.1 a Compound of formula I(B), wherein Alk is C₁₋₂alkylene (e.g., methylene or ethylene);
3.2 a Compound of Formula I(B) or 3.1, wherein Alk is ethylene;
3.3 a Compound of Formula I(B) or any of 3.1-3.2, wherein A is selected from a group consisting of:
   —C₁₋₄alkyl-N(R₅)(R₆),
   —C₀₋₄alkyl¹-aryl (e.g., phenyl, naphthyl, benzyl), or —C₀₋₄alkyl-heteroaryl¹ (e.g., isoxazolyl, tetrazolyl, pyridyl, indolyl, 1,2,5-oxadiazolyl, pyrrolyl), wherein the alkyl group of said -alkylaryl¹ and -alkylheteroaryl¹ is optionally substituted with hydroxy or another aryl¹ (e.g., phenyl), and the aryl¹ and heteroaryl¹ group of said -alkylaryl¹ and -alkylheteroaryl¹ are independently substituted with one or more:
      —N(Rₐ)—C(O)—C₁₋₄alkyl (e.g., —NHC(O)CH₃), wherein Rₐ is H or C₁₋₄alkyl,
      —OH,
      Heteroaryl¹ (e.g., imidazolyl),
      heteroC₃₋₈cycloalkyl¹ (e.g., morpholinyl),
      aryl (e.g., phenyl),
      —O-halo-C₁₋₄alkyl (e.g., —OCF₃),
      —NO₂,
      —N(Rₐ)(R_{b}), wherein Rₐ is H or C₁₋₄alkyl and R_{b} is C₁₋₄alkyl,
      —SO₂—C₁₋₄alkyl (e.g., —SO₂—CH₃);
   —C₀₋₄alkyl-pyridyl substituted with one or more hydroxy (e.g., 2-hydroxypyrid-4-ylmethyl or 2-hydroxypyrid-3-yl);
   —C₀₋₄alkyl-benzotriazolyl (e.g., 1H-benzotriazol-5-yl);
   —C₀₋₄alkyl-indolyl (e.g., -indol-5-ylmethyl, indol-2-ylmethyl, indol-3-ylethyl);
   —C₀₋₄alkyl-tetrazolyl (e.g., 1,2,3,5-tetrazol-4-ylethyl);
   —C₀₋₄alkyl-oxadiazolyl (e.g., 1,2,5-oxadiazol-3-yl);
   —C₀₋₄alkyl-benzodioxolyl (e.g., 1,3-benzodioxol-5-ylmethyl);
   —C₀₋₄alkyl-benzimidazolyl optionally substituted with —C₀₋₄alkyl (e.g., 1-methylbenzimidazol-2-ylmethyl, benzimidazol-5-ylmethyl);
   —C₀₋₄alkyl-imidazolyl optionally substituted with C₁₋₄alkyl (e.g., 1-methyl-imidazol-5-ylmethyl);
   —C₀₋₄alkyl-pyrrolyl optionally substituted with —C₀₋₄alkyl (e.g., 1-methylpyrrolidin-2-ylmethyl);
   para-phenylbenzyl;
3.4 a Compound of Formula I(B) or any of 3.1-3.2, wherein A is selected from a group consisting of:
   —C₀₋₄alkyl-aryl¹ (e.g., phenyl, naphthyl, benzyl), or —C₀₋₄alkyl-heteroaryl¹ (e.g., isoxazolyl, tetrazolyl, pyridyl, indolyl, 1,2,5-oxadiazolyl, pyrrolyl), wherein the alkyl group of said -alkylaryl¹ and -alkylheteroaryl¹ is optionally substituted with hydroxy or another aryl (e.g., phenyl), and the aryl¹ and heteroaryl¹ group of said -alkylaryl¹ and -alkylheteroaryl¹ are independently substituted with one or more:
      —N(Rₐ)—C(O)—C₁₋₄alkyl (e.g., —NHC(O)CH₃), wherein Rₐ is H or C₁₋₄alkyl,
      —OH,
      heteroaryl¹ (e.g., imidazolyl),
      heteroC₃₋₈cycloalkyl¹ (e.g., morpholinyl),
      aryl¹ (e.g., phenyl),
      —O-halo-C₁₋₄alkyl (e.g., —OCF₃),
      —NO₂,
      —N(Rₐ(R_{b}), wherein Rₐ is H or C₁₋₄alkyl and R_{b} is C₁₋₄alkyl,
      —SO₂—C₁₋₄alkyl (e.g., —SO₂—CH₃);
   —C₀₋₄alkyl-benzotriazolyl (e.g., 1H-benzotriazol-5-yl);
   —C₀₋₄alkyl-indolyl (e.g., -indol-5-ylmethyl, indol-2-ylmethyl, indol-3-ylethyl);
   —C₀₋₄alkyl-tetrazolyl (e.g., 1,2,3,5-tetrazol-4-ylethyl);
   —C₀₋₄alkyl-oxadiazolyl (e.g., 1,2,5-oxadiazol-3-yl);
   —C₀₋₄alkyl-benzodioxolyl (e.g., 1,3-benzodioxol-5-ylmethyl);
   —C₀₋₄alkyl-benzimidazolyl optionally substituted with —C₀₋₄alkyl (e.g., 1-methylbenzimidazol-2-ylmethyl, benzimidazol-5-ylmethyl);
   —C₀₋₄alkyl-pyrrolyl optionally substituted with —C₀₋₄alkyl (e.g., 1-methylpyrrol-2-ylmethyl);
3.5 a Compound of Formula I(B) or any of 3.1-3.2, wherein A is selected from a group consisting of:
   —C₀₋₄alkyl-aryl¹ (e.g., phenyl, naphthyl, benzyl), wherein the alkyl group of said -alkylaryl¹ is optionally substituted with hydroxy or another aryl¹ (e.g., phenyl), and the aryl group of said -alkylaryl¹ is independently substituted with one or more:
      —N(Rₐ)—C(O)—C₁₋₄alkyl (e.g., —NHC(O)CH₃), wherein Rₐ is H or C₁₋₄alkyl,
      —OH,
      heteroaryl¹ (e.g., imidazolyl),
      heteroC₃₋₈cycloalkyl¹ (e.g., morpholinyl),
      aryl¹ (e.g., phenyl),
      —O-halo-C₁₋₄alkyl (e.g., —OCF₃),
      —NO₂,
      —N(Rₐ)(R_{b}), wherein Rₐ is H or C₁₋₄alkyl and R_{b} is C₁₋₄alkyl,
      —SO₂—C₁₋₄alkyl (e.g., —SO₂—CH₃);
   —C₀₋₄alkyl-benzotriazolyl (e.g., 1H-benzotriazol-5-yl);
   —C₀₋₄alkyl-indolyl (e.g., -indol-5-ylmethyl, indol-2-ylmethyl, indol-3-ylethyl);
   —C₀₋₄alkyl-tetrazolyl (e.g., 1,2,3,5-tetrazol-4-ylethyl);
   —C₀₋₄alkyl-oxadiazolyl (e.g., 1,2,5-oxadiazol-3-yl);
   —C₀₋₄alkyl-benzodioxolyl (e.g., 1,3-benzodioxol-5-ylmethyl);
   —C₀₋₄alkyl-benzimidazolyl optionally substituted with —C₀₋₄alkyl (e.g., 1-methylbenzimidazol-2-ylmethyl, benzimidazol-5-ylmethyl);
   —C₀₋₄alkyl-pyrrolyl optionally substituted with —C₀₋₄alkyl (e.g., 1-methylpyrrol-2-ylmethyl);
3.6 Compound of Formula I(B) or any of 3.1-3.2, wherein A is —C₁₋₄alkyl-N(R₅)(R₆) and R₅ and R₆ are independently H or C₁₋₄alkyl (e.g., methyl);
3.7 formula 3.6, wherein A is —CH₃(CH₂)₃—NH₂;
3.8 formula 3.6, wherein A is dimethylaminoethyl (—CH₃CH₂—N(CH₃)₂);
3.9 a Compound of Formula I(B) or any of 3.1-3.2, wherein A is —C₀₋₄alkyl-aryl¹ (e.g., phenyl, naphthyl, benzyl), or —C₀₋₄alkyl-heteroaryl¹ (e.g., isoxazolyl, tetrazolyl, pyridyl, indolyl, 1,2,5-oxadiazolyl, pyrrolyl), wherein the alkyl group of said -alkylaryl¹ and -alkylheteroaryl¹ is optionally substituted with hydroxy or another aryl¹ (e.g., phenyl), and the aryl¹ and heteroaryl¹ group of said -alkylaryl¹ and -alkylheteroaryl¹ are independently substituted with one or more:
   —N(Rₐ)—C(O)—C₁₋₄alkyl (e.g., —NHC(O)CH₃), wherein Rₐ is H or C₁₋₄alkyl,
   —OH,
   heteroaryl¹ (e.g., imidazolyl),
   heteroC₃₋₈cycloalkyl¹ (e.g., morpholinyl),
   aryl¹ (e.g., phenyl),
   —O-halo-C₁₋₄alkyl (e.g., —OCF₃),
   —NO₂,
   —N(Rₐ)(R_{b}), wherein Rₐ is H or C₁₋₄alkyl and R_{b} is C₁₋₄alkyl,
   —SO₂—C₁₋₄alkyl (e.g., —SO₂—CH₃);
3.10 a Compound of Formula I(B) or any of 3.1-3.2, wherein A is —C₀₋₄alkyl-aryl¹ (e.g., phenyl, naphthyl, benzyl), wherein the alkyl group of said -alkylaryl is optionally substituted with hydroxy or another aryl (e.g., phenyl), and the aryl group of said -alkylaryl is substituted with one or more:
   —N(Rₐ)—C(O)—C₁₋₄alkyl (e.g., —NHC(O)CH₃), wherein Rₐ is H or C₁₋₄alkyl,
   —OH,
   Heteroaryl² (e.g., imidazolyl),
   heteroC₃₋₈cycloalkyl² (e.g., morpholinyl),
   aryl² (e.g., phenyl),
   —O-halo-C₁₋₄alkyl (e.g., —OCF₃),
   —NO₂,
   —N(Rₐ)(R_{b}), wherein Rₐ is H or C₁₋₄alkyl and R_{b} is C₁₋₄alkyl,
   —SO₂—C₁₋₄alkyl (e.g., —SO₂—CH₃);
3.11 Formula 3.10, wherein A is —C₀₋₄alkylphenyl (e.g., phenyl or benzyl) wherein the phenyl is substituted with one or more:
   —N(Rₐ)—C(O)—C₁₋₄alkyl (e.g., —NHC(O)CH₃), wherein Rₐ is H or C₁₋₄alkyl,
   —OH,
   heteroaryl¹ (e.g., imidazolyl),
   heteroC₃₋₈cycloalkyl¹ (e.g., morpholinyl),
   aryl¹ (e.g., phenyl),
   —O-halo-C₁₋₄alkyl (e.g., —OCF₃),
   —NO₂,
   —N(Rₐ)(R_{b}), wherein Rₐ is H or C₁₋₄alkyl and R_{b} is C₁₋₄alkyl,
   —SO₂—C₁₋₄alkyl (e.g., —SO₂—CH₃);
3.12 Formula 3.10, wherein A is —C₀₋₄alkylphenyl (e.g., phenyl or benzyl) wherein the phenyl is substituted with —O-halo-C₁₋₄alkyl (e.g., —OCF₃);
3.13 Formula 3.10, wherein A is benzyl substituted with —OCF₃;
3.14 Formula 3.10, wherein A is —C₀₋₄alkylphenyl (e.g., phenyl or benzyl) wherein the phenyl is substituted with —NO₂;
3.15 Formula 3.10, wherein A is m-nitrobenzyl;
3.16 Formula 3.10, wherein A is —C₀₋₄alkylphenyl (e.g., phenyl or benzyl) wherein the phenyl is substituted with another aryl² (e.g., phenyl);
3.17 Formula 3.10, wherein A is p-phenylbenzyl;
3.18 Formula 3.10, wherein A is —C₀₋₄alkylphenyl (e.g., phenyl or benzyl) wherein the phenyl is substituted with —SO₂—C₁₋₄alkyl (e.g., —SO₂—CH₃);
3.19 Formula 3.10, wherein A is 3-methylsulfonylbenzyl;
3.20 a Compound of Formula I(B) or any of 3.1-3.10, wherein A is —C₀₋₄alkyl-heteroaryl¹ (e.g., isoxazolyl, tetrazolyl, pyridyl, indolyl, 1,2,5-oxadiazolyl, pyrrolyl), wherein the alkyl group of said -alkylheteroaryl1 is optionally substituted with hydroxy or another aryl¹ (e.g., phenyl), and the heteroaryl¹ group of said -alkylheteroaryl is substituted with one or more:
   —N(Rₐ)—C(O)—C₁₋₄alkyl (e.g., —NHC(O)CH₃), wherein Rₐ is H or C₁₋₄alkyl,
   —OH,
   Heteroaryl¹ (e.g., imidazolyl),
   heteroC₃₋₈cycloalkyl¹ (e.g., morpholinyl),
   aryl (e.g., phenyl),
   —O-halo-C₁₋₄alkyl (e.g., —OCF₃),
   —NO₂,
   —N(Rₐ)(R_{b}), wherein Rₐ is H or C₁₋₄alkyl and R_{b} is C₁₋₄alkyl,
   —SO₂—C₁₋₄alkyl (e.g., —SO₂—CH₃);
3.21 Formula 3.20, wherein A is —C₀₋₄alkyl-1,2,5-oxadiazolyl substituted with one or more of the substituents as set forth above in 3.20;
3.22 Formula 3.20, wherein A is 3-methyl-1,2,5-oxadiazol-4-ylmethyl;
3.23 Formula 3.20, wherein A is —C₀₋₄alkyl-pyridyl substituted with one or more hydroxy (e.g., 2-hydroxypyrid-4-ylmethyl or 2-hydroxypyrid-3-yl);
3.24 Formula 3.23, wherein A is 2-hydroxypyrid-4-ylmethyl;
3.25 a Compound of Formula I(B) or any of 3.1-3.10, wherein A is —C₀₋₄alkyl-benzotriazolyl (e.g., 1H-benzotriazol-5-yl);
3.26 a Compound of Formula I(B) or any of 3.1-3.10, wherein A is —C₀₋₄alkyl-indolyl (e.g., -indol-5-ylmethyl, indol-2-ylmethyl, indol-3-ylethyl);
3.27 a Compound of Formula I(B) or any of 3.1-3.10, wherein A is —C₀₋₄alkyl-tetrazolyl (e.g., 1,2,3,5-tetrazol-4-ylethyl);
3.28 a Compound of Formula I(B) or any of 3.1-3.10, wherein A is —C₀₋₄alkyl-oxadiazolyl (e.g., 1,2,5-oxadiazol-3-yl);
3.29 a Compound of Formula I(B) or any of 3.1-3.10, wherein A is —C₀₋₄alkyl-benzodioxolyl (e.g., 1,3-benzodioxol-5-ylmethyl);
3.30 a Compound of Formula I(B) or any of 3.1-3.10, wherein A is —C₀₋₄alkyl-benzimidazolyl optionally substituted with —C₀₋₄alkyl (e.g., 1-methylbenzimidazol-2-ylmethyl, benzimidazol-5-ylmethyl);
3.31 a Compound of Formula I(B) or any of 3.1-3.10, wherein A is —C₀₋₄alkyl-imidazolyl optionally substituted with C₁₋₄alkyl (e.g., 1-methyl-imidazol-5-ylmethyl);
3.32 a Compound of Formula I(B) or any of 3.1-3.10, wherein A is —C₀₋₄alkyl-pyrrolyl optionally substituted with —C₀₋₄alkyl (e.g., 1-methylpyrrol-2-ylmethyl);
3.33 a Compound of Formula I(B) or any of 3.1-3.32, wherein R₁ is H or C₁₋₄alkyl (e.g., methyl);
3.34 a Compound of Formula I(B) or any of 3.1-3.32, wherein R₁ is methyl;
3.35 a Compound of Formula I(B) or any of 3.1-3.34, wherein R₂ is selected from a group consisting of H, C₁₋₄alkyl (e.g., methyl) and —O—C₃₋₈cycloalkyl¹ (e.g., —O-cyclopentyl);
3.36 formula 3.35, wherein R₂ is H;
3.37 formula 3.35, wherein R₂ is C₁₋₄alkyl (e.g., methyl);
3.38 formula 3.35, wherein R₂ is −0-C₃₋₈cycloalkyl¹ (e.g., —O-cyclopentyl);
3.39 formula 3.35, wherein R₁ is C₁₋₄alkyl (e.g., methyl) and R₂ is —O—C₃₋₈cycloalkyl¹ (e.g., —O-cyclopentyl);
3.40 formula 3.35, wherein R₁ and R₂ are C₁₋₄alkyl (e.g., methyl);
3.41 formula 3.35, wherein R₁ and R₂ are methyl;
3.42 a Compound of Formula I(B) or any of 3.1-3.41 wherein R₄ is H or C₁₋₄alkyl (e.g., methyl);
3.43 formula 3.42, wherein R₄ is H,
3.44 formula 3.42, wherein R₄ is C₁₋₄alkyl (e.g., methyl);
3.45 a Compound of Formula I(B) or any of 3.1-3.44, wherein R₅ and R₆ are independently H or C₁₋₄alkyl (e.g., methyl);
3.46 a Compound of Formula I(B) or any of 3.1-3.45, wherein R₅ and R₆ are H,
3.47 a Compound of Formula I(B) or any of 3.1-3.45, wherein R₅ and R₆ are C₁₋₄alkyl (e.g., methyl);
3.48 a Compound of Formula I(B) or any of 3.1-3.45, wherein R₅ is H and R₆ is C₁₋₄alkyl (e.g., methyl);
3.49 a Compound of Formula I(B) or any of 3.1-3.10 or 3.33-3.48, wherein A is selected from a group consisting of:
   benzyl meta or para substituted with —O-halo-C₁₋₄alkyl (e.g., —OCF₃);
   indol-3-ylethyl;
   1,3-benzodioxol-5-ylmethyl;
   1-methylpyrrolidin-2-ylmethyl;
   para-phenylbenzyl;
3.50 a Compound of Formula I(B) or any of 3.1-3.10 or 3.33-3.48, wherein A is selected from a group consisting of:
   1,3-benzodioxol-5-ylmethyl,
   benzyl ortho or meta-substituted with —OCF₃,
   benzyl substituted with —NO₂,
   para-phenylbenzyl;
3.51 any of the preceding formulae wherein the Compound of Formula I(B) is selected from any of the following:

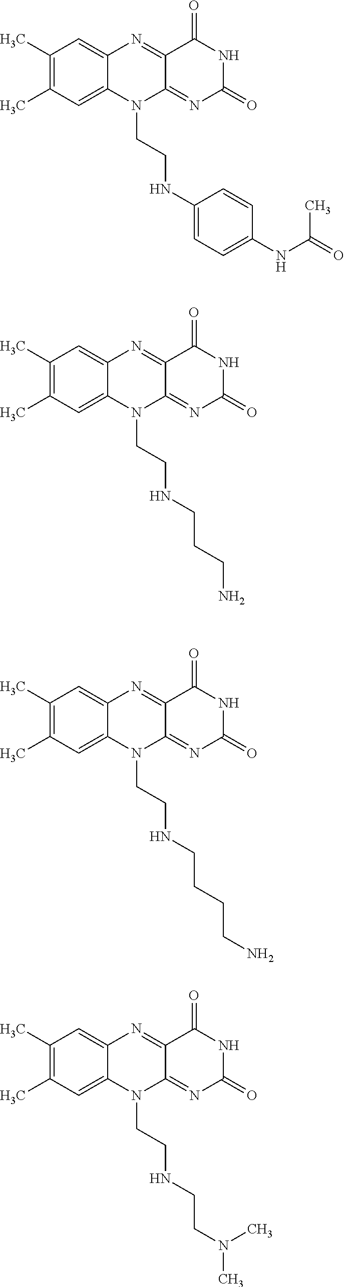

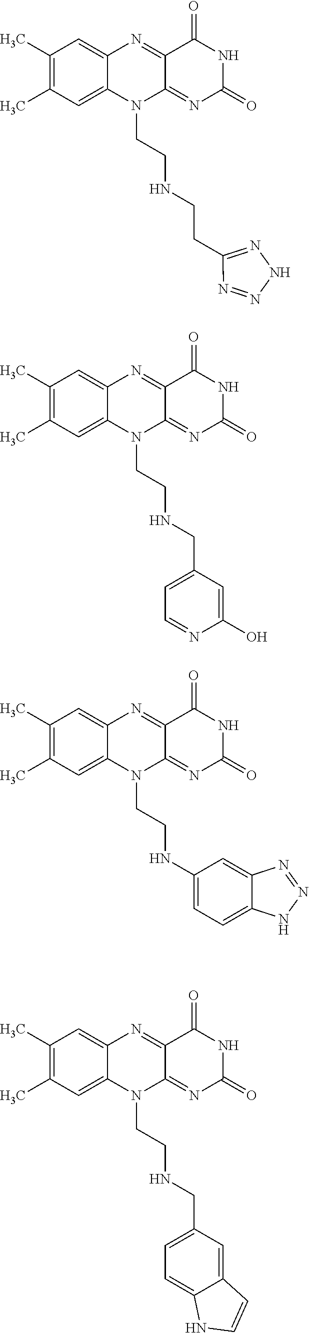

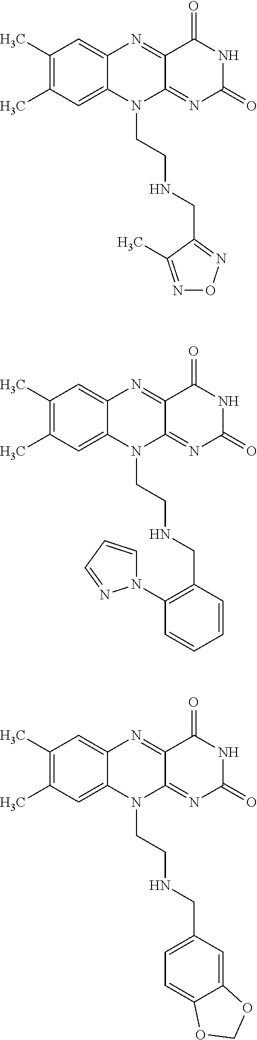

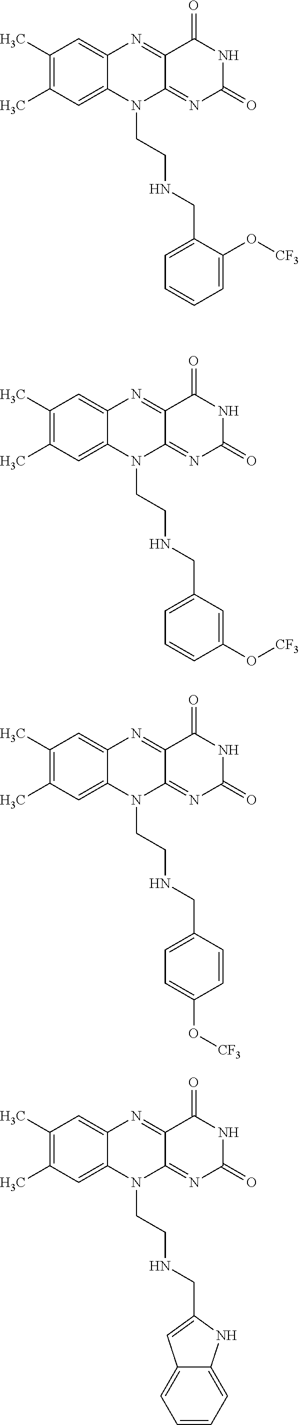

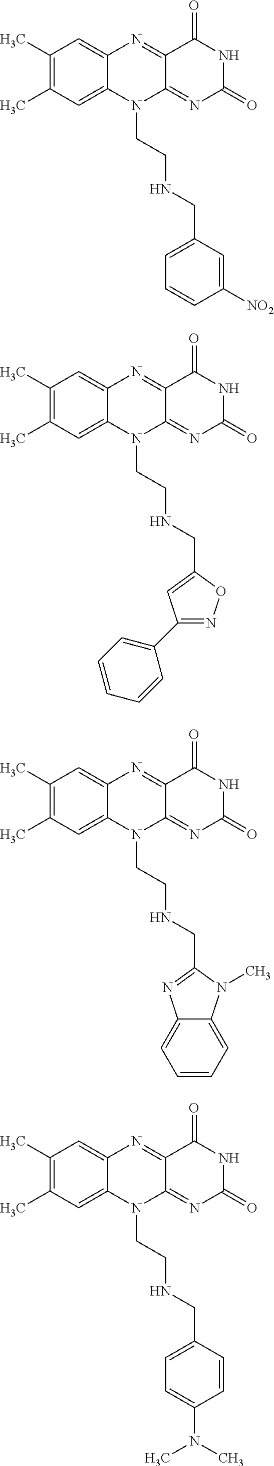

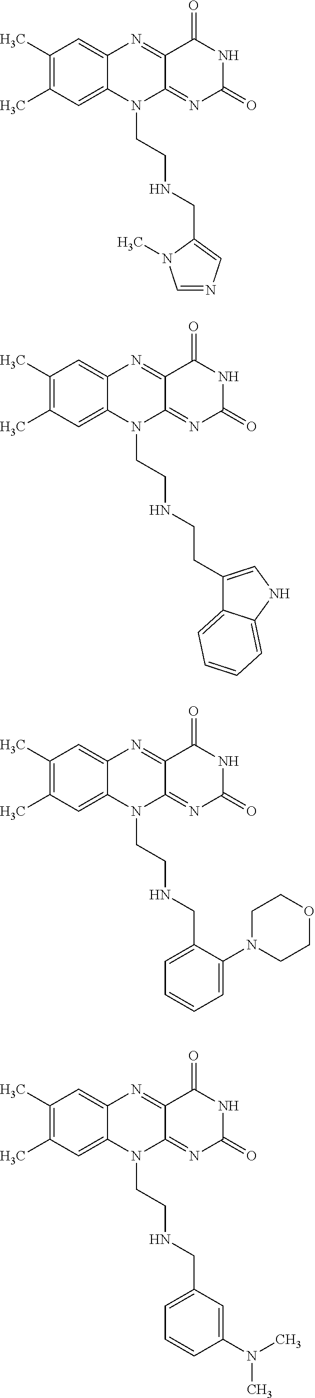

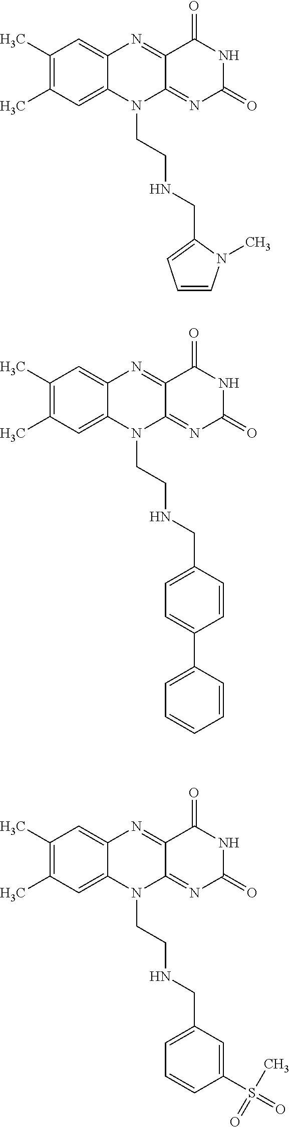

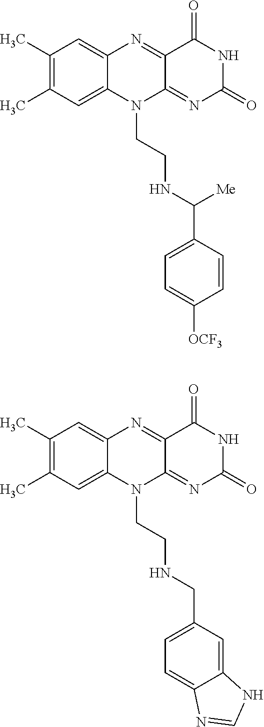

3.52 any of the preceding formulae wherein the Compound of Formula I(B) is selected from any of the following:

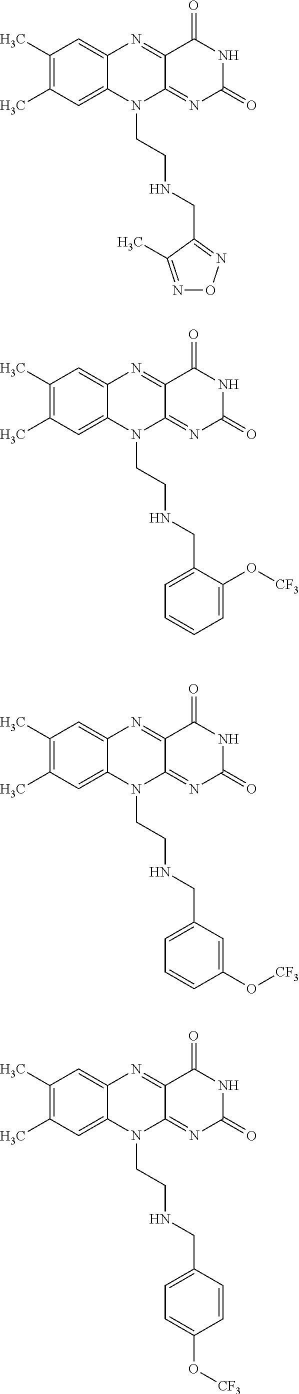

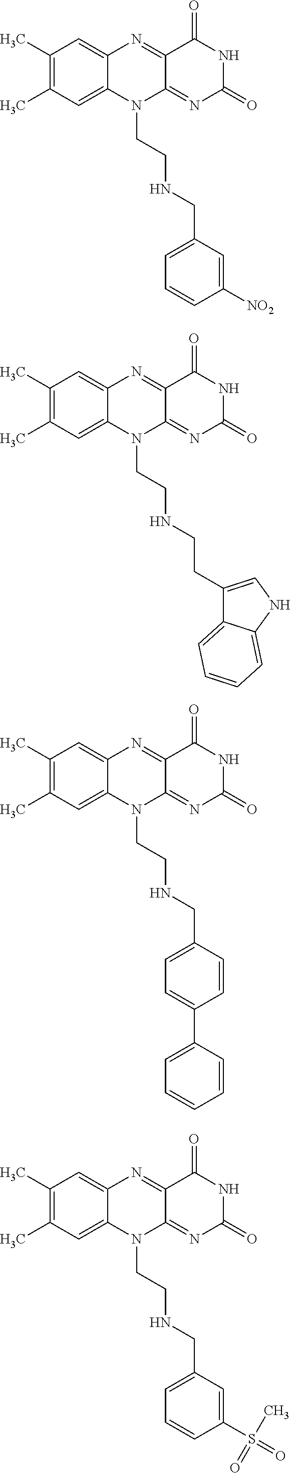

3.53 any of formulae 3.1-3.51, wherein the Compound of Formula I(B) is selected from any of the following:

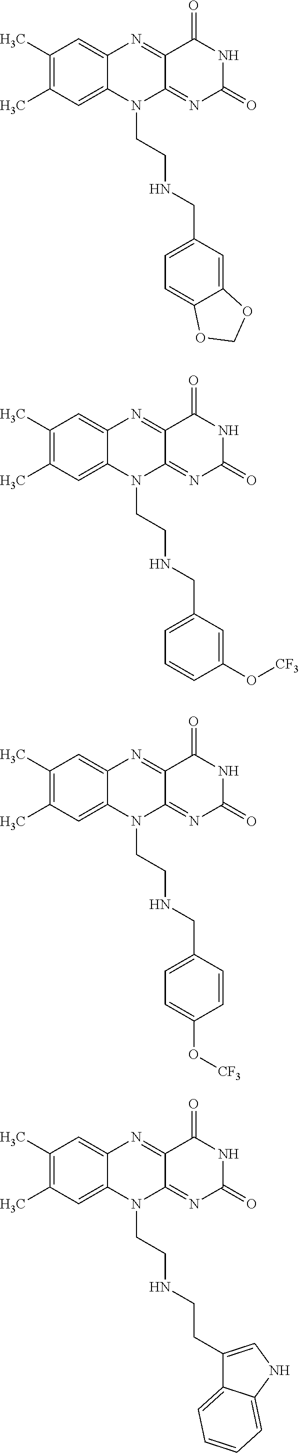

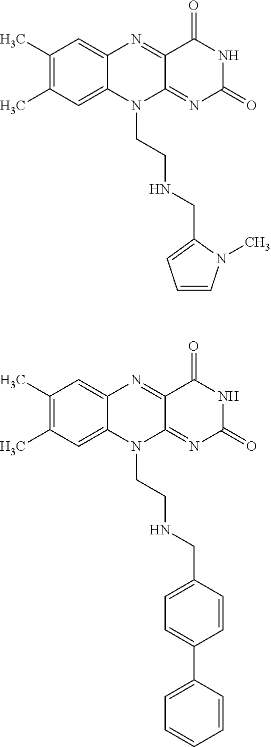

3.54 any of formulae 3.1-3.51, wherein the Compound of Formula I(B) is selected from any of the following:

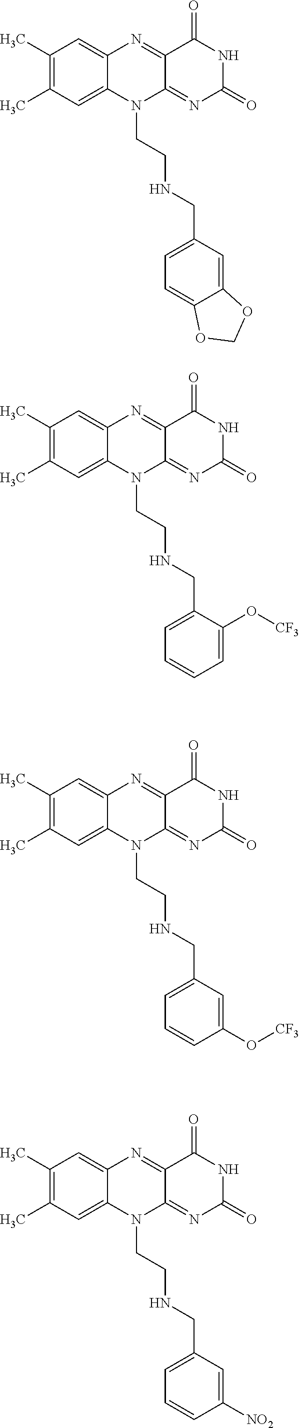

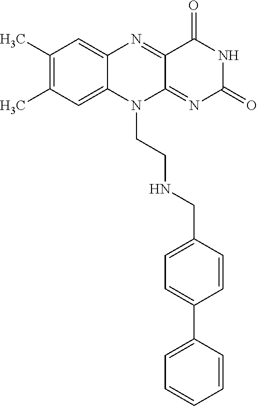

3.55 any of the preceding formulae wherein the Compound binds to FMN and/or CD3299 riboswitch, e.g., greater than 20%, preferably greater than 30%, more preferably greater than 40%, still more preferably greater than 50% in an assay, for example, as described in Example 1, and/or has a Minimum Inhibitory Concentration (MIC) of less than or equal to 64 μg/mL, more preferably less than or equal to 32 μg/mL, still more preferably less than or equal to 16 μg/mL, for example, in an assay as described in Example 2,

in free or salt form.

In the fifth aspect, the invention provides a compound of Formula II(B):

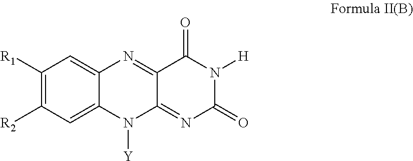

wherein:
(i) R₁ is H or C₁₋₄alkyl (e.g., methyl)
(ii) R₂ is selected from a group consisting of H, C₁₋₄alkyl (e.g., methyl) and —O—C₃₋₈cycloalkyl¹ (e.g., —O-cyclopentyl);
(iii) Y is selected from a group consisting of:

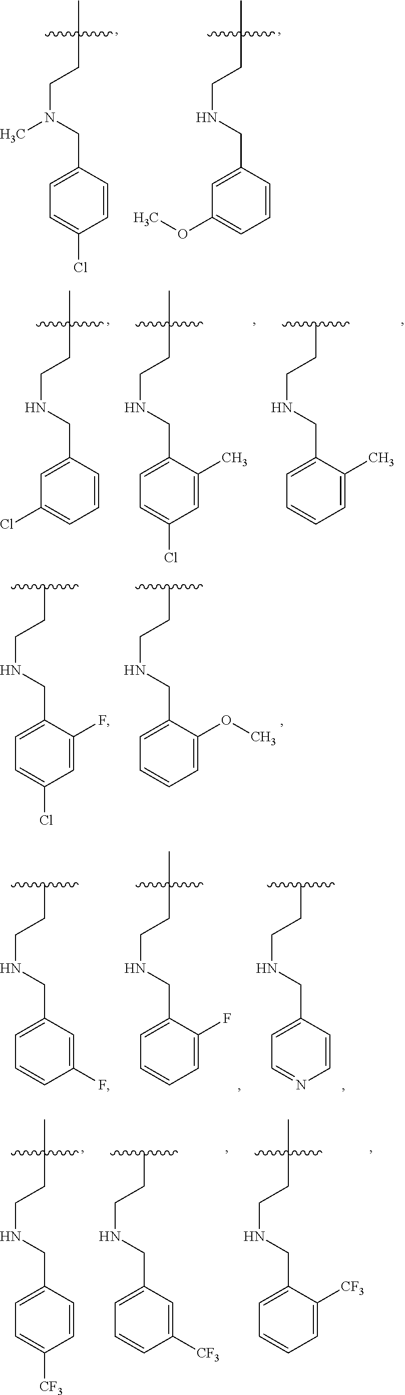

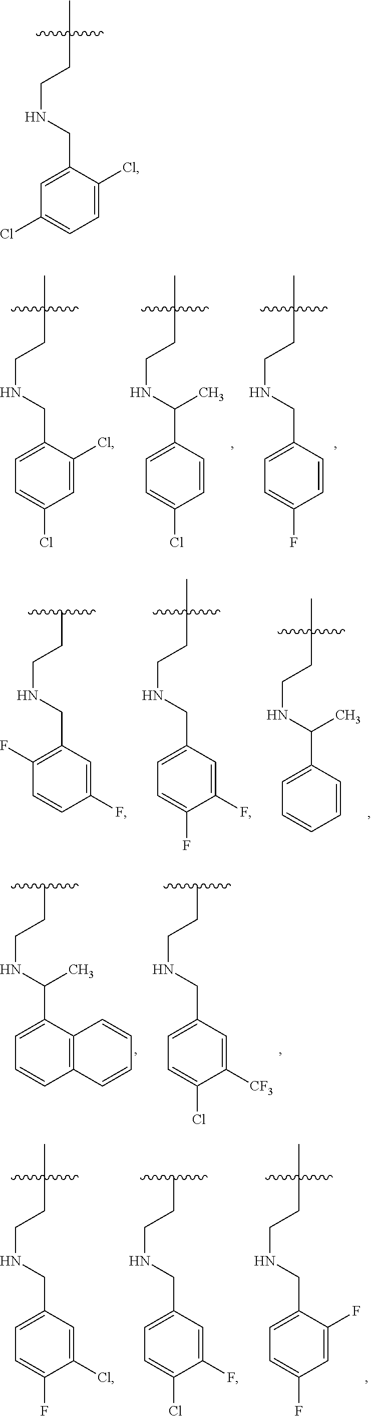

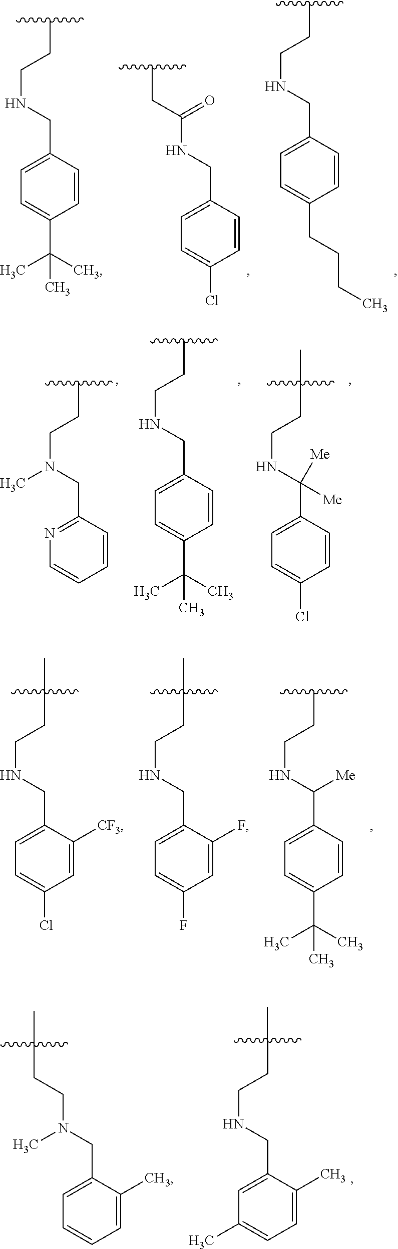

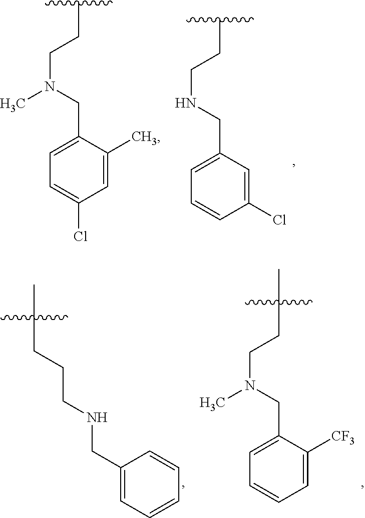

in free or salt form.

In a further embodiment of the fifth aspect, the invention provides a Compound of Formula II(B) selected from any of the following:

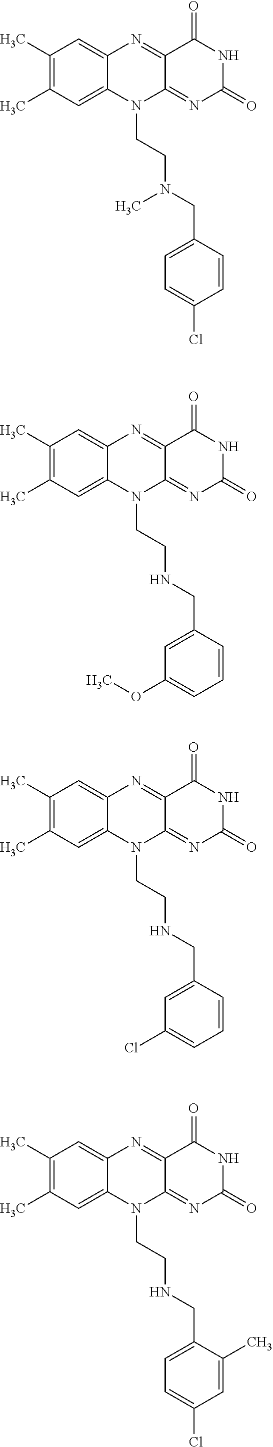

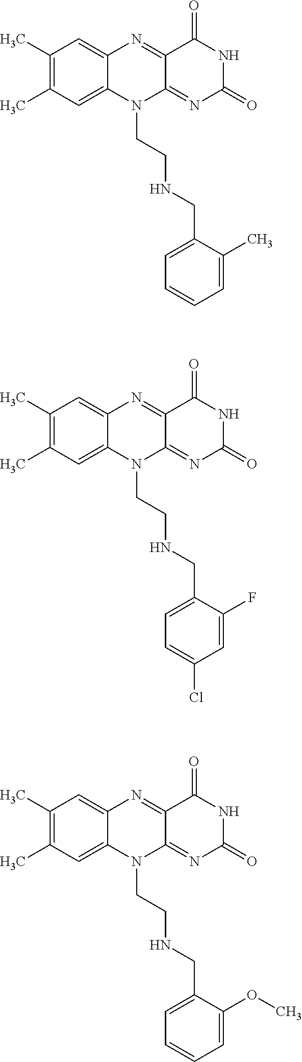

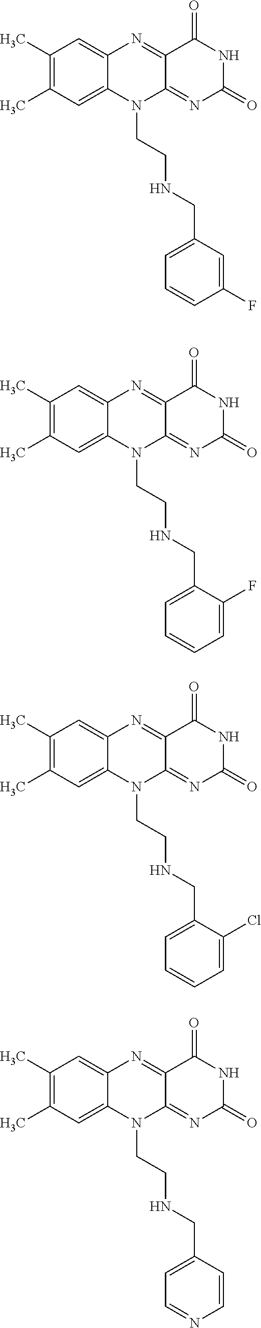

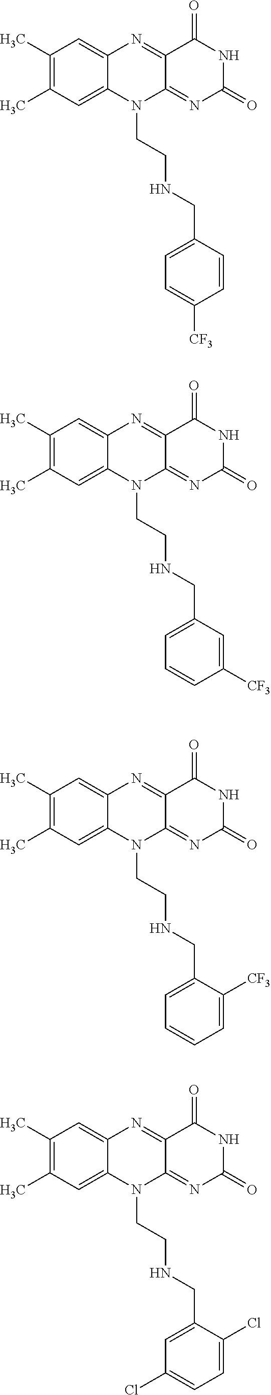

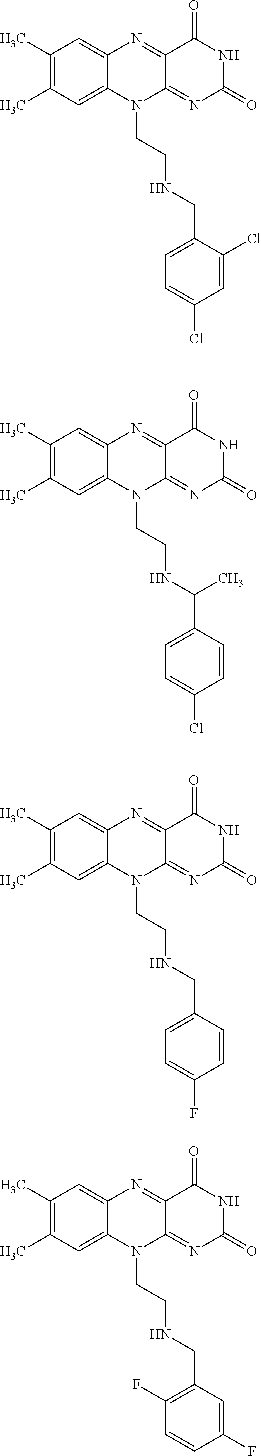

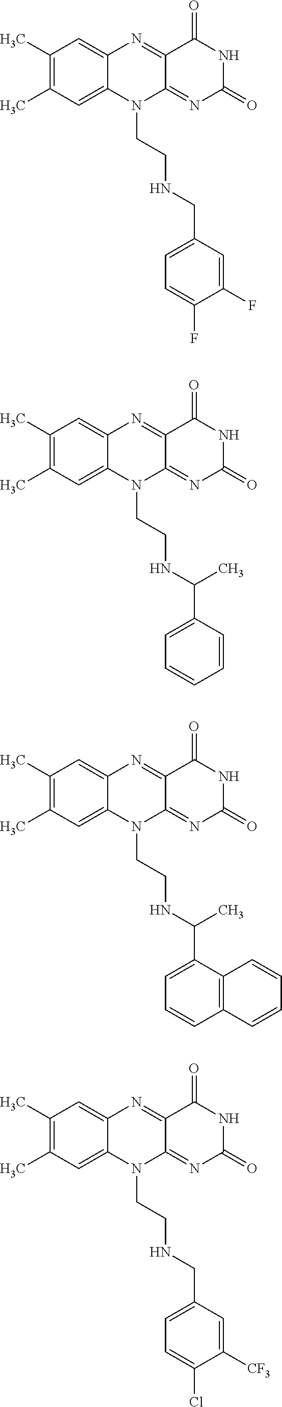

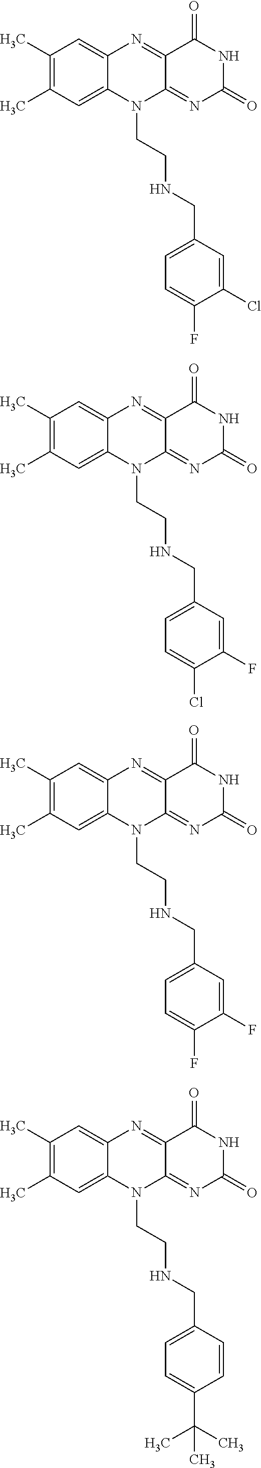

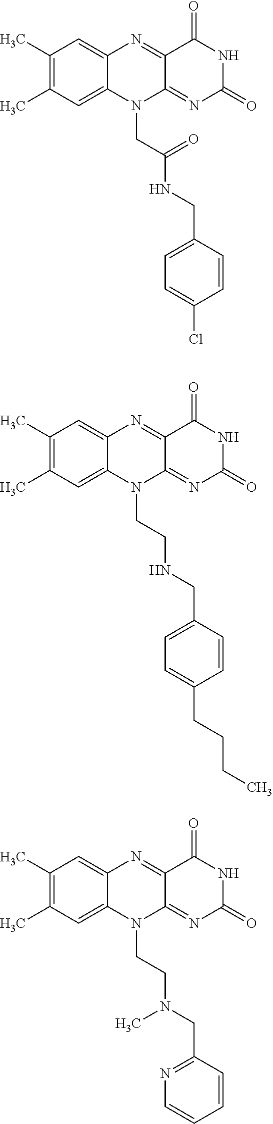

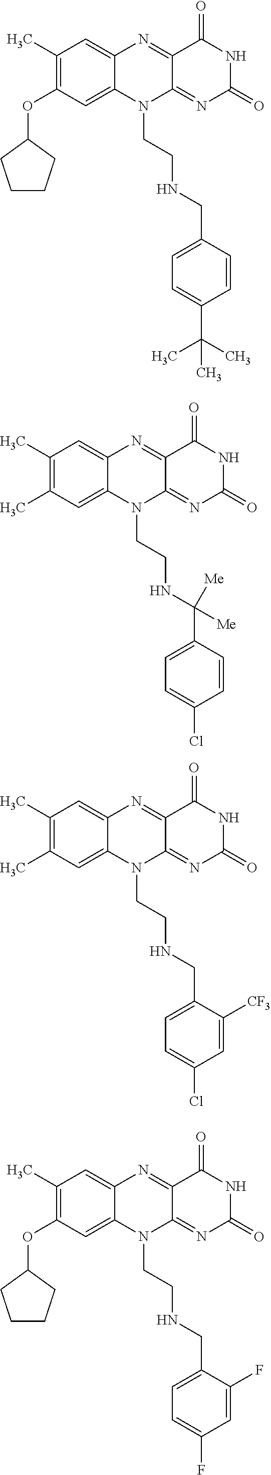

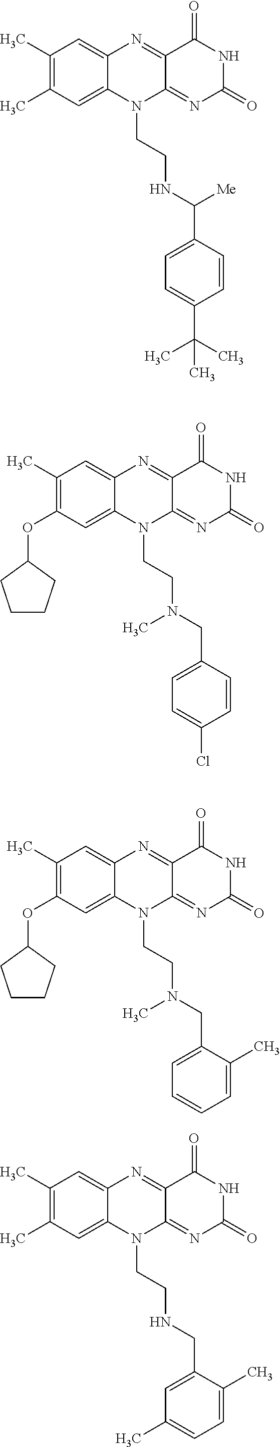

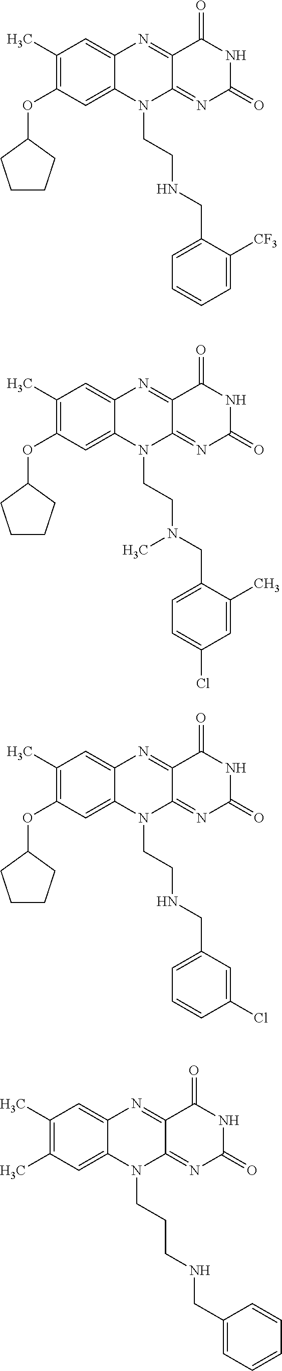

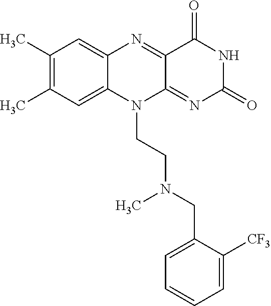

in free or salt form.

In a still further embodiment of the fifth aspect, the invention provides a Compound of Formula II(B) selected from any of the following:

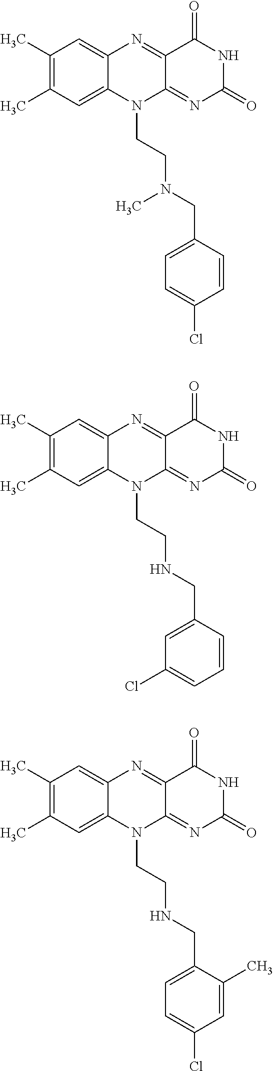

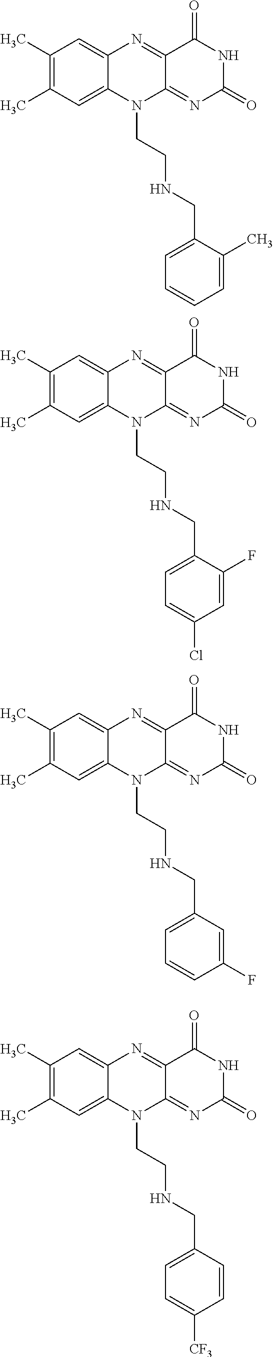

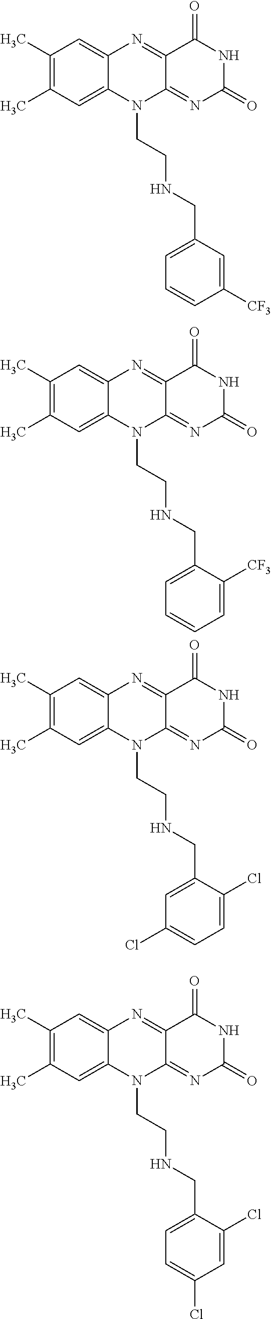

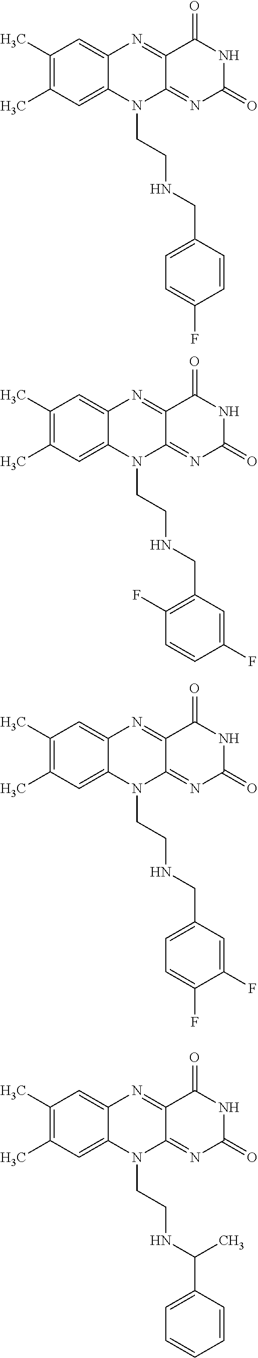

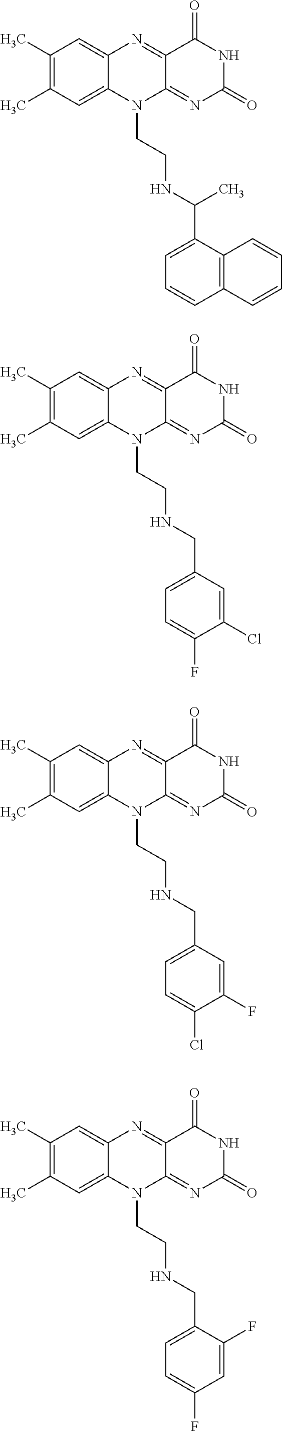

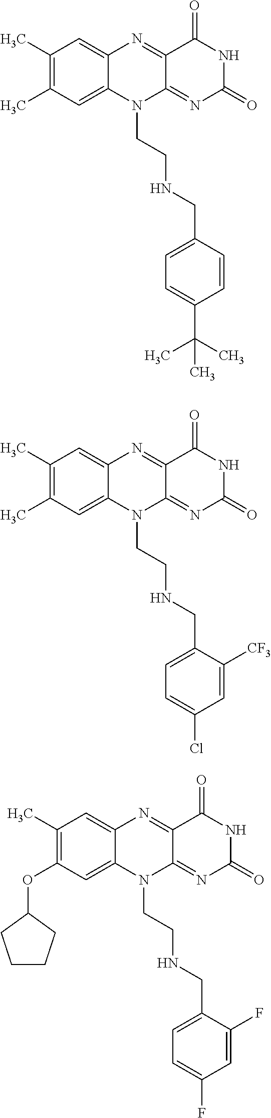

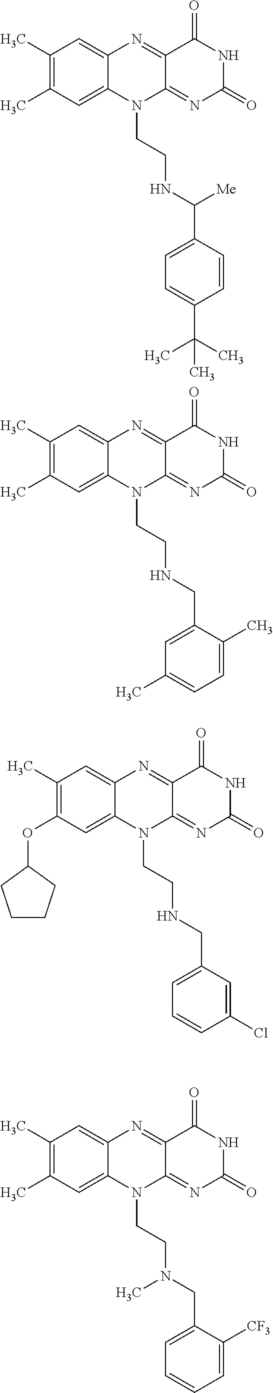

in free or salt form.

In a further embodiment of the fifth aspect, the invention provides a Compound of Formula II(B) selected from any of the following:

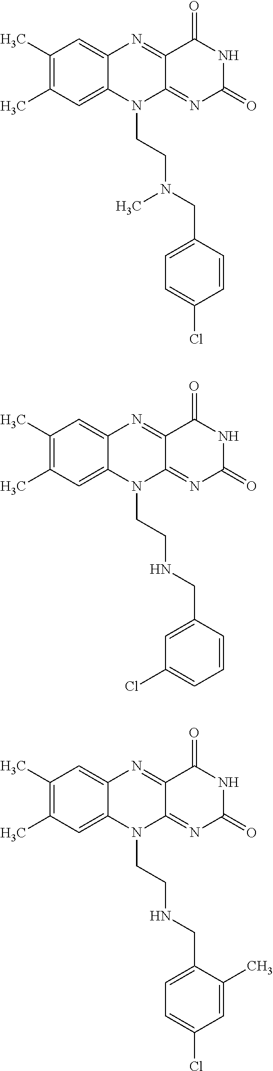

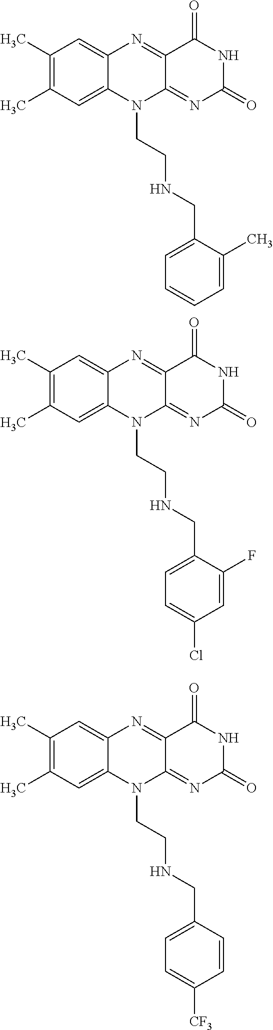

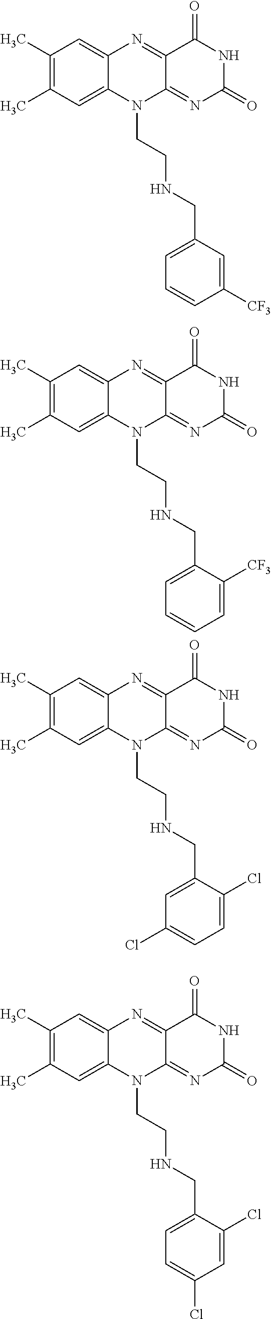

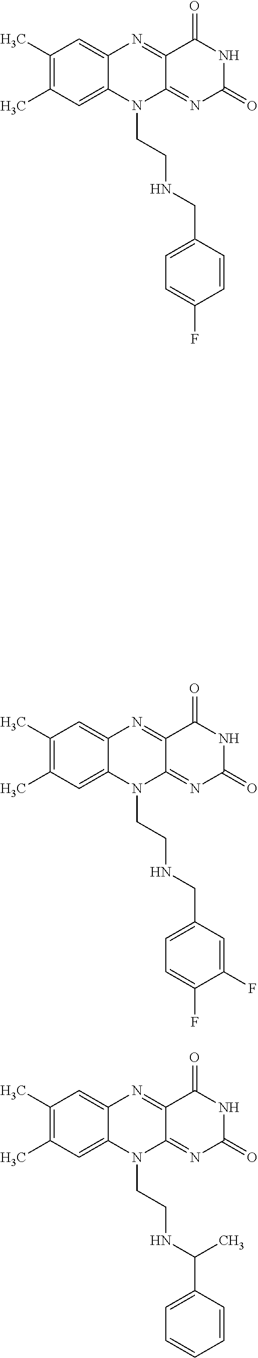

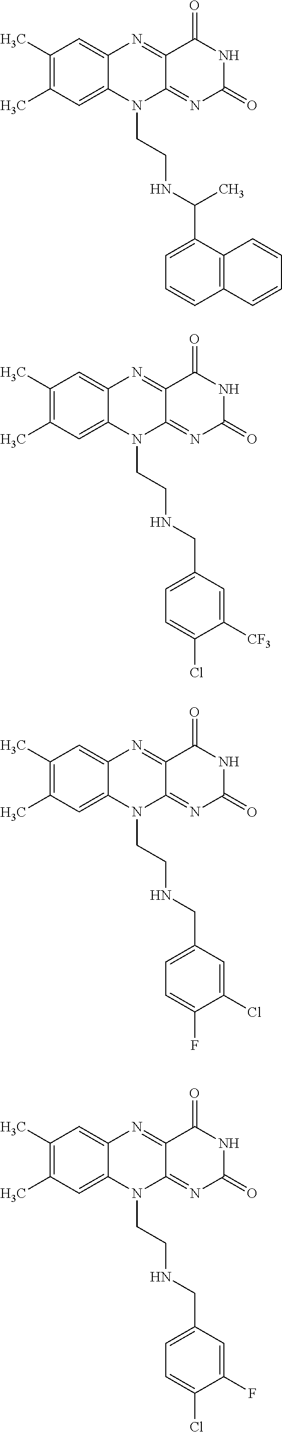

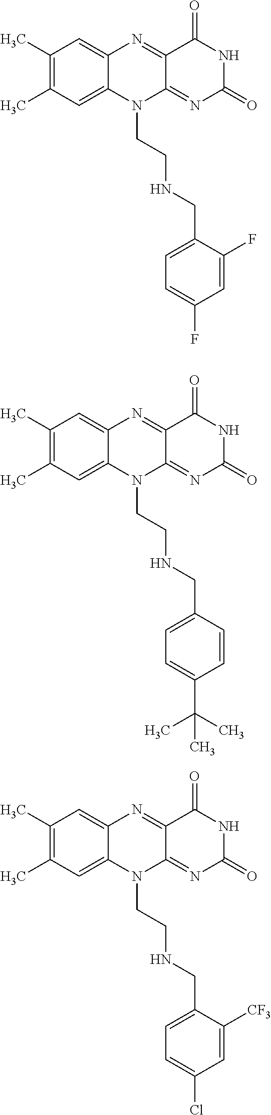

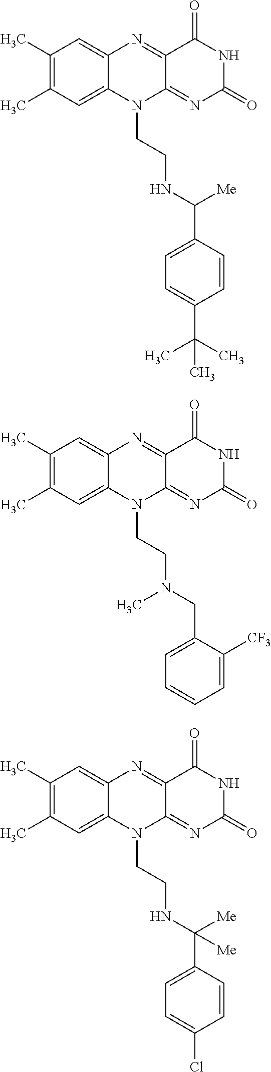

in free or salt form.

In a still another further embodiment of the fifth aspect, the invention provides a Compound of Formula II(B) selected from any of the following:

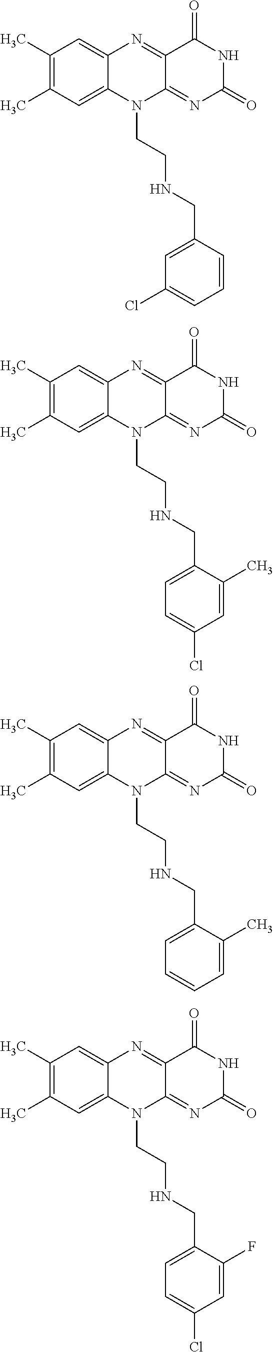

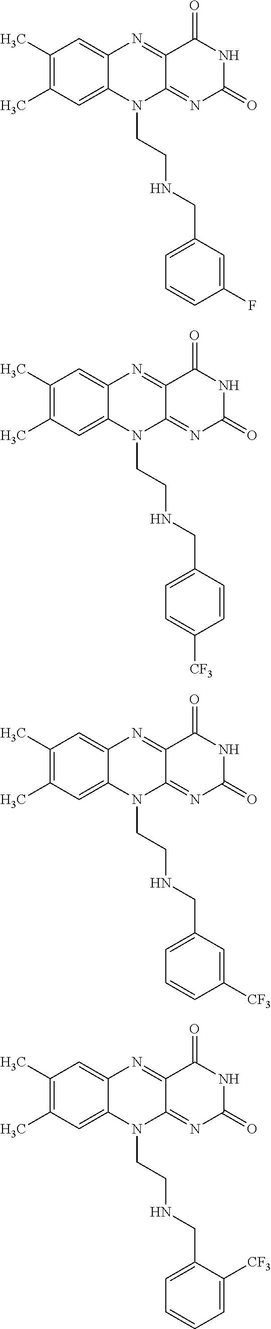

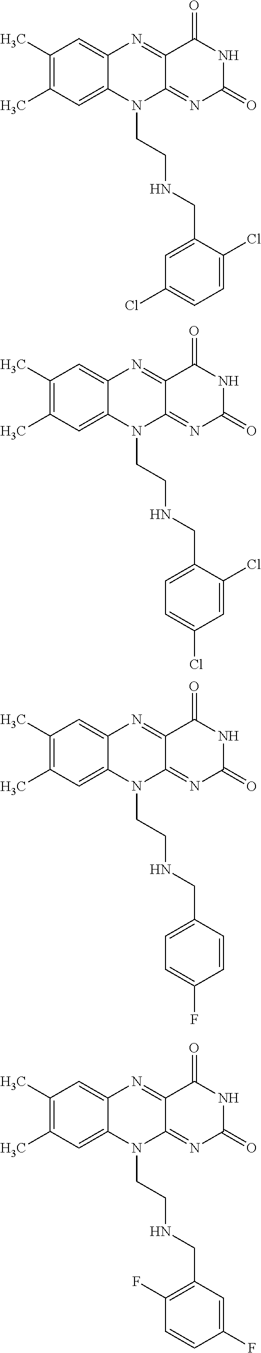

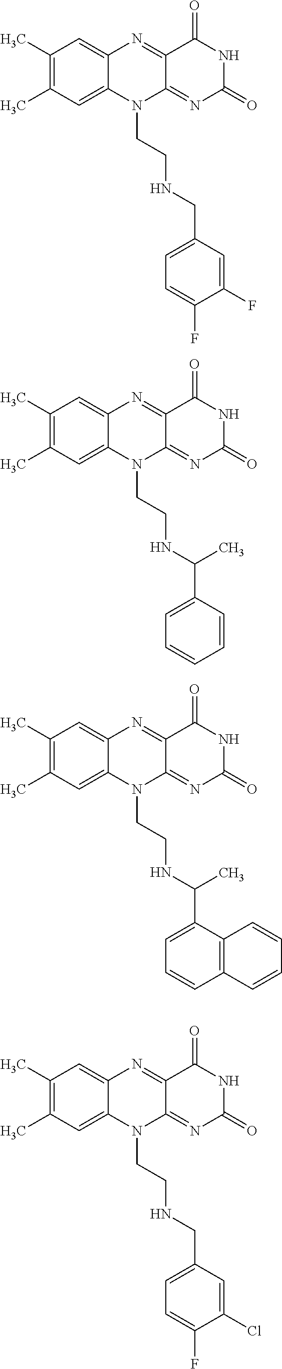

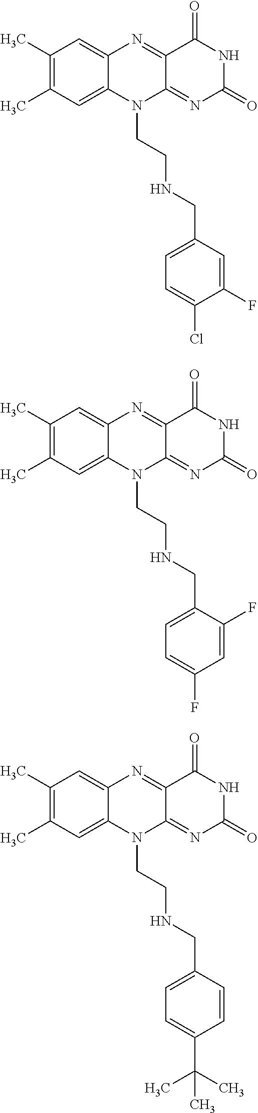

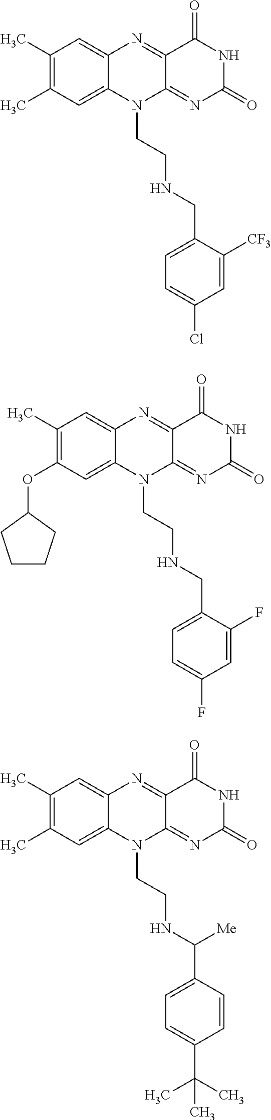

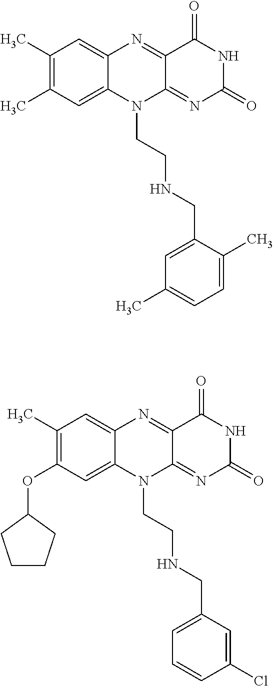

in free or salt form.

In yet another embodiment, the Compound of Formula II(B) as described above, binds to FMN and/or CD3299 riboswitch, e.g., with an Imax of greater than 20%, preferably greater than 30%, more preferably greater than 40%, still more preferably greater than 50%, in an assay, for example, as described in Example 1, and/or has a Minimum Inhibitory Concentration (MIC) of less than or equal to 64 μg/mL, more preferably less than or equal to 32 μg/mL, still more preferably less than or equal to 16 μg/mL, for example, in an assay as described in Example 2.

In the sixth aspect, the invention provides a Compound of Formula III(B):

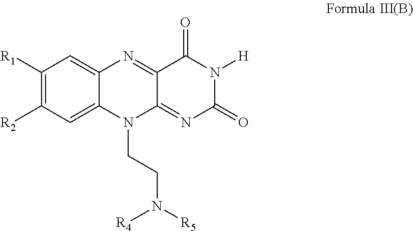

wherein:
(i) R₁ is H or C₁₋₄alkyl (e.g., methyl);
(ii) R₂ is selected from a group consisting of H, C₁₋₄alkyl (e.g., methyl) and —O—C₃₋₈cycloalkyl (e.g., —O-cyclopentyl);
(iii) R₄ is benzyl;
(iv) R₅ is selected from aryl¹-C₀₋₄alkyl (e.g., phenyl, benzyl, phenylpropyl), hydroxyC₁₋₄alkyl (hydroxybutyl), C₁₋₄alkyl (e.g., n-butyl), C₃₋₈cycloalkyl¹ (e.g., cyclopentyl), wherein R₅ is optionally substituted with one or more hydroxy or C₁₋₄alkyl (e.g., methyl);
(v) or R₄ is H and R₅ is 1,2-diphenylethyl or 1-hydroxy-2-hydroxymethyl-2-phenyl (—C(H)(CH₂OH)—C(H)(OH)—C₆H₅);
   in free or salt form.

In a further embodiment of the sixth aspect, the invention provides a Compound of Formula III(B) selected from any of the following:

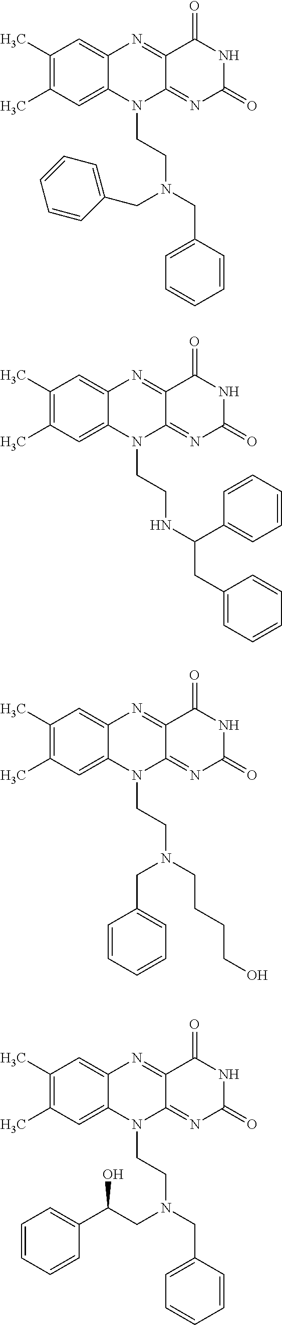

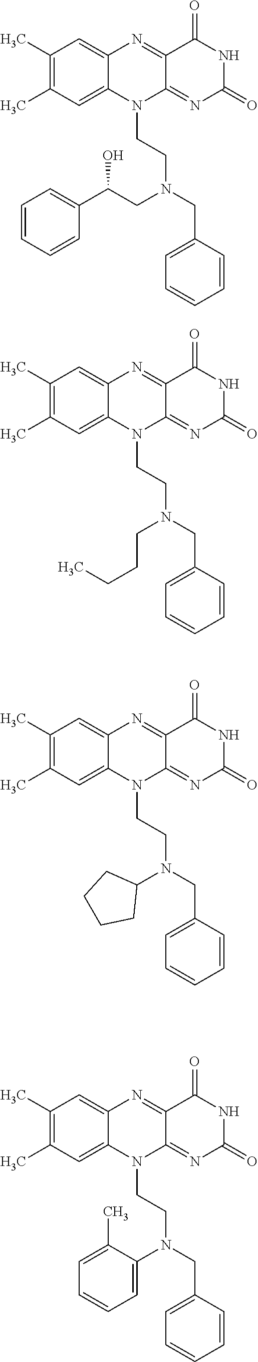

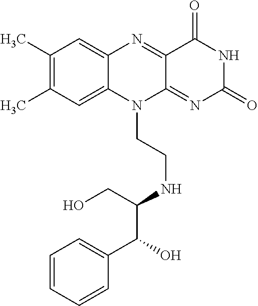

in free or salt form.

In a yet another embodiment of the sixth aspect, the invention provides a Compound of Formula III(B) selected from any of the following:

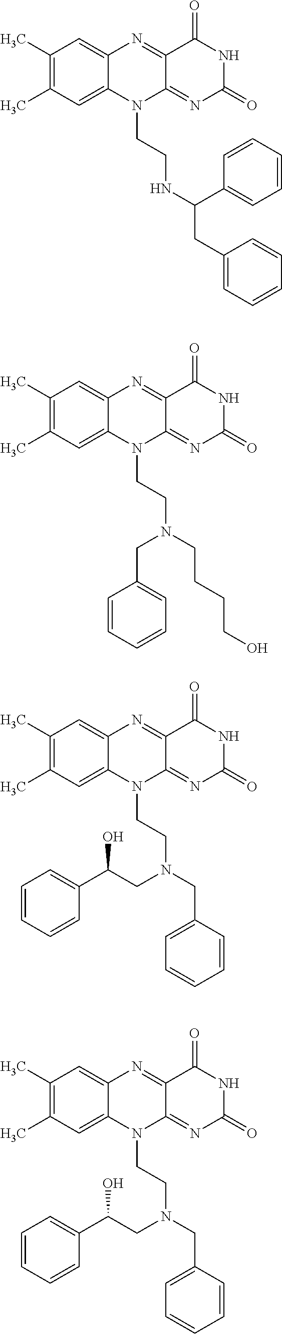

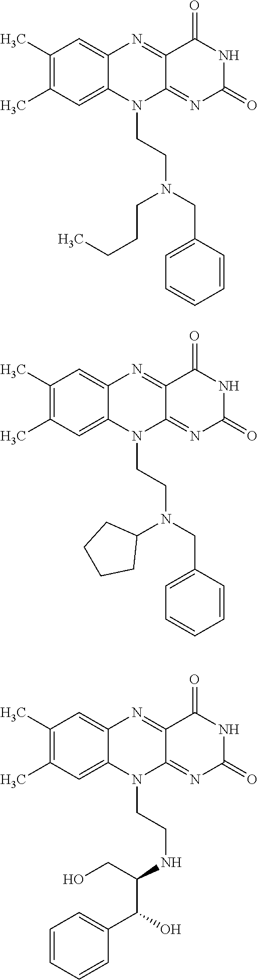

in free or salt form.

In another embodiment of the sixth aspect, the invention provides a Compound of Formula III(B) selected from any of the following:

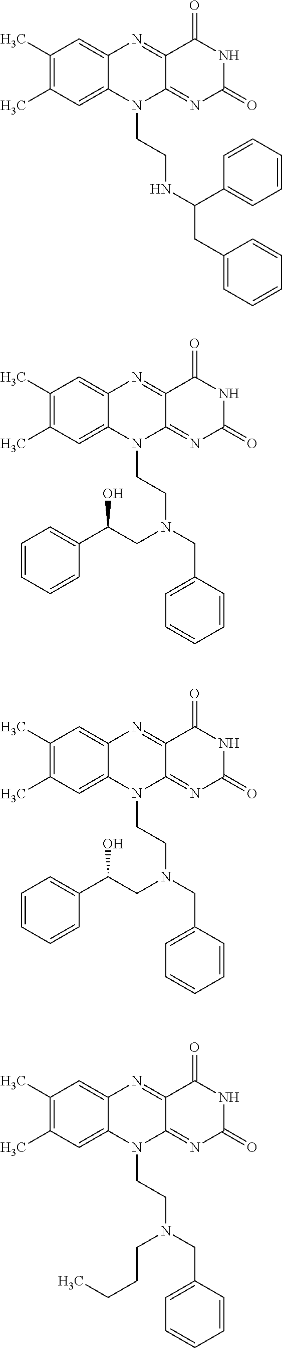

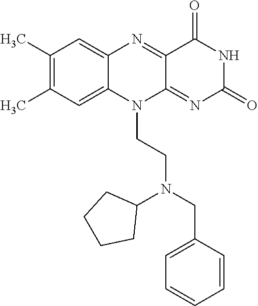

in free or salt form.

In yet another embodiment of the sixth aspect, the invention provides a Compound of Formula III(B) selected from the following:

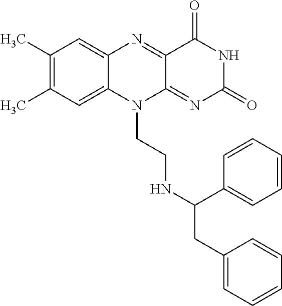

in free or salt form.

In yet another embodiment, the Compound of Formula III(B) as described above, binds to FMN and/or CD3299 riboswitch, e.g., with an Imax of greater than 20%, preferably greater than 30%, more preferably greater than 40%, in an assay, for example, as described in Example 1, and/or has a Minimum Inhibitory Concentration (MIC) of less than or equal to 64 μg/mL, in an assay as described in Example 2.

In the seventh aspect, the invention provides a compound of Formula IV(B) selected from any of the following:

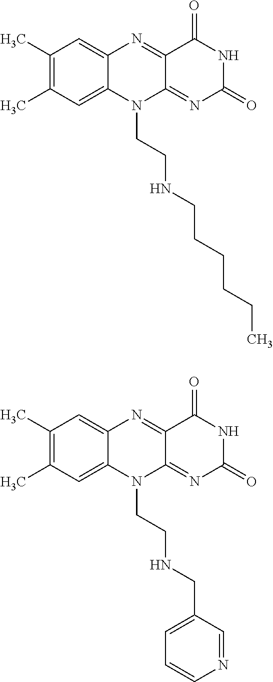

in free or salt form.

The Compounds of Formula IV(B) as described above, binds to FMN and/or CD3299 riboswitch, e.g., with an Imax of greater than 20% in an assay, for example, as described in Example 1, and/or has a Minimum Inhibitory Concentration (MIC) of less than or equal to 64 μg/mL, in an assay as described in Example 2.

In the eighth aspect, the invention provides a Compound of Formula V(B):

selected from any of the following:

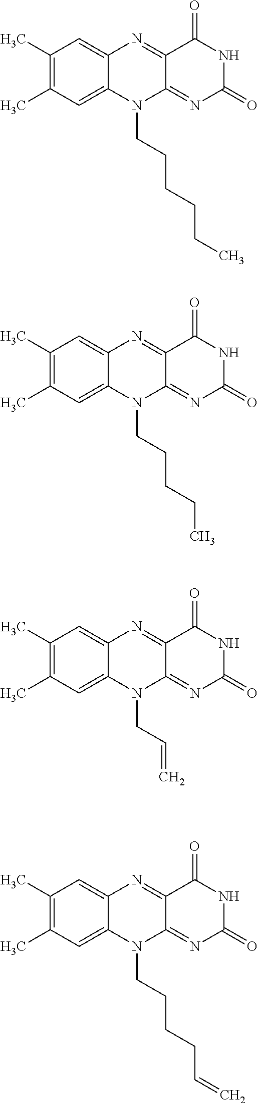

in free or salt form.

The compounds described herein, i.e., the compounds of Formula Q, Q-I, Q-II, Q-III, Q-IV, Q-V, Q(i), Q-I(i), Q-II(i), Q-III(i), Q-IV(i), Q-V(i), or any of Q.1-Q.42, I(A) or any of 1.1-1.44, II(A) or any of 2.1-2.9, I(B) or any of 3.1-3.55, II(B), III(B), IV(B) or V(B), in free or salt form, shall be referred to as the Compounds of the Invention.

In the ninth aspect, the invention provides a pharmaceutical composition comprising a Compound of the Invention, in free or pharmaceutically acceptable salt form, as herein before described, in admixture with a pharmaceutically acceptable diluent or carrier. In a further embodiment, the pharmaceutical composition of the invention comprises the following:
(a) a Compound of Formula Q(i) or any of Q.1-Q.42, in free or pharmaceutically acceptable salt form; (Composition Q(i))
(b) a Compound of Formula Q-I(i) or any of Q.1-Q.42, in free or pharmaceutically acceptable salt form; (Composition Q-I(i))
(c) a Compound of Formula Q-II(i) or any of Q.1-Q.42, in free or pharmaceutically acceptable salt form; (Composition Q-II(i))
(d) a Compound of Formula Q-III(i) or any of Q.1-Q.42, in free or pharmaceutically acceptable salt form; (Composition Q-III(i))
(e) a Compound of Formula Q-IV(i) or any of Q.1-Q.42, in free or pharmaceutically acceptable salt form; (Composition Q-IV(i))
(f) a Compound of Formula Q-V(i) or any of Q.1-Q.42, in free or pharmaceutically acceptable salt form; (Composition Q-V(i))
(g) a Compound of Formula I(A)(i), e.g., any of formulae 1.1-1.44 in free or pharmaceutically acceptable salt form; (Composition I(A)(i))
(h) a Compound of Formula II(A), e.g., any of 2.1-2.9 in free or pharmaceutically acceptable salt form; (Composition II(A))
(i) a Compound of Formula I(B) or any of 3.1-3.55 in free or pharmaceutically acceptable salt form; (Composition I(B))
(j) a Compound of Formula II(B) in free or pharmaceutically acceptable salt form; (Composition II(B))
(k) a Compound of Formula III(B) in free or pharmaceutically acceptable salt form; (Composition III(B))
(l) a Compound of Formula IV(B) in free or pharmaceutically acceptable salt form; (Composition IV(B)) or
(m) a Compound of Formula V(B) in free or pharmaceutically acceptable salt form, (Composition V(B))
   in admixture with a pharmaceutically acceptable diluent or carrier. In one embodiment, the Pharmaceutical Composition of the Invention comprises a compound selected from any of those described in formula 1.39, 1.41, 1.42 or 1.43, in free or pharmaceutically acceptable salt form. In another embodiment, the Pharmaceutical Composition of the Invention comprises a compound selected from any of those described in formula Q.35, Q.36, Q.37, Q.38, Q.39, Q.40 or Q.41, in free or pharmaceutically acceptable salt form.

In the tenth aspect, the invention provides a method for the treatment or prophylaxis of a bacterial infection (Methods of the Invention) comprising administering to a subject in need thereof an effective amount of a Compound or a Pharmaceutical Composition of the Invention, e.g., comprising administering an effective amount of a:
(a) a Compound or Pharmaceutical Composition of Formula Q or any of Q.1-Q.42, in free or pharmaceutically acceptable salt form; (Method Q)
(b) a Compound or Pharmaceutical Composition of Formula Q-I or any of Q.1-Q.42, in free or pharmaceutically acceptable salt form; (Method Q-I)
(c) a Compound or Pharmaceutical Composition of Formula Q-II or any of Q.1-Q.42, in free or pharmaceutically acceptable salt form; (Method Q-II)
(d) a Compound or Pharmaceutical Composition of Formula Q-III or any of Q.1-Q.42, in free or pharmaceutically acceptable salt form; (Method Q-III)
(e) a Compound or Pharmaceutical Composition of Formula Q-IV or any of Q.1-Q.42, in free or pharmaceutically acceptable salt form; (Method Q-IV)
(f) a Compound or Pharmaceutical Composition of Formula Q-V or any of Q.1-Q.42, in free or pharmaceutically acceptable salt form; (Method Q-V)
(g) a Compound or Pharmaceutical Composition of Formula I(A), e.g., any of formulae 1.1-1.44 in free or pharmaceutically acceptable salt form; (Method I(A))
(h) a Compound or Pharmaceutical Composition of Formula II(A), e.g., any of 2.1-2.9 in free or pharmaceutically acceptable salt form; (Method II(A))
(i) a Compound or Pharmaceutical Composition of Formula I(B) or any of 3.1-3.55 in free or pharmaceutically acceptable salt form; (Method I(B))
(j) a Compound or Pharmaceutical Composition of Formula II(B) in free or pharmaceutically acceptable salt form; (Method II(B))
(k) a Compound or Pharmaceutical Composition of Formula III(B) in free or pharmaceutically acceptable salt form; (Method III(B))
(l) a Compound or Pharmaceutical Composition of Formula IV(B) in free or pharmaceutically acceptable salt form; (Method IV(B)) or
(m) a Compound or Pharmaceutical Composition of Formula V(B) in free or pharmaceutically acceptable salt form, (Method V(B))
   In a further embodiment, the invention provides a method for the treatment or prophylaxis of a bacterial infection comprising administering to a subject in need thereof an effective amount of a compound or a pharmaceutical composition comprising a Compound of any of formulae 1.41, 1.42 or 1.43, in free or pharmaceutically acceptable salt form, as herein before described. In another embodiment, Method of the Invention comprises any of the compounds described in any of formula Q.35, Q.36, Q.37, Q.38, Q.39, Q.40 or Q.41 in free or pharmaceutically acceptable salt form.

In a further embodiment of the tenth aspect, the Methods of the Invention as hereinbefore described, are useful for the treatment or prophylaxis of a Gram-positive or Gram-negative bacterial infection (Method Q-A, Q-I-A, Q-II-A, Q-III-A, Q-IV-A, Q-V-A, I(A)-A, II(A)-A, I(B)-A, II(B)-A, III(B)-A, IV(B)-A, V(B)-A respectively). In a specific embodiment, Methods of the Invention are useful for treating a bacterial infection including, but not limited to, an infection by one or more of the following bacteria: Clostridium difficile (or C. difficile), Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and/or Borrelia burgdorferi bacteria (Method Q-B, Q-I -B, Q-II-B, Q-III-B, Q-IV-B, Q-V-B, I(A)-B, II(A)-B, I(B)-B, II(B)-B, III(B)-B, IV(B)-B, V(B)-B respectively). In another specific embodiment, Methods of the Invention are useful for treating a bacterial infection including, but not limited to, an infection by one or more of the following bacteria: Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and/or Borrelia burgdorferi bacteria (Method Q-B′, Q-I-B′, Q-II-B′, Q-III-B′, Q-IV-B′, Q-V-B′, I(A)-B′, II(A)-B′, I(B)-B′, II(B)-B′, III(B)-B′, IV(B)-B′, V(B)-B′ respectively). In one embodiment, Methods of the Invention are useful for treating an infection by one or more of the following bacteria: Clostridium difficile (or C. difficile), Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii. In another embodiment, Methods of the Invention are useful for treating an infection by the Staphylococcus aureus and/or Staphylococcus epidermidis bacteria. In a particular embodiment, Methods of the Invention are useful for treating a Staphylococcus aureus infection (Method Q-C, Q-I-C, Q-II-C, Q-III-C, Q-IV-C, Q-V-C, I(A)-C, II(A)-C, I(B)-C, II(B)-C, III(B)-C, IV(B)-C, V(B)-C respectively). Patients taking antibiotics, particularly those with a broad spectrum activity, are particularly vulnerable to C. difficile infection as a result of the use of antibiotics which disrupts the normal intestinal flora, leading to an overgrowth of C. difficile, causing an infection ranging from asymptomatic to severe and life-threatening condition. Various Compounds of the Invention are particularly active against the CD3299 riboswitch and selectively inhibits C. difficile bacteria. Therefore, in a particular embodiment, Methods of the Invention are particularly useful for treating an infection caused by Clostridium difficile.

In another embodiment of the tenth aspect, the invention provides Method of the Invention as hereinbefore described, useful for the treatment or prophylaxis of a disease, infection or condition selected from a group consisting of anthrax, staphylococcal scalded skin syndrome (staph infections), pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea, conjunctivitis and clostridium difficile associated disease (CDAD), comprising administering to a subject in need thereof an effective amount of a Compound of the Invention as hereinbefore described, in free or pharmaceutically acceptable salt form (Method Q-D, Q-I-D, Q-II-D, Q-III-D, Q-IV-D, Q-V-D, I(A)-D, II(A)-D, I(B)-D, II(B)-D, III(B)-D, IV(B)-D, V(B)-D respectively). In another further embodiment of the tenth aspect, the invention provides the method Q-D of the Invention, wherein the compound selected from any of those described in formula 1.39, 1.41, 1.42 or 1.43, in free or pharmaceutically acceptable salt form. In one particular embodiment, the invention provides Method Q-D which comprises a compound selected from any of those described in formula Q.35, Q.36, Q.37, Q.38, Q.39, Q.40 or Q.41, in free or pharmaceutically acceptable salt form.

Without being bound to any particular theory, it is believed that the current invention provides methods of treating a bacterial infection via a novel mechanism, e.g., by utilizing riboswitch-ligand binding to alter gene expression, thereby affecting downstream riboflavin biosynthesis. In another aspect, various compounds of the invention are active against the CD3299 riboswitch, thereby affecting expression of the adjacent coding region. Compounds that are active against CD3299 riboswitch are particularly selectively against C. difficile. As such, the Compounds of the Invention as hereinbefore described, in free or pharmaceutically acceptable salt form, e.g., a compound selected from any of those described in formula 1.41 or 1.43, are effective in treating an infection wherein traditional antibiotics are rendered ineffective due to drug resistance. Therefore, in a particular embodiment, the invention provides Methods of the Invention as hereinbefore described wherein the infection is by an infectious agent which is resistant to a drug that is not a riboswitch ligand (Method Q-E, Q-I-E, Q-II-E, Q-III-E, Q-IV-E, Q-V-E, I(A)-E, II(A)-E, I(B)-E, II(B)-E, III(B)-E, IV(B)-E, V(B)-E respectively). In a further embodiment, the infection is resistant to one or more drugs selected from a group consisting of a penicillin, vancomycin, cephalosporin and methicillin. In a particular embodiment, the infection is a methicillin-resistant Staphylococcus aureus infection. In still another embodiment, the infection is resistant to fluoroquinolone (e.g., ciprofloxacin- and/or levofloxacin-resistant infection), metronidazole and/or vancomycin.

It will be noted that various compounds of the Invention have a low CC₅₀ value in an assay as disclosed in Example 2a and therefore, may have anti-metabolite activities which may interfere with DNA biosynthesis. Therefore, in one embodiment, these compounds may be useful as an anti-cancer or anti-viral agent. In another embodiment, the compounds that have a high Iₘₐₓ value and/or a low MIC in an assay as disclosed in Example 1 and 2 respectively, and a low CC₅₀ value in an assay as disclosed in Example 2a are used as an antibacterial, for topical administration.

In the eleventh aspect, the invention provides use of a Compound or use of a Pharmaceutical Composition comprising a Compound of the Invention as hereinbefore described, in free or pharmaceutically acceptable salt form (in the manufacture of a medicament) for the treatment or prophylaxis of an infection, e.g., a bacterial infection (Use of the Invention). In a further embodiment of the eleventh aspect, the invention provides the following:
a) use as hereinbefore described wherein the compound is a compound of any of formulae 1.41, 1.42 or 1.43, as hereinbefore described, in free or pharmaceutically acceptable salt form.
b) use as hereinbefore described wherein the compound is a compound of any of formulae Q.35, Q.36, Q.37, Q.38, Q.39, Q.40 or Q.41 as hereinbefore described, in free or pharmaceutically acceptable salt form.
c) use as hereinbefore described wherein the infection is a Gram-positive or Gram-negative infection.
d) use as hereinbefore described wherein the infection is an infection of one or more bacteria selected from a group consisting of Clostridium difficile (or C. difficile), Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and/or Borrelia burgdorferi bacteria.
e) use as hereinbefore described wherein the infection is an infection of one or more bacteria selected from a group consisting of Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and/or Borrelia burgdorferi bacteria.

In a preferred embodiment, the infection is by one or more bacteria selected from any one of the following: Clostridium difficile (or C. difficile), Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii. In another preferred embodiment, the infection is by the Clostridium difficile (or C. difficile), Staphylococcus aureus and/or Staphylococcus epidermidis bacteria.

In a further embodiment of the eleventh aspect, the invention provides use as herein described (in the manufacture of a medicament) for the treatment or prophylaxis of a condition, disease or infection selected from anthrax, staphylococcal scalded skin syndrome (staph infections), pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis. In addition, the invention provides use as described in this eleventh aspect, wherein the condition, disease or infection is additionally selected from the clostridium difficile associated disease (CDAD).

In yet another embodiment of the eleventh aspect, the invention provides use as hereinbefore described, wherein said infection is resistant to a drug that is not a riboswitch ligand. In a further embodiment, the infection is resistant to one or more drugs selected from a group consisting of penicillin, vancomycin, cephalosporin and methicillin. In a particular embodiment, the infection is a methicillin-resistant Staphylococcus aureus infection. In yet another embodiment, the infection is resistant to fluoroquinolone (e.g., ciprofloxacin- and/or levofloxacin-resistant infection), metronidazole and/or vancomycin.

In the twelfth aspect, the invention provides a method for the treatment of an infection in a plant comprising administering to such plant an effective amount of a Compound of the Invention as hereinbefore described, in free or pharmaceutically acceptable salt form. In a further embodiment of the twelfth aspect, the compound is a compound selected from any of those described in formula 1.39, or any of formula 1.41, 1.42 or 1.43, as hereinbefore described, in free or salt form. In another embodiment, the infection in such plants is a bacterial infection. In a particular embodiment, the compound is selected from any of those described in formula 1.41 or 1.43.

In still another embodiment, the methods according to the twelfth aspect of the invention comprises administering to such plant an effective amount of a compound of formula Q.35, Q.36, Q.37, Q.38, Q.39, Q.40 or Q.41, in free or pharmaceutically acceptable salt form.

### DETAILED DESCRIPTION OF THE INVENTION

The term “riboswitch” or “riboswitches” is an art recognized term and refers to an mRNA which comprises a natural aptamer that binds target metabolite and an expression platform which changes in the RNA structure to regulate genes.

The term “FMN riboswitch” refers to a riboswitch that binds a metabolite such as flavin mono-nucleotide (FMN) or binds ligands such as various Compounds of the Invention, including but not limited to various compounds of Formula I(A) or 1.1-1.44, e.g. a compound selected from any of those described in formula 1.41 or 1.43 or Formula II(A), e.g., various compounds of formulae 2.1-2.9, as hereinbefore described, or various compounds of Formula Q, Q-I to Q-V or Q.1-Q.42, or formula I(B) to V(B) or various compounds of formulae 3.1-3.55 as hereinbefore described, in free or salt form, and which affects downstream FMN biosynthesis and transport proteins.

“FMN riboswitch ligand” refers to FMN or roseoflavin, various compounds of the Invention such as a compound selected from any of those described in formula 1.41 or 1.43 or various compounds of Formula II, or 2.1-2.9, or various compounds of Formula Q, Q-I to Q-V or Q.1-Q.42, or various compounds of Formulae I(B) to V(B), various compounds of formulae 3.1-3.55 as hereinbefore described, in free or salt form, which compounds bind to the FMN riboswitch, e.g., via the FMN-binding aptamer called the RFN element, which is a highly conserved domain in the 5′-untranslated regions of prokaryotic mRNA. Without intended to be bound by any particular theory, it is believed the binding of the ligand to its riboswitch induces a conformational change in the bacterial mRNA such that the expression of the ORF is repressed, for example, such that the expression of enzymes responsible for riboflavin and FMN biosynthesis is repressed. This is achieved by inducing the mRNA to form (1) a terminator hairpin that halts RNA synthesis before the ORF can be synthesized or (2) a hairpin that sequesters the Shine-Dalgarno sequence and prevents the ribosome from binding to the mRNA so as to translate the ORF.

“CD3299 riboswitch” refers to a riboswitch found in C. difficile, controlling the gene designated CD3299. The 5′UTR and beginning of ORF from CD3299 gene of C. difficile 630, accession number AM180355 is as follows:

SEQ ID NO: 1:
TTACAGCTTTCTGATTTTGATAAATTTAAAACTTACCATCTAATACTAAT
AACAGGTTAATTTTATCTAATTATTATAGATTCTCATACTGTGCCTTATT
CTATCTATAAATACAATTTAAGTGTCCATATTGAAATATTTGTATTGTA
ATACAGCTGGATATTACTTAAATCCAATTGTTTCCATTATAATTTTATGT
TAAAATAATATTACAAAATACATCTGTTTTTCTTCATAAACGGGTGAA
ATTCCCTATCGGCGGTAAAAGCCCGCGAGCCTTATGGCATAATTTG
GTCATATTCCAAAGCCAACAGTAAAATCTGGATGGTAGAAGAAAAT
AGTATATGAGTACCTTTATGTAATTTTACATGAGTAATCTATACAAATC
CTTCAACTACCGTATTTATTCATGAAATTAGACACATTCAAG [symbol] [symbol]
TTTTTTTGTTGTTTATTTTACAATTATATCGTACTTATA
AAATCTATTAAGATTGGAGTGTTATC [symbol] AATGGATAGTATT
GATTATCATCTGTATTGGTGTATTTATGTCTACTCTTGATGGAAGTATAC
TAAATATCGCAAA

In the above depiction of the sequence, the riboswitch is highlighted in bold, and is

SEQ ID N0: 2
GTTTTTCTTCATAAACGGGTGAAATTCCCTATCGGCGGTAAAAGCC
CGCGAGCCTTATGGCATAATTTGGTCATATTCCAAAGCCAACAGTA
AAATCTGGATGGTAGAAGAAAATA

The ORF start site in the above sequence is downstream from the riboswitch and is depicted in italics and is:

SEQ ID NO: 3

[symbol]

The putative terminator hairpin is in bold italics and is:

SEQ ID NO: 4

[symbol]

[symbol]

The hairpin can form a loop having a structure as depicted in Formula 1:

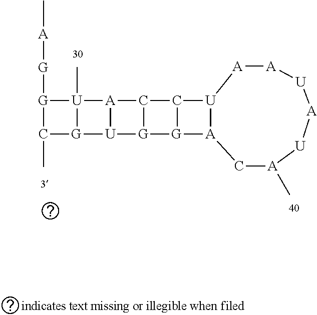

A possible antiterminator has a structure as depicted in Formula 2:

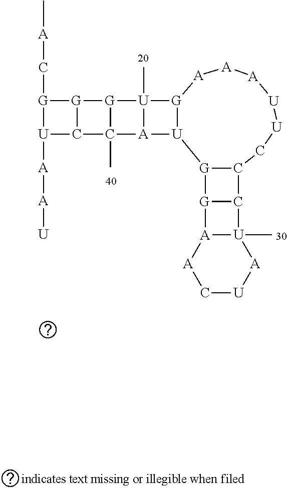

We have shown that various Compounds of the Invention, particularly various compounds of Formula Q, or any of Q-I to Q-V, for example, compounds of formula Q.39, Q.40 or Q.41, in free or salt form, bind well to the CD3299 riboswitch and have antibacterial activity against C. difficile.

The term “infection” refers to a bacterial infection. In another embodiment, the infection is a Gram-positive or Gram-negative infection. In still another embodiment, the infection is an infection by one or more bacteria selected from a group consisting of Clostridium difficile, Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and/or Borrelia burgdorferi. In a preferred embodiment, the infection is a Clostridium difficile, and/or Staphylococcus aureus and/or Staphylococcus epidermidis infection. In a further embodiment, the infection is a Staphylococcus aureus and/or Clostridium difficile infection. In a particular embodiment, the infection is an infection which is resistant to a drug which is not a riboswitch ligand. In a further aspect of this particular embodiment, the infection is an infection which is resistant to one or more drugs selected from a group consisting of penicillin, vancomycin, cephalosporin and methicillin. In a particular embodiment, the infection is a methicillin-resistant Staphylococcus aureus (MRSA) infection. In another particular embodiment, the infection is a fluoroquinolone-resistant (e.g., ciprofloxacin- and/or levofloxacin-resistant), metronidazole and/or vancomycin—resistant C. difficile infection.

The term “bacteria” or “bacterial” include, but are not limited to Clostridium difficile, Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and/or Borrelia burgdorferi. Preferably, the bacteria referred to in the current invention include but not limited to Clostridium difficile, Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis and Streptococcus pyogenes. More preferably, the bacteria referred to in the current the invention include but not limited to Clostridium difficile, Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii, most preferably, the bacteria referred to in the current the invention include Clostridium difficile, Staphylococcus aureus and/or Staphylococcus epidermidis.

If not otherwise specified or clear from context, the following terms as used herein have the following meetings:
a. “Alkyl” as used herein is a saturated or unsaturated hydrocarbon moiety, preferably saturated, e.g., one to eight, e.g., one to six, e.g., one to four, in some instances one to two carbon atoms in length, which may be linear or branched (e.g., n-butyl or tert-butyl) unless otherwise specified, and may be optionally substituted, e.g., mono-, di-, or tri-substituted on any one of the carbon atoms, e.g., with C₁₋₄alkyl (e.g., methyl), C₁₋₄alkoxy, halogen (e.g., chloro or fluoro), haloC₁₋₄alkyl (e.g., trifluoromethyl), hydroxy, and carboxy. For example, “C₁-C₈ alkyl” denotes alkyl having 1 to 8 carbon atoms. Examples of alkyl include, but are not limited to, methyl, ethyl, n-propyl, i-propyl, n-butyl, i-butyl, sec-butyl, t-butyl, 3-methylpentyl, 4-methylpentyl, n-pentyl, n-hexyl and n-heptyl.
b. “Aryl²” as used herein is a mono-cyclic aromatic hydrocarbon, preferably phenyl, optionally substituted, e.g., with C₁₋₄alkyl (e.g., methyl), C₁₋₄alkoxy, halogen (e.g., chloro or fluoro), haloC₁₋₄alkyl (e.g., trifluoromethyl), hydroxy, carboxy, or an additional aryl² or heteroaryl².
c. “Cycloalkyl²” is intended to include monocyclic, fully or partially saturated aliphatic (non-aromatic) ring system, for example C₃₋₈cycloalkyl² (e.g., cyclopentyl or cyclohexyl). Therefore, “cycloalkyl²” may denote simply a cyclopropyl, cyclobutyl, cyclopentyl, cyclopentenyl, cyclohexyl, cyclohexenyl and the like. Wherein the carbon atom of said cycloalkyl² is optionally replaced with one or more N, O, S, S(O)₂ or —C(O)—, i.e., a heterocycloalkyl², said heterocycloalkyl² may denote, for example, piperidinyl (e.g., piperidin-1-yl), pyrrolidinyl (e.g., pyrrolidin-1-yl), piperazinyl (e.g., 2,5-dioxopiperazin-1-yl), isoxazolidinyl (isoxazolidin-5-yl), 1,1-dioxo-1,4-thiazinan-4-yl, 2-oxopyrimidin-1-yl or 2,4-dioxo-imidazol-3-yl. The cycloalkyl² or heterocycloalkyl² of the invention may be substituted with, for example, C₁₋₈alkyl (e.g., methyl); The foregoing list of substituents is intended to provide concrete examples and not intended to be exhaustive.
d. “Heteroaryl²” as used herein refers to a mono-cyclic aromatic ring system containing at least one heteroatom independently selected from the group consisting of N, O and S. The heteroaryl² ring may be attached to its pendant group at any heteroatom or carbon atom which results in a stable structure. The heteroaryl² rings described herein may be substituted on carbon or on a nitrogen atom if the resulting compound is stable. Examples of heteroaryl² group include, but are not limited to pyridyl (e.g., pyrid-4-yl or pyrid-3-yl), imidazolyl, thiazolyl, pyrazinyl, pyrimidinyl and the like. The heteroaryl² group may also be optionally substituted with, for example, C₁₋₄alkyl (e.g., methyl), C₁₋₄alkoxy, halogen, hydroxy, haloC₁₋₄alkyl or carboxy.
e. “Aryl¹” as used herein is a monocyclic or polycyclic aromatic hydrocarbon, preferably phenyl, optionally substituted, e.g., with C₁₋₄alkyl (e.g., methyl), C₁₋₄alkoxy, halogen (e.g., chloro or fluoro), haloC₁₋₄alkyl (e.g., trifluoromethyl), hydroxy, carboxy, or an additional aryl or heteroaryl.
f. “Cycloalkyl¹” is intended to include monocyclic or polycyclic, preferably three to eight carbon atoms in length, fully or partially saturated non-aromatic ring system. Therefore, “cycloalkyl¹” may denote simply a cyclopropyl, cyclobutyl, cyclopentyl, cyclopentenyl, cyclohexyl, cyclohexenyl and the like.
g. “Heteroaryl¹” as used herein refers to a monocyclic or polycyclic aromatic ring system containing at least one heteroatom independently selected from the group consisting of N, O and S. The heteroaryl¹ ring may be attached to its pendant group at any heteroatom or carbon atom which results in a stable structure. The heteroaryl rings described herein may be substituted on carbon or on a nitrogen atom if the resulting compound is stable. Examples of heteroaryl group include, but are not limited to pyridyl (e.g., pyrid-4-yl or pyrid-3-yl), imidazolyl, thiazolyl, pyrazinyl, pyrimidinyl and the like. The heteroaryl group may also be optionally substituted with C₁₋₄alkyl (e.g., methyl), C₁₋₄alkoxy, halogen, hydroxy, haloC₁₋₄alkyl or carboxy.
h. Wherein the substituent is connected via an alkyl chain, for example —C₀₋₄alkyl-aryl, —C₀₋₄alkyl-heteroaryl, —C₀₋₄alkyl-benzotriazolyl or any similar type of substituents set forth herein, it is to be understood that the substituents may be linked via any position on the alkyl chain and not necessarily at the terminal carbon of the chain. For example, wherein the substituent is —C₃alkylaryl (e.g., phenylpropyl), the substituent may be —CH₂CH₂CH₂—C₅H₆ or —C(H)(CH₃)—CH₂—C₅H₆, —C(CH₃)(CH₃)—C₅H₆ or —C(H)(CH₃)—CH₂—C₅H₆. Wherein the substituent is —C₀alkylaryl, C₀ is intended to be a single bond.

The substituents on the Compounds of the Invention, e.g., Alk, X, Y, A and R₁-R₁₂ may be specifically or generally defined. Unless specified otherwise, Alk, X, A and R₁-R₁₂ are defined as in Formula Q, Q-I, Q-II, Q-III, Q-IV, Q-V, Q(i), Q-I(i), Q-II(i), Q-III(i), Q-IV(i), Q-V(i), or any of Q.1-Q.42, I(A) or any of 1.1-1.44, II(A) or any of 2.1-2.9, I(B) or any of 3.1-3.55, II(B), III(B), IV(B) or V(B).

The Compounds of the Invention (e.g., a Compound of Formula I(A), e.g., any of 1.1-1.44, a compound of Formula I(A)(i) or a Compound of Formula II(A), e.g., any of 2.1-2.9, or a Compound of Formula Q, Q-I through Q-V, Q(i), Q-I(i) through Q-V(i), or any of Q.1-Q.42, or a Compound of Formula I(B) through V(B) or any of formulae 3.1-3.55, as hereinbefore described may exist in free, salt, e.g., as acid addition salts, or prodrug form. An acid-addition salt of a compound of the invention which is sufficiently basic, for example, an acid-addition salt with, for example, an inorganic or organic acid, for example hydrochloric, hydrobromic, sulphuric, phosphoric, acid acetic, trifluoroacetic, citric, maleic acid, toluene sulfonic, propionic, succinic, glycolic, stearic, lactic, malic, tartaric, citric, ascorbic, pamoic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid, and the like. In addition a salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine. In a particular embodiment, the salt of the compound of the invention is a trifluoroacetic acid addition salt. In another embodiment, the salt of the compound of the invention is an acetic acid addition salt.

In this specification, unless otherwise indicated, language such as Compounds of the Invention is to be understood as embracing such Compounds in any form, for example free or acid addition salt or prodrug form, or where the compounds contain acidic substituents, in base addition salt form. The Compounds of the Invention are intended for use as pharmaceuticals, therefore pharmaceutically acceptable salts are preferred. Salts which are unsuitable for pharmaceutical uses may be useful, for example, for the isolation or purification of free Compounds of the Invention, and are therefore also included.

The Compounds of the Invention may comprise one or more chiral carbon atoms. The compounds thus exist in individual isomeric, e.g., enantiomeric or diasteriomeric form or as mixtures of individual forms, e.g., racemic/diastereomeric mixtures. Any isomer may be present in which the asymmetric center is in the (R)-, (S)-, or (R,S)-configuration. The invention is to be understood as embracing both individual optically active isomers as well as mixtures (e.g., racemic/diasteromeric mixtures) thereof. Accordingly, the Compound of the Invention may be a racemic mixture or it may be predominantly, e.g., in pure, or substantially pure, isomeric form, e.g., greater than 70% enantiomeric excess (“ee”), preferably greater than 80% ee, more preferably greater than 90% ee, most preferably greater than 95% ee. The purification of said isomers and the separation of said isomeric mixtures may be accomplished by standard techniques known in the art (e.g., column chromatography, preparative TLC, preparative HPLC, simulated moving bed and the like).

Geometric isomers by nature of substituents about a double bond or a ring may be present in cis (Z) or trans (E) form, and both isomeric forms are encompassed within the scope of this invention.

As will be appreciated by those skilled in the art, the Compounds of the Invention may exhibit keto-enol tautomerization. Therefore, the invention as defined in the present invention is to be understood as embracing both the structures as setforth herewith and their tautomeric forms.

It is also intended that the Compounds of the Invention encompass their stable isotopes. For example, the hydrogen atom at a certain position on the Compounds of the Invention may be replaced with deuterium. It is expected that the activity of compounds comprising such isotopes would be retained and/or it may have altered pharmacokinetic or pharmacodynamic properties. In addition to therapeutic use, compounds comprising such isotopes and having altered pharmacokinetic or pharmacodynamic properties would also have utility for measuring pharmacokinetics of the non-isotopic analogs.

Compounds of the Invention may in some cases also exist in prodrug form. The term “prodrug” is an art recognized term and refers to a drug precursors prior to administration, but generate or release the active metabolite in vivo following administration, via some chemical or physiological process. For example, when the Compounds of the Invention contain a carboxy group, these substituents may be esterified to form physiologically hydrolysable and acceptable esters (e.g., carboxylic acid esters, e.g., —C(O)OR₇). As used herein, “physiologically hydrolysable and acceptable esters” means esters of Compounds of the Invention which are hydrolysable under physiological conditions to yield acids, e.g., carboxylic acid (in the case of Compounds of the Invention which have a carboxy substituent) on the one hand and HOR₇ on the other hand, which are themselves physiologically tolerable at doses to be administered. Similarly, wherein the compounds of the invention contain an amine group, prodrug of such amine, e.g., amino acid, carbamic acid ester, amide prodrugs may also exist wherein the prodrug is cleaved to release the active amine metabolite in vivo following administration. Further details of amine prodrugs may may be found in Jeffrey P. Krise and Reza Oliyai, Biotechnology: Pharmaceutical Aspects, Prodrugs, Volume 5, Part 3, pages 801-831, the contents of which are herein incorporated by reference in their entirety. As will be appreciated, the term thus embraces conventional pharmaceutical prodrug forms.

For clarification, the Compound of Formula I(A)(i) is intended to cover the compounds described in Formula I(A), e.g., any of formulae 1.1-1.44, containing the proviso that when R₂ is chloro, Alk is propylene, X is a single bond and A is pyrrolidin-1-yl, then R₁ is C₁₋₈ alkyl (e.g., methyl) or R₁₀ is —C₁₋₄alkyl-OC(O)CH₃ (e.g., —CH₂OC(O)CH₃), i.e., the compound of Formula I(A) is not 8-chloro-10-(3-pyrrolidin-1-ylpropyl)benzo[g]pteridine-2,4-dione. The Compound of Formula I(A) is intended to cover similar compounds except that Compound of Formula I(A) does not contain any proviso. Similarly, the compound of Formula Q-I(i) is intended to cover compounds described in formula Q, e.g., any of Q.1-Q.42 as hereinbefore described, containing the proviso that:
(a) when R₂ is chloro, Alk is propylene, X is a single bond and A is pyrrolidin-1-yl, then R₁ is C₁₋₈ alkyl (e.g., methyl) or R₁₀ is —C₁₋₄alkyl-OC(O)CH₃ (e.g., —CH₂OC(O)CH₃), i.e., the compound is not 8-chloro-10-(3-pyrrolidin-1-ylpropyl)benzo[g]pteridine-2,4-dione;
(b) the compound is not 10-[3-(3,6-dioxo-1,4-cyclohexadien-1-yl)propyl)-3,7,8-trimethyl-benzo[g]pteridine-2,4-(3H,10H)-dione;
(c) A is not purinyl, e.g., the compound is not optionally substituted 10-[2-(9H-purin-9-yl)ethyl]-, 10-[3-(9H-purin-9-yl)propyl]- or 10-[6-(9H-purin-9-yl)hexyl]-7,8-dimethyl-benzo[g]pteridine-2,4-(3H,10H)-dione;
(d) A is not indol-3-yl, e.g., the compound is not 10-[3-(1H-indol-3-yl)ethyl]- or 10-[3-(1H-indol-3-yl)propyl]-7,8-dimethyl-benzo[g]pteridine-2,4-(3H,10H)-dione;
(e) -Alk-X-A is not 2-(2-oxocylopentylidene)ethyl.

### Methods of Using Compounds of the Invention

The Compounds of the Invention are useful for the treatment of an infection, particularly an infection by bacteria including but not limited to Clostridium difficile, Moraxella catarrhalis, Klebsiella pneumoniae, Staphylococcus epidermidis, Streptococcus viridians, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenza, Listeria monocytogenes, Salmonella enterica, Vibrio cholerae, Enterococcus faecalis, Yersinia pestis, Bacillus subtilis, Streptococcus pyogenes and/or Borrelia burgdorferi bacteria. In a preferred embodiment, the bacteria is selected from any one of the following: Clostridium difficile, Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii. In another preferred embodiment, the infection is by the Clostridium difficile, Staphylococcus aureus and/or Staphylococcus epidermidis bacteria.

The invention therefore provides methods of treatment of any one or more of the following conditions: anthrax infection, staphylococcal scalded skin syndrome (staph infections), pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea, conjunctivitis and clostridium difficile associated disease (CDAD); comprising administering an effective amount of a Compound of Formula I(A), e.g., any of 1.1-1.44, Formula II(A), e.g., any of 2.1-2.9, or Formula I(B), e.g., any of 3.1-3.55, or any of Formulae II(B)-V(B), or Formula Q, or any of Q-I to Q-V or any of Q.1-Q.42, in free or pharmaceutically acceptable salt form, to a subject in need thereof.

The words “treatment” and “treating” are to be understood accordingly as embracing prophylaxis and treatment or amelioration of symptoms of disease as well as treatment of the cause of the disease.

The term “subject” as used herein encompasses human and/or non-human (e.g., animal).

Dosages employed in practicing the present invention will of course vary depending, e.g. on the particular disease or condition to be treated, the particular Compound of the Invention used, the mode of administration, and the therapy desired. Administration of a therapeutically active amount of the therapeutic compositions is defined as an amount effective, at dosages and for periods of time necessary to achieve the desired result. For example, a therapeutically effective amount of a Compound of the Invention reactive with at least a portion of FMN riboswitch or the CD3299 riboswitch may vary according to factors such as the disease state, age, sex, and weight of the individual, and the ability of the compound to elicit a desired response in the individual. Dosage regiment may be adjusted to provide the optimum therapeutic response. For example, several divided doses may be administered daily or the dose may be proportionally reduced as indicated by the exigencies of the therapeutic situation. In general, satisfactory results, e.g. for the treatment of diseases as hereinbefore set forth are indicated to be obtained on oral administration at dosages of the order from about 0.01 to 2.0 mg/kg. In larger mammals, for example humans, an indicated daily dosage for oral administration will accordingly be in the range of from about 0.75 to 1500 mg, conveniently administered once, or in divided doses 2 to 4 times, daily or in sustained release form. Unit dosage forms for oral administration thus for example may comprise from about 0.2 to 75, 250 mg, 1,500 mg, e.g. from about 0.2 or 2.0 to 50, 75, 100, 250, 500, 750, 1000 or 1,500 mg of a Compound of the Invention, together with a pharmaceutically acceptable diluent or carrier therefor. Pharmaceutical compositions comprising the Compounds of the Invention may be prepared using conventional diluents or excipients and techniques known in the galenic art. Thus oral dosage forms may include tablets, capsules, solutions, suspensions, spray-dried dispersions [e.g. Eudragit L100]and the like. The term “pharmaceutically acceptable carrier” as used herein is intended to include diluents such as saline and aqueous buffer solutions. The Compounds of the Invention may be administered in a convenient manner such as by injection such as subcutaneous, intravenous, by oral administration, inhalation, transdermal application, intravaginal application, topical application, intranasal, sublingual or rectal administration. Depending on the route of administration, the active compound may be coated in a material to protect the compound from the degradation by enzymes, acids and other natural conditions that may inactivate the compound. In a preferred embodiment, the compound may be orally administered. In another embodiment, the compound is administered via topical application.

In certain embodiment, the Compounds of the Invention may be administered alone or in conjunction, e.g., at or about the same time or simultaneously and separately or simultaneously in an admixture, with another agent, e.g., an agent to facilitate entry or permeability of the Compounds of the Invention into the cell (i.e., a membrane enhancer), e.g., an antimicrobial cationic peptide. For example, the Compounds of the Invention with low or weak MIC activities may be administered alone or in conjunction with a membrane enhancer such as an antimicrobial cationic peptide. Antimicrobial cationic peptides include peptides which contain (1) a disulfide-bonded β-sheet peptides; (2) amphipathic α-helical peptides; (3) extended peptides; or (4) loop-structured peptides. Examples of cationic peptide include but are not limited to defensins, cecropins, melittins, magainins, indolicidins, bactenecin and protegrins. Other examples of antimicrobial cationic peptides include but are not limited to human neutrophil defensin-1 (HNP-1), platelet microbicidal protein-1 (tPMP), inhibitors of DNA gyrase or protein synthesis, CP26, CP29, CP11CN, CP10A, Bac2A-NH₂ as disclosed in Friedrich et al., Antimicrob. Agents Chemother. (2000) 44(8):2086, the contents of which are hereby incorporated by reference in its entirety. Further examples of antibacterial cationic peptides include but are not limited to polymyxin e.g., polymixin B, polymyxin E or polymyxin nonapeptide. Therefore, in another embodiment, the Compounds of the Invention may be administered in conjunction with polymyxin, e.g., polymixin B, polymyxin E or polymyxin nonapeptide, preferably polymyxin B.

In still another embodiment, the Compounds of the Invention may be administered alone or in conjunction, e.g., at or about the same time, simultaneously and separately, or simultaneously in an admixture, with other antimicrobial agents, e.g., other antifungal or other systemic antibacterial (bactericidal or bacteriostatic) agents. Examples of bacterial agents include agents which inhibit bacterial cell wall synthesis (e.g., penicillins, cephalosporins, carbapenems, vancomycin), agents which damage cytoplasmic membrane (e.g., polymixins as discussed above), agents which modify the synthesis or metabolism of nucleic acids (e.g., quinolones, rifampin, nitrofurantoin), agents which inhibit protein synthesis (aminoglycosides, tetracyclines, chloramphenicol, erythomycin, clindamycin), agents which interfer with the folate synthesis (e.g., folate-inhibitors), agents which modify energy metabolism (e.g., sulfonamides, trimethoprim) and/or other antibiotics (beta-lactam antibiotic, beta-lactamase inhibitors). Specific anti-infective agents, particularly antibacterial and antifungal agents, are discussed in Remington: The Science and Practice of Pharmacy, Chapter 90, pp. 1626-1684 (21^{st} Ed., Lippincott Williams & Wilkins 2005), the contents of which are hereby incorporated by reference.

### Methods of Making the Compounds of the Invention:

The compounds of the Invention may be made using the methods as described and exemplified herein and by methods similar thereto and by methods known in the chemical art. Such methods include, but not limited to, those described below. In the description of the synthetic methods described herein, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. Therefore, at times, the reaction may require to be run at elevated temperature or for a longer or shorter period of time. It is understood by one skilled in the art of organic synthesis that functionality present on various portions of the molecule must be compatible with the reagents and reactions proposed. If not commercially available, starting materials for these processes may be made by procedures, which are selected from the chemical art using techniques which are similar or analogous to the synthesis of known compounds. All references cited herein are hereby incorporated in their entirety by reference.

The synthetic methods for the Compounds of the Invention are illustrated below. The significances for the substituents are as set forth above for Formula I(A) or any of 1.1-1.44, Formula II(A), e.g., any of 2.1-2.9 or Formula Q, or any of Q-I to Q-V or any of Q.1-Q.42, unless otherwise indicated.

The Compound of Formula I(A) wherein X is —N(R₆)— and A is as defined in Formula I(A) or X is a single bond and A is C₅₋₆cycloalkyl wherein the atom attached to X is a nitrogen (e.g., —X-A is piperidin-1-yl or pyrrolidin-1-yl), may be prepared by first preparing Intermediate (B) by reacting riboflavin with orthoperiodic acid followed by reductive amination of intermediate (B) with H—X-A wherein X is HN(R₆)— or X is a single bond and A is a cycloalkyl containing one or more nitrogen atom:

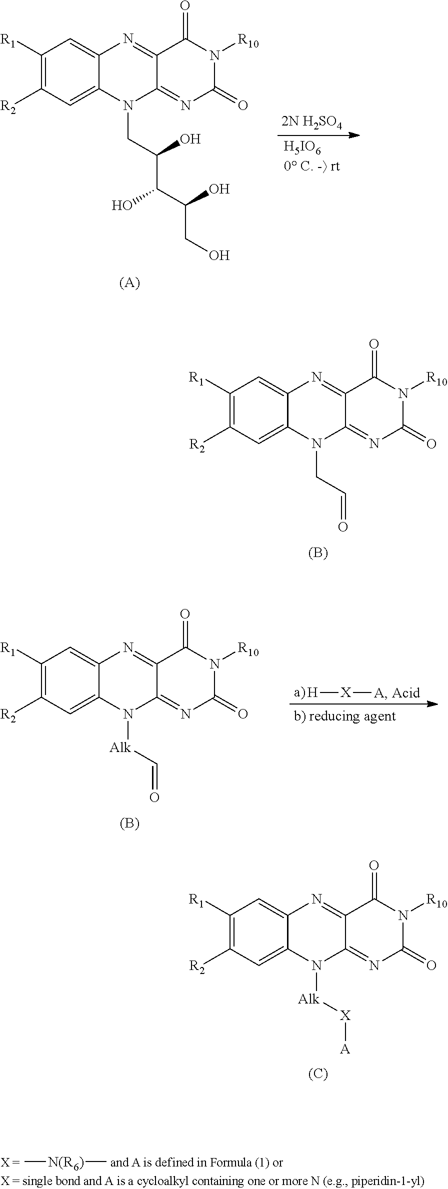

Therefore, in one embodiment, the invention provides a method of preparing a compound of Formula I(A) wherein X is —N(R₆)— and A is previously defined in Formula I(A) or X is a single bond and A is C₅₋₆cycloalkyl² wherein at least the atom attached to X is a nitrogen (e.g., —X-A is piperidin-1-yl or pyrrolidin-1-yl), comprising reductive amination of a compound of Formula (B):

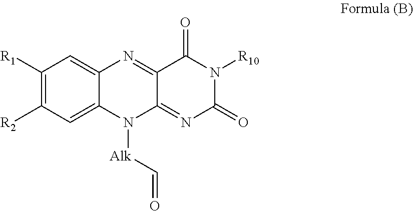

with H—X-A, wherein X is —N(R₆)— and A is previously defined in Formula I(A) or X is a single bond and A is C₅₋₆cycloalkyl wherein at least the atom attached to X is a nitrogen (e.g., —X-A is piperidin-1-yl or pyrrolidin-1-yl). In a further embodiment, the amination step involves the use of an acid, e.g., acetic acid and the reduction step involves the use of, e.g., sodium cyanoborohydride or sodium borohydride.

The Compound of Formula I(A) wherein X is —N(R₆)—CH₂— may be prepared by reacting a Compound of Formula (C′) with A-C(O)—H, e.g., methoxyisonicotinaldehyde, in the presence of an acid, e.g., acetic acid followed by a reducing agent, e.g., sodium cyanoborohydride, sodium borohydride, lithium hydride, or the like.

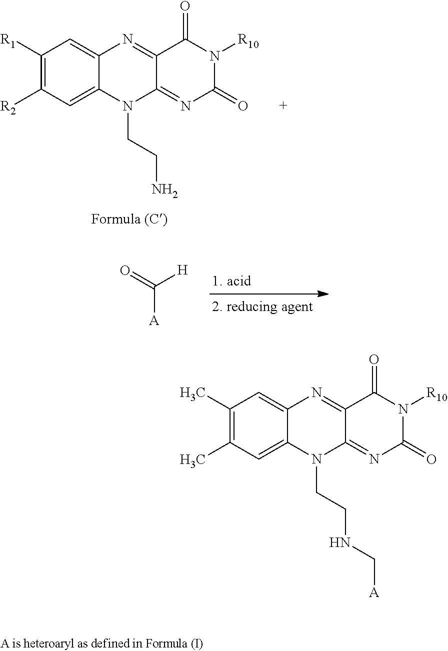

The Compound of Formula II(A) wherein Y is —N(R₆)—C(O)— may be prepared by reacting a compound of Formula (D) with A-C(O)OH wherein A is a heteroaryl as defined in Formula II(A), in the presence of an activating or coupling agent, e.g., HATU, BOP, HOBt, HOAt, dicyclohexylcarbodiimide, diisopropylcarbodiimide, POCl₃, or the like, and a base, e.g., organic base, e.g., triethylamine or DIPEA.

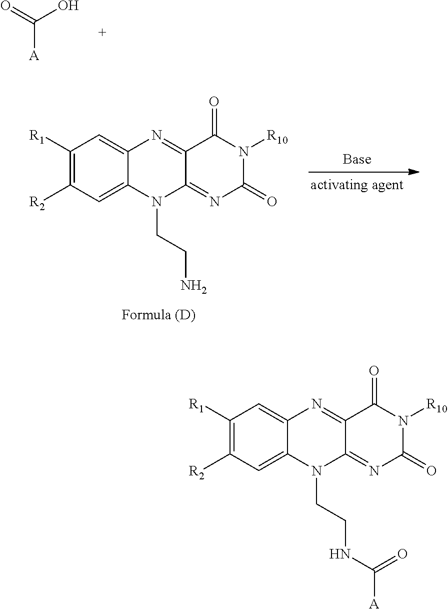

The Compound of Formula Q wherein Alk is previously defined in Formula Q, X is a single bond and A is a monocyclic heteroaryl² or C₃₋₈cycloalkyl² wherein one or more carbon atoms of said cycloalkyl² are optionally and independently replaced with substituted nitrogen, may be prepared by first alkylating an optionally substituted aryl² nitro amine with an electrophile [e.g. LG-Alk-X-A, where LG=Br or OMs] to provide a compound of Formula (E) and then reducing the nitro group to provide a diamine of Formula (F). Reaction of the diamine with alloxan in the presence of boric acid or diboron trioxide provides the desired product of Formula Q.

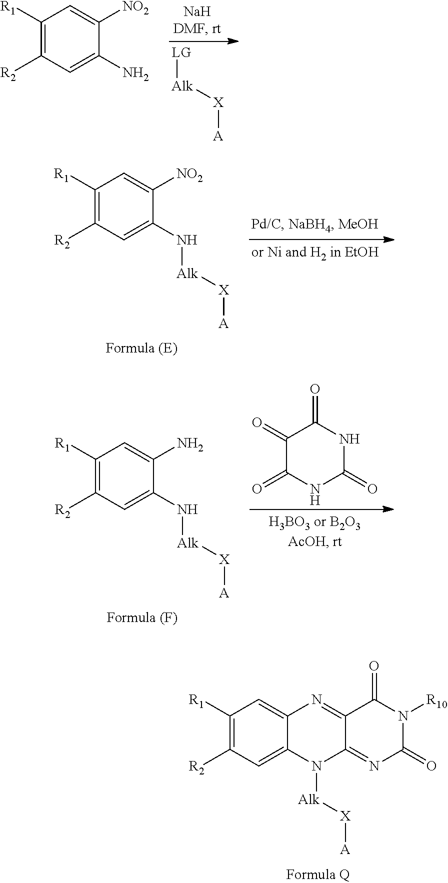

Alternatively, the Compound of Formula Q wherein Alk and A a previously defined in Formula Q, and X is a single bond, may be prepared by first alkylating an optionally substituted aryl diamine with an electrophile in the presence of a base [e.g. sodium carbonate] and n-butyl ammonium iodide to provide a diamine of Formula (F). Reaction of the diamine with alloxan in the presence of boric acid provides the desired product of Formula Q.

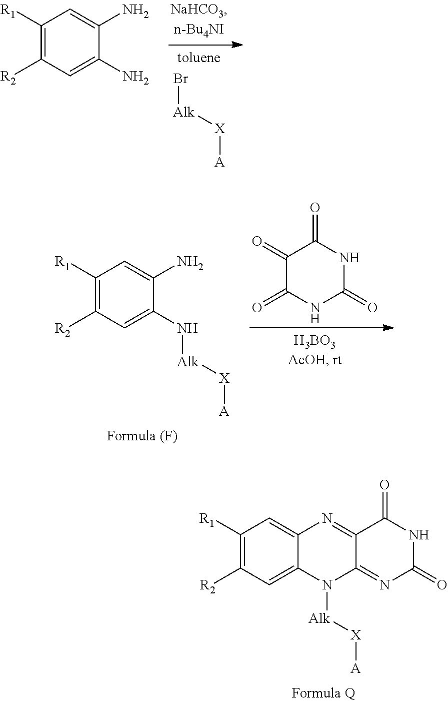

Alternatively, the Compound of Formula Q wherein Alk and A is defined in Formula Q, and X is a single bond, may be prepared by first reacting an appropriate amine [A-X-Alk-NH₂] in the presence of a base [e.g. CsCO₃] and a palladium catalyst with an optionally substituted aryl nitro bromide, or alternatively, reacting the amine neat with an optionally substituted aryl nitro bromide to provide a compound of Formula (E). Reduction [e.g. using palladium on carbon with sodium borohydride, or Raney Nickel and hydrogen] provides the corresponding diamine of Formula (F). Reaction of the diamine with alloxan in the presence of boric acid provides the desired product of Formula Q.

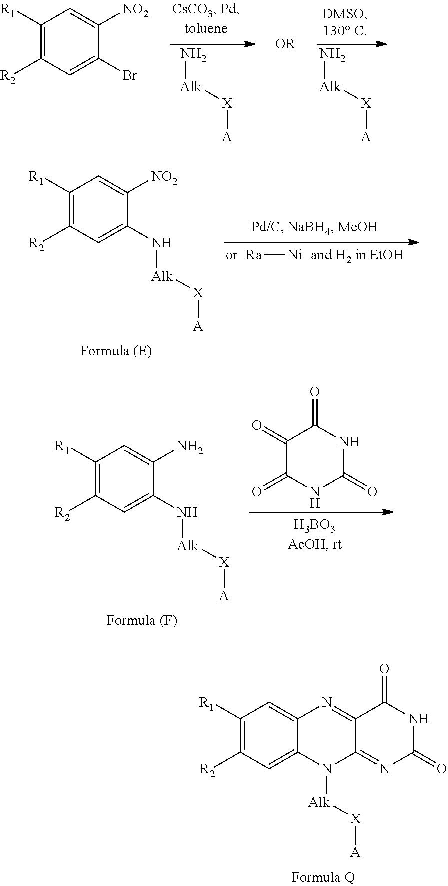

### EXAMPLES

### Binding of Ligand to Riboswitch

### Example 1

An in-line probing assay, as described in Regulski and Breaker, “In-line probing analysis of riboswitches”, (2008), Methods in Molecular Biology, Vol 419, pp 53-67, the contents of which are incorporated by reference in its entirety, is used to estimate the dissociation binding constants for the interaction of each of the ligands described herein with an FMN riboswitch amplified from the genome of Bacillus subtilis or a CD3299 riboswitch amplified from Clostridium difficile. Precursor mRNA leader molecules are prepared by in vitro transcription from templates generated by PCR and [5′-³²P]-labeling using methods described previously (Regulski and Breaker, In-line probing analysis of riboswitches (2008), Methods in Molecular Biology Vol 419, pp 53-67). Approximately 5 nM of labeled RNA precursor is incubated for 41 hours at 25° C. in 20 mM MgCl₂, 50 mM Tris HCl (pH 8.3 at 25° C.) in the presence or absence of increasing concentrations of each ligand. In-line cleavage products are separated on 10% polyacrylamide gel electrophoresis (PAGE), and the resulting gel is visualized using a Molecular Dynamics Phosphorimager. The location of products bands corresponding to cleavage are identified by comparison to a partial digest of the RNA with RNase T1 (G-specific cleavage) or alkali (nonspecific cleavage).

In-line probing exploits the natural ability of RNA to self-cleave at elevated pH and metal ion concentrations (pH≈8.3, 25 mM MgCl₂) in a conformation-dependent manner. For self-cleavage to occur, the 2′-hydroxyl of the ribose must be “in-line” with the phosphate-oxygen bond of the internucleotide linkage, facilitating a S_{N}2P nucleophilic transesterification and strand cleavage. Typically, single-stranded regions of the Riboswitch are dynamic in the absence of an active ligand, and the internucleotide linkages in these regions can frequently access the required in-line conformation. Binding of an active ligand to the Riboswitch generally reduces the dynamics of these regions, thereby reducing the accessibility to the in-line conformation, resulting in fewer in-line cleavage events within those regions. These ligand-dependent changes in RNA cleavage can be readily detected by denaturing gel electrophoresis. The relative binding affinity of each ligand is expressed as Iₘₐₓ, wherein Iₘₐₓ represents the percent inhibition of in-line cleavage at selected internucleotide ligands in the presence of a fixed ligand concentration (20 μM for the FMN riboswitch and 100 μM for the CD3299 riboswitch) normalized to the percent inhibition in the absence of ligand and the percent inhibition in the presence of a saturation concentration of a control ligand. 100 μM FMN is used as a control ligand for estimating binding to the FMN riboswitch and 100 μM of a standard compound A (which is a compound which has a high affinity against the CD3299 riboswitch) is used as a control ligand for estimating binding to the CD3299 riboswitch.

The experiments show that various Compounds of the Invention, particularly compounds described in formula 1.41 and 1.43, have a binding affinity to FMN riboswitch with an IC₅₀ value of less than, or equal to, 75 μM, preferably less than or equal to 50 μM, more preferably, less than or equal to 25 μM, still more preferably, less than or equal to 10 μM. The experiments also show that various Compounds of the invention, e.g., have a binding affinity to FMN riboswitch with an Imax value of greater than or equal to 20% compared to the control (i.e., 100 μM of FMN), or a binding affinity to CD3299 riboswitch with an Imax of greater than 20% compared to the control (i.e., 100 μM of Compound A). In still other instances, the experiments show that various compounds of the Invention at 100 μM bind to the CD3299 riboswitch with an Iₘₐₓ value of approximately 100%, meaning that they bind approximately as well as the control compound.

### MIC Assay

### Example 2

The MIC assays are carried out in a final volume of 100 μL in 96-well clear round-bottom plates according to methods established by the Clinical Laboratory Standards Institute (CLSI). Briefly, test compound suspended in 100% DMSO (or another suitable solubilizing buffer) is added to an aliquot of media appropriate for a given pathogen to a total volume of 50 μL. This solution is serially diluted by 2-fold into successive tubes of the same media to give a range of test compound concentrations appropriate to the assay. To each dilution of test compound in media is added 50 μl of a bacterial suspension from an overnight culture growth in media appropriate to a given pathogen. Final bacterial inoculum is approximately 10⁵-10⁶ CFU/well. After growth for 18-24 hours at 37° C., the MIC is defined as the lowest concentration of antimicrobial agent that completely inhibits growth of the organism as detected by the unaided eye, relative to control for bacterial growth in the absence of added antibiotic. Ciprofloxacin is used as an antibiotic-positive control in each screening assay. Each of the bacterial cultures that are available from the American Type Culture Collection (ATCC, www.atcc.org) is identified by its ATCC number.

The experiments show that various compounds of the invention have a minimum inhibitory concentration (MIC) of less than 130 μg/mL, in particular instance, less than or equal to 64 μg/mL, in other instances 32 μg/mL against at least one of the bacteria selected from Clostridium difficile (e.g., C. difficile MMX3581 (clinical) and C. Difficile ATCC43596)), Staphylococcus epidermidis, Staphylococcus aureus (e.g., Staphylococcus aureus ATCC29213 and Stephylococcus aureus RN4220), Streptococcus pneumoniae, Pseudomonas aeruginosa, Acinetobacter baumannii, Haemophilus influenzae, Enterococcus faecalis and Streptococcus pyogenes. For examples, this experiments shows that the compounds of Formula I(B) or compounds of formula Q.39 have an MIC of less than 64 μg/mL.

All of the exemplified compounds of the invention have either an Iₘₐₓ value of greater than or equal to 20% in an assay as described in Example 1 (compared to at least one of the two controls at 100 μM) or an IC₅₀ value of less than or equal to 10 μM against the FMN riboswitch in an assay as described in Example 1 and/or a MIC of less than or equal to 1284 mL against at least one of the bacterial strains as described in Example 2. In certain embodiment, certain compounds of the invention have either an Iₘₐₓ, value of greater than 20% in an assay as described in Example 1 (compared to at least one of the two controls) or an IC₅₀ value of less than or equal to 10 μM against the FMN riboswitch and a MIC of less than or equal to 64 μg/mL against at least one of the bacterial strains as described in Example 2.

### Cytotoxic Assay

### Example 2a

The cytotoxic effects of test compounds on HepG2 are measured with a commercially available cell viability assay kit from Promega. On day 1, HepG2 cells (˜1×10⁴ cells) are seeded into each well in 96-well plate and cultured for approximately 24 h at 37° C. in a 5% CO₂ atmosphere under saturating humidity. On day 2, test compounds and DMSO controls are added to appropriate wells to give a range of test compound concentrations appropriate to the assay. Terfenadine is also added to each plate as a positive cytotoxic control. Control wells containing medium without cell are prepared to obtain a value for background luminescence. Assay plates are then cultured for approximately 24 h at 37° C. in a 5% CO₂ atmosphere under saturating humidity. On day 3, assay plates are removed from 37° C. incubator and equilibrated to 22° C. Once equilibrated, CellTiter-Glo® reagent is added to each well containing cell culture medium, followed by mixing to allow cell lysis. The CellTiter-Glo® Assay measures the number of viable cells in culture based on quantitation of the ATP present, an indicator of metabolically active cells. This assay generates a luminescent signal proportional to the amount of ATP present. The amount of ATP is directly proportional to the number of cells present in culture. After the assay plate is incubated at room temperature for approximately 10 min to stabilize luminescent signal, luminescence is recorded on PerkinElmer luminometer. CC₅₀ is defined as the concentration of test compounds in μM to result in 50% reduction in luminescence signal relative to the signal for untreated cells.

The experiments show that various compounds of the invention have a CC₅₀ value of 7 μg/mL to greater than 45 μg/mL, in some instances greater than or equal to 30 μM, and in particular instances, greater than or equal to 45 μM, in still other instances, greater than or equal to 65 μM. In certain instances, various compounds of the Invention have a CC₅₀ value of greater than 30 μM and MIC of less than 8 μg/mL.

### Synthesis of Compounds of the Invention

Temperatures are given in degrees Celsius (° C.); unless otherwise stated, operations are carried out at room or ambient temperature, that is, at a temperature in the range of 18-25° C. Chromatography means flash chromatography on silica gel; thin layer chromatography (TLC) is carried out on silica gel plates. Samples were dissolved in deuterated solvents for NMR spectroscopy. NMR data is in the delta values of major diagnostic protons, given in parts per million (ppm) relative to the appropriate solvent signals. Conventional abbreviations for signal shape are used. For mass spectra (MS), the lowest mass major ion is reported for molecules where isotope splitting results in multiple mass spectral peaks. Solvent mixture compositions are given as volume percentages or volume ratios. In cases where the NMR spectra are complex, only diagnostic signals are reported.

### General Methods for Analytical HPLC Analysis:

Method A: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: 95% aqueous (0 to 0.5 min); a gradient from 95% aqueous to 98% organic (0.5 to 10.5 min); 98% organic (2 min); a gradient from 98% organic to 95% aqueous (5.5 min); 95% aqueous (1 min).

Method B: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: 95% aqueous (0 to 0.5 min); a gradient from 95% aqueous to 100% organic (0.5 to 10.5 min); a gradient from 100% organic to 95% aqueous (2 min); 95% aqueous (4 min).

Method C: Analytical LCMS is performed using a YMC Combiscreen ODS-AQ, 5 μm, 4.6×50 mm column. The aqueous phase is 1% 2 mM NH₄OAc in 90:10 IPA:H₂O, 0.03% TFA in USP water. The organic phase is 1% 2 mM NH₄OAc in 90:10 IPA:H₂O, 0.03% TFA in acetonitrile. The elution profile is as follows: a gradient from 95% aqueous to 100% organic (0 to 10 min); 100% organic (2 min); a gradient from 100% organic to 95% aqueous (0.1 min); 95% aqueous (3 min).

Method D: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: a gradient from 95% aqueous to 75% aqueous (0 to 10 min); a second gradient from 75% aqueous to 98% organic (2.5 min); a third gradient to 95% aqueous (over 1 min).

Method E: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: a gradient from 95% aqueous to 40% aqueous (0 to 10 min); a second gradient from 40% aqueous to 2% aqueous (2 min); 2% aqueous (1 min); 2% aqueous to 95% aqueous (4 min).

Method F: Analytical HPLC is performed using a Luna Prep C₁₈, 100 Å 5 □m, 4.6×100 mm column. The aqueous phase is 0.1% TFA in USP water. The organic phase is 0.1% TFA in acetonitrile. The elution profile is as follows: a gradient from 95% aqueous to 60% aqueous (0 to 10 min); a second gradient from 60% aqueous to 2% aqueous (2 min); 2% aqueous (1 min); 2% aqueous to 95% aqueous (4 min).

System D: Agilent 1100 HPLC, Agilent XDB C18 50×4.6 mm 1.8 micron column, 1.5 mL/min, Solvent A—Water (0.1% TFA), Solvent B—Acetonitrile (0.07% TFA), Gradient—5 min 95% A to 95% B; 1 min hold; then recycle, UV Detection @ 210 and 254 nm.

System E: Agilent 1100 HPLC, Agilent XDB C18 150×4.6 mm 1.8 micron column, 1.5 mL/min, Solvent A—Water (0.1% TFA), Solvent B—Acetonitrile (0.07% TFA), Gradient—7 min 95% A to 95% B; 1 min hold; then recycle, UV Detection @ 210 and 254 nm.

### General Procedure for Preparative HPLC Conditions.

Method 1 Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: 100% aqueous (0 to 3 min); a gradient from 100% aqueous to 98% organic (3 to 21 min); 98% organic (1 min); a gradient from 98% organic to 95% aqueous (1 min); 95% aqueous (1 min).

Method 2 Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: a gradient from 95% aqueous to 25% organic (0 to 10 min); a second gradient from 25% organic to 98% organic (over 2.5 min min); a third gradient to 95% aqueous (over 1 min).

Method 3 Preparatory HPLC is performed using aSunFire™ Prep C18 OBD™ 5 μm, 30×100 mm column. The aqueous phase is 0.1% TFA in USP water. The organic phase is acetonitrile. The elution profile is as follows: 100% aqueous (0 to 3 min); a gradient from 100% aqueous to 60% organic (3 to 21 min); then to 98% organic (21 to 24 min); a gradient from 98% organic to 95% aqueous (1 min); 95% aqueous (1 min).

Method 4 Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: a gradient from 100% aqueous to 60% organic (0 to 29 min); then to 98% organic (29 to 31 min); 98% organic (2 min); a gradient from 98% organic to 100% aqueous (2 min); 100% aqueous (2 min).

### Terms and Abbreviations

ACN=acetonitrile,
AcOH=acetic acid
Ar=argon
Bn=benzyl,
br=broad,
t-BuOH=tert-butyl alcohol,
cat.=catalytic,
CAN=ammonium cerium (IV) nitrate,
CBzCl=benzyl chloroformate
conc.=concentrated,
d=doublet,
DCM=dichloromethane,
D-ribose=(2R,3R,4R)-2,3,4,5-tetrahydroxypentane,
DIAD=diisopropyl azodicarboxylate,
DIPEA=diisopropylethylamine,
DMF=N,N-dimethylformamide,
DCM=dichloromethane
DMAP=N,N-dimethylaminopyridine,
DMSO=dimethyl sulfoxide,
Et₂O=diethyl ether,
Et₃N=triethyl amine,
EtOAc=ethyl acetate,
EtOH=ethyl alcohol,
equiv.=equivalent(s),
flash chromatography; as described in Still, W. C, Kahn, M.; Mitra, A. J. Org. Chem 1978, 43, 2923,
h=hour(s),
H₂O=water,
HATU=2-(1H-7-Azabenzotriazol-1-yl)-1,1,3,3-tetramethyl uronium hexafluorophosphate methanaminium,
HBTU=2-(1H-Benzotriazole-1-yl)-1,1,3,3-tetramethyluronium hexafluorophosphate,
HCl=hydrochloric acid
HPLC=high performance liquid chromatography,
HOAc=acetic acid,
IPA=isopropyl alcohol,
ISCO=normal phase silica gel cartridges supplied by Teledyne ISCO,
K₂CO₃=potassium carbonate,
LiBH₄=lithium tetrahydroborate,
LAH=lithium tetrahydroaluminate,
m=multiplet,
min.=minute(s),
MgCl₂=magnesium chloride
MeOH=methanol,
NaHCO₃=sodium bicarbonate,
Na₂SO₄=sodium sulfate,
NH₄OH=ammonium hydroxide,
NH₄OAc=ammonium acetate,
NMP=N-methylpyrrolidinone,
NMR=nuclear magnetic resonance,
p=pentet
PMB=p-methoxybenzyl,
POCl₃=phosphorous oxychloride,
POMCl=pivaloyloxymethylchloride,
PPh₃=triphenylphosphine,
PyBOP=benzotriazol-1-yl-oxytripyrrolidinophosphonium hexafluorophosphate,
rt=room temperature,
RNA=ribonucleic acid,
RNase T1=an endoribonuclease that specifically degrades single-stranded RNA at G residues,
s=singlet,
SOCl₂=thionyl chloride,
t=triplet,
TBAI=tetrabutylammonium iodide,
TFA=trifluoroacetic acid,
TFAA=trifluoroacetic anhydride,
THF=tetrahydrofuran,
TLC=thin layer chromatography,
TMSBr=trimethylsilyl bromide,
Tris HCl=Tris (hydroxymethyl)aminomethane hydrochloride
USP water=US Pharmacopeia (USP) grade water.

### Intermediate 1

### Preparation of: 14]-(2-aminoethyl)-7,8-dimethylbenzo[g]pteridine-2,4(3H,10H)-dione

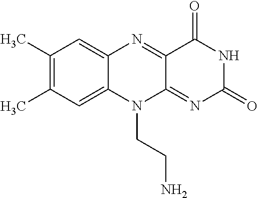

### Step 1 Preparation of 10-(2-(benzylamino)ethyl)-7,8-dimethylbenzo[g]pteridine-2,4(3H,10H)-dione

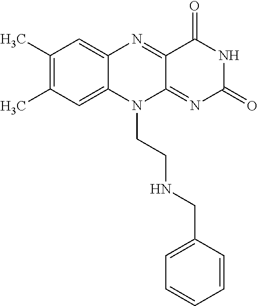

Prepared by reductive amination using a procedure similar to that of Example 3 using 2-(7,8-dimethyl-2,4-dioxo-3,4-dihydrobenzo[g]pteridin-10(2H)-yl)acetaldehyde (prepared by the method of step 1, Example 3) and benzylamine. This product is contaminated with 10-(2-(benzyl(methyl)amino)ethyl)-7,8-dimethylbenzo[g]pteridine-2,4(3H,10H)-dione. Next two steps are performed to isolate the product.

### Step 2 Preparation of tert-butyl benzyl(2-(7,8-dimethyl-2,4-dioxo-3,4-dihydrobenzo[g]pteridin-10(2H)-yl)ethyl)carbamate

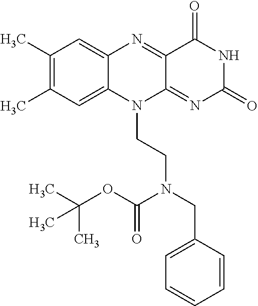

To a solution of crude 10-(2-(benzylamino)ethyl)-7,8-dimethylbenzo[g]-pteridine-2,4(3H,10H)-dione (7.53 mmol) in MeOH (200 mL) is added di-tert-butyl dicarbonate (5.2 g, 23.8 mmol) and Et₃N (4 mL). The reaction was concentrated under reduced pressure and purified via silica gel chromatography (ISCO) (100% DCM to 10% MeOH/DCM) over 1 h to obtain desired product (1.85 g, 54%) as a brown solid.

### Step 3 Preparation of 10-(2-(benzylamino)ethyl)-7,8-dimethylbenzo[g]pteridine-2,4(3H,10H)-dione 2,2,2-trifluoroacetate

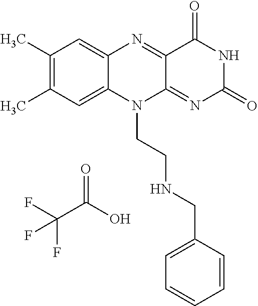

To a solution of tert-butyl benzyl(2-(7,8-dimethyl-2,4-dioxo-3,4-dihydrobenzo[g]pteridin-10(2H)-yl)ethyl)carbamate (50 mg, 0.11 mmol) in DCM (2 mL) is added TFA (2 mL) at rt. After 2 h, the reaction mixture is concentrated and the residual material is dissolved in MeOH (10 ml) and purified by preparative HPLC (Method 2). Lyophilization of combined pure fractions (LCMS) affords desired product (33.6 mg, 65%) as a brown solid. ¹H NMR (400 MHz, DMSO-d₆) δ 2.42 (s, 3H), 2.53 (s, 3H), 4.35 (s, 3H), 5.00 (m, 2H), 7.43 (m, 3H), 7.52 (m, 2H), 7.83 (s, 1H), 7.96 (s, 1H), 9.02 (s, 2H), 11.49 (s, 1H).

### Step 4: Preparation of 10-(2-Aminoethyl)-7,8-dimethylbenzo[g]pteridine-2,4(3H,10H)-dione-2,2,2-trifluoroacetate salt

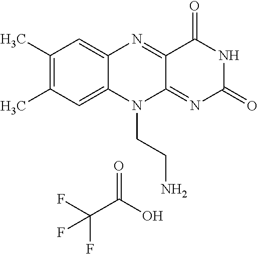

A solution of flavin ethyl benzyl amine (step 3) (395 mg, 1.05 mmol) and Pd/C (75 mg) in absolute EtOH (100 mL) is hydrogenated at 30 psi and 45° C. overnight. The mixture is filtered through a celite pad. The filtrate is concentrated under reduced pressure to dryness to obtain a crude product (230 mg, 76.6%). Crude product (19.5 mg, 0.07 mmol) is dissolved in MeOH (8 mL) and purified by preparative HPLC (Method 2). Lyophilization of the combined pure fractions (LCMS) affords desired product (5.0 mg, 14.3%) as a brown solid. ¹H NMR (400 MHz, DMSO-d₆) δ 2.42 (s, 3H), 2.50 (s, 3H), 4.20 (m, 2H), 4.87 (m, 2H), 7.81 (s, 1H), 7.88 (m, 2H), 7.97 (s, 1H), 11.45 (s, 1H).

### Intermediate 2

### 1-(3-Bromopropyl)-1H-pyrrole

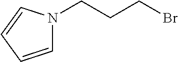

To a cooled (0-5° C.) solution 3-(1H-pyrrol-1-yl)propan-1-ol (800 mg, 6.39 mmol) in CH₂Cl₂ (30 mL) is added triphenylphosphine dibromide (3.091 g, 7.03 mmol) with stirring. After 10 min, the ice bath is removed and the mixture is stirred an additional 3 h at rt. Water is added and the mixture is diluted with CH₂Cl₂. The layers are separated and the organic layer is washed with brine, dried (anhydrous sodium sulfate), filtered and concentrated at reduced pressure. The residue is purified by flash chromatography (230-400 mesh, hexane/ethyl acetate (5%) containing 0.1% isopropanol as eluant) to afford 630 mg (52%) of the desired product as a clear oil. ¹H NMR (400 MHz, CDCl₃) δ 2.82 (p, 2H), 3.33 (t, 2H), 4.10 (t, 2H), 6.18 (m, 2H), 6.70 (m, 2H); HPLC retention time: 3.91 min. (Method G).

### Intermediate 3: 1-(3-Bromopropyl)-1H-imidazole

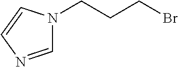

### Step 1 Preparation of methyl 3-(1H-imidazol-1-yl)propanoate

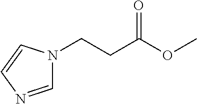

To a solution of 1H-imidazole (1.000 g, 14.7 mmol) in acetonitrile (20 mL) in a pressure tube is added methyl acrylate (2.65 mL, 29.4 mmol). The tube is sealed and heated at 80° C. Additional methyl acrylate (1.32 mL, 14.7 mmol) is added after 8 h and 12 h, respectively. After 17 h, volatiles are removed at reduced pressure and the residue is dissolved in ethyl acetate. The solution is washed with brine, dried (anhydrous sodium sulfate), filtered and concentrated at reduced pressure to afford 2.13 g (94%) of the desired product as oil. ¹H NMR (400 MHz, CDCl₃) δ 2.80 (t, 2H), 3.71 (s, 3H), 4.29 (t, 2H), 6.94 (s, 1H), 7.06 (s, 1H), 7.52 (s, 1H); MS (ESI+) for C₇H₁₀N₂O₂ m/z 155.2 (M+H)⁺.

### Step 2 Preparation of 3-(1H-imidazol-1-yl)propan-1-ol

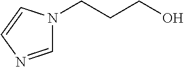

To a flask containing lithium aluminum hydride (379 mg, 9.99 mmol) is slowly added tetrahydrofuran (8 mL). The mixture is stirred for 10 min. at rt then cooled (0-5° C.). A solution of methyl 3-(1H-imidazol-1-yl)propanoate (770 mg, 4.99 mmol) in THF (3 mL) is added drop wise and the mixture is stirred an additional 5 min. at 0-5° C. The mixture is heated to 70° C. for 3 h. The mixture is cooled to rt and with vigorous stirring the reaction is quenched by the sequential addition of water (0.38 mL), 15% aqueous NaOH (0.38 mL), and water (1.14 mL). The solids are removed by filtration through a pad of Celite and the filtrate is dried (anhydrous sodium sulfate), filtered and concentrated at reduced pressure. Purification of the residue by flash chromatography (230-400 mesh, CH₂Cl₂/methanol (3-5%) as eluant) afforded 554 mg (88%) of the desired product as an oil. ¹H NMR (400 MHz, CDCl₃) δ 2.02 (p, 2H), 3.63 (t, 2H), 4.13 (t, 2H), 6.95 (s, 1H), 7.07 (s, 1H), 7.49 (s, 1H); MS (ESI+) for C₆H₁₀N₂O m/z 127.1 (M+H)⁺.

### Step 3 Preparation of 1-(3-bromopropyl)-1H-imidazole

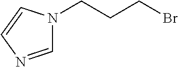

To a 0-5° C. solution 3-(1H-imidazol-1-yl)propan-1-ol (300 mg, 2.38 mmol) in CH₂Cl₂ (10 mL) is added triphenylphosphine dibromide (1.150 g, 2.62 mmol) with stirring. After 10 min., the ice bath is removed and the mixture is stirred an additional 3 h at rt. Water is added and the reaction mixture is diluted with CH₂Cl₂. The layers are separated and the organic layer is washed with saturated, aqueous sodium bicarbonate, brine, dried (anhydrous sodium sulfate), filtered and partially concentrated at reduced pressure to an approximate volume of 3 mL. This solution is used immediately in the next step. ¹H NMR (400 MHz, CDCl₃) δ 2.29 (p, 2H), 2.33 (t, 2H), 4.18 (t, 2H), 6.95 (s, 1 H), 7.09 (s, 1H), 7.54 (s, 1H).

### Intermediate 4: 5-(3-Bromopropyl)-3-methylisoxazole

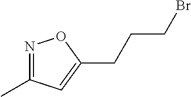

### Step 1 Preparation of 3-(3-methylisoxazol-5-yl)propan-1-ol

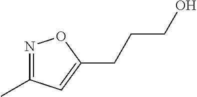

n-Butyllithium (2.5 M in hexane) (8.24 mL, 20.6 mmol) is added to a solution of 3,5-dimethylisoxazole (2.02 mL, 20.6 mmol) in 20 mL of THF which is cooled to −78° C. under N₂. The mixture is stirred at −78° C. for 2 h. A solution of ethylene oxide (0.907 g, 20.6 mmol) in 10 mL of THF is added to the mixture at −78° C. and the mixture is stirred at −78° C. for 30 min. Saturated, aqueous NH₄Cl is added and the mixture is warmed to rt. The pH of the aqueous phase is adjusted to ˜7 with 1.0 N HCl and the THF is evaporated. The solution is extracted with 3×20 mL of CH₂Cl₂ and the combined organic layers are dried over Na₂SO₄. Evaporation of the organic layer gives 1.7 g of an oil. Residual 3,5-dimethylisoxazole is removed by drying under high vacuum at rt for 2 h to give 1.3 g (45%) of the desired product as an orange oil. ¹H NMR (400 MHz, CDCl₃) δ 5.86 (s, 1H), 3.72 (m, 2H), 2.85 (t, 2H), 2.28 (s, 3H), 1.91-2.00 (m, 2H), 1.65 (m, 1H).

### Step 2 Preparation of 5-(3-bromopropyl)-3-methylisoxazole

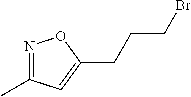

Bromine (0.109 mL, 2.12 mmol) is added to a solution of triphenylphosphine (0.557 g, 2.12 mmol) and pyridine (0.172 mL, 2.12 mmol) in 20 mL of CH₂Cl₂ which is cooled in an ice bath under N₂. Triphenylphosphine is added until the yellow color disappears. 3-(3-Methylisoxazol-5-yl)propan-1-ol (0.200 g, 1.42 mmol) is added and the mixture is stirred with ice bath cooling for 15 min. The ice bath is removed and the mixture is stirred at rt for 1 h. The mixture is extracted with 3×20 mL of 1.0 N aqueous HCl followed by 20 mL of saturated, aqueous NaHCO₃. The organic layer is dried over Na₂SO₄ and evaporation gives 0.4 g of a white solid. The solid is taken up in 20 mL of hexane and the solid is removed by filtration through a pad of silica gel (20 g). The pad is eluted with 200 mL of 50% EtOAc/hexane. Evaporation of the eluant gives 0.22 g (70%) of desired product as a clear oil. ¹H NMR (400 MHz, CDCl₃) δ 5.90 (s, 1H), 3.45 (t, 2H), 2.93 (t, 2H), 2.29 (s, 3H), 2.26 (m, 2H).

### Example 3

### Preparation of: 3-(S)-[2-(7,8-Dimethyl-2,4-dioxo-3,4-dihydro-2H-benzo[g]pteridin-10-yl)-ethylamino]-(R)-cyclopentanecarboxylic acid trifluoroacetic acid salt

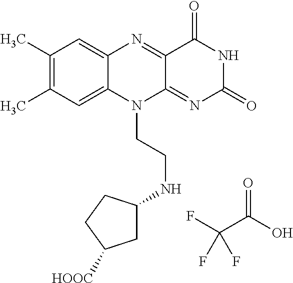

### Step 1: Preparation of 2-(7,8-Dimethyl-2,4-dioxo-3,4-dihydrobenzo[g]pteridin-10(2H)-yl)acetaldehyde

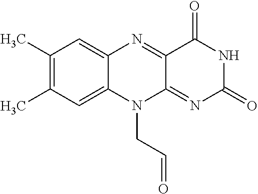

To a suspension of riboflavin (8.5 g, 0.0023 mol) in 2 N aqueous sulfuric acid (225 mL), cooled to 0° C. in a flask covered with tinfoil, is added orthoperiodic acid (18.9 g, 0.0825 mmol) dissolved in water (200 mL). After 30 min., the reaction is allowed to warm to room temperature. Once the reaction mixture becomes clear (a transparent yellow solution), the pH of the reaction solution is adjusted carefully to 3.8-3.9 (using a pH meter) by addition of solid sodium carbonate. [It is extremely important that the pH is monitored carefully, if one goes over a pH of 3.9 the product does not precipitate out of solution.] The precipitate is then filtered off and washed liberally with cold water, ethanol, and diethyl ether to yield 6.04 g of the desired product as an orange solid (Yield: 94%). LC-MS m/z 285.1 [M+H]⁺, retention time 1.63 min.

### Step 2: Preparation of 3-(S)-[2-(7,8-Dimethyl-2,4-dioxo-3,4-dihydro-2H-benzo[g]pteridin-10-yl)-ethylamino]-(R)-cyclopentanecarboxylic acid trifluoro-acetic acid salt

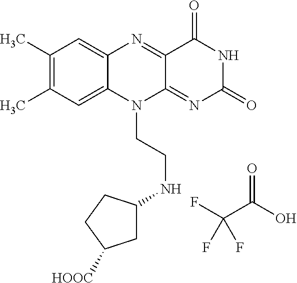

To a suspension of 2-(7,8-dimethyl-2,4-dioxo-3,4-dihydrobenzo[g]pteridin-10(2H)-yl)acetaldehyde (100 mg, 0.35 mmol) in methanol (10 mL) is added 3-amino-cyclopentanecarboxylic acid (100 mg, 0.77 mmol) at room temperature. Glacial acetic acid (7 drops) is added and allowed to stir at room temperature for 3 h. Sodium cyanoborohydride (48 mg, 0.77 mmol) is added and the solution is stirred for 16 h. The reaction mixture is concentrated, and the residue is dissolved in DMSO (5 mL), filtered, and purified by preparative HPLC (Method 1). 3-(S)-[2-(7,8-Dimethyl-2,4-dioxo-3,4-dihydro-2H-benzo[g]pteridin-10-yl)-ethylamino]-(R)-cyclopentanecarboxylic acid (8.2 mg) is isolated following lyophilization of the appropriate fractions (Yield: 6.0%). ¹H NMR (300 MHz, DMSO-d₆) δ 1.7 (m, 2H), 1.85 (m, 1H), 2.03 (m, 2H), 2.10 (m, 1H), 2.43 (s, 3H), 2.53 (s, 3H), 2.90 (m, 2H), 3.73 (m, 2H), 4.92 (m, 2H), 7.78 (s, 1H), 7.98 (s, 1H), 8.66 (m, 2H), 11.49 (s, 1H), 12.35 (s, 1H).

### Example 4

### Preparation of: 1-[2-(7,8-Dimethyl-2,4-dioxo-3,4-dihydro-2H-benzo[g]pteridin-10-yl)-ethyl]-pyrrolidine-3-carboxylic acid

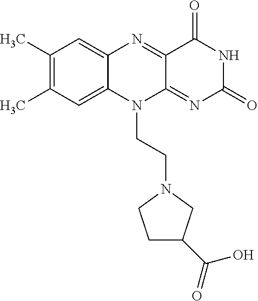

To a suspension of 1-[2-(7,8-dimethyl-2,4-dioxo-3,4-dihydro-2H-benzo[g]pteridin-10-yl)-ethyl]-pyrrolidine-3-carboxylic acid methyl ester (25 mg, 0.063 mmol) [prepared using the same procedure as Step 2 for the preparation of Example 3 with methylpyrrolidine-3-carboxylate in a 1:1 solution of THF:H₂O (10 mL) is added lithium hydroxide (15 mg, 0.63 mmol) at room temperature. The reaction mixture is allowed to stir at room temperature for 15 h, at which point 1M aqueous HCl (10 drops) is added. The reaction mixture is then concentrated, dissolved in MeOH (6 mL), water (2 mL), and purified by preparative HPLC (Method 1). Lyophilization of appropriate fractions provides 19 mg of desired product as a yellow fluffy solid (Yield: 79%). LC-MS m/z 383.0 [M+H]% retention time 1.53 min.

### Example 5

### 10-(2-((2-Methoxypyridin-4-yl)methylamino)ethyl)-7,8-dimethylbenzo[g]pteridine-2,4(3H,10H)-dione

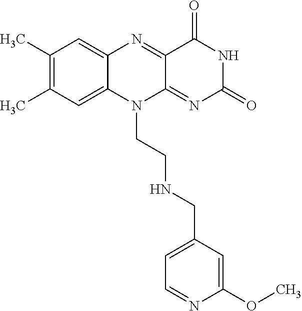

To a suspension of 10-(2-aminoethyl)-7,8-dimethylbenzo[g]pteridine-2,4(3H,10H)-dione (see Intermediate 1 for preparation) (46 mg, 0.16 mmol) in MeOH (5 mL) is added 2-methoxyisonicotinaldehyde (prepared as in C. Subramanyam, M. Noguchi and S. M. Weinreb, J. O. C., 1989, 54, 5580) (22 mg, 0.16 mmol), followed with acetic acid (0.1 mL) at rt. After 30 min., sodium cyanoborohydride (30 mg, 0.47 mmol) is added, and the solution is stirred for 16 h. The reaction mixture is concentrated, and the residue is dissolved in DMF (4 mL)/water (3 mL), filtered, and purified by preparative HPLC (Method 2). Lyophilization of the combined pure fractions affords desired product (6.5 mg, 9.7%). ¹H NMR (400 MHz, CD₃OD) δ 2.50 (s, 3H), 2.63 (s, 3H), 3.70 (m, 2H), 3.94 (s, 3H), 4.37 (s, 2H), 5.10 (m, 2H), 6.95 (s, 1H), 7.08 (d, 1H), 7.81 (s, 1H), 7.96 (s, 1H), 8.21 (d, 1H).

### Example 6

### 2-((1R,3S)-3-(2-(7,8-dimethyl-2,4-dioxo-3,4-dihydrobenzo[g]pteridin-10(2H)-yl)ethylamino)cyclopentyl)acetic acid

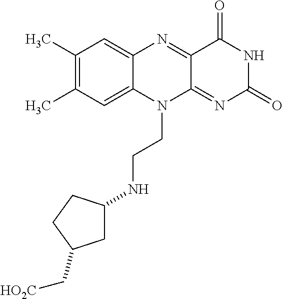

Prepared by reductive amination using a procedure similar to that of Example 3, using 2-(7,8-dimethyl-2,4-dioxo-3,4-dihydrobenzo[g]pteridin-10(2H)-yl)acetaldehyde (prepared by the method of step 1, Example 3) and 2-((1R,3S)-3-aminocyclopentyl)acetic acid as starting materials. ¹H NMR (400 MHz, DMSO-d₆) δ 1.30 (m, 2H), 1.80 (m, 3H), 2.20 (m, 2H), 2.32 (d, 2H), 2.43 (s, 3H), 2.53 (s, 3H), 3.00 (m, 1H), 3.65 (m, 2H), 4.92 (m, 2H), 7.78 (s, 1H), 7.98 (s, 1H), 8.65 (m, 1H), 11.48 (s, 1H), 12.14 (s, 1H).

### Example 7

### (1R,3R)-3-(2-(7,8-dimethyl-2,4-dioxo-3,4-dihydrobenzo[g]pteridin-10(2H)-yl)ethylamino)cyclopentanecarboxylic acid with 2,2,2-trifluoroacetic acid (1:1)

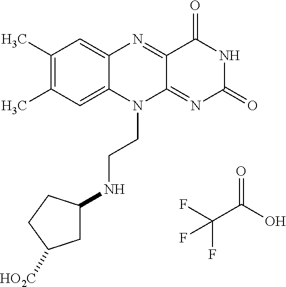

Prepared by reductive amination using a procedure similar to that of Example 3, using 2-(7,8-dimethyl-2,4-dioxo-3,4-dihydrobenzo[g]pteridin-10(2H)-yl)acetaldehyde (prepared by the method of step 1, Example 3) and (1R,3R)-3-aminocyclopentanecarboxylic acid as starting materials. ¹H NMR (400 MHz, DMSO-d₆) δ 1.70 (m, 2H), 1.85 (m, 1H), 2.03 (m, 2H), 2.10 (m, 1H), 2.43 (s, 3H), 2.53 (s, 3H), 2.90 (m, 2H), 3.73 (m, 2H), 4.92 (m, 2H), 7.78 (s, 1H), 7.98 (s, 1H), 8.66 (m, 2H), 11.49 (s, 1H), 12.35 (s, 1H).

### Example 8

### 7,8-Dimethyl-10-(2-piperidin-1-yl-ethyl)-10H-benzo[g]pteridine-2,4-dione TFA salt

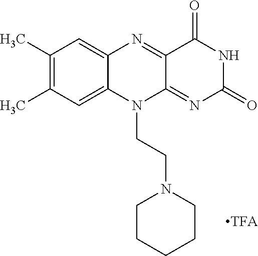

To a solution of 2-(7,8-dimethyl-2,4-dioxo-3,4-dihydrobenzo[g]pteridin-10(2H)-yl)acetaldehyde (prepared by the method of step 1, Example 3)(48 mg, 0.17 mmol) in methanol (20 mL) are added piperidine (40 μL, 0.4 mmol) and AcOH (0.5 mL) at room temperature. The reaction is stirred at 50° C. for 1 h. Then the reaction is cooled to room temperature and sodium cyanoborohydride (20 mg, 0.20 mmol) is added. After 17 h piperidine (0.1 mL) is added again and stirring continued at room temperature for 24 h. The solvent is removed under vacuum and the crude product is dissolved in DMSO/H₂O (1/7 mL), filtered, and purified by preparative HPLC (Method 1). 8-Dimethyl-10-(2-piperidin-1-yl-ethyl)-10H-benzo[g]pteridine-2,4-dione TFA salt (33 mg) is isolated following lyophilization of the appropriate fractions (Yield: 55%). ¹H NMR (400 MHz, DMSO-d6) δ 1.38 (m, 1H), 1.62 (m, 3H), 1.85 (m, 2H), 2.43 (s, 3H), 2.54 (s, 3H), 3.05 (m, 2H), 3.49 (brs, 2H), 3.83 (m, 2H), 4.98 (m, 2H), 7.85 (s, 1H), 7.99 (s, 1H), 8.60 (brs, 1H) 11.51 (s, 1H).

### Example 9

### 10-[2-(2-Hydroxymethyl-pyrrolidin-1-yl)-ethyl]-7,8-dimethyl-10H-benzo[g]pteridine-2,4-dione

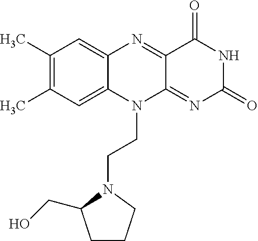

10-[2-(2-Hydroxymethyl-pyrrolidin-1-yl)-ethyl]-7,8-dimethyl-10H-benzo[g]pteridine-2,4-dione (5 mg; yield: 3.8%) is isolated by preparative HPLC (Method 1) as the by-product of the reductive amination reaction of 2-(7,8-dimethyl-2,4-dioxo-3,4-dihydrobenzo[g]pteridin-10(2H)-yl)acetaldehyde (prepared by the method of step 1, Example 3) with L-glutamic acid (see Example 3, step 2 for preparation although with heating of the imine formation at 40° C. for 30 min instead of rt for 3 h). ¹H NMR (400 MHz, DMSO-d6) δ 1.57 (m, 1H), 1.90 (m, 1H), 2.03 (m, 1H), 2.17 (m, 1H), 2.41 (s, 3H), 3.54 (m, 1H), 3.71 (m, 1H), 4.64 (m, 3H), 4.95 (m, 2H), 5.23 (m, 2H), 7.90 (s, 1H), 7.94 (s, 1H), 11.38 (s, 1H).

### Example 10

### 1-[2-(7,8-Dimethyl-2,4-dioxo-3,4-dihydro-2H-benzo[g]pteridin-10-yl)-acetyl]piperidine-4-carboxylic acid

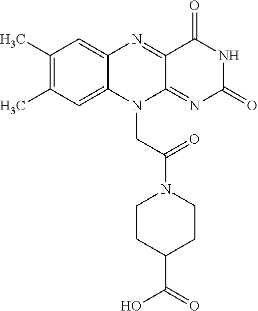

Step 1 Preparation of (7,8-Dimethyl-2,4-dioxo-3,4-dihydro-2H-benzo[g]pteridin-10-yl)-acetic acid

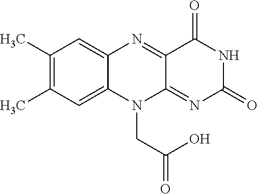

To a suspension of 2-(7,8-dimethyl-2,4-dioxo-3,4-dihydrobenzo[g]pteridin-10(2H)-yl)acetaldehyde (prepared by the method of step 1, Example 3) (50 mg, 0.18 mmol) in acetonitrile (2 mL), tert-butanol (8 mL), and methyl-1-cyclohexene (3 mL) at 0° C., a solution of sodium chlorite (122 mg, 1.35 mmol) and sodium dihydrogen phosphate (148 mg, 1.23 mmol) in 2 mL of water is added dropwise over 5 min. After 2 h the reaction mixture is diluted with water and the organic layer is discarded. The aqueous phase is concentrated under vacuum and the resultant crude mixture is purified via preparative HPLC. (7,8-Dimethyl-2,4-dioxo-3,4-dihydro-2H-benzo[g]pteridin-10-yl)-acetic acid (36 mg) was isolated following lyophilization of the appropriate fractions (Yield: 68%). LC-MS m/z 301.1 [M+H], retention time=1.68 min.

### Step 2 Preparation of 1-[2-(7,8-Dimethyl-2,4-dioxo-3,4-dihydro-2H-benzo[g]pteridin-10-yl)-acetyl]-piperidine-4-carboxylic acid tert-butyl ester

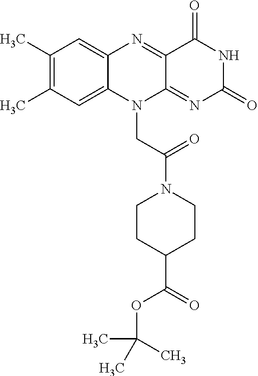

To a suspension of (7,8-dimethyl-2,4-dioxo-3,4-dihydro-2H-benzo[g]pteridin-10-yl)-acetic acid (50 mg, 0.17 mmol) and piperidine-4-carboxylic acid tert-butyl ester (32 mg, 0.17 mmol) in DMF (3 mL), i-PrNEt₂ (0.06 mL, 0.34 mmol) and HATU (65 mg, 0.17 mmol) are added sequentially at room temperature. After 17 h the temperature is increased to 50° C. for 3 h. The reaction mixture is cooled to room temperature, diluted with water (3 mL) and purified using preparative HPLC purification (Method 1). 1-[2-(7,8-Dimethyl-2,4-dioxo-3,4-dihydro-2H-benzo[g]pteridin-10-yl)-acetyl]-piperidine-4-carboxylic acid tert-butyl ester (4.9 mg) is isolated following lyophilization of the appropriate fractions (Yield: 7%). ¹H NMR (400 MHz, DMSO-d6) δ 11.39 (s, 1H), 7.95 (s, 1H), 7.61 (s, 1H), 5.63 (m, 2H), 4.08 (m, 2H), 2.88 (m, 1H), 2.48 (s, 3H), 1.76 (m, 4H), 1.44 (s, 9H). LC-MS m/z 468.0 [M+H]⁺, retention time=6.79 min.

### Step 3 Preparation of 1-[2-(7,8-Dimethyl-2,4-dioxo-3,4-dihydro-2H-benzo[g]pteridin-10-yl)-acetyl]-piperidine-4-carboxylic acid

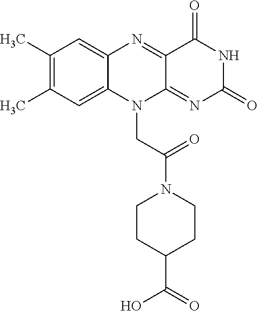

To a suspension of 1-[2-(7,8-dimethyl-2,4-dioxo-3,4-dihydro-2H-benzo[g]pteridin-10-yl)-acetyl]-piperidine-4-carboxylic acid tert-butyl ester (10 mg, 0.02 mmol) in CH₂Cl₂ (2 mL) is added trifluoroacetic acid (2 mL) at room temperature. After 2 h of stirring, the reaction mixture is concentrated and the residual material is dissolved in water/acetonitrile and lyophilized. 1-[2-(7,8-Dimethyl-2,4-dioxo-3,4-dihydro-2H-benzo[g]pteridin-10-yl)-acetyl]-piperidine-4-carboxylic acid (7.2 mg) is isolated (Yield: 80%). LC-MS m/z 410.1 [M−H]⁻, retention time=4.99 min.

### Example 11

### N-1,2-(7,8-Dimethyl-2,4-dioxo-3,4-dihydro-2H-benzo[g]pteridin-10-yl)-ethyl]-2-methoxy-nicotinamide

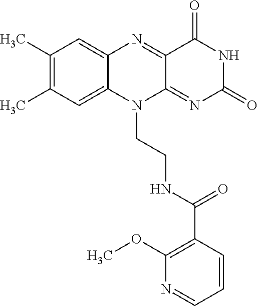

### Step 1 Preparation of 2-Methoxy-nicotinic acid

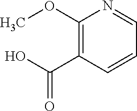

2-Methoxy-nicotinic acid methyl ester (500 mg, 3.0 mmol) is dissolved in methanol (5 mL) and water (1 mL). Sodium hydroxide (600 mg, 15 mmol) is added and the reaction mixture is refluxed for 2 h. The solution is neutralized with 1N HCl to pH 7 and concentrated under vacuum. The solid is washed with 30 mL of DCM/MeOH (1/1). The filtrate is concentrated under vacuum to yield 2-methoxy-nicotinic acid (407 mg) as a white solid (Yield: 88%). ¹H NMR (400 MHz, DMSO-d6) δ 8.05 (m, 1H), 7.87 (m, 1H), 6.91 (m, 1H), 3.83 (s, 3H).

### Step 2 Preparation of N-[2-(7,8-Dimethyl-2,4-dioxo-3,4-dihydro-2H-benzo[g]pteridin-10-yl)-ethyl]-2-methoxy-nicotinamide

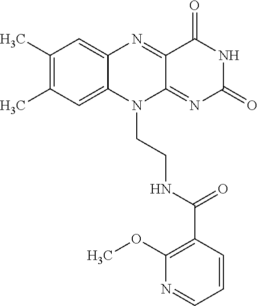

2-Methoxy-nicotinic acid (20 mg, 0.12 mmol) and Hunig's base (0.024 mL, 0.14 mmol) are dissolved in DMF (1 mL) followed by addition of HATU (53 mg, 0.14 mmol) at room temperature and is stirred for one hour. 10-(2-Amino-ethyl)-7,8-dimethyl-10H-benzo[g]pteridine-2,4-dione (39 mg, 0.14 mmol) (see Intermediate 1 for preparation) is dissolved in DMF (1 mL) and added to the reaction mixture. After 3 h the reaction mixture is diluted with water (2 mL) and purification is performed using preparatory HPLC (Method 3). 342-(7,8-Dimethyl-2,4-dioxo-3,4-dihydro-2H-benzo[g]pteridin-10-yl)-ethylamino]-benzoic acid (12 mg) is isolated following lyophilization of the appropriate fractions (Yield: 24%). ¹H NMR (400 MHz, DMSO-d6) δ 11.37 (s, 1H), 8.48 (m, 1H), 8.28 (m, 1H), 8.02 (m, 1H), 7.89 (m, 2H), 4.81 (m, 2H), 3.78 (m, 5H), 2.33 (s, 3H), 2.30 (s, 3H). LC-MS m/z 421.2 [M+H]⁺. Retention time=5.31 min.

### Example 12

### (S)-1-(2-(7,8-Dimethyl-2,4-dioxo-3,4-dihydrobenzo[g]pteridin-10(2H)-yl)ethyl)-N-(methylsulfonyl)pyrrolidine-2-carboxamide

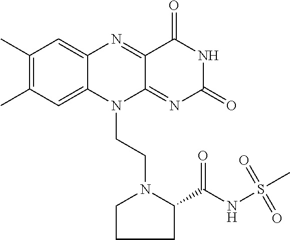

Methanesulfonamide is added to a mixture of (S)-1-(2-(7,8-dimethyl-2,4-dioxo-3,4-dihydrobenzo[g]pteridin-10(2H)-yl)ethyl)pyrrolidine-2-carboxylic acid (prepared by reductive amination using a procedure similar to that of step 2, Example 3, using 2-(7,8-dimethyl-2,4-dioxo-3,4-dihydrobenzo[g]pteridin-10(2H)-yl)acetaldehyde (prepared by the method of step 1, Example 3) and (S)-pyrrolidine-2-carboxylic acid) (35 mg, 0.09 mmol), HATU (130 mg, 0.34 mmol) and DIPEA (0.2 mL, 1.14 mmol) in DMF (3 mL) at rt. The reaction is stirred for 1 h. The solution is concentrated under reduced pressure, dissolved in ACN (6 mL)/water (2 mL) and purified by preparative HPLC (Method 2). Lyophilization of the combined pure fractions (LCMS) affords desired product (6.1 mg, 14.5%) as a brown solid. ¹H NMR (400 MHz, DMSO-d₆) δ 2.00 (m, 4H), 2.42 (s, 3H), 2.50 (s, 3H), 3.11 (s, 3H), 3.20 (m, 2H), 3.81 (m, 1H), 4.16 (m, 1H), 4.87 (m, 1H), 4.99 (m, 1H), 7.76 (s, 1H), 7.96 (s, 1H), 11.45 (s, 1H).

### Example 13

### (R)-1-(2-(7,8-Dimethyl-2,4-dioxo-3,4-dihydrobenzo[g]pteridin-10(2H)-yl)ethyl)-N-(methylsulfonyl)pyrrolidine-2-carboxamide

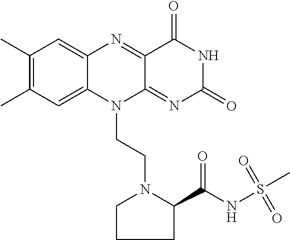

Methanesulfonamide (74 mg, 0.77 mmol) is added to a mixture of (R)-1-(2-(7,8-dimethyl-2,4-dioxo-3,4-dihydrobenzo[g]pteridin-10(2H)-yl)ethyl)pyrrolidine-2-carboxylic acid (prepared by reductive amination using a procedure similar to that of step 2, Example 3, using 2-(7,8-dimethyl-2,4-dioxo-3,4-dihydrobenzo[g]pteridin-10(2H)-yl)acetaldehyde (prepared by the method of step 1, Example 3) and (R)-pyrrolidine-2-carboxylic acid) (38 mg, 0.10 mmol), HATU (98 mg, 0.25 mmol) and diisopropylethylamine (100 mg, 0.77 mmol) in DMF (8 mL) at rt. The reaction is stirred for 1 h. The solution is concentrated under reduced pressure, dissolved in ACN (6 mL)/water (2 mL) and is purified by preparative HPLC (Method 2). Lyophilization of the combined pure fractions (LCMS) affords desired product (9.3 mg, 22.1%). ¹H NMR (400 MHz, CD₃OD) δ 2.21 (m, 2H), 2.26 (m, 2H), 2.50 (s, 3H), 2.63 (s, 3H), 3.19 (s, 3H), 3.50 (m, 1H), 3.83 (m, 2H), 4.16 (m, 1H), 4.48 (m, 1H), 5.05 (m, 2H), 7.77 (s, 1H), 8.01 (s, 1H).

### Example 14

### 7,8-Dimethyl-10-[5-(2-oxo-1,2-dihydro-pyridin-3-ylamino)-pentyl]-10H-benzo[g]pteridine-2,4-dione

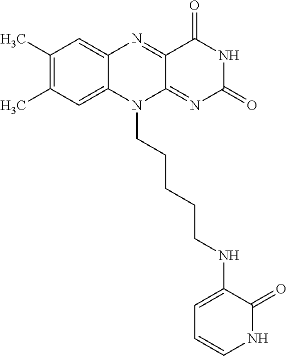

### Step 1 Preparation 5-(4,5-dimethyl-2-nitrophenylamino)pentan-1-ol

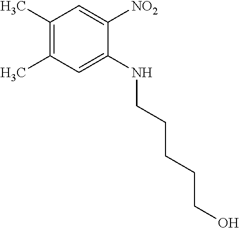

To a solution of 1-bromo-4,5-dimethyl-2-nitrobenzene (200 mg, 0.870 mmol) in anhydrous DMSO (1 mL), is added 5-aminopentan-1-ol (170 mg, 2.608 mmol). The reaction mixture is heated in a microwave at 140° C. for 20 min. The reaction mixture is concentrated under vacuum and diluted with water (5 mL) and the aqueous layer is extracted with DCM (3×5 mL). The organic layer is dried over Na₂SO₄, filtered, and concentrated under reduced pressure. Desired product (147 mg) is isolated (yield: 67%). ¹H NMR (400 MHz, CDCl₃) δ 1.55 (m, 2H), 1.66 (m, 2H), 1.79 (m, 2H), 2.20 (s, 3H), 2.29 (s, 3H), 2.38 (s, 2H), 3.32 (m, 2H), 3.71 (m, 2H), 6.64 (s, 1H), 7.95 (s, 1H).

### Step 2 Preparation of 5-(2-amino-4,5-dimethylphenylamino)pentan-1-ol

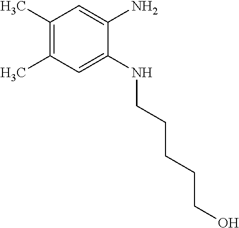

To a solution of 5-(4,5-dimethyl-2-nitrophenylamino)pentan-1-ol (147 mg, 0.583 mmol) in anhydrous MeOH (6 mL) under argon, is added Pd/C (8.4 mg) and sodium borohydride (64 mg, 1.68 mmol). Hydrogen is introduced via a balloon and the reaction mixture is stirred at room temperature for 30 min. The reaction mixture is filtered through celite, which is washed liberally with EtOH, and the solution is then concentrated to obtain the crude product as a clear, colourless oil which is used in the next step.

### Step 3 Preparation of 10-(5-hydroxypentyl)-7,8-dimethylbenzo[g]pteridine-2,4(3H,10H)-dione

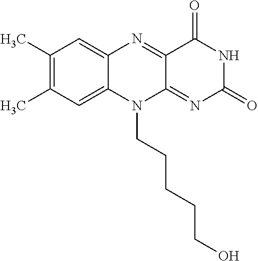

Crude 5-(2-amino-4,5-dimethylphenylamino)pentan-1-ol (0.583 mmol) is dissolved in glacial acetic acid (13 mL) under argon. Alloxan monohydrate (94 mg, 0.583 mmol) and boron oxide (81 mg, 1.165 mmol) are added to the stirring solution and the reaction is maintained under an argon atmosphere at 25° C. with stirring for 2 h. The reaction mixture is evaporated under vacuum and the residue is dry loaded on silica gel using DCM as a solvent and purified by Biotage flash column chromatography using a gradient from 0 to 10% MeOH in DCM as eluent. Desired product (70 mg) is isolated (yield: 37%). ¹H NMR (400 MHz, DMSO) δ 1.48 (m, 4H), 1.70 (m, 2H), 2.38 (s, 3H), 2.49 (s, 3H), 3.40 (m, 2H), 4.40 (t, 1H), 4.55 (m, 2H), 7.78, (s, 1H), 7.88 (s, 1H), 11.28 (s, 1H). ESI(+) [M+Na]⁺=351.2.

### Step 4 Preparation of 10-(5-bromopentyl)-7,8-dimethylbenzo[g]pteridine-2,4(3H,10H)-dione

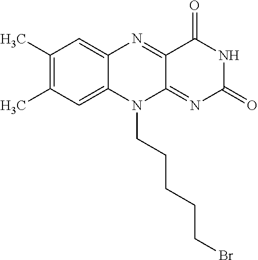

To a solution of 10-(5-hydroxypentyl)-7,8-dimethylbenzo[g]pteridine-2,4(3H,10H)-dione (72 mg, 0.219 mmol) and carbon tetrabromide (80 mg, 0.241 mmol) in anhydrous DMF (5 mL) at 0° C., is added triphenyl phosphine (152 mg, 0.460 mmol) portion-wise. The reaction mixture is stirred at room temperature for 18 h. The reaction mixture is concentrated under reduced pressure and the residue is dry loaded on silica gel using DCM:MeOH (50:50) as a solvent and purified by Biotage flash column chromatography using a gradient from 0 to 2% MeOH in DCM as eluent. Desired product (60 mg) is isolated (yield: 70%). ¹H NMR (400 MHz, DMSO) δ 1.60 (m, 2H), 1.73 (m, 2H), 1.90 (m, 2H), 2.40 (s, 3H), 2.50 (s, 3H), 3.58 (t, 2H), 4.59 (m, 2H), 7.80 (s, 1H), 7.90 (s, 1H), 11.31 (s, 1H). ESI(+) m/z=391.1, 393.1.

### Step 5 7,8-Dimethyl-10-[5-(2-oxo-1,2-dihydro-pyridin-3-ylamino)-pentyl]-10H-benzo[g]pteridine-2,4-dione

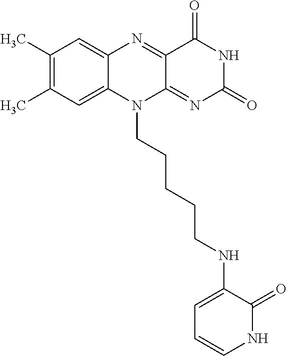

To a suspension of 3-aminopyridin-2(1H)-one (40 mg, 0.36 mmol) and Huning's base (35 mg, 0.27 mmol) containing the catalytic amount of sodium iodide in anhydrous DMF (5 mL) is added 10-(5-bromopentyl)-7,8-dimethylbenzo[g]pteridine-2,4(3H,10H)-dione (Step 4 of this Example) (70 mg, 0.18 mmol) at room temperature. The mixture is then heated to 80° C. and stirred for 5 h, concentrated (50° C.), dissolved in DMF/water (1/3) and purified by preparative HPLC (Method 1) to give after isolation and lyophilization 7,8-dimethyl-10-[5-(2-oxo-1,2-dihydro-pyridin-3-ylamino)-pentyl]-10H-benzo[g]pteridine-2,4-dione (5 mg, yield: 6.6%). ¹H NMR (400 MHz, DMSO-d6) δ 1.52 (m, 2H), 1.64 (m, 2H), 1.76 (m, 2H), 2.08 (s, 3H), 3.04 (m, 2H), 4.59 (m, 2H), 6.09 (m, 1H), 6.19 (m, 1H), 6.58 (m, 1H), 7.81 (s, 1H), 7.91 (s, 1H), 11.31 (s, 1H), 11.32 (brs., 1H).

### Example 15

### 10-[5-(4-Amino-2-oxo-2H-pyrimidin-1-yl)-pentyl]-7,8-dimethyl-10H-benzo[g]pteridine-2,4-dione

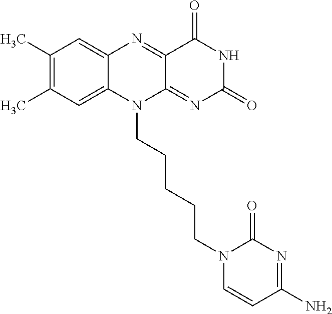

To a suspension of 4-aminopyrimidin-2(1H)-one (36 mg, 0.32 mmol) in anhydrous DMF (5 mL) cooled to 5° C. is added sodium hydride. The reaction mixture is stirred at room temperature for 30 min, then 10-(5-bromopentyl)-7,8-dimethylbenzo[g]pteridine-2,4(3H,10H)-dione (see Step 4 of Example 14) (50 mg, 0.128 mmol) is added and the reaction mixture is stirred for 6 h. The reaction mixture is concentrated (50° C.), dissolved in DMF/water (1/3) and purified by preparative HPLC (Method 1) to give, after isolation and lyophilization, 10-[5-(4-amino-2-oxo-2H-pyrimidin-1-yl)-pentyl]-7,8-dimethyl-10H-benzo[g]pteridine-2,4-dione (14 mg, yield: 26%). ¹H NMR (400 MHz, DMSO-d6) δ 1.46 (m, 2H), 1.74 (m, 4H), 2.40 (s, 3H), 2.51 (s, 3H), 3.78 (m, 2H), 4.57 (m, 2H), 6.05 (d, 1H), 7.81 (s, 1H), 7.89 (s, 1H), 8.07 (d, 1H), 8.83 (s, 1H), 9.44 (s, 1H), 11.33 (s, 1H); LC-MS m/z 422.1 [M+H]⁺

### Example 16

### Preparation of 1-[2-(8-Chloro-7-methyl-2,4-dioxo-3,4-dihydro-2H-benzo[g]pteridin-10-yl)-ethyl]-piperidine-4-carboxylic acid

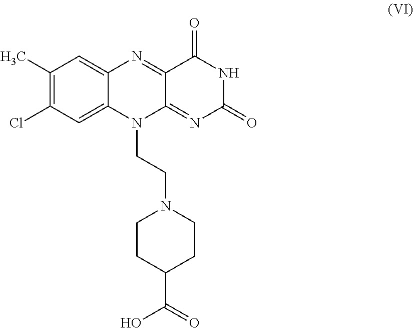

### Step 1: Preparation of 2-(5-Chloro-4-methyl-2-nitro-phenylamino)-tetrahydro-pyran-3,4,5-triol

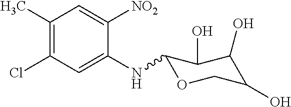

A solution of 5-chloro-4-methyl-2-nitro-phenylamine (19.8 g, 0.1 mol), ammonium chloride (0.1 g), and D-ribose (15.9 g, 0.1 mol) in EtOH (200 mL) is refluxed and stirred overnight. The reaction solution is concentrated under reduced pressure and resuspended in DCM:MeOH (1:1) and the precipitated unreacted staring material is removed by filtration. The mother liquor is dry loaded on silica gel using DCM:MeOH (1:1) and ISCO flash column chromatography is performed. 100% DCM is used until the first peak elutes, then 20% MeOH/DCM is used to elute the 11.5 g of pure orange product as a sticky solid (Yield: 40%) and 9.58 g of unreacted starting material is recovered. LC-MS m/z 318.7 [M+H], retention time 2.83 min. This crude material is used in the next step without further purification.

### Step 2: Preparation of 5-(5-Chloro-4-methyl-2-amino-phenylamino)-pentane-1,2,3,4-tetraol

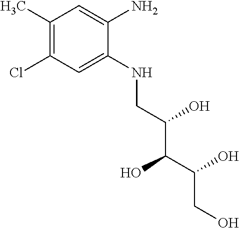

To a solution of 2-(5-chloro-4-methyl-2-nitro-phenylamino)-tetrahydro-pyran-3,4,5-triol (6.87 g, 0.02 mol) in EtOH (125 mL) is added sodium borohydride (1.65 g, 0.043 mol) portionwise such that the evolution of gas is controlled as to not overflow the contents of the flask. The resulting mixture is heated at reflux for 4 h. The reaction mixture is then cooled to 0° C. at which point Pd/C (300 mg) is added along with additional sodium borohydride (1.65 g, 0.043 mol). The reaction mixture is then allowed to stir at room temperature for 2 h. The reaction mixture is filtered through celite and washed liberally with MeOH, and finally concentrated to obtain the crude product, (as a clear purple oil) to be used directly in the next step. LC-MS: m/z 290.9 [M+H], retention time 1.38 min.

### Step 3: Preparation of 8-Chloro-7-methyl-10-(2,3,4,5-tetrahydroxy-pentyl)-10H-benzo[g]pteridine-2,4-dione

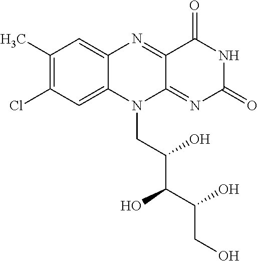

Crude 5-(2-amino-5-chloro-4-methyl-phenylamino)-pentane-1,2,3,4-tetraol (0.022 mol) is dissolved in glacial acetic acid (80 mL), covered in foil, and stirred at room temperature. At which point, the flask is purged with argon for 20 min, and alloxan monohydrate (3.45 g, 0.022 mol), boron oxide (1.35 g, 0.022 mol) are added to the stirring solution. The reaction is maintained under an argon atmosphere and stirred at room temperature for 3 h. The solution is concentrated under reduced pressure and the residue is dissolved in water (300 mL) and chilled in an ice bath. The precipitate is then filtered. The resulting filtrate is purified by preparatory HPLC in 10 mL segments (30 injections) using Method 1. 8-Chloro-7-methyl-10-(2,3,4,5-tetrahydroxy-pentyl)-10H-benzo[g]pteridine-2,4-dione (455 mg) is isolated following lyophilization (Yield: 5.3%). LC-MS m/z 397.1 [M+H], retention time 1.58 min. ¹H NMR (400 MHz, DMSO-d6) δ 2.51 (s, 3H), 3.46 (m, 1H), 3.64 (m, 2H), 4.23 (m, 1H), 4.49 (m, 1H), 4.67 (m, 1H), 4.78 (m, 2H), 4.88 (m, 1H), 5.15 (m, 2H), 8.13 (s, 1H), 8.20 (s, 1H), 11.47 (s, 1H).

### Step 4: Preparation of (8-Chloro-7-methyl-2,4-dioxo-3,4-dihydro-2H-benzo[g]pteridin-10-yl)-acetaldehyde

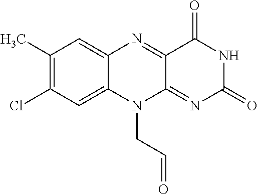

To a cooled (0° C.) suspension of 8-chloro-7-methyl-10-(2,3,4,5-tetrahydroxy-pentyl)-10H-benzo[g]pteridine-2,4-dione (0.235 g, 0.0006 mol) in 2 N aqueous sulfuric acid (60 mL) (in a flask covered with foil), is added (dropwise) a solution of orthoperiodic acid (0.41 g, 0.0018 mol) in water (25 mL). After 30 min., the reaction is allowed to warm to rt and is stirred until it becomes clear, yellow solution. The pH of the reaction solution is then adjusted carefully to 3.8-3.9 (using a pH meter) by addition of solid sodium carbonate [it is extremely important that the pH is monitored carefully, otherwise going over a pH of 3.9 does not allow for the product to precipitate out of solution.] The precipitate is then filtered off and washed liberally with cold water, ethanol, and diethyl ether to yield 0.089 g of the desired product as an orange solid (Yield: 49%). LC-MS m/z 305.1 [M+H] retention time: 1.69 min.

### Step 5: Preparation of 1-[2-(8-Chloro-7-methyl-2,4-dioxo-3,4-dihydro-2H-benzo[g]pteridin-10-yl)-ethyl]-piperidine-4-carboxylic acid

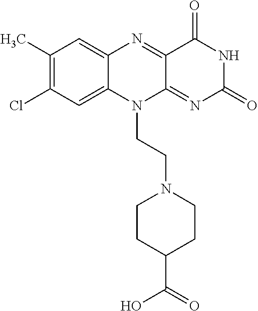

Piperidine-4-carboxylic acid (0.14 g, 0.0011 mol) is added to a stirred mixture of (8-chloro-7-methyl-2,4-dioxo-3,4-dihydro-2H-benzo[g]pteridin-10-yl)-acetaldehyde (0.11 g, 0.0004 mol) and MeOH (10 mL). The reaction mixture is heated to 40° C. and then 8 drops of glacial acetic acid are added. After two hours, NaCNBH₃ (0.05 g, 0.0008 mol) is added to the reaction mixture and allowed to stir at 40° C. for 23 h. The precipitate that forms is isolated by filtration to provide an orange solid yielding 0.061 g of the desired product (Yield: 50%). LC-MS m/z 418.1 [M+H]; retention time: 1.59 min.

### Example 17

### Preparation of 1-[2-(8-Cyclopentylamino-7-methyl-2,4-dioxo-3,4-dihydro-2H-benzo[g]pteridin-10-yl)-ethyl]-piperidine-4-carboxylic acid

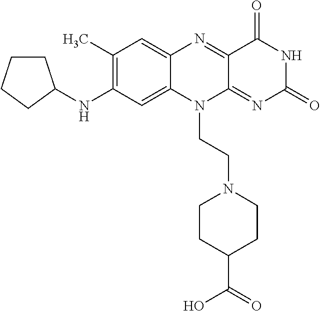

To a solution of 1-[2-(8-chloro-7-methyl-2,4-dioxo-3,4-dihydro-2H-benzo[g]pteridin-10-yl)-ethyl]-piperidine-4-carboxylic acid (see Example 16 preparation) (12 mg, 0.029 mmol) in DMSO (5 mL) at room temperature, is added cyclopentylamine (12 mg, 0.15 mmol), and the solution is stirred under argon at 70° C. for 20 h. Cyclopentylamine is added (0.1 mL, 1.36 mmol) and the mixture is stirred for an additional 8 h. The reaction is cooled to room temperature, diluted with water (3 mL), and purified by preparatory HPLC (Method 1). The desired product (9 mg) is isolated as a fluffy red solid after lyophilization of the appropriate fractions (Yield: 69%). ¹H NMR (400 MHz, CD₃OD) δ 7.75 (s, 1H), 6.6 (s, 1H), 5.05 (m, 3H), 4.32 (m, 1H), 3.7 (m, 2H), 3.5 (m, 1H), 2.37 (s, 3H), 2.3-2.08 (m, 8H), 1.90-1.78 (m, 8H); LC-MS m/z 467.2 (M+H), retention time 2.13 min.

### Example 18

### 1-(2-(3-(Acetoxymethyl)-7,8-dimethyl-2,4-dioxo-3,4-dihydrobenzo[g]pteridin-10(2H)-yl)ethyl)piperidine-4-carboxylic acid

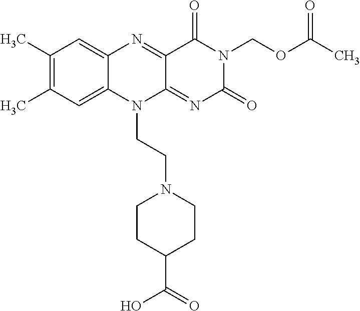

### Step 1 Preparation of 1-(2-(7,8-dimethyl-2,4-dioxo-3,4-dihydrobenzo[g]pteridin-10(2H)-yl)ethyl)piperidine-4-carboxylic acid

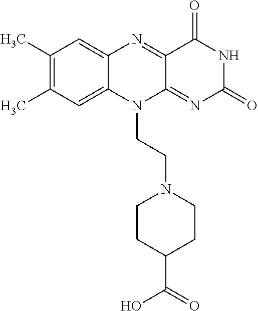

Prepared by reductive amination using a procedure similar to that of step 5, Example 16 using 2-(7,8-dimethyl-2,4-dioxo-3,4-dihydrobenzo[g]pteridin-10(2H)-yl)acetaldehyde (prepared by the method of step 1, Example 3) and piperidine-4-carboxylic acid as starting materials.

### Step 2 Preparation of benzyl 1-(2-(7,8-dimethyl-2,4-dioxo-3,4-dihydrobenzo[g]pteridin-10(2H)-yl)ethyl)piperidine-4-carboxylate

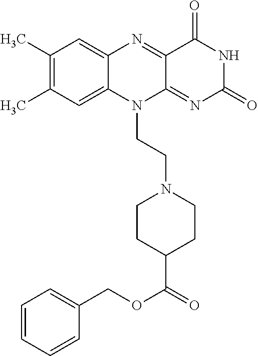

A mixture of 1-(2-(7,8-dimethyl-2,4-dioxo-3,4-dihydrobenzo[g]pteridin-10(2H)-yl)ethyl)piperidine-4-carboxylic acid (224 mg, 0.56 mmol), benzylchloride (4 mL, 34 mmol) and DIPEA (1.5 mL) are stirred at 60° C. for 3 days. The reaction is concentrated under reduced pressure, dissolved in water, and washed with Et₂O (2×10 mL) to remove the benzyl chloride. The product is extracted with chloroform (3×20 mL). The organic phase is dried (Na₂SO₄), filtered and evaporated to afford 142 mg of crude product. This crude product is used in the next reaction without further purification. LC-MS m/z 488.0 [M+H]⁺, retention time 2.56 min.

### Step 3 Preparation of benzyl 1-(2-(3-(acetoxymethyl)-7,8-dimethyl-2,4-dioxo-3,4-dihydrobenzo[g]pteridin-10(2H)-yl)ethyl)piperidine-4-carboxylate

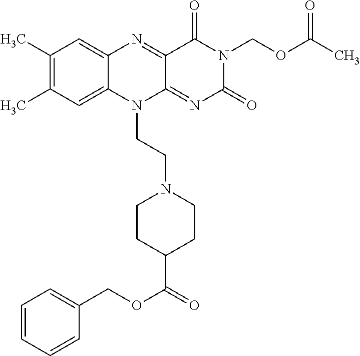

A mixture of benzyl 1-(2-(7,8-dimethyl-2,4-dioxo-3,4-dihydrobenzo[g]pteridin-10(2H)-yl)ethyl)piperidine-4-carboxylate (66 mg, 0.13 mmol), potassium carbonate (1.02 g, 7.3 mmol) and chloromethyl acetate (0.5 g, 4.6 mmol) in DMSO (5 mL) are stirred at rt for 3 h. Water is added to the reaction and the mixture is extracted with EtOAc. The organic phase is dried (Na₂SO₄), filtered and evaporated to afford crude product. This crude product is used in the next reaction without further purification. LC-MS m/z 560.1 [M+H]⁺, retention time 2.89 min.

### Step 4 Preparation of 1-(2-(3-(acetoxymethyl)-7,8-dimethyl-2,4-dioxo-3,4-dihydrobenzo[g]pteridin-10(2H)-yl)ethyl)piperidine-4-carboxylic acid

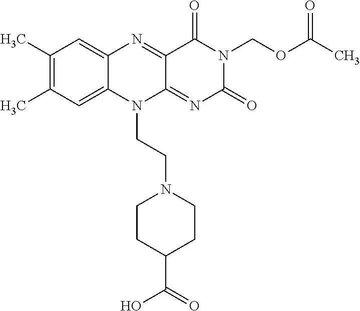

A solution of benzyl 1-(2-(3-(acetoxymethyl)-7,8-dimethyl-2,4-dioxo-3,4-dihydrobenzo[g]pteridin-10(2H)-yl)ethyl)piperidine-4-carboxylate (0.13 mmol) and Pd/C (catalytic) in MeOH (10 mL) is stirred under an atmosphere of hydrogen at 1 atm for 1 h. The mixture is filtered through a celite pad. The filtrate is concentrated under reduced pressure to dryness. The crude product is dissolved in ACN (6 mL)/water (2 mL) and purified by preparative HPLC (Method 2). Lyophilization of the combined pure fractions (LCMS) affords 0.52 mg of desired product as a yellow solid. ¹H NMR (400 MHz, CD₃OD) δ 1.95 (m, 2H), 2.09 (s, 3H), 2.32 (m, 2H), 2.52 (s, 3H), 2.65 (s, 3H), 2.71 (m, 1H), 3.20 (m, 2H), 3.70 (m, 2H), 4.03 (m, 2H), 5.15 (m, 2H), 6.08 (s, 2H), 7.86 (s, 1H), 8.08 (s, 1H).

### Example 19

### Preparation of 1-{2-[8-(2-tert-Butoxycarbonyl-ethylamino)-7-methyl-2,4-dioxo-3,4-dihydro-2H-benzo[g]pteridin-10-yl]-ethyl}-piperidine-4-carboxylic acid

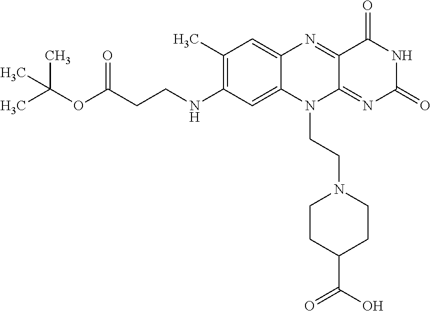

To a solution of 1-[2-(8-chloro-7-methyl-2,4-dioxo-3,4-dihydro-2H-benzo[g]pteridin-10-yl)-ethyl]-piperidine-4-carboxylic acid (see Example 16 for preparation) (15 mg, 0.036 mmol) in DMSO (6 mL) at room temperature, is added tert-butyl 3-aminopropanoate (0.1 mL, 1.3 mmol), and the solution is stirred under argon at 70° C. for 24 h. The reaction is cooled to room temperature, diluted with water (3 mL) and purified by preparatory HPLC (Method 1). The desired product (7.85 mg) is isolated as the TFA salt, as a fluffy red solid after lyophilization (Yield: 41%). ¹H NMR (400 MHz, DMSO-d6) δ 12.6 (s, 1H), 11.13 (s, 1H), 8.8 (s, 1H), 7.75 (s, 1H), 7.2 (s, 1H), 6.6 (s, 1H), 4.98 (m, 2H), 3.97 (m, 2H), 3.7 (m, 2H), 3.53 (b s, 3H), 3.01 (b s, 2H), 2.7 (t, 2H), 2.27 (s, 3H), 2.01 (m, 2H), 1.76 (BR, 2H), 1.41 (s, 9H); LC-MS m/z 527.1 (M+H), retention time 2.38 min.

### Example 20

### (S)-1-(2-(7,8-Dimethyl-2,4-dioxo-3,4-dihydrobenzo[g]pteridin-10(2H)-yl)ethyl)pyrrolidine-2-carboxylic acid

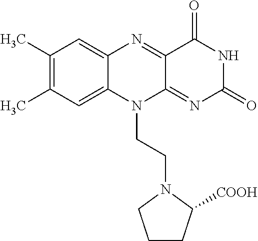

To a solution of 2-(7,8-dimethyl-2,4-dioxo-3,4-dihydrobenzo[g]pteridin-10(2H)-yl)acetaldehyde (prepared by the method of step 1, Example 3) (50 mg, 0.176 mmol) in methanol (5 mL) are added (S)-pyrrolidine-2-carboxylic acid (20.3 mg, 0.176 mmol) and acetic acid (75 μL) at rt. The reaction is stirred at 50° C. for 30 min. The reaction is cooled to room temperature and sodium cyanoborohydride (25 mg, 0.39 mmol) is added and the reaction mixture is stirred at 50° C. for 24 h. The reaction mixture is concentrated under vacuum and the resulting residue is purified by column chromatography (silica gel) using gradient elution (DCM:MeOH:Et₃N 85:14:1 to 80:19:1) to afford the desired product (40.0 mg, 59% yield). ¹H NMR (400 MHz, DMSO) δ 1.75 (m, 3H), 1.99 (m, 1H), 2.39 (s, 3H), 2.59 (s, 3H), 2.62 (m, 1H), 2.88 (m, 1H), 3.13 (m, 1H), 3.28 (m, 2H), 3.44 (bs, water), 4.71 (m, 2H), 7.82 (s, 1H), 7.86 (s, 1H), 11.32 (s, 1H). ESI(−) m/z=382.3.

### Example 21

### Ethyl 1-(2-(7,8-dimethyl-2,4-dioxo-3,4-dihydrobenzo[g]pteridin-10(2H)-yl)ethyl)piperidine-3-carboxylate

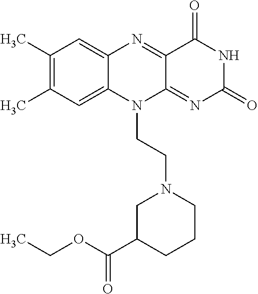

Ethyl 1-(2-(7,8-dimethyl-2,4-dioxo-3,4-dihydrobenzo[g]pteridin-10(2H)-yl)ethyl)piperidine-3-carboxylate is synthesized from 2-(7,8-dimethyl-2,4-dioxo-3,4-dihydrobenzo[g]pteridin-10(2H)-yl)acetaldehyde (prepared by the method of step 1, Example 3)(50 mg, 0.176 mmol) and ethyl piperidine-3-carboxylate (28 mg, 0.176 mmol) following the procedure described for Example 20. The reaction mixture is concentrated under vacuum and the resulting residue is purified by column chromatography (silica gel) using isocratic elution (DCM:MeOH 97:3) to afford the desired product (35.1 mg, 47% yield). ¹H NMR (400 MHz, DMSO) δ 1.12 (t, 3H), 1.38 (m, 2H), 1.60 (m, 1H), 1.71 (m, 1H), 2.31 (m, 6H), 2.4 (s, 3H), 2.74 (m, 3H), 2.94 (m, 1H), 3.97 (m, 2H), 4.70 (m, 2H), 7.76 (s, 1H), 7.88 (s, 1H), 11.30 (s, 1H); ESI(−) m/z=426.4.

### Example 22

### tert-Butyl 1-(2-(2,4-dioxo-3,4-dihydrobenzo[g]pteridin-10(2H)-yl)ethyl)piperidine-4-carboxylate

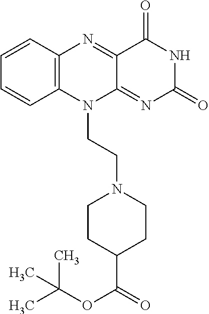

### Step 1 Preparation of tert-butyl 1-(cyanomethyl)piperidine-4-carboxylate

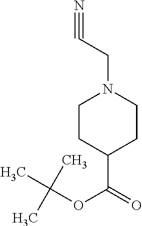

To a solution of tert-butyl piperidine-4-carboxylate (750 mg, 4.05 mmol) in anhydrous DCM (15 mL), is added 2-chloroacetonitrile (333 μL, 5.26 mmol) and potassium carbonate (1.7 g, 12.15 mmol). The reaction mixture is stirred at room temperature for 18 h. The reaction mixture is diluted with water (100 mL) and the aqueous layer is extracted with DCM (100 mL). The organic layer is dried over Na₂SO₄, filtered, and concentrated under reduced pressure. The residue is dry loaded on silica gel and purified by Biotage flash column chromatography using a gradient from 0 to 10% MeOH in DCM as eluent. Desired product (463 mg) is isolated (yield: 51%). %). ¹H NMR (400 MHz, CDCl₃) δ 1.44 (s, 9H), 1.73 (m, 2H), 1.93 (m, 2H), 2.19 (m, 1H), 2.35 (m, 2H), 2.79 (m, 2H), 3.51 (s, 2H).

### Step 2 Preparation of tert-Butyl 1-(2-aminoethyl)piperidine-4-carboxylate

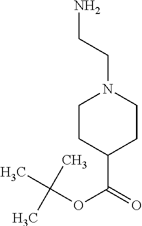

To a solution of tert-butyl 1-(cyanomethyl)piperidine-4-carboxylate (430 mg, 1.91 mmol) in EtOH (15 mL), is added Raney-Nickel (catalytic). The reaction mixture is placed in a parr hydrogenator apparatus at 50 psi of H₂ for 24 h at room temperature. After the reaction is complete (as monitored by TLC, 95:5 DCM: MeOH) the mixture is filtered through a celite pad, and the pad is rinsed with ethanol. The filtrate is concentrated under reduced pressure and the resulting material is used in the next step without further purification. ¹H NMR (400 MHz, CDCl₃) δ 1.44 (s, 9H), 1.71 (2H), 1.88 (m, 2H), 2.22 (m, 3H), 2.69 (m, 2H), 2.84 (m, 2H), 2.98 (m, 2H), 3.53 (bs, 2H).

### Step 3 Preparation of tert-butyl 1-(2-(2-nitrophenylamino)ethyl)piperidine-4-carboxylate

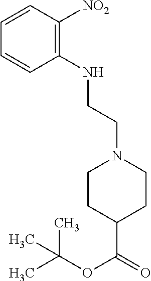

To a solution of tert-butyl 1-(2-aminoethyl)piperidine-4-carboxylate (150 mg, 0.66 mmol) and sodium bicarbonate (110 mg, 1.31 mmol) in 2.5 mL of DMF under argon is added 2-fluoronitrobenzene (77 μL, 0.72 mmol). The reaction mixture is stirred at 70° C. for 18 h. After the reaction is complete (as monitored by TLC, hexanes:EtOAc, 4:6) the DMF is evaporated and the residue is dissolved in water (10 mL). The aqueous layer is extracted with EtOAc (20 mL) and the organic layer is dried over Na₂SO₄, filtered, and concentrated under reduced pressure. The residue is dry loaded on silica gel and purified by Biotage flash column chromatography using gradient 0 to 60% EtOAc in hexanes as eluent. Pure product (84 mg) is isolated (yield: 36%). NMR (400 MHz, CDCl₃) δ 1.44 (s, 9H), 1.78 (m, 2H), 1.88 (m, 2H), 2.13 (m, 2H), 2.20 (m, 1H), 2.68 (m, 2H), 2.87 (m, 2H), 3.35 (m, 2H), 6.63 (t, 1H), 6.81 (d, 1H), 7.42 (t, 1H), 8.17 (d, 1H), 8.43 (b s, 1H).

### Step 4 Preparation of tert-butyl 1-(2-(2-aminophenylamino)ethyl)piperidine-4-carboxylate

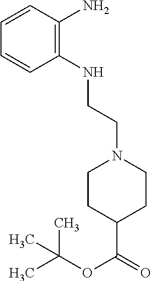

To a solution of tert-butyl 1-(2-(2-nitrophenylamino)ethyl)piperidine-4-carboxylate (84 mg, 0.24 mmol) in anhydrous MeOH (6 mL) under argon, is added Pd/C (8.4 mg) and sodium borohydride (64 mg, 1.68 mmol), and the mixture is stirred under an atmosphere of hydrogen (via a balloon) and at room temperature for 30 min. The reaction mixture is filtered through celite, which is washed liberally with EtOH, and the solution is then concentrated to obtain the crude product as a clear, colourless oil to be used directly in the next step.

### Step 5 tert-Butyl 1-(2-(2,4-dioxo-3,4-dihydrobenzo[g]pteridin-10(2H)-yl)ethyl)piperidine-4-carboxylate

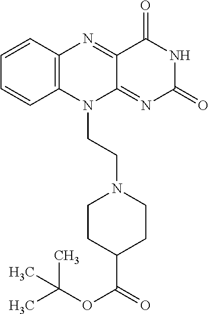

Crude tert-butyl 1-(2-(2-aminophenylamino)ethyl)piperidine-4-carboxylate (0.24 mmol) is dissolved in glacial acetic acid (6 mL) under argon. Alloxan monohydrate (39 mg, 0.24 mmol) and boron oxide (34 mg, 0.48 mmol) are added to the stirring solution and the reaction is maintained under an argon atmosphere at 25° C. with stirring for 2 h. The residue is dry loaded on silica gel and purified by Biotage flash column chromatography using gradient from 0 to 5% MeOH in DCM as eluent. Desired product (33 mg) is isolated (yield: 32%). ¹H NMR (400 MHz, DMSO) δ 1.38 (m, 10H), 1.72 (m, 2H), 2.11 (m, 3H), 2.48 (m, 1H), 2.64 (m, 2H), 2.88 (m, 2H), 4.68 (m, 2H), 7.62 (m, 1H), 7.82 (m, 2H), 8.10 (m, 1H), 11.39 (s, 1H). ESI(+) m/z=4.26.0.

### Example 23

### 1-(2-(2,4-Dioxo-3,4-dihydrobenzo[g]pteridin-10(2H)-yl)ethyl)piperidine-4-carboxylic acid trifluoroacetic acid

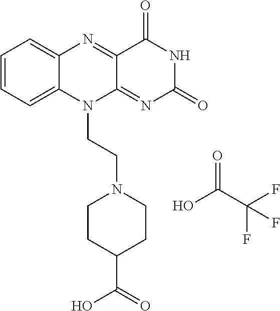

To a solution of tert-butyl 1-(2-(2,4-dioxo-3,4-dihydrobenzo[g]pteridin-10(2H)-yl)ethyl)piperidine-4-carboxylate (see Example 22 for preparation) (23 mg, 0.053 mmol) in anhydrous DCM (2 mL) is added TFA (200 μL, 2.58 mmol) and the mixture is stirred at 25° C. for 16 h. The solution is concentrated under reduced pressure, and the residue is dissolved in DMSO (1 mL), filtered, and purified by preparatory HPLC (Method 1). The desired product (12.7 mg) is isolated following lyophilization (Yield: 48.8%). ¹H NMR (400 MHz, DMSO) δ 1.78 (m, 2H), 2.06 (m, 2H), 3.09 (m, 3H), 3.44 (m, 2H), 3.89 (m, 2H), 4.99 (m, 2H), 7.68 (m, 1H), 7.97 (m, 1H), 8.06 (m, 1H), 8.17 (m, 1H), 9.43 (b s, 1H), 11.60 (s, 1H), 12.61 (b s, 1H). ESI(+) m/z=370.0.

For Examples 24, 25, and 26, the following analytical HPLC methods are used: Agilent 1100 HPLC, Agilent XDB C18 50×4.6 mm 1.8 micron column, 1.5 mL/min, Solvent A—Water (0.1% TFA), Solvent B—Acetonitrile (0.07% TFA), Gradient—5 min 95% A to 95% B; 1 min hold; then recycle, UV Detection @ 214 and 254 nm.
For Examples 25 and 26, the following preparative reverse phase chromatography methods are used: Varian PrepStar, Phenomenex Luna(2) C18 250×21.2 mm 10 micron column, 20 mL/min, Solvent B—Water (0.1% TFA), Solvent A—Acetonitrile (0.07% TFA), Gradient—10 min 5% A to 80% A; 5 min 80% A to 100% A; 5 min hold; then recycle, UV Detection @ 254 nm.

### Example 24

### Ethyl 1-[2-(8-chloro-7-methyl-2,4-dioxo-3,4-dihydrobenzo[g]pteridin-10(2H)-yl)ethyl]piperidine-4-carboxylate

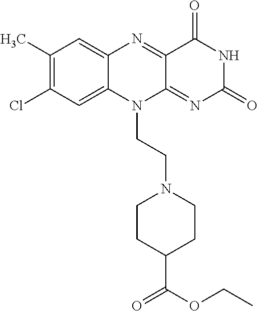

Step 1 Preparation of ethyl 1-{2-[(5-chloro-4-methyl-2-nitrophenyl)amino]ethyl}piperidine-4-carboxylate

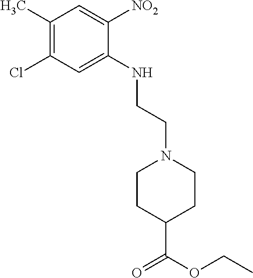

1,8-Diazabicyclo[5.4.0]undec-7-ene (1.2 mL, 7.8 mmol) is added to a solution of 1,5-dichloro-2-methyl-4-nitrobenzene (0.81 g, 3.9 mmol) and ethyl 1-(2-aminoethyl)piperidine-4-carboxylate (1.6 g, 7.8 mmol) in DMSO (7.8 mL) and the red solution is stirred under an atmosphere of nitrogen at 100° C. After 3.5 h, it is taken up in ethyl acetate/hexanes and the organic layer is washed with water then brine. It is dried with sodium sulfate, filtered, and concentrated in vacuo. The brown residue is purified by silica gel flash chromatography, eluted with 15%, 20%, and 25% ethyl acetate/hexanes to give 0.66 g of desired product as an amorphous orange solid after concentration in vacuo. (Yield: 45.7%). Mass spec (ESI+) for C₁₇H₂₄ClN₃O₄ m/z 370.0 (M+H)⁺. HPLC retention time 3.29 min. (System D).

### Step 2 Preparation of ethyl 1-{2-[(2-amino-5-chloro-4-methylphenyl)amino]ethyl}piperidine-4-carboxylate

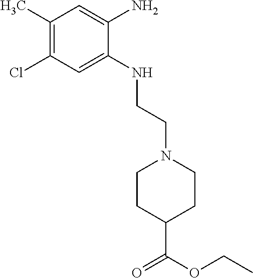

A well-stirred slurry of Raney nickel (50 mg, 0.85 mmol) and ethyl 1-{2-[(5-chloro-4-methyl-2-nitrophenyl)amino]ethyl}piperidine-4-carboxylate (0.66 g, 1.79 mmol) in ethanol (20 mL) is alternately evacuated then covered with 1 atmosphere of hydrogen (3×) (balloon). After 3 hours at rt, the mixture was filtered through Celite®, and concentrated in vacuo to give the desired product as a brown oil, 0.59 g (Yield: 99%). Mass spec (ESI+) for C₁₇H₂₆ClN₃O₂ m/z 340.1 (M+H)⁺. HPLC retention time 2.53 min. (System D).

### Step 3 Preparation of ethyl 1-[2-(8-chloro-7-methyl-2,4-dioxo-3,4-dihydrobenzo[g]pteridin-10(2H)-yl)ethyl]piperidine-4-carboxylate

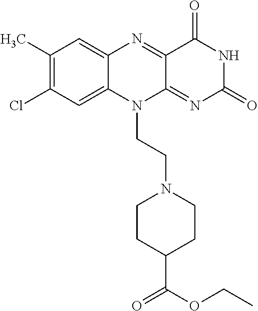

A mixture of ethyl 1-{2-[(2-amino-5-chloro-4-methylphenyl)amino]ethyl}piperidine-4-carboxylate (56 mg, 0.16 mmol), alloxan monohydrate (26.4 mg, 0.165 mmol), and boric acid (20.4 mg, 0.330 mmol) in acetic acid (2 mL) is stirred at room temperature under nitrogen and covered with foil. After 16 hours, it is concentrated in vacuo to give brown oil that is then stirred rapidly as a suspension with saturated, aqueous sodium bicarbonate and ethyl acetate (10 mL each) for an hour. The precipitate is filtered and rinsed with ethyl acetate and diethyl ether, and air dried to give 58 mg (Yield: 79%) of desired product. ¹H NMR (400 MHz, CD₃CN) δ 1.22 (3H, t), 1.57 (2H, m), 1.82 (2H, m), 2.22 (3H, m), 2.54 (3H, s), 2.76 (2H, m), 2.93 (2H, m), 4.09 (2H), 4.68 (2H, m), 7.98 (1H, s), 8.06 (1H, s), 9.27 (1H, br s). Mass spec (ESI+) for C₂₁H₂₄ClN₅O₄ m/z 446.0 (M+H)⁺. HPLC retention time 2.44 min. (System D).

### Example 25

### 1-[2-(8-Amino-7-methyl-2,4-dioxo-3,4-dihydrobenzo[g]pteridin-10-(2H)-yl)ethyl]piperidine-4-carboxylic acid

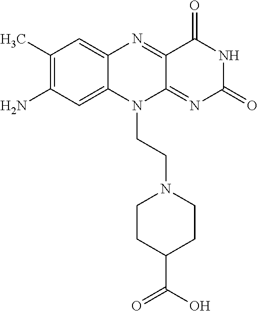

### Step 1 Preparation of 1-[2-(8-Benzylamino-7-methyl-2,4-dioxo-3,4-dihydrobenzo Id pteridin-10(2H)-yl)ethyl]piperidine-4-carboxylic acid acetate

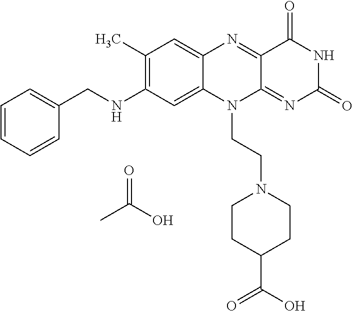

A mixture of benzylamine (98.0 uL, 0.90 mmol) and ethyl 1-[2-(8-chloro-7-methyl-2,4-dioxo-3,4-dihydrobenzo[g]pteridin-10(2H)-yl)ethyl]piperidine-4-carboxylate (40.0 mg, 0.090 mmol) in N,N-dimethylacetamide (1 mL) is heated at 80° C. for 19 hours. Tetrahydrofuran (3.8 mL, 47 mmol) and lithium hydroxide (1 M aqueous, 0.9 mL) are added to the crude material and stirred at room temperature. After an hour, the mixture is concentrated in vacuo to give a red oil. Celite® was added and 100 mL methanol. The mixture is dried in vacuo and the Celite® mixture loaded onto a 28×55 mm C-18 reversed phase silica gel column (18.4 g) then eluted with 2% acetic acid/water and (5% to 20% acetonitrile+5% acetic acid)/water. Concentration of appropriate fractions in vacuo gives 43.2 mg of desired product as an amorphous red solid. (Yield: 87%). Mass spec (ESI+) for C₂₆H₂₈N₆O₄ m/z 489.0 (M+H)⁺. HPLC retention time 2.29 min. (System D).

### Step 2 Preparation of 1-[2-(8-Amino-7-methyl-2,4-dioxo-3,4-dihydrobenzo [g]pteridin-10-(2H)-yl)ethyl]piperidine-4-carboxylic acid acetate

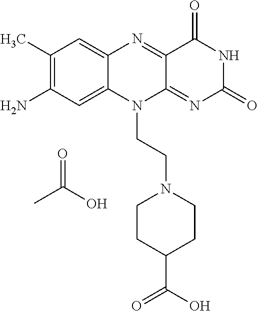

1-[2-(8-Benzylamino-7-methyl-2,4-dioxo-3,4-dihydrobenzo[g]pteridin-10(2H)-yl)ethyl]piperidine-4-carboxylic acid (30.6 mg, 0.056 mmol) is dissolved in water (4.0 mL) and acetic acid (0.4 mL) and 10% palladium on carbon (2 mg,) is added. The rapidly stirred mixture is alternately evacuated then covered with 1 atmosphere hydrogen (3×) (balloon). After 2 hours, the mixture is filtered, rinsed with 5% acetic acid in water, and concentrated under vacuum. The red residue is flash chromatographed on a column (28 mm diameter, 13.5 g) of C-18 reversed phase silica gel and eluted with water, 5% acetic acid/water, and (5% acetonitrile+5% acetic acid)/water to provide desired product as an amorphous red solid, 16.5 mg. (Yield: 64%). ¹H NMR (400 MHz, DMSO-d₆) δ 12.06 (br s, 1H), 10.94 (s, 1H), 7.66 (s, 1H), 7.27 (s, 2H), 6.76 (s, 1H), 4.35 (m, 2H), 2.94 (m, 2H), 2.62 (m, 2H), 2.23 (s, 3H), 2.16 (m, 3H), 1.91 (s, 3H), 1.80 (m, 2H), 1.53 (m, 2H). Mass spec (ESI+) for C₁₉H₂₂N₆O₄ m/z 399.0 (M+H)⁺. HPLC retention time 1.69 min. (System D).

### Example 26

### 1-[2-(8-Dimethylamino-7-methyl-2,4-dioxo-3,4-dihydrobenzo[g]pteridin-10-(2H)-yl)ethyl]piperidine-4-carboxylic acid

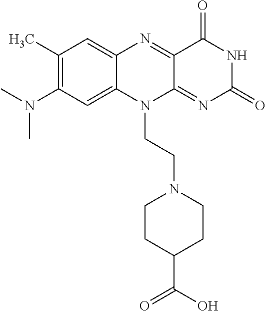

N,N-Dimethylformamide (0.2 mL) and dimethylamine (0.3 mL, 2 M in THF) are added to ethyl 1-[2-(8-chloro-7-methyl-2,4-dioxo-3,4-dihydrobenzo[g]pteridin-10(2H)-yl)ethyl]piperidine-4-carboxylate (25.0 mg, 0.0561 mmol) and the mixture is heated at 80° C. for an hour. It is blown dry under a nitrogen stream, to provide a dark red residue. THF (2.4 mL) and lithium hydroxide (0.6 mL, 1M aqueous) are added and the mixture is stirred at room temperature for an hour. Glacial acetic acid (0.345 mL) is added and solvent removed in vacuo. The residue is dissolved in water (7 mL) and the red solution is chromatographed on a 28×80 mm column of C-18 reversed phase silica gel. Elution is with water, 25% methanol/water, and 45% methanol/water. Concentration of appropriate fractions in vacuo provides 15.9 mg of desired product as an amorphous red solid. (Yield: 66%). ¹H NMR (400 MHz, DMSO-d₆) δ 11.13 (br s, 1H), 7.78 (s, 1H), 6.98 (s, 1H), 4.69 (m, 2H), 3.06 (s, 6H), 2.92 (m, 2H), 2.64 (m, 2H), 2.45 (s, 3H), 2.09 (m, 2H), 1.96 (m, 1H), 1.72 (m, 2H), 1.45 (m, 2H). Mass spec (ESI+) for C₂₁H₂₆N₆O₄ m/z 427.0 (M+H)⁺. HPLC retention time 2.00 minutes (System D).

### Example 27

### Preparation of 1-(2-(7,8-dimethyl-2,4-dioxo-3,4-dihydrobenzo[g]pteridin-10(2H)-yl)ethyl)piperidine-4-carboxylic acid

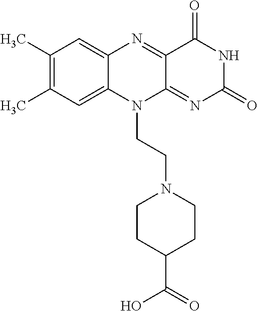

Prepared by reductive amination using a procedure similar to that of step 5, Example 16 using 2-(7,8-dimethyl-2,4-dioxo-3,4-dihydrobenzo[g]pteridin-10(2H)-yl)acetaldehyde (prepared by the method of step 1, Example 3) and piperidine-4-carboxylic acid as starting materials.

### Example 28

### 7,8-Dimethyl-10-[5-(2-oxo-2H-pyrimidin-1-yl)-pentyl]-10H-benzo[g]pteridine-2,4-dione

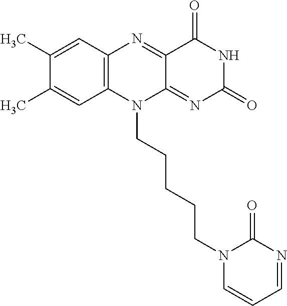

To a suspension of pyrimidin-2(1H)-one hydrochloride (100 mg, 0.755 mmol) and potassium carbonate (104 mg, 0.755 mmol) in anhydrous DMF (5 mL) is added 10-(5-bromopentyl)-7,8-dimethylbenzo[g]pteridine-2,4(3H,10H)-dione (see step 4 of Example 14 for preparation) (50 mg, 0.128 mmol) at room temperature. The mixture is then heated to 50° C. and stirred for 8 h, concentrated (50° C.), dissolved in DMF/water (1/3) and purified by preparative HPLC (Method 1) to give, after lyophilization, 7,8-dimethyl-1045-(2-oxo-2H-pyrimidin-1-yl)-pentyl]-10H-benzo[g]pteridine-2,4-dione (I) (10.9 mg, yield: 21%). ¹H NMR (400 MHz, DMSO-d6) δ 1.45 (m, 2H), 1.76 (m, 4H), 2.39 (s, 3H), 2.51 (s, 3H), 3.95 (m, 2H), 4.57 (m, 2H), 6.58 (dd, 1H), 7.83 (s, 1H), 7.90 (s, 1H), 8.50 (dd, 1H), 8.61 (m, 1H), 11.31 (s, 1H); LC-MS m/z 407.1 [M+H]⁺ and 7,8-Dimethyl-10-[5-(pyrimidin-2-yloxy)-pentyl]-10H-benzo[g]pteridine-2,4-dione (II) (4 mg, yield: 7.7%). ¹H NMR (400 MHz, DMSO-d6) δ 1.59 (m, 2H), 1.81 (m, 4H), 2.40 (s, 3H), 4.32 (m, 2H), 4.61 (m, 2H), 7.12 (m, 1H), 7.83 (s, 1H), 7.91 (s, 1H), 8.58 (d, 2H), 11.30 (s, 1H).

By using the methods described above and by selecting the appropriate starting materials, other compounds of the invention are prepared and characterized. These compounds, together with the Examples described above, are summarized in Table 1.

TABLE 1

| | | | HPLC | | | |
|---|---|---|---|---|---|---|
| | | LC-MS | retention | | | |
| | | MH+ | time | HPLC | | |
| Entry | Structure | (m/z) | (min) | method | Preparation | Name |
| 1 | 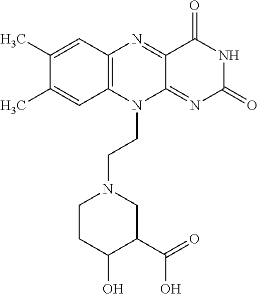 | 414.2 | 7.22 | Method F | Prepared using synthesis of Example 8 | 1-(2-(7,8-dimethyl-2,4- dioxo-3,4- dihydrobenzo[g]pteridin- 10(2H)-yl)ethyl)-4- hydroxypiperidine-3- carboxylic acid |
| 2 | 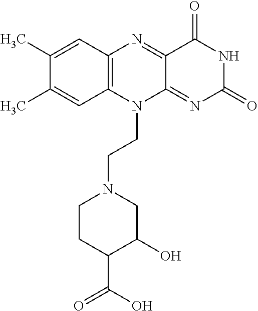 | 414.27 | 3.32 | Method C | Prepared using synthesis of Example 8 | 1-(2-(7,8-dimethyl-2,4- dioxo-3,4- dihydrobenzo[g]pteridin- 10(2H)-yl)ethyl)-3- hydroxypiperidine-4- carboxylic acid compound with 2,2,2- trifluoroacetic acid (1:1) |
| 3 | 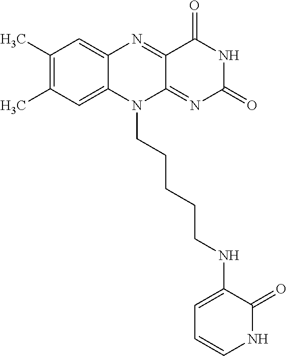 | 421.1 | 2.65 | Method C | Prepared using the synthesis of Example 14 | 7,8-Dimethyl-10-[5-(2- oxo-1,2-dihydro- pyridin-3-ylamino)- pentyl]-10H- benzo[g]pteridine-2,4- dione |
| 4 | 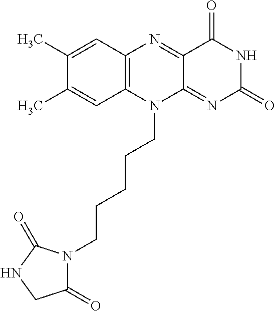 | 411.1 | 2.37 | Method C | Prepared using the synthesis of Example 15 | 10-[5-(2,5-Dioxo- imidazolidin-1-yl)- pentyl]-7,8-dimethyl- 10H- benzo[g]pteridine-2,4- dione |
| 5 | 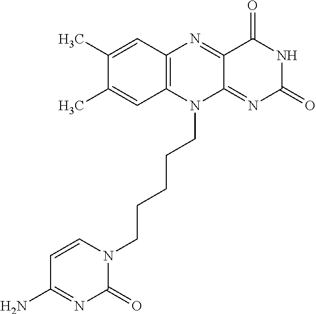 | 422.1 | 3.8 | Method C | Prepared using the synthesis of Example 15 | 10-[5-(4-Amino-2-oxo- 2H-pyrimidin-1-yl)- pentyl]-7,8-dimethyl- 10H- benzo[g]pteridine-2,4- dione |
| 6 | 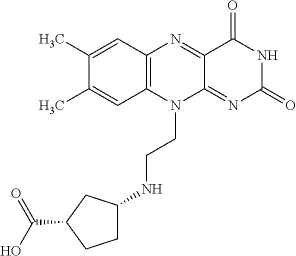 | 398 | 1.72 | Method C | Prepared using the synthesis of Example 3 | 2-[2-(7,8-Dimethyl-2,4- dioxo-3,4-dihydro-2H- benzo[g]pteridin-10- yl)-ethylamino]- cyclopentanecarboxylic acid |
| 7 | 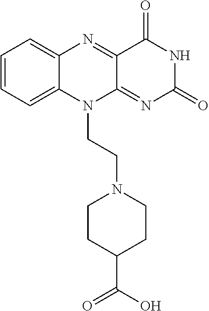 | 370.0 | 4.34 | Method A | Prepared using the synthesis of Example 23 | 1-(2-(2,4-Dioxo-3,4- dihydrobenzo[g]pteridin- 10(2H)- yl)ethyl)piperidine-4- carboxylic acid trifluoroacetic acid salt |
| 8 | 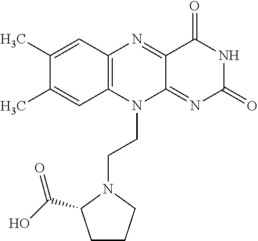 | 384 | 7.48 | Method B | Prepared using synthesis of Example 8 | (R)-1-[2-(7,8-Dimethyl- 2,4-dioxo-3,4-dihydro- 2H-benzo[g]pteridin- 10-yl)-ethyl]- pyrrolidine-2- carboxylic acid |
| 9 | 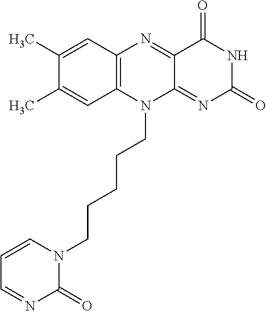 | 407.1 | 2.26 | Method C | Prepared using the synthesis of Example 28 | 7,8-Dimethyl-10-[5-(2- oxo-2H-pyrimidin-1- yl)-pentyl]-10H- benzo[g]pteridine-2,4- dione |
| 10 | 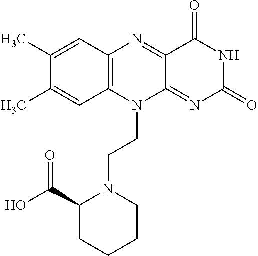 | 398.1 | 1.72 | Method C | Prepared using synthesis of Example 8 | (S)-1-(2-(7,8-dimethyl- 2,4-dioxo-3,4- dihydrobenzo[g]pteridin- 10(2H)- yl)ethyl)piperidine-2- carboxylic acid |
| 11 | 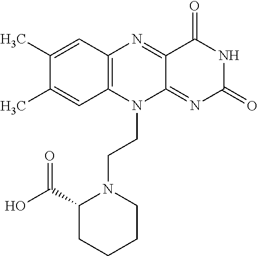 | 398.1 | 1.72 | Method C | Prepared using synthesis of Example 8 | (R)-1-(2-(7,8-dimethyl- 2,4-dioxo-3,4- dihydrobenzo[g]pteridin- 10(2H)- yl)ethyl)piperidine-2- carboxylic acid |
| 12 | 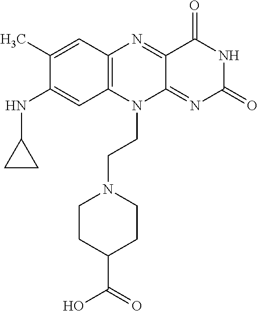 | 439.1 | 3.46 | Method A | Prepared using synthesis of Example 19 | 1-[2-(8- Cyclopropylamino-7- methyl-2,4-dioxo-3,4- dihydro-2H- benzo[g]pteridin-10- yl)-ethyl]-piperidine-4- carboxylic acid |
| 13 | 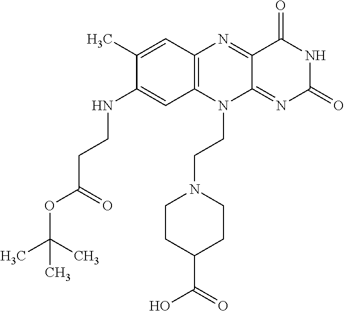 | 527.1 | 2.38 | Method C | Prepared using synthesis of Example 19 | 1-{2-[8-(2-tert- Butoxycarbonyl- ethylamino)-7-methyl- 2,4-dioxo-3,4-dihydro- 2H-benzo[g]pteridin- 10-yl]-ethyl}- piperidine-4-carboxylic acid |
| 14 | 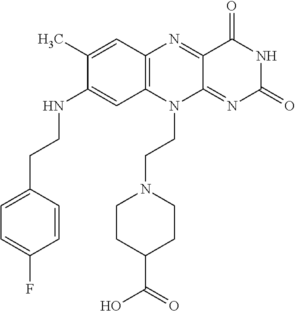 | 521.1 | 2.46 | Method C | Prepared using synthesis of Example 19 | 1-(2-{8-[2-(4-Fluoro- phenyl)-ethylamino]-7- methyl-2,4-dioxo-3,4- dihydro-2H- benzo[g]pteridin-10- yl}-ethyl)-piperidine-4- carboxylic acid |
| 15 | 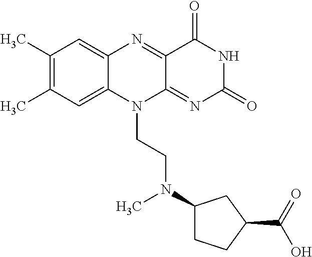 | 412 | 1.73 | Method C | Prepared using synthesis of Example 3 Step 2 | 3-{[2-(7,8-Dimethyl- 2,4-dioxo-3,4-dihydro- 2H-benzo[g]pteridin- 10-yl)-ethyl]-methyl- amino}- cyclopentanecarboxylic acid |
| 16 | 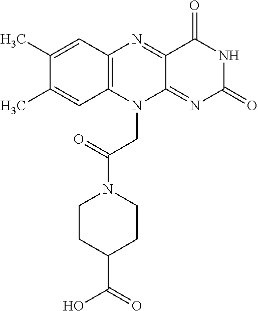 | MH− 410.1 | 5.993 | Method A | Prepared using the synthesis of Example 10 | 1-[2-(7,8-Dimethyl-2,4- dioxo-3,4-dihydro-2H- benzo[g]pteridin-10- yl)-acetyl]-piperidine- 4-carboxylic acid |
| 17 | 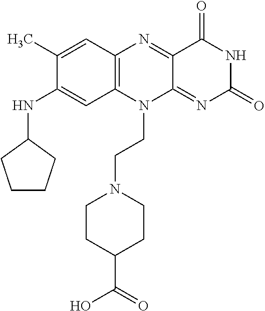 | 467.2 | 2.13 | Method C | Prepared using the synthesis of Example 17 | 1-[2-(8- Cyclopentylamino-7- methyl-2,4-dioxo-3,4- dihydro-2H- benzo[g]pteridin-10- yl)-ethyl]-piperidine-4- carboxylic acid |
| 18 | 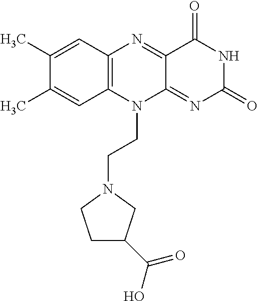 | 384.1 | 1.53 | Method C | Prepared using the synthesis of Example 4 | 1-[2-(7,8-Dimethyl-2,4- dioxo-3,4-dihydro-2H- benzo[g]pteridin-10- yl)-ethyl]-pyrrolidine- 3-carboxylic acid |
| 19 | 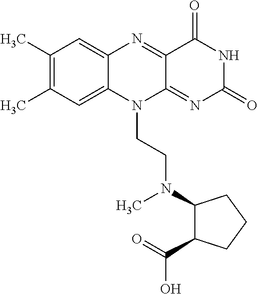 | 412.1 | 1.9 | Method C | Prepared using synthesis of Example 3 Step 2 | 2-{[2-(7,8-Dimethyl- 2,4-dioxo-3,4-dihydro- 2H-benzo[g]pteridin- 10-yl)-ethyl]-methyl- amino}- cyclopentanecarboxylic acid |
| 20 | 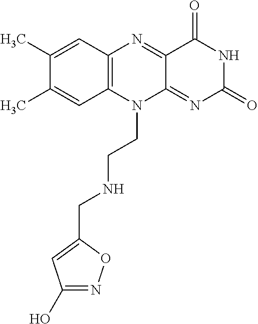 | 383 | 1.55 | Method C | Prepared using the synthesis of Example 9 | 10-{2-[(3-Hydroxy- isoxazol-5-ylmethyl)- amino]-ethyl}-7,8- dimethyl-10H- benzo[g]pteridine-2,4- dione trifluoroacetic acid salt |
| 21 | 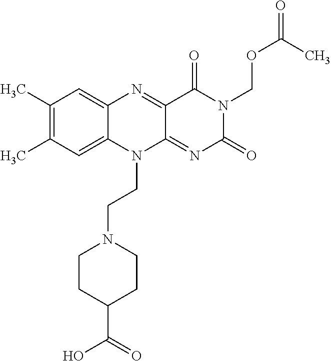 | 470.1 | 2.02 | Method C | Prepared using the synthesis of Example 18 | 1-(2-(3- (acetoxymethyl)-7,8- dimethyl-2,4-dioxo- 3,4- dihydrobenzo[g]pteridin- 10(2H)- yl)ethyl)piperidine-4- carboxylic acid |
| 22 | 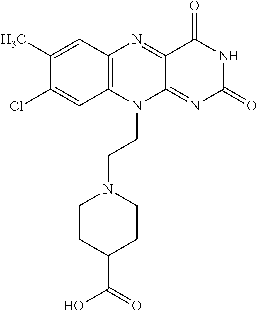 | 418.1 | 1.59 | Method C | Prepared using the synthesis of Example 16 | 1-[2-(8-Chloro-7- methyl-2,4-dioxo-3,4- dihydro-2H- benzo[g]pteridin-10- yl)-ethyl]-piperidine-4- carboxylic acid |
| 23 | 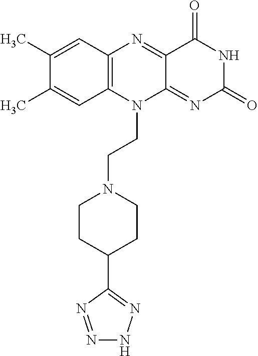 | 422.1 | 6.33 | Method E | Prepared using synthesis of Example 8 | 10-(2-(4-(1H-tetrazol- 5-yl)piperidin-1- yl)ethyl)-7,8- dimethylbenzo[g] pteridine-2,4(3H,10H)- dione |
| 24 | 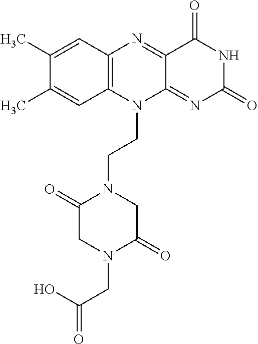 | 440.9 | 1.91 | Method C | Prepared using the synthesis of Example 9 | {4-[2-(7,8-Dimethyl- 2,4-dioxo-3,4-dihydro- 2H-benzo[g]pteridin- 10-yl)-ethyl]-2,5-dioxo- piperazin-1-yl}-acetic acid |
| 25 | 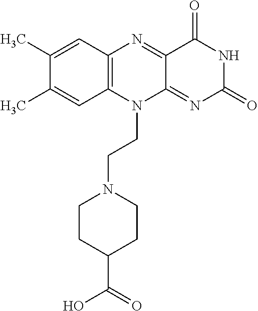 | 398.1 | 1.61 | Method C | Prepared using the synthesis of Example 9 | 1-[2-(7,8-Dimethyl-2,4- dioxo-3,4-dihydro-2H- benzo[g]pteridin-10- yl)-ethyl]-piperidine-4- carboxylic acid |
| 26 | 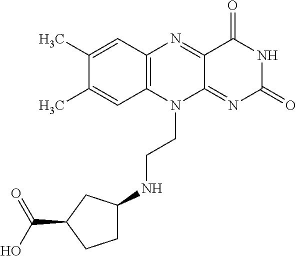 | MH− 396.13 | 4.969 | Method A | Prepared using the synthesis of Example 8 | (1R,3S)-3-(2-(7,8- dimethyl-2,4-dioxo- 3,4- dihydrobenzo[g]pteridin- 10(2H)- yl)ethylamino)cyclo- pentanecarboxylic acid |
| 27 | 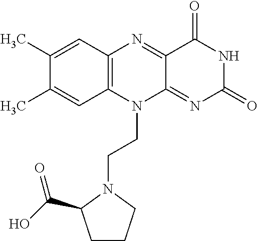 | 382.3 | 4.19 | Method A | Prepared using the synthesis of Example 20 | (S)-1-(2-(7,8-Dimethyl- 2,4-dioxo-3,4- dihydrobenzo[g]pteridin- 10(2H)- yl)ethyl)pyrrolidine-2- carboxylic acid |
| 28 | 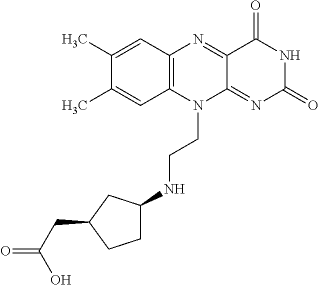 | 412.1 | 1.77 | Method C | Prepared using the synthesis of Example 6 | 2-((1R,3S)-3-(2-(7,8- dimethyl-2,4-dioxo- 3,4- dihydrobenzo[g]pteridin- 10(2H)- yl)ethylamino)cyclo- pentyl)acetic acid |
| 29 | 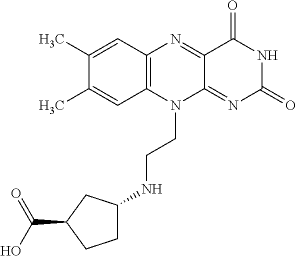 | 398 | 1.6 | Method C | Prepared using the synthesis of Example 7 | (1R,3R)-3-(2-(7,8- dimethyl-2,4-dioxo- 3,4- dihydrobenzo[g]pteridin- 10(2H)- yl)ethylamino)cyclo- pentanecarboxylic acid |
| 30 | 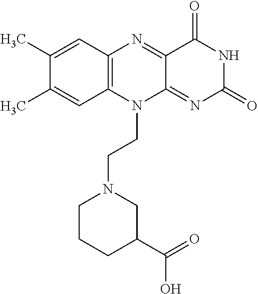 | 398.2 | 4.47 | Method A | Prepared using synthesis of Example 8 | 1-(2-(7,8-dimethyl-2,4- dioxo-3,4- dihydrobenzo[g]pteridin- 10(2H)- yl)ethyl)piperidine-3- carboxylic acid |
| 31 | 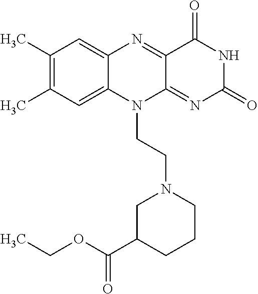 | 426.2 | 5.48 | Method A | Prepared using synthesis of Example 21 | Ethyl 1-(2-(7,8- dimethyl-2,4-dioxo- 3,4- dihydrobenzo[g]pteridin- 10(2H)- yl)ethyl)piperidine-3- carboxylate |
| 32 | 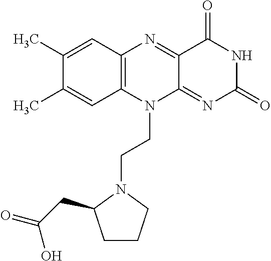 | 398.2 | 4.57 | Method A | Prepared using synthesis of Example 8 | (S)-2-(1-(2-(7,8- dimethyl-2,4-dioxo- 3,4- dihydrobenzo[g]pteridin- 10(2H)- yl)ethyl)pyrrolidin-2- yl)acetic acid |
| 33 | 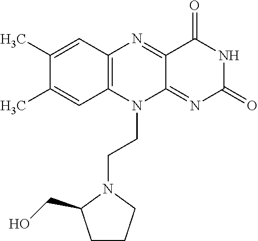 | 368.1 | 1.38 | Method B | Prepared using the synthesis of Example 9 | 10-[2-(2- Hydroxymethyl- pyrrolidin-1-yl)-ethyl]- 7,8-dimethyl-10H- benzo[g]pteridine-2,4- dione |
| 34 | 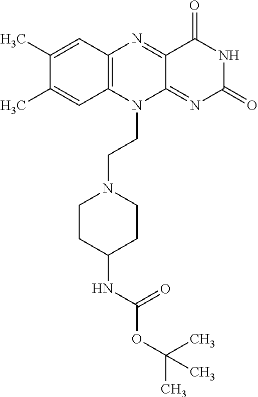 | 469.1 | 2.28 | Method C | Prepared using synthesis of Example 8 | tert-butyl 1-(2-(7,8- dimethyl-2,4-dioxo- 3,4- dihydrobenzo[g]pteridin- 10(2H)- yl)ethyl)piperidin-4- ylcarbamate |
| 35 | 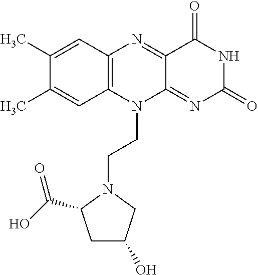 | 400 | 1.3 | Method C | Prepared using the synthesis of Example 3 Step 2 | 1-[2-(7,8-Dimethyl-2,4- dioxo-3,4-dihydro-2H- benzo[g]pteridin-10- yl)-ethyl]-4-hydroxy- pyrrolidine-2- carboxylic acid |
| 36 | 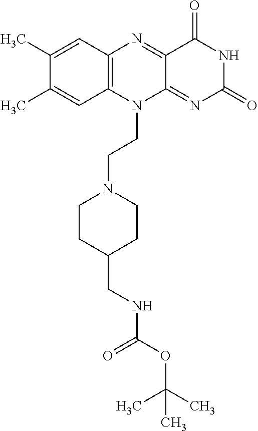 | 483 | 2.37 | Method B | Prepared using synthesis of Example 8 | 1-[2-(7,8-Dimethyl-2,4- dioxo-3,4-dihydro-2H- benzo[g]pteridin-10- yl)-ethyl]-piperidin-4- ylmethyl}-carbamic acid tert-butyl ester |
| 37 | 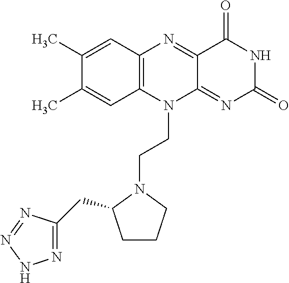 | 422.1 | 1.7 | Method C | Prepared using synthesis of Example 8 | (R)-10-(2-(2-((1H- tetrazol-5- yl)methyl)pyrrolidin-1- yl)ethyl)-7,8- dimethylbenzo[g] pteridine-2,4(3H,10H)- dione |
| 38 | 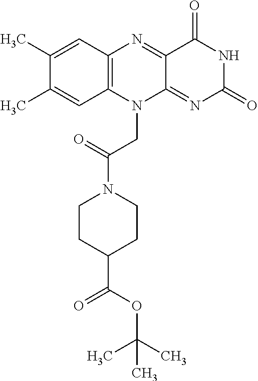 | 468 | 6.792 | Method A | Prepared using the synthesis of Example 26 | 1-[2-(7,8-Dimethyl-2,4- dioxo-3,4-dihydro-2H- benzo[g]pteridin-10- yl)-acetyl]-piperidine- 4-carboxylic acid tert- butyl ester |
| 39 | 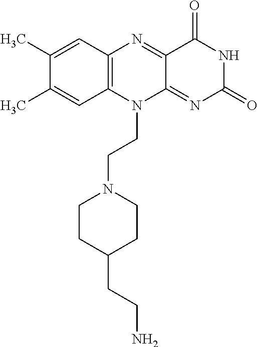 | 397.1 | 3.14 | Method B | Prepared using the synthesis of Intermediate 1, Step 3 | 10-{2-[4-(2-Amino- ethyl)-piperidin-1-yl]- ethyl}-7,8-dimethyl- 10H- benzo[g]pteridine-2,4- dione |
| 40 | 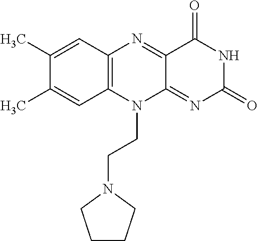 | 340 | 5.26 | Method A | Prepared using synthesis of Example 8 | 7,8-Dimethyl-10-(2- pyrrolidin-1-yl-ethyl)- 10H- benzo[g]pteridine-2,4- dione |
| 41 | 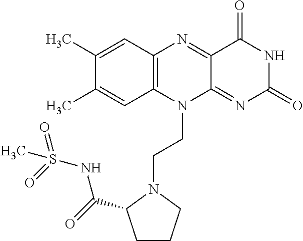 | 461 | 1.71 | Method C | Prepared using the synthesis of Example 13 | (R)-1-(2-(7,8-dimethyl- 2,4-dioxo-3,4- dihydrobenzo[g]pteridin- 10(2H)-yl)ethyl)-N- (methylsulfonyl)pyrrolidine- 2-carboxamide |
| 42 | 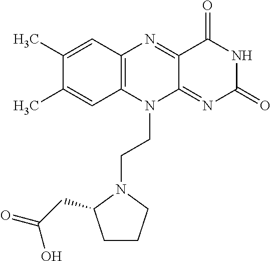 | 398.1 | 1.79 | Method C | Prepared using the synthesis of Example 9 | {1-[2-(7,8-Dimethyl- 2,4-dioxo-3,4-dihydro- 2H-benzo[g]pteridin- 10-yl)-ethyl]- pyrrolidin-2-yl}-acetic acid |
| 43 | 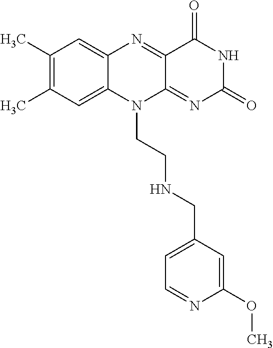 | 407.1 | 1.92 | Method C | Prepared using the synthesis of Example 5 | 10-(2-((2- methoxypyridin-4- yl)methylamino)ethyl)- 7,8- dimethylbenzo[g]pteridine- 2,4(3H,10H)- dione |
| 44 | 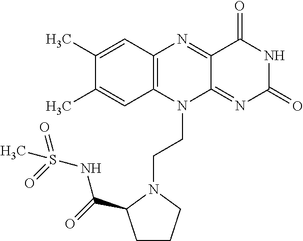 | 461.1 | 1.69 | Method C | Prepared using the synthesis of Example 12 | (S)-1-(2-(7,8-dimethyl- 2,4-dioxo-3,4- dihydrobenzo[g]pteridin- 10(2H)-yl)ethyl)-N- (methylsulfonyl) pyrrolidine-2-carboxamide |
| 45 | 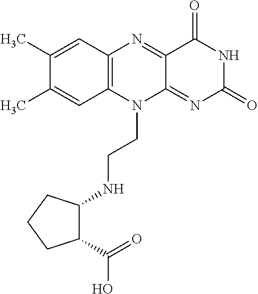 | 398.1 | 1.78 | Method C | Prepared using the synthesis of Example 3 Step 2 | 2-[2-(7,8-Dimethyl-2,4- dioxo-3,4-dihydro-2H- benzo[g]pteridin-10- yl)-ethylamino]- cyclopentanecarboxylic acid |
| 46 | 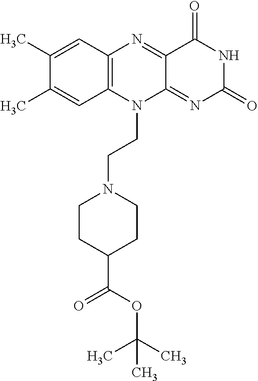 | 454.1 | 4.24 | Method B | Prepared using the synthesis of Example 3 Step 2 | 1-[2-(7,8-Dimethyl-2,4- dioxo-3,4-dihydro-2H- benzo[g]pteridin-10- yl)-ethyl]-piperidine-4- carboxylic acid tert- butyl ester |
| 47 | 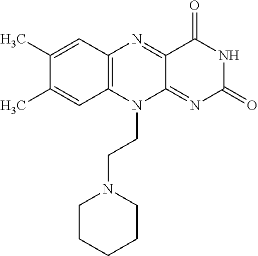 | 354.1 | 3.53 | Method C | Prepared using the synthesis of Example 8 | 7,8-dimethyl-10-(2- (piperidin-1- yl)ethyl)benzo[g]pteridine- 2,4(3H,10H)-dione |
| 48 | 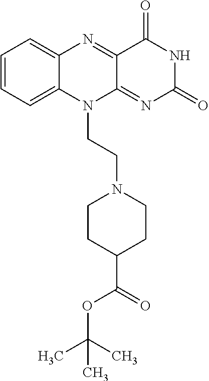 | 426.0 | 5.51 | Method A | Prepared using the synthesis of Example 22 | tert-Butyl 1-(2-(2,4- dioxo-3,4- dihydrobenzo[g]pteridin- 10(2H)- yl)ethyl)piperidine-4- carboxylate |
| 49 | 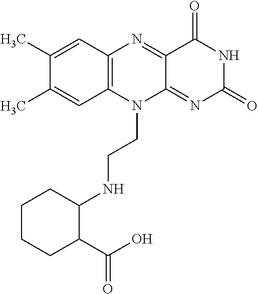 | MH− 410.07 | 9.566 | Method A | Prepared using the synthesis of Example 8 | 2-[2-(7,8-Dimethyl-2,4- dioxo-3,4-dihydro-2H- benzo[g]pteridin-10- yl)-ethylamino]- cyclohexanecarboxylic acid |
| 50 | 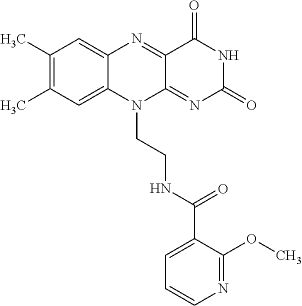 | 421.2 | 6.388 | Method A | Prepared using the synthesis of Example 11 | N-[2-(7,8-Dimethyl- 2,4-dioxo-3,4-dihydro- 2H-benzo[g]pteridin- 10-yl)-ethyl]-2- methoxy-nicotinamide |

### Example 29

### 10-(2-(4-(Aminomethyl)piperidin-1-yl)ethyl)-7,8-dimethylbenzo[g]pteridine-2,4(3H,10H)-dione bis(2,2,2-trifluoroacetate)

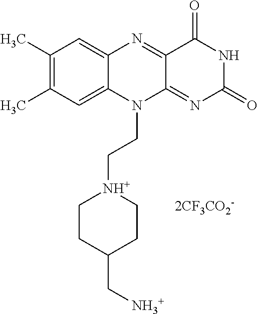

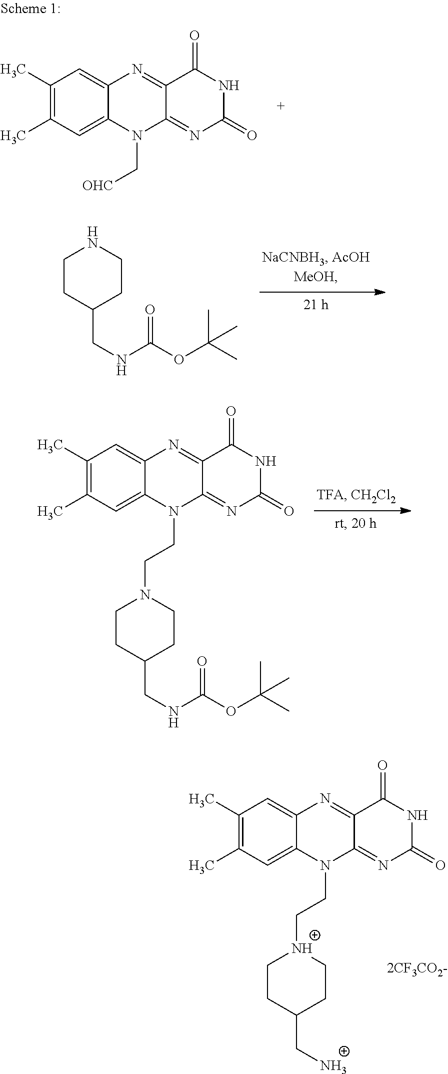

### Step 1 Preparation of 2-(7,8-Dimethyl-2,4-dioxo-3,4-dihydrobenzo[g]pteridin-10(2H)-yl)acetaldehyde

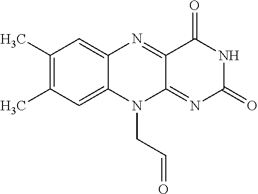

To a suspension of riboflavin (8.5 g, 0.0023 mol) in 2 N aqueous sulfuric acid (225 mL), cooled to 0° C. in a flask covered with tinfoil, is added orthoperiodic acid (18.9 g, 0.0825 mmol) dissolved in water (200 mL). After 30 min., the reaction is allowed to warm to room temperature. Once the reaction mixture becomes clear (a transparent, yellow solution), the pH of the reaction solution is adjusted carefully to 3.8-3.9 (using a pH meter) by addition of solid sodium carbonate. [It is extremely important that the pH is monitored carefully, if one goes over a pH of 3.9 the product does not precipitate out of solution.] The precipitate is then isolated by filtration and washed liberally with cold water, ethanol, and diethyl ether to yield desired product (6.04 g, 94%) as an orange solid. LC-MS m/z 285.1 [M+H]⁺, retention time 1.63 min.

### Step 2 Preparation of tert-butyl ((1-(2-(7,8-dimethyl-2,4-dioxo-3,4-dihydrobenzo[g]pteridin-10(2H)-yl)ethyl)piperidin-4-yl)methyl)carbamate

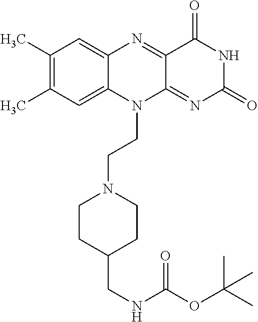

To a suspension of 2-(7,8-dimethyl-2,4-dioxo-3,4-dihydro-2H-benzo[g]pteridin-10-yl)-acetaldehyde (100 mg, 0.352 mmol) in MeOH (15 mL) are added tert-butyl (piperidin-4-ylmethyl)carbamate (226 mg, 1.056 mmol), and AcOH (0.1 mL) respectively and stirred at room temperature for 2 h. NaCNBH₃ (66 mg, 1.056 mmol) is then added to the reaction mixture and stirred at room temperature for 24 h. The solvent is removed under vacuum and the crude is purified by preparative HPLC (Method 2). Lyophilization of combined fractions affords desired product, tert-butyl ((1-(2-(7,8-dimethyl-2,4-dioxo-3,4-dihydrobenzo[g]pteridin-10(2H)-yl)ethyl)piperidin-4-yl)methyl)carbamate (104 mg, 62%) as a bright, yellow solid. ¹H NMR (400 MHz, MeOH-d₄): δ 1.46 (s, 9H), 1.61 (m, 2H), 1.84 (m, 1H), 2.04 (d, 2H), 2.51 (s, 3H), 2.63 (s, 3H), 3.03 (d, 2H), 3.13 (t, 2H), 3.68 (t, 2H), 3.97 (d, 2H), 5.12 (t, 2H), 7.82 (s, 1H), 8.03 (s, 1H). LC-MS m/z 483.1 (M+H)⁺, retention time: 2.40 min (Method A).

### Step 3 Preparation of 10-(2-(4-(aminomethyl)piperidin-1-yl)ethyl)-7,8-dimethylbenzo[g]pteridine-2,4(3H,10H)-dione bis(2,2,2-trifluoroacetate)

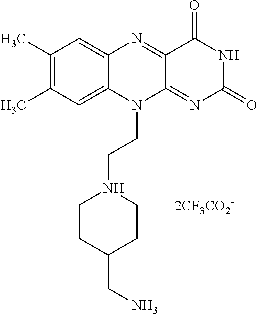

To a solution of tert-butyl benzyl(2-(7,8-dimethyl-2,4-dioxo-3,4-dihydrobenzo[g]pteridin-10(2H)-yl)ethyl)carbamate (96 mg, 0.2 mmol) in DCM (2 mL) is added TFA (2 mL). The reaction mixture is stirred at room temperature for 2 h. After 2 h, the reaction mixture is concentrated and the residual material is dissolved in MeOH (10 mL) and purified by preparative HPLC (Method 2). Lyophilization of combined fractions affords desired product, 10-[2-(4-aminomethyl-piperidin-1-yl)-ethyl]-7,8-dimethyl-10H-benzo[g]pteridine-2,4-dione di-trifluoroacetic acid salt (52 mg, 68%) as a bright, yellow solid. ¹H NMR (400 MHz, DMSO-d₆): δ 1.38 (m, 2H), 1.96 (m, 1H), 2.0 (d, 2H), 2.43 (s, 3H), 2.53 (s, 3H), 2.76 (m, 2H), 3.09 (t, 2H), 3.68 (t, 2H), 3.97 (d, 2H), 5.00 (t, 2H), 7.86 (s, 1H), 7.95 (m, 4H), 9.14 (br s, 1H), 11.47 (br s, 1H).

### Example 30

### N-Cyano-1-(2-(7,8-dimethyl-2,4-dioxo-3,4-dihydrobenzo[g]pteridin-10(2H)-yl)ethyl)piperidine-4-carboxamide

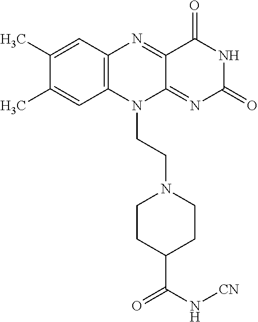

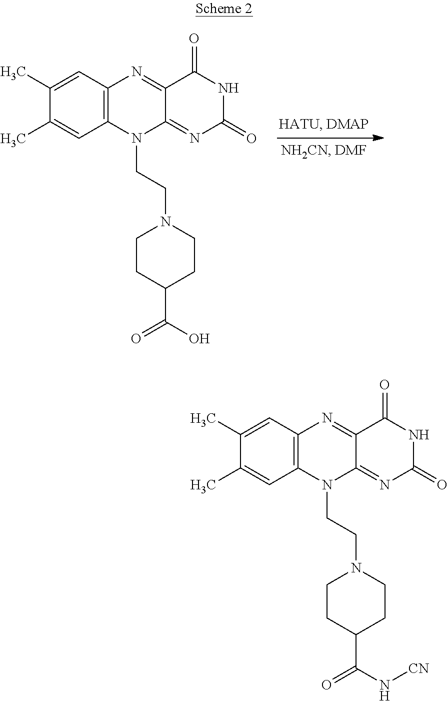

1-(2-(7,8-Dimethyl-2,4-dioxo-3,4-dihydrobenzo[g]pteridin-10(2H)-yl)ethyl)piperidine-4-carboxylic acid (10 mg, 0.0254 mmol) (prepared using the method of Example 29, step 2, and using 2-(7,8-dimethyl-2,4-dioxo-3,4-dihydro-2H-benzo[g]pteridin-10-yl)-acetaldehyde and piperidine-4-carboxylic acid as the starting materials), DMAP (3.1 mg, 0.0254 mmol) and cyanamide (21 mg, 0.05 mmol) is dissolved in anhydrous DMF (1 mL). HATU (11.5 mg, 0.03 mmol) is added to the reaction mixture. The mixture is allowed to stir for 24 h at 20° C., diluted with water (2 mL) and then purified by preparative HPLC (Method 1). N-Cyano-1-(2-(7,8-dimethyl-2,4-dioxo-3,4-dihydrobenzo[g]pteridin-10(2H)-yl)ethyl)piperidine-4-carboxamide is isolated in 46% (6 mg) yield. LC-MS m/z 422.0 [M+H]⁺, retention time 1.66 min. ¹H NMR (400 MHz, DMSO-d₆) δ 1.76 (m, 2H), 2.05 (m, 2H), 2.42 (s, 3H), 2.53 (s, 3H), 2.60 (m, 1H), 3.05 (m, 2H), 3.49 (s, 2H), 3.90 (m, 2H), 4.96 (s, 2H), 7.97 (s, 1H), 7.99 (s, 1H), 9.35 (br s, 1H), 11.48 (s, 1H).

### Example 31

### N-(1-(2-(7,8-Dimethyl-2,4-dioxo-3,4-dihydrobenzo[g]pteridin-10(2H)-yl)ethyl)piperidin-4 yl)methanesulfonamide 2,2,2-trifluoroacetate

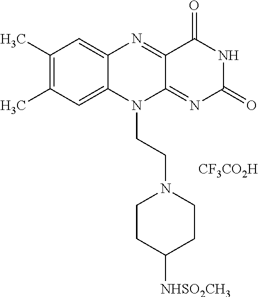

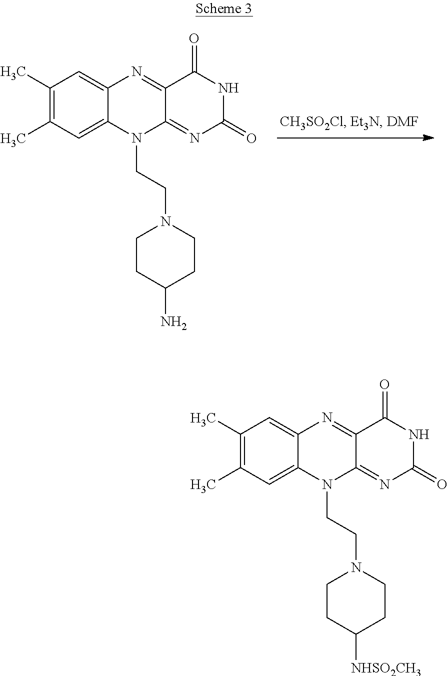

Step 1 Preparation of 10-(2-(4-aminopiperidin-1-yl)ethyl)-7,8-dimethylbenzo[g]pteridine-2,4(3H,10H)-dione bis(2,2,2-trifluoroacetate)

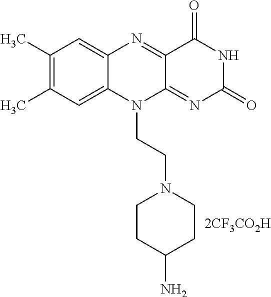

10-(2-(4-Aminopiperidin-1-yl)ethyl)-7,8-dimethylbenzo[g]pteridine-2,4(3H,10H)-dione is synthesized by following the procedure of Example 29 and using (7,8-dimethyl-2,4-dioxo-3,4-dihydro-2H-benzo[g]pteridin-10-yl)-acetaldehyde (102 mg, 0.36 mmol) and piperidin-4-amine (216 mg, 1.08 mmol) in 88% yield. ¹H NMR (400 MHz, DMSO-d₆) δ 1.73 (br m, 2H), 2.12 (br m, 2H), 2.43 (s, 3H), 2.53 (s, 3H), 3.17 (br m, 2H), 3.32 (br m, 2H), 3.54 (m, 1H), 3.99 (br m, 2H), 4.98 (bt, 2H), 7.86 (s, 1H), 7.98 (s, 1H), 8.14 (br s, 3H), 8.95 (br s, 1H), 11.50 (s, 1H).

### Step 2 Preparation of N-(1-(2-(7,8-dimethyl-2,4-dioxo-3,4-dihydrobenzo[g]pteridin-10(2H)-yl)ethyl)piperidin-4-yl)methanesulfonamide 2,2,2-trifluoroacetate

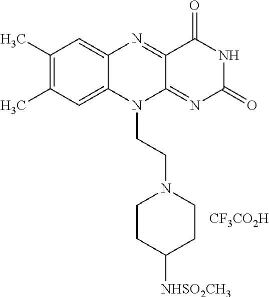

To a 0° C. solution of 10-(2-(4-aminopiperidin-1-yl)ethyl)-7,8-dimethylbenzo[g]pteridine-2,4(3H,10H)-dione (32 mg, 0.087 mmol) in anhydrous DMF (3 mL) under an Argon atmosphere is added methane sulfonylchloride (0.1 mL) dropwise. Trimethylamine (0.1 mL) is then added to the reaction mixture, causing it to become cloudy. After stirring the reaction mixture for 1.25 h at 0° C., it is warmed to rt and is stirred for 3.25 h. The crude reaction mixture is diluted with water (7 mL) and is purified by preparative HPLC (Method 4). The desired fractions are combined and lyophilized, and the desired product is obtained in 24% (12 mg) yield. ¹H NMR (400 MHz, CDCl₃) δ 1.63 (q, 2H), 1.94 (br s, 1H), 2.10 (d, 2H), 2.43 (s, 3H), 2.54 (s, 3H), 2.97 (s, 3H), 3.51 (br s, 4H), 3.93 (br m, 2H), 4.98 (br s, 2H), 7.37 (m, 1H), 7.84 (s, 1H), 7.99 (s, 1H), 8.54 (br m, 1H), 11.52 (s, 1H).

### Example 32

### (S)-10-(2-(2-(2H-Tetrazol-5-yl)pyrrolidin-1-yl)ethyl)-7,8-dimethylbenzo[g]pteridine-2,4(3H,10H)-dione

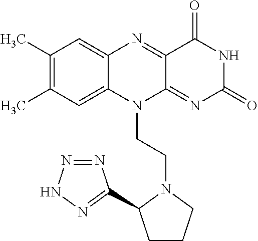

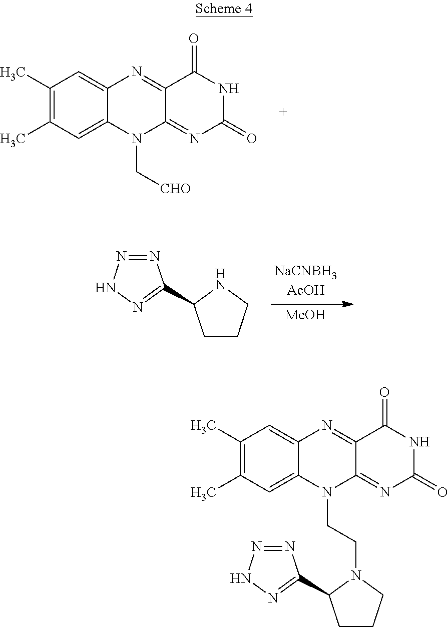

### Step 1 Preparation of (S)-5-(pyrrolidin-2-yl)-2H-tetrazole

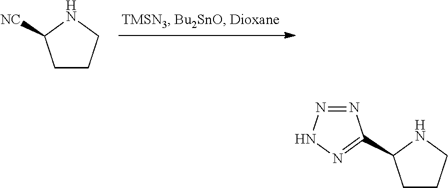

(S)-Pyrrolidine-2-carbonitrile hydrochloride (250 mg, 1.88 mmol) and dibutyl tinoxide (140 mg, 0.56 mmol) are suspended in dioxane (10 mL) and azidotrimethyl silane (0.9 mL, 7.52 mmol) is added to this mixture under Ar. The resultant mixture is heated to 110° C. for 21 h. Dibutyl tinoxide (140 mg, 0.56 mmol) and azidotrimethyl silane (0.9 mL, 7.52 mmol) are added and the heating is continued at 110° C. for another 21 h. Solvent is removed by evaporation and the crude (207 mg) is used in the synthesis of (5)-10-(2-(2-(2H-tetrazol-5-yl)pyrrolidin-1-yl)ethyl)-7,8-dimethylbenzo pteridine-2,4(3H,10H)-dione without further purification.

### Step 2 Preparation of (S)-10-(2-(2-(2H-tetrazol-5-yl)pyrrolidin-1-yl)ethyl)-7,8-dimethylbenzo[g]pteridine-2,4(3H,10H)-dione

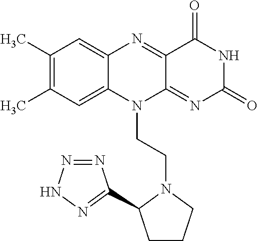

The title compound is prepared in 14% (14.5 mg) yield using the procedure of Example 29, step 2, using 2-(7,8-dimethyl-2,4-dioxo-3,4-dihydrobenzo[g]pteridin-10(2H)-yl)acetaldehyde (77 mg, 0.27 mmol), and (S)-5-(pyrrolidin-2-yl)-2H-tetrazole (207 mg, crude) as the starting materials. ¹H NMR (400 MHz, CD₃OD) δ 2.40 (m, 2H), 2.42 (s, 3H), 2.52 (m, 1H), 2.54 (s, 3H), 2.61 (m, 1H), 3.75 (br m, 2H), 3.95 (br m, 1H), 4.07 (br m, 1H), 5.09 (br m, 2H), 5.34 (br m, 1H), 7.77 (s, 1H), 7.89 (s, 1H).

### Example 33

### Preparation of 10-(2-((2-(1,1-dioxido-3-oxo-1,2,5-thiadiazolidin-2-yl)ethyl)amino)ethyl)-7,8-dimethylbenzo[g]pteridine-2,4(3H,10H)-dione

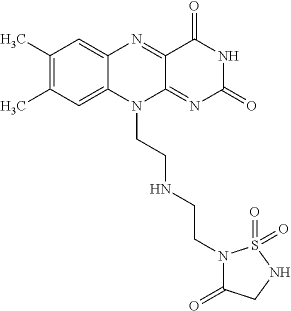

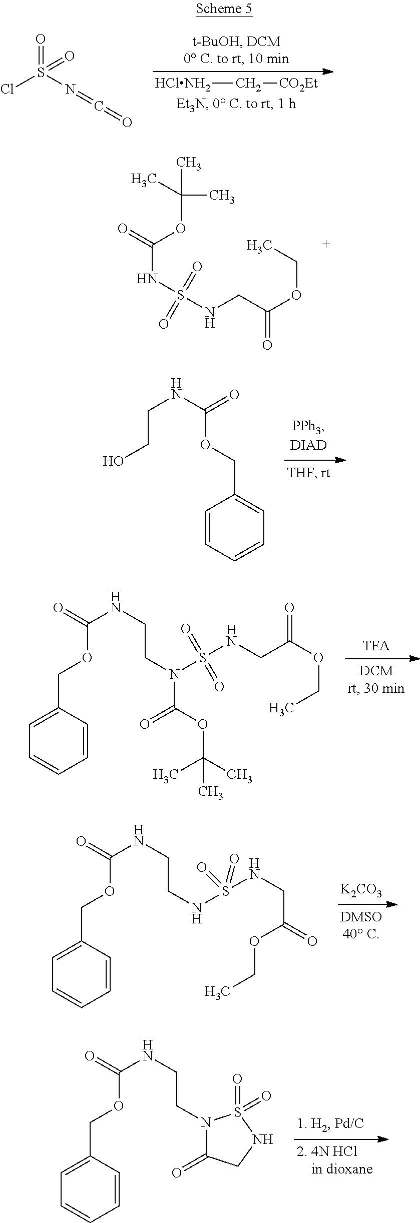

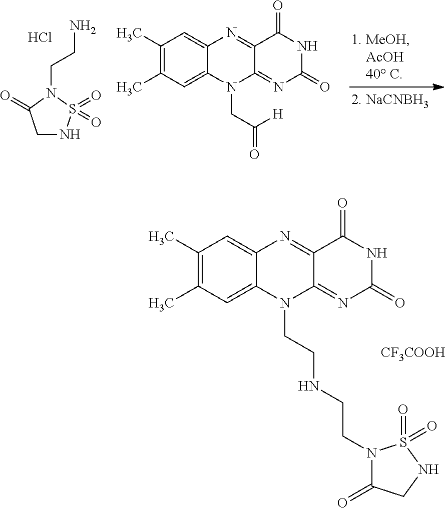

### Step 1 Preparation of ethyl 2-((N-(tert-butoxycarbonyl)sulfamoyl)amino)acetate

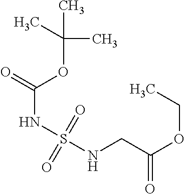

2-Methylpropan-2-ol (1.07 g, 14.4 mmol) is added to a solution of sulfurisocyanatidic chloride (2.06 g, 14.5 mmol) in anhydrous DCM (50 mL) at 0° C. The reaction mixture is warmed to rt, stirred for 10 min. and then cooled to 0° C. To this mixture, a solution of ethyl 2-aminoacetate hydrochloride (2.03 g, 14.5 mmol) and triethylamine (1.42 g, 14 mmol) in 30 mL of DCM is added, followed by triethylamine (1.93 g, 19 mmol). The resulting mixture is stirred for 1 h at it and 0.1N HCl (20 mL) is added and separated into two layers. The organic layer is washed with H₂O (10 mL), dried over Na₂SO₄, filtered, and concentrated under reduced pressure. The crude product is purified by BIOTAGE flash column chromatography using a gradient from 0 to 100% EtOAc in DCM as eluent. The desired product is isolated in 55% (2.3 g) yield. ¹H NMR (400 MHz, MeOH-d4) δ 1.32 (t, 3H), 1.52 (s, 9H), 3.98 (d, 2H), 4.25 (q, 2H), 5.68 (t, 1H), 7.31 (s, 1H).

### Step 2 Preparation of ethyl 2-((N-(2-(((benzyloxy)carbonyl)amino)ethyl)-N-(tert butoxycarbonyl)sulfamoyl)amino)acetate

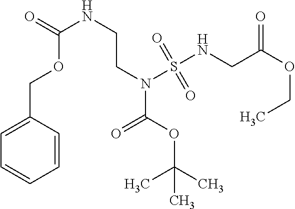

A mixture of ethyl 2-((N-(tert-butoxycarbonyl)sulfamoyl)amino)acetate (616 mg, 2.18 mmol), benzyl (2-hydroxyethyl)carbamate (678 mg, 3.47 mmol), DIAD (478 mg, 2.36 mmol) and triphenylphosphine (590 mg, 2.24 mmol) is stirred in THF at it for 1 h. The solvent is evaporated and the residue is purified by BIOTAGE flash column chromatography using a gradient from 0 to 100% EtOAc in DCM as eluent. The desired product is isolated as a light yellow oil (820 mg, 82%). ESI(+) [M+H]⁺=459.7.

### Step 3 Preparation of ethyl 2-((N-(2-(((benzyloxy)carbonyl)amino)ethyl)sulfamoyl)amino)acetate

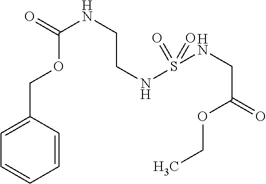

A mixture of ethyl 2-((N-(2-(((benzyloxy)carbonyl)amino)ethyl)-N-(tert-butoxycarbonyl)sulfamoyl)amino)acetate (820 mg, 1.78 mmol) and TFA (6 mL) in DCM (2 mL) is stirred for 30 min at rt. The solvent is reduced under vacuum and the crude is dissolved in EtOAc (100 mL) and washed with sat. aq. NaHCO₃ (20 mL×2), dried over Na₂SO₄, filtered, and concentrated under reduced pressure to obtain desired product as a light yellow solid (618 mg, 96%). This compound is used in the next step without further purification.

### Step 4 Preparation of benzyl (2-(1,1-dioxido-3-oxo-1,2,5-thiadiazolidin-2-yl)ethyl)carbamate

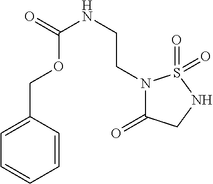

A mixture of ethyl 2-((N-(2-(((benzyloxy)carbonyl)amino)ethyl)sulfamoyl)amino)acetate (528 mg, 1.46 mmol) and K₂CO₃ (3.4 g, 24.6 mmol) in anhydrous DMSO (5 mL) is stirred overnight at 40° C. EtOAc (200 mL) is added to the reaction mixture at rt and solid is removed by filtration. The filtrate is washed with sat. aq. NaHCO₃ (50 mL) then brine (50 mL×2). The organic layer is dried over Na₂SO₄, filtered, and concentrated under reduced pressure to obtain product as a light yellow solid (310 mg, 67%). This compound is used without further purification.

### Step 5 Preparation of 2-(2-aminoethyl)-1,2,5-thiadiazolidin-3-one 1,1-dioxide hydrochloride

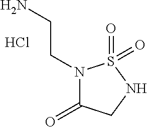

A solution of benzyl (2-(1,1-dioxido-3-oxo-1,2,5-thiadiazolidin-2-yl)ethyl)carbamate (56 mg, 0.17 mmol) in methanol (5 mL) is purged with argon for 10 min. Then, 4N HCl/dioxane (0.05 mL, 0.2 mmol) is added followed by palladium/carbon and the reaction mixture is placed under an atmosphere of hydrogen for 2 h. The reaction mixture is filtered through a celite pad and the filtrate is concentrated under reduced pressure to dryness. The residue is redissolved in MeOH (0.5 mL). Hexanes are added to precipitate the desired product which is filtered to obtain 2-(2-aminoethyl)-1,2,5-thiadiazolidin-3-one 1,1-dioxide hydrochloride (33 mg, 86%). ¹H NMR (400 MHz, DMSO-d₆) δ 3.02 (t, 2H), 3.36 (s, 1H), 3.80 (t, 2H), 4.09 (s, 2H), 8.38 (s, 3H).

### Step 6 Preparation of 10-(2-((2-(1,1-dioxido-3-oxo-1,2,5-thiadiazolidin-2-yl)ethyl)amino)ethyl)-7,8-dimethylbenzo[g]pteridine-2,4(3H,10H)-dione 2,2,2-trifluoroacetate

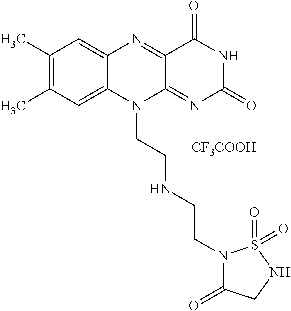

A mixture of 2-(2-aminoethyl)-1,2,5-thiadiazolidin-3-one 1,1-dioxide hydrochloride (23 mg, 0.10 mmol), 2-(7,8-dimethyl-2,4-dioxo-3,4-dihydrobenzo[g]pteridin-10(2H)-yl)acetaldehyde (23 mg, 0.08 mmol) and 2 drops of acetic acid in MeOH (5 mL) is stirred at 40° C. for 40 min. To this solution, NaCNBH₃ (27 mg, 0.42 mmol) is added and the mixture is stirred overnight. The mixture is concentrated, and the residue is dissolved in water (3 mL)-DMF (2 mL) and purified by preparative HPLC (Method 2). ¹H NMR (400 MHz, DMSO-d₆) δ 2.41 (s, 3H), 2.50 (s, 3H), 3.00 (m, 2H), 3.56 (d, 2H), 3.63 (m, 2H), 3.72 (s, 2H), 4.76 (m, 2H), 7.34 (m, 1H), 7.66 (m, 1H), 7.91 (s, 2H), 11.37 (s, 1H). ESI(+) [M+H]⁺=448.0.

### Example 34

### 10-((1-Benzylpiperidin-2-yl)methyl)-7,8-dimethylbenzo[g]pteridine-2,4(3H,10H)-dione

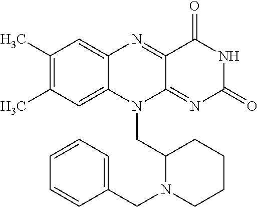

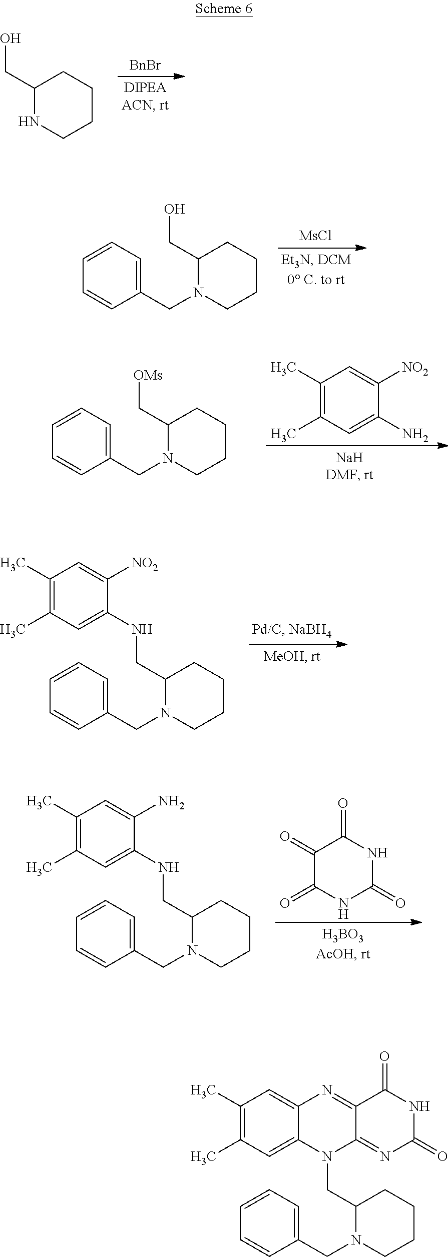

### Step 1 Preparation of (1-benzylpiperidin-2-yl)methanol

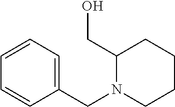

(1-Benzylpiperidin-2-yl)methanol is prepared by stirring piperidin-2-ylmethanol (1.16 g, 10 mmol) in acetonitrile (50 mL) at room temperature. Benzyl bromide (1.88 g, 11 mmol) and diisopropylethylamine (2.60 g, 20 mmol) are added in one portion and the resulting solution is stirred at room temperature for 2 h. The mixture is then evaporated under reduced pressure. DCM (50 mL) is added and washed sequentially with saturated, aqueous NaHCO₃ (50 mL) and 1 M KOH (10 mL). The aqueous phase is extracted with DCM (25 mL) and the combined organic portions are dried over Na₂SO₄, filtered and evaporated to give 2.02 g (9.86 mmol, 99% yield) of desired product as a yellow oil. LC-MS m/z 206.1 [M+H]⁺, retention time 3.22 min.

### Step 2 Preparation of (1-benzylpiperidin-2-yl)methyl methanesulfonate

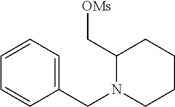

(1-Benzylpiperidin-2-yl)methyl methanesulfonate is prepared by stirring (1-benzylpiperidin-2-yl)methanol (2.02 g, 9.86 mmol) in DCM (50 mL) at 0° C., followed by dropwise addition of methanesulfonyl chloride (1.55 mL, 20.0 mmol) over 2.5 min., then dropwise addition of triethylamine (2.65 mL, 20 mmol) over 5 min. The reaction mixture is allowed to warm to room temperature with stirring over 165 min. The reaction mixture is poured into DCM (25 mL) and H₂O (75 mL), then the organic phase is washed sequentially with saturated, aqueous NH₄Cl (25 mL) and brine (40 mL), dried over Na₂SO₄, filtered and evaporated to give desired product (2.77 g, 99% yield) as an orange-yellow oil which is used in the next step without further purification.

### Step 3 Preparation of N-((1-benzylpiperidin-2-yl)methyl)-4,5-dimethyl-2-nitroaniline

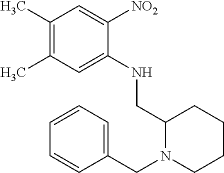

To a solution of 4,5-dimethyl-2-nitroaniline (0.91 g, 5.5 mmol) in DMF (25 mL) under Ar at rt is added sodium hydride (132 mg, 5.5 mmol) over 3 min. The resulting solution is stirred at rt for 10 min, then (1-benzylpiperidin-2-yl)methyl methanesulfonate (1.41 g, 5.0 mmol) is added in one portion at rt. The reaction mixture is allowed to stir for 18 h at rt under Ar. Water is added (50 mL) and the mixture is poured into DCM (200 mL) and H₂O (400 mL). The organic phase is washed sequentially with H₂O (2×200 mL) and brine (150 mL), dried over Na₂SO₄, filtered and evaporated. The crude product is then purified by column chromatography (0 to 100% EtOAc in hexanes) to give N-((1-benzylpiperidin-2-yl)methyl)-4,5-dimethyl-2-nitroaniline (480 mg, 27%) as a dark red solid. LC-MS m/z 354.1 [M+H]⁺, retention time 5.03 min.

### Step 4 Preparation of N¹-((1-benzylpiperidin-2-yl)methyl)-4,5-dimethylbenzene-1,2-diamine

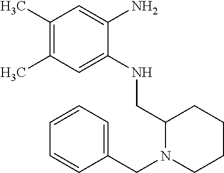

N-((1-Benzylpiperidin-2-yl)methyl)-4,5-dimethyl-2-nitroaniline (480 mg, 1.36 mmol) is dissolved in MeOH (25 mL). The reaction vessel is placed under vacuum, then repressurized with Ar, and this process is repeated. Pd/C (10% Pd/C, 3% Pd w/w) is added to the solution, and the mixture is cooled to 0° C. under Ar. NaBH₄ (216 mg, 5.7 mmol) is added portion-wise over 10 min. at 0° C., after which the reaction is stirred at 0° C. for 1 h, at which time the reaction mixture is filtered through celite using MeOH (50 mL) to elute the product. The solvent is then evaporated to give N¹-((1-benzylpiperidin-2-yl)methyl)-4,5-dimethylbenzene-1,2-diamine, 766 mg (quantitative) as a mixture of borate salts which is taken onto the next step without further purification.

### Step 5 Preparation of 10-((1-benzylpiperidin-2-yl)methyl)-7,8-dimethylbenzo[g]pteridine-2,4(3H,10H)-dione

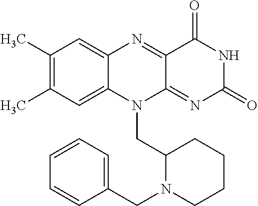

Crude N¹-((1-benzylpiperidin-2-yl)methyl)-4,5-dimethylbenzene-1,2-diamine (1.36 mmol), alloxan monohydrate (229 mg, 1.43 mmol) and boric acid (168 mg, 2.72 mmol) are dissolved in AcOH (15 mL) at rt, and the mixture is stirred at rt for 4 h. The reaction mixture is then evaporated to dryness, dissolved in DCM (50 mL) and H₂O (45 mL) is added, and the aqueous phase is extracted with DCM (2×20 mL). The combined organic portions are washed with brine (75 mL), and then dried over Na₂SO₄, filtered and evaporated to give a solid which is purified by column chromatography (0% to 15% MeOH in DCM). 10-((1-Benzylpiperidin-2-yl)methyl)-7,8-dimethylbenzo[g]pteridine-2,4(3H,10H)-dione is isolated as a yellow-orange powder in 22% (127 mg) yield. ¹H NMR (400 MHz, DMSO-d₆) δ 1.41 (br s, 3H), 1.57 (m, 2H), 1.77 (br s, 1H), 2.39 (s, 3H), 2.41 (s, 3H), 2.46 (s, 1H), 3.10 (m, 2H), 3.64 (d, 1H), 3.91 (d, 1H), 4.70 (br s, 1H), 4.95 (br s, 1H), 7.05 (s, 2H), 7.11 (s, 3H), 7.62 (s, 1H), 7.88 (s, 1H), 11.27 (s, 1H). LC-MS m/z 430.0 [M+H]⁺, retention time 2.67 min.

### Example 35

### 10-((1-Benzylpyrrolidin-2-yl)methyl)-7,8-dimethylbenzo[g]pteridine-2,4(3H,10H)-dione

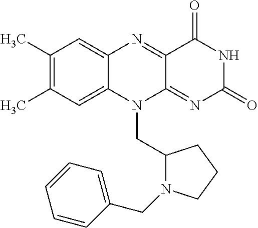

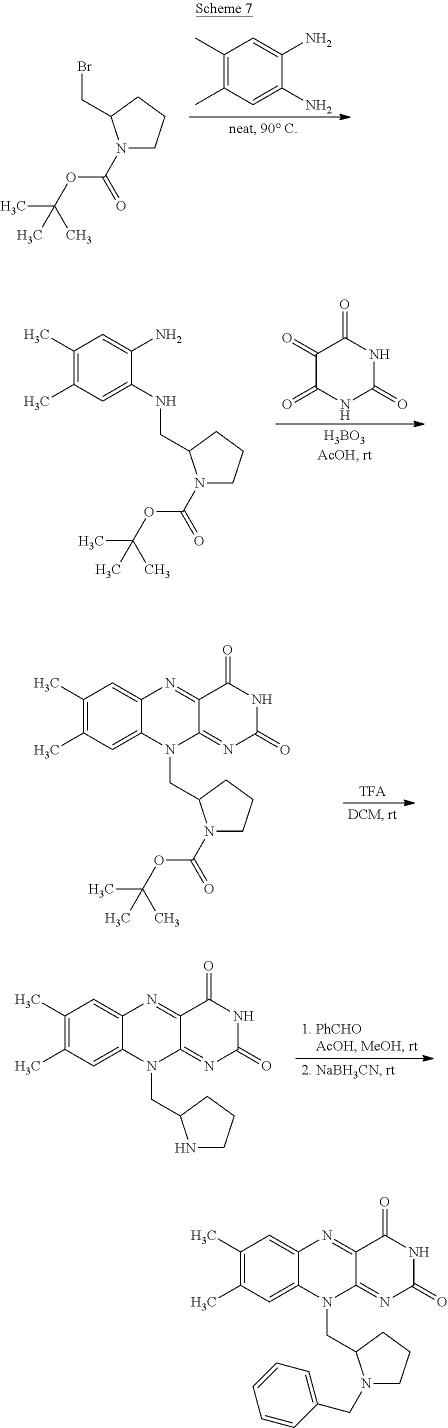

Step 1 Preparation of tert-butyl 2-(((2-amino-4,5-dimethylphenyl)amino)methyl)pyrrolidine-1-carboxylate

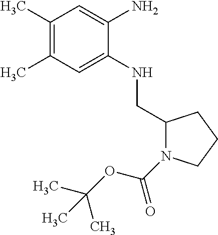

tert-Butyl 2-(((2-amino-4,5-dimethylphenyl)amino)methyl)pyrrolidine-1-carboxylate is synthesized by preparing a neat mixture of tert-butyl 2-(bromomethyl)pyrrolidine-1-carboxylate (475 mg, 1.8 mmol) and 4,5-dimethylbenzene-1,2-diamine (272 mg, 2.0 mmol), and heating the resulting paste to 90° C. for 1.5 h. The resulting liquid is cooled to room temperature and taken onto the next step without further purification. LC-MS m/z 319.9 [M+H], retention time 3.57 min.

### Step 2 Preparation of tert-butyl 2-((7,8-dimethyl-2,4-dioxo-3,4-dihydrobenzo[g]pteridin-10(2H)-yl)methyl)pyrrolidine-1-carboxylate

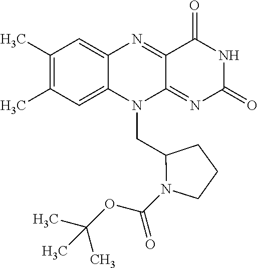

The crude tert-butyl 2-(((2-amino-4,5-dimethylphenyl)amino)methyl)pyrrolidine-1-carboxylate prepared above (2.0 mmol) is dissolved in AcOH (20 mL) at rt with alloxan monohydrate (336 mg, 2.1 mmol) and boric acid (247 mg, 4.0 mmol), and the resulting mixture is stirred at room temperature for 2.5 h. The reaction mixture is evaporated to dryness and then dry loaded onto silica gel (10 g) with MeOH. Column chromatography (0-20% MeOH in DCM) is performed, and the product is isolated as an impure mixture. This mixture is loaded onto preparatory TLC plates with 10% MeOH in DCM, and the plates are run using 5% MeOH in DCM as the mobile phase. The product is extracted from the silica with MeOH and evaporated to give tert-butyl 2-((7,8-dimethyl-2,4-dioxo-3,4-dihydrobenzo[g]pteridin-10(2H)-yl)methyl)pyrrolidine-1-carboxylate (65 mg, 8% yield over 2 steps) as an oily film. LC-MS m/z 425.9 [M+H], retention time 6.40 min.

### Step 3 7,8-dimethyl-10-(pyrrolidin-2-ylmethyl)benzo[g]pteridine-2,4(3H,10H)-dione 2,2,2-trifluoroacetic acid salt

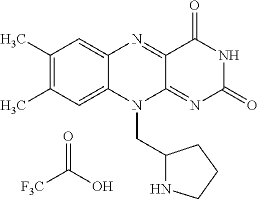

To a solution of tert-butyl 2-((7,8-dimethyl-2,4-dioxo-3,4-dihydrobenzo[g]pteridin-10(2H)-yl)methyl)pyrrolidine-1-carboxylate (65 mg, 0.15 mmol) in DCM (8 mL) at rt is added TFA (2 mL). The resulting solution is stirred at room temperature for 4 h, and then the mixture is evaporated to give 66 mg (0.15 mmol, quantitative) of the TFA salt of 7,8-dimethyl-10-(pyrrolidin-2-ylmethyl)benzo[g]pteridine-2,4(3H,10H)-dione as an oily film. LC-MS m/z 325.9 [M+H], retention time 3.83 min.

### Step 4 Preparation of 10-((1-benzylpyrrolidin-2-yl)methyl)-7,8-dimethylbenzo[g]pteridine-2,4(3H,10H)-dione

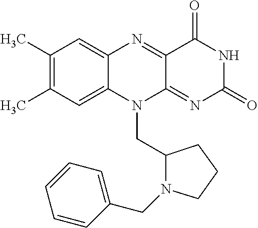

The TFA salt of 7,8-dimethyl-10-(pyrrolidin-2-ylmethyl)benzo[g]pteridine-2,4(3H,10H)-dione (25 mg, 0.077 mmol) is dissolved in a 1:1 mixture of MeOH and Et₃N (15 mL), dried under reduced pressure, and then dissolved in MeOH (5 mL) at room temperature. Benzaldehyde (10 mg, 0.094 mmol) and AcOH (1 drop) are added and the solution is stirred at room temperature for 3.5 h, and then NaBH₃CN (10 mg, 0.15 mmol) is added in one portion and the resulting solution is stirred at room temperature for 16 h. The reaction is quenched with H₂O (3 drops), and the reaction mixture is evaporated. The crude product is purified by preparative TLC (10% MeOH in DCM) to give 10-((1-benzylpyrrolidin-2-yl)methyl)-7,8-dimethylbenzo[g]pteridine-2,4(3H,10H)-dione (4.5 mg, 14%) as a yellow powder. ¹H NMR (400 MHz, MeOD) δ 1.58 (m, 1H), 1.73 (m, 1H), 2.08 (m, 5H), 2.48 (s, 3H), 2.56 (s, 3H), 3.43 (s, 2H), 4.51 (m, 2H), 6.68 (d, 2H), 6.89-6.97 (m, 3H), 7.71 (s, 1H), 7.88 (s, 1H). LC-MS m/z 416.1 [M+H], retention time 2.35 min.

### Example 36

### 7,8-Dimethyl-10-(3-(tetrahydro-2H-pyran-4-yl)propyl)benzo[g]pteridine-2,4(3H,10H)-dione

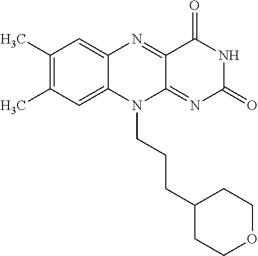

### Step 1 Preparation of 4,5-dimethyl-2-nitro-N-(3-(tetrahydro-2H-pyran-4-yl)propyl)aniline

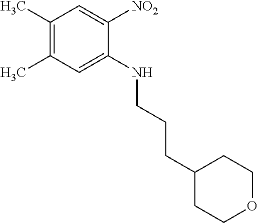

4,5-Dimethyl-2-nitro-N-(3-(tetrahydro-2H-pyran-4-yl)propyl)aniline is prepared by heating a solution of 1-bromo-4,5-dimethyl-2-nitrobenzene (115 mg, 0.5 mmol) and 3-(tetrahydro-2H-pyran-4-yl)propan-1-amine (commercially available) (143 mg, 1.0 mmol) in DMSO (1 mL) at 130° C. for 35 min, then at 160° C. for 10 min. The resulting mixture is diluted in EtOAc (25 mL) and H₂O (75 mL), and basified to pH 9 with 1N NaOH. The organic phase is then washed with H₂O (100 mL), dried over Na₂SO₄, filtered and evaporated. The resulting solid is dry-loaded onto silica gel and purified by column chromatography (EtOAc/hexane, gradient from 0-100% EtOAc) to give 18 mg (12% yield) of desired product as an orange solid. LC-MS m/z 293.0 [M+H], retention time 5.55 min.

### Step 2 Preparation of 4,5-dimethyl-N¹-(3-(tetrahydro-2H-pyran-4-yl)propyl)benzene-1,2-diamine

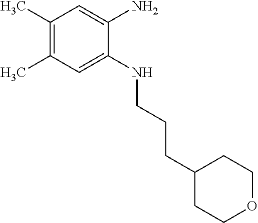

4,5-Dimethyl-N¹-(3-(tetrahydro-2H-pyran-4-yl)propyl)benzene-1,2-diamine is prepared from 4,5-dimethyl-2-nitro-N-(3-(tetrahydro-2H-pyran-4-yl)propyl)aniline (18 mg, 0.062 mmol) by catalytic reduction with Pd/C (10% Pd/C, 3% Pd w/w) and NaBH₄ (5 mg, 0.13 mmol) in MeOH (5 mL) and EtOAc (5 mL) at room temperature under Ar. After 30 min., at which time the reaction mixture is filtered through celite using EtOAc (15 mL), then MeOH (15 mL) to elute the product. The solvent is then evaporated to give 4,5-dimethyl-N¹-(3-(tetrahydro-2H-pyran-4-yl)propyl)benzene-1,2-diamine (quantitative) as a mixture of borate salts which is taken onto the next step without further purification.

### Step 3 Preparation of 7,8-dimethyl-10-(3-(tetrahydro-2H-pyran-4-yl)propyl)benzo[g]pteridine-2,4(3H,10H)-dione

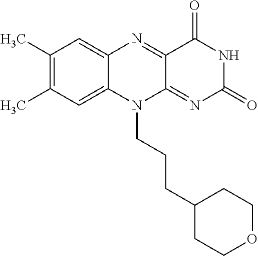

7,8-Dimethyl-10-(3-(tetrahydro-2H-pyran-4-yl)propyl)benzo[g]pteridine-2,4(3H,10H)-dione is prepared by stirring the crude 4,5-dimethyl-N-1-(3-(tetrahydro-2H-pyran-4-yl)propyl)benzene-1,2-diamine (0.062 mmol), alloxan monohydrate (11 mg, 0.069 mmol) and boric acid (8 mg, 0.13 mmol) in AcOH (5 mL) at rt for 16 h. The reaction mixture is then evaporated to dryness, dissolved in DCM (25 mL) and H₂O (50 mL), and the organic phase is washed with brine (2×40 mL) and then dried over Na₂SO₄, filtered and evaporated to give a solid which is purified by preparatory TLC (5% MeOH/DCM, then 10% MeOH/DCM) to provide desired product (11 mg, 48%) as a yellow solid. ¹H NMR (400 MHz, CDCl₃) δ 1.15 (m, 2H), 1.24 (s, 1H), 1.42 (t, 2H), 1.60 (d, 2H), 1.74 (t, 2H), 2.41 (s, 3H), 3.28 (t, 2H), 3.84 (t, 2H), 4.57 (t, 2H), 7.81 (s, 1H), 7.92 (s, 1H), 11.31 (s, 1H). LC-MS m/z 369.2 [M+H], retention time 3.25 min.

### Example 37

### 10-(3-Cyclohexylpropyl)-7,8-dimethylbenzo[g]pteridine-2,4(3H,10H)-dione

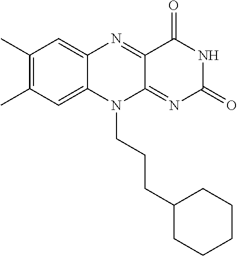

### Step 1 Preparation of N-(3-cyclohexylpropyl)-4,5-dimethylbenzene-1,2-diamine

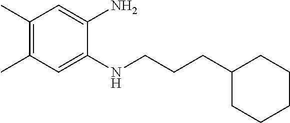

A well-stirred slurry of (3-bromopropyl)cyclohexane (1.20 g, 5.8 mmol), 4,5-dimethyl-o-phenylenediamine (3.18 g, 23.4 mmol), sodium bicarbonate (0.98 g, 11.7 mmol) and tetra-n-butylammonium iodide (0.22 g, 0.58 mmol) in toluene (30 mL) is heated at 70° C. under nitrogen for 18 h. The reaction is cooled to rt, partitioned between water and ethyl acetate (100 mL each), the layers are separated and the aqueous layer is extracted with ethyl acetate (3×20 mL). The organic layers are combined, dried with anhydrous sodium sulfate and concentrated. The residue is subjected to silica gel chromatography (230-400 mesh, 150 g, elution with 20% ethyl acetate/hexane) to give 1.0 g (66%) of the desired product as an oil. ¹H NMR (400 MHz, CDCl₃) δ 0.90 (2H, m), 1.24 (6H, m), 1.67 (7H, m), 2.13 (3H, s), 2.18 (3H, s), 3.05 (2H, t), 3.18 (3H, br s), 6.46 (1H, s), 6.53 (1H, s); MS (ESI⁺) for C₁₇H₂₈N₂ m/z 261.2 (M+H)⁺, HPLC retention time: 3.93 min. (System D).

### Step 2 Preparation of 10-(3-cyclohexylpropyl)-7,8-dimethylbenzo[g]pteridine-2,4(3H,10H)-dione

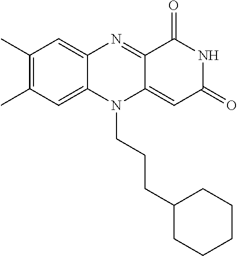

To a mixture of N-(3-cyclohexylpropyl)-4,5-dimethylbenzene-1,2-diamine (0.165 g, 0.63 mmol), alloxan (101 mg, 0.63 mmol) and boric acid (118 mg, 1.9 mmol) is added acetic acid (5 mL). The reaction is then stirred at rt for 18 h. The acetic acid is removed in vacuo. The residue is suspended in water and the precipitate collected, washed with water and air dried. The solid is subjected to silica gel chromatography (Silicycle, 230-400 mesh, 50 g, elution with 2% MeOH/DCM) to give 134 mg (58%) of the product as an amorphous yellow solid. ¹H NMR (400 MHz, DMSO-d₆) δ□ 0.88 (2H, m), 1.26 (6H, m), 1.67 (7H, m), 2.40 (3H, s), 4.54 (2H, m), 7.77 (1H, s), 7.90 (1H, s), 11.29 (1H, s); MS (ESI⁺) for C₂₁H₂₆N₄O₂ m/z 367.3 (M+H)⁺, HPLC retention time: 4.20 min. (System D).

### Example 38

### 7,8-dimethyl-10-(3-pyridin-3-yl)propyl)benzo[g]pteridine-2,4(3H,10H)-dione

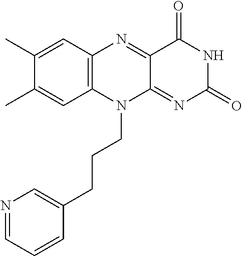

### Step 1 Preparation of 4,5-dimethyl-2-nitro-N-(3-pyridin-3-ylpropyl)aniline

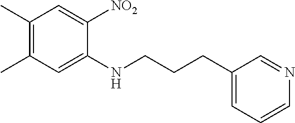

Sodium hydride, 60% in mineral oil (60:40, Sodium hydride:mineral oil, 0.146 g, 3.64 mmol) is added to a solution of 4,5-dimethyl-2-nitroaniline (0.55 g, 3.3 mmol) in 20 mL of DMF which is cooled in an ice bath under N₂. When gas evolution stops, the ice bath is removed and the mixture is stirred for 30 min at rt. 3-(3-Bromopropyl)pyridine [see Helv. Chim. Acta, 1982, 65(6), 1864] (0.795 g, 3.97 mmol) is added and the mixture is stirred at rt overnight. The DMF is evaporated and the residue is partitioned between 30 mL of EtOAc and 30 mL of saturated, aqueous NH₄Cl. The layers are separated and the aqueous phase is extracted with 3×20 mL of EtOAc. The organic layer is dried over Na₂SO₄ and evaporation gives 1.0 g of a dark, red solid. Silica gel chromatography (50 g, elution with 5% EtOAc/CH₂Cl₂) gives 0.25 g (26%) of the desired product as a red solid. MS (ESI+) for C₁₆H₁₉N₃O₂ m/z 286 (M+H)⁺.

### Step 2 Preparation of 4,5-dimethyl-N-(3-pyridin-3-ylpropyl)benzene-1,2-diamine

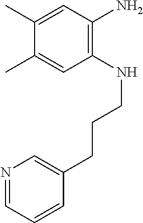

4,5-Dimethyl-2-nitro-N-(3-pyridin-3-ylpropyl)aniline (0.255 g, 0.894 mmol) is added as a solution in EtOH (10 mL) to nickel (0.0262 g, 0.447 mmol) and the mixture is stirred at rt under 1 atmosphere of H₂. After 1 hr, the nickel is removed by filtration through Celite 545 and the filtrate is evaporated to provide the desired product (0.22 g, 96%) as an oil. ¹H NMR (400 MHz, CDCl₃) δ 8.51 (d, 1H), 8.48 (dd, 1H), 7.55 (d, 1H), 7.24 (dd, 1H), 6.56 (s, 1H), 6.44 (s, 1H), 3.21 (m, 3H), 3.15 (t, 2H), 2.79 (t, 2 H), 2.18 (s, 3H), 2.15 (s, 3H), 2.02 (d, 2H).

### Step 3 Preparation of 7,8-dimethyl-10-(3-pyridin-3-yl)propyl)benzo[g]pteridine-2,4(3H,10H)-dione

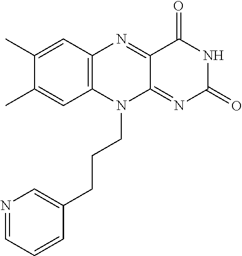

To a mixture of 4,5-dimethyl-N-(3-pyridin-3-ylpropyl)benzene-1,2-diamine (0.220 g, 0.862 mmol), alloxan (0.14 g, 0.86 mmol) and diboron trioxide (0.18 g, 2.6 mmol) is added 5 mL of HOAc. The mixture is then shaken at 60° C. for 1 hr. The acetic acid is removed in vacuo, and the remaining solid is taken up in 20 mL of H₂O. The pH of the mixture is adjusted to ˜7 by addition of saturated, aqueous NaHCO₃ and the mixture is extracted with 3×20 mL of CH₂Cl₂. The organics are combined, dried with anhydrous sodium sulfate and concentrated. Silica gel chromatography (15 g, elution with 3% MeOH/CH₂Cl₂) provides desired product (10 mg) as a yellow solid. HPLC retention time: 2.28 min (System D). ¹H NMR (400 MHz, DMSO-d₆) δ 11.32 (s, 1H), 8.49 (d, 1H), 8.41 (d, 1H), 7.91 (s, 1H), 7.66 (m, 2H), 7.32 (dd, 1H), 4.63 (t, 2H), 2.82 (d, 2H), 2.50 (m, 6 H), 2.40 (m, 2H).

### Example 39

### 8-(Dimethylamino)-7-methyl-10-[3-(1H-pyrrol-1-yl)propyl]benzo[g]pteridine-2,4(3H,10H)-dione

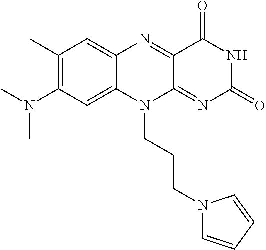

To a pressure tube containing a solution of 8-chloro-7-methyl-10-[3-(1H-pyrrol-1-yl)propyl]benzo[g]pteridine-2,4(3H,10H)-dione (120 mg, 0.32 mmol) in N-methylpyrrolidinone (2.50 mL) is added a solution of dimethylamine in tetrahydrofuran (2.0 M, 0.985 mL, 1.97 mmol). The tube is sealed and the mixture is stirred for 8 h at 80° C. Concentration of the reaction mixture at reduced pressure provided a residue that is purified by flash chromatography (230-400 mesh, CH₂Cl₂/0.07N methanolic ammonia (0.5-1.5%) as eluant) to afford 55 mg (45%) of the desired product as an amorphous, red solid. ¹H NMR (DMSO-d₆) δ 2.18 (p, 2H), 2.42 (s, 3H), 2.98 (s, 3H), 3.33 (s, 3H), 4.11 (t, 2H), 4.47 (br t, 2H), 6.01 (d, 2H), 6.52 (s, 1H), 6.89 (d, 2H), 7.78 (s, 1H), 11.13 (s, 1H); MS (ESI+) for C₂₀H₂₂N₆O₂ m/z 379.1 (M+H)⁺, HPLC retention time: 3.31 min. (Method G).

### Example 40

### 7,8-Dimethyl-10-(3-pyridin-2-ylpropyl)benzo pteridine-2,4(3H,10H)-dione

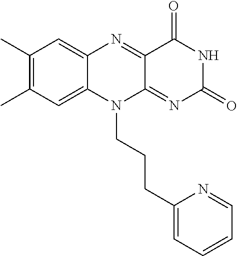

Step 1 Preparation of 4,5-dimethyl-2-nitro-N-(3-pyridin-2-yl)propyl)aniline

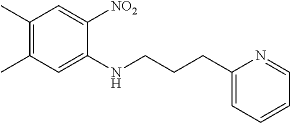

A well-stirred slurry of 1-bromo-4,5-dimethyl-2-nitrobenzene (1.04 g, 4.54 mmol; prepared as described in Chemistry—A European Journal, 2005, 11, 6254), 3-pyridin-2-ylpropan-1-amine (412 mg, 3.02 mmol; prepared as described in J Med Chem, 1969, 10(3), 498-499), cesium carbonate (1.97 g, 6.05 mmol) and oxydi-2,1-phenylene)bis[diphenylphosphine (244 mg, 0.454 mmol) in toluene (16 mL) is flushed with nitrogen (4×). Tris(dibenzylideneacetone)dipalladium(0) (138 mg, 0.151 mmol) is added and the mixture is then heated to 80° C. overnight. The reaction is cooled to room temperature and filtered (4×5 mL toluene rinses). The filtrate is shaken with 0.2N HCl (6×30 mL), and the combined aqueous layers (red) are made basic (pH 10-11) with aqueous K₂CO₃ and then extracted with DCM (6×40 mL). The combined DCM layers are stripped to dryness, giving 755 mg (83%) of desired product as a red solid. ¹H NMR (400 MHz, CDCl₃) δ 8.50-8.63 (1H, m), 8.06 (1H, br s), 7.92 (1H, s), 7.56-7.65 (1H, m), 7.10-7.22 (2H, m), 6.61 (1H, s), 3.31-3.42 (2H, m), 2.95 (2H, t), 2.25 (3H, s), 2.19-2.24 (2 H, m), 2.17 (3H, s); MS (ESI+) for C₁₆H₁₉N₃O₂ m/z 286.19 (M+H)⁺.

### Step 2 Preparation of 4,5-dimethyl-N-(3-pyridin-2-yl)propyl)benzene-1,2-diamine

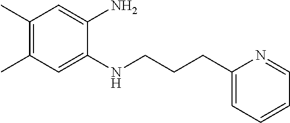

A stirring mixture of 4,5-dimethyl-2-nitro-N-(3-pyridin-2-ylpropyl)aniline, EtOH (30 mL), and Raney Nickel (1 mL of a slurry, 200 mmol) is flushed with N₂ and then stirred under H₂ (1 atmosphere). After overnight stirring, the mixture is filtered through celite (5×5 mL MeOH rinses) and stripped to a brown solid (0.63 g, 99%) and used without further purification in the next step. MS (ESI+) for C₁₆H₂₁N₃ m/z 256.23 (M+H)⁺.

### Step 3 Preparation of 7,8-dimethyl-10-(3-pyridin-2-ylpropyl)benzo[g]pteridine-2,4(3H,10H)-dione

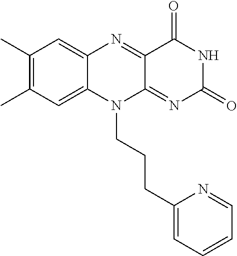

A N₂-flushed mixture of 4,5-dimethyl-N-(3-pyridin-2-ylpropyl)benzene-1,2-diamine (0.63 g, 2.5 mmol), alloxan monohydrate (0.434 g, 2.71 mmol), boric acid (0.458 g, 7.40 mmol), and acetic acid (40 mL) is stirred at rt. After overnight stirring, the reaction mixture is filtered through a sintered glass funnel and the remaining solid is washed with AcOH (4×1 mL), DCM (5×1 mL), EtOAc (5×1 mL), water (5×1 mL), and finally acetone (5×1 mL). The remaining solid is dried under high vacuum, giving the desired product as a yellow solid (0.37 g, 41%). ¹H NMR (400 MHz, DMSO-d₆) δ 11.31 (1H, s), 8.49-8.54 (1H, m), 7.91 (1H, br s), 7.77 (1H, br s) 7.68-7.74 (1H, m), 7.29-7.36 (1H, m), 7.20-7.27 (1H, m), 4.59-4.79 (2H, m), 2.90-3.06 (2H, m), 2.49 (3H, s), 2.42 (3H, s), 2.07-2.26 (2H, m); MS (ESI+) for C₂₀H₁₉N₅O₂ m/z 362.09 (M+H)⁺. HPLC retention time: 2.32 min. (System E).

By using the methods described above and by selecting the appropriate starting materials, other compounds of the invention are prepared and characterized. These compounds, together with the Examples described above, are summarized in Table 2.

TABLE 2

| | | | HPLC | | |
|---|---|---|---|---|---|
| | | LC-MS | retention | | |
| | | MH+ | time | HPLC | |
| Entry | Structure | (m/z) | (min.) | method | Preparation |
| 1 | 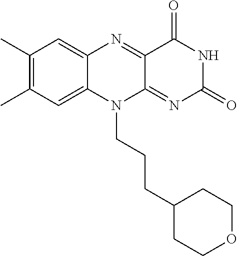 | 369.2 | 3.25 | C | See Example 36 |
| 2 | 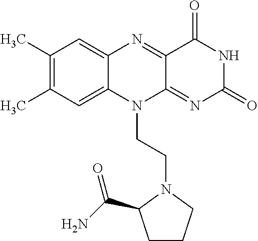 | 383.1 | 1.54 | C | Prepared using the synthesis of Step 2, Example 29 |
| 3 | 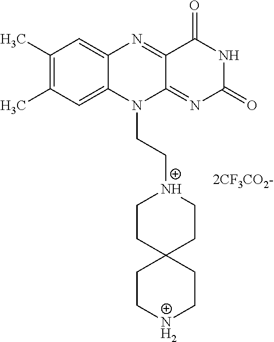 | 423.1 | 3.43 | B | Prepared using the synthesis of Step 2, Example 29 |
| 4 | 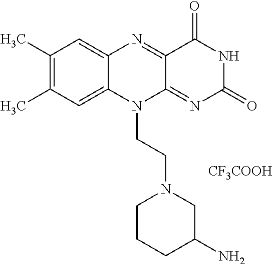 | 369.1 | 4.65 | A | Prepared using the synthesis of Step 2, Example 29 |
| 5 | 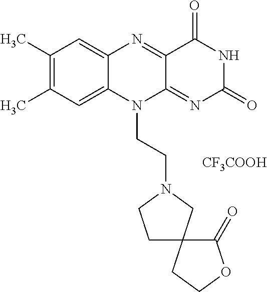 | 410.1 | 169 | C | Prepared using the synthesis of Step 2, Example 29 |
| 6 | 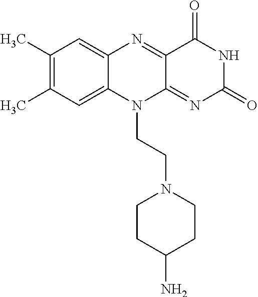 | 369.1 | 4.73 | A | Prepared using the synthesis of Step 2, Example 29 |
| 7 | 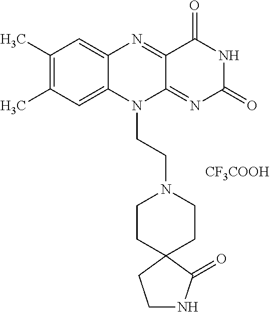 | 423.1 | 1.78 | C | Prepared using the synthesis of Step 2, Example 29 |
| 8 | 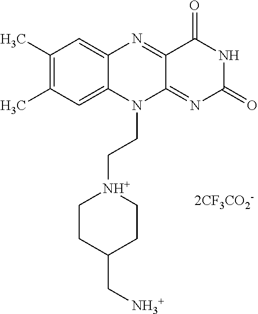 | 383.1 | 1.2 | C | See Example 29 |
| 9 | 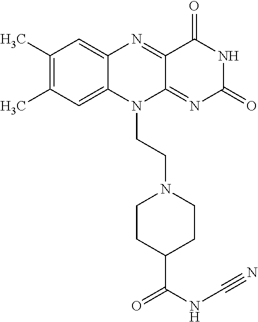 | 422 | 1.66 | C | See Example 30 |
| 10 | 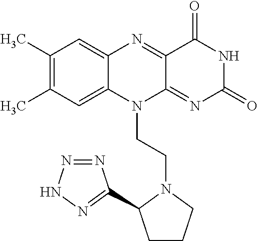 | 408 | 3.37 | C | See Example 32 |
| 11 | 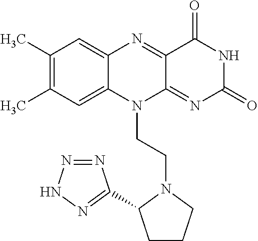 | 408.13 | 5.24 | A | Prepared using the synthesis of Example 32 |
| 12 | 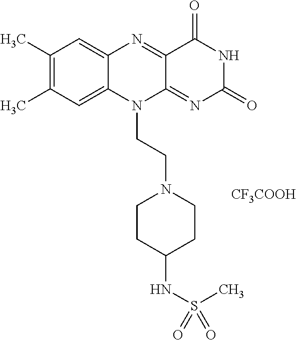 | 447.2 | 8.57 | A | See Example 31 |
| 13 | 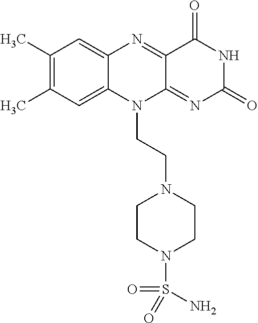 | 434.2 | 8.16 | A | Prepared using the synthesis of Step 2, Example 29 |
| 14 | 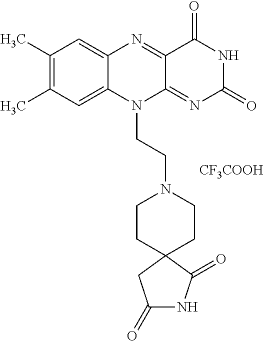 | 436.9 | 3.37 | C | Prepared using the synthesis of Step 2, Example 29 |
| 15 | 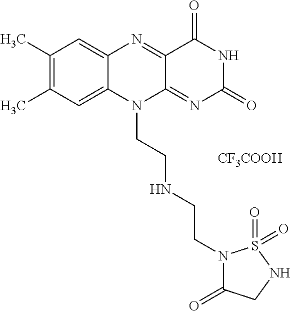 | 448 | 1.97 | C | See Example 33 |
| 16 | 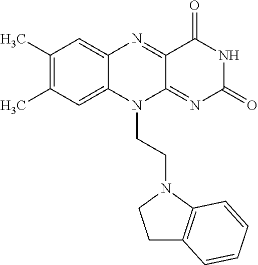 | 388 | 3.7 | C | Prepared using the synthesis of Step 2, Example 29 |
| 17 | 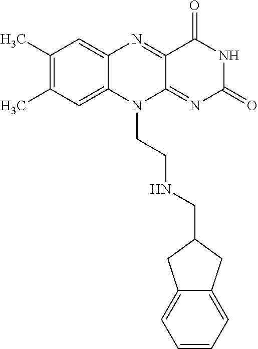 | 415.9 | 4.17 | C | Prepared using the synthesis of Step 2, Example 29 |
| 18 | 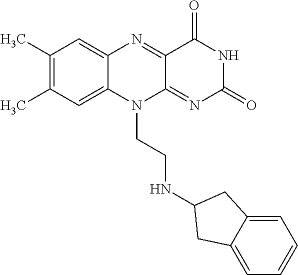 | 401.9 | 3.93 | C | Prepared using the synthesis of Step 2, Example 29 |
| 19 | 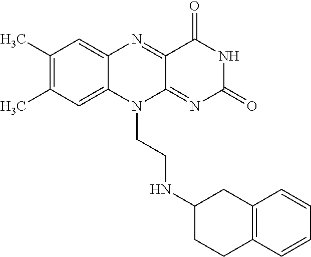 | 416.1 | 3.76 | C | Prepared using the synthesis of Step 2, Example 29 |
| 20 | 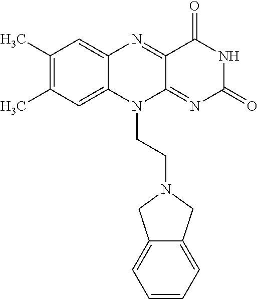 | 388 | 2.29 | C | Prepared using the synthesis of Step 2, Example 29 |
| 21 | 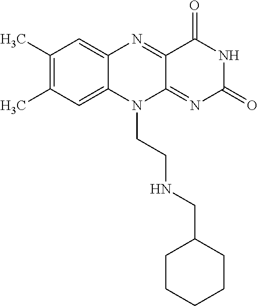 | 382 | 2.61 | C | Prepared using the synthesis of Step 2, Example 29 |
| 22 | 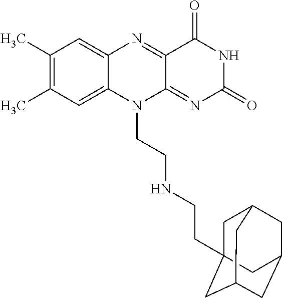 | 448.1 | 4.56 | C | Prepared using the synthesis of Step 2, Example 29 |
| 23 | 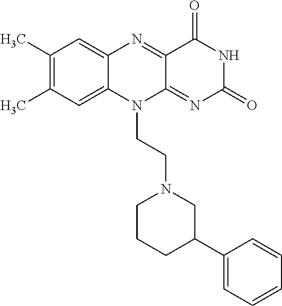 | 430.1 | 5.02 | C | Prepared using the synthesis of Step 2, Example 29 |
| 24 | 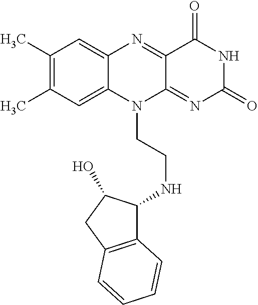 | 418 | 2.37 | C | Prepared using the synthesis of Step 2, Example 29 |
| 25 | 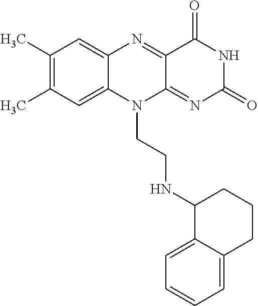 | 415.9 | 4.86 | C | Prepared using the synthesis of Step 2, Example 29 |
| 26 | 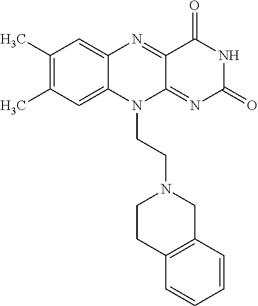 | 402.1 | 4.65 | C | Prepared using the synthesis of Step 2, Example 29 |
| 27 | 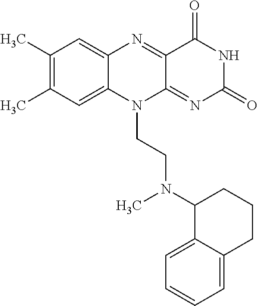 | 430 | 4.89 | C | Prepared using the synthesis of Step 2, Example 29 |
| 28 | 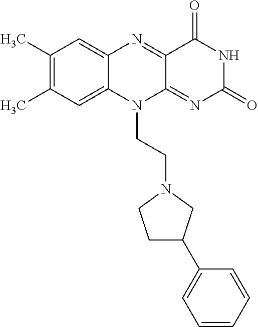 | 416.1 | 2.72 | C | Prepared using the synthesis of Step 2, Example 29 |
| 29 | 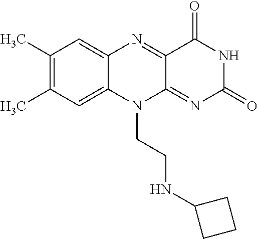 | 339.9 | 2.03 | C | Prepared using the synthesis of Step 2, Example 29 |
| 30 | 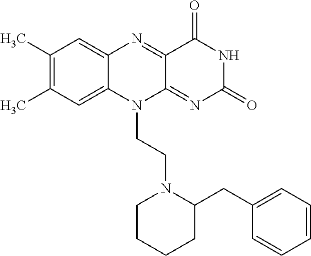 | 444.1 | 2.7 | C | Prepared using the synthesis of Step 2, Example 29 |
| 31 | 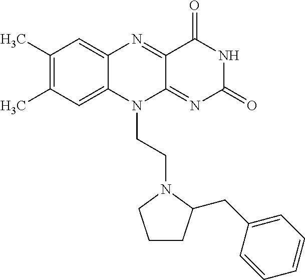 | 430.1 | 4.24 | C | Prepared using the synthesis of Step 2, Example 29 |
| 32 | 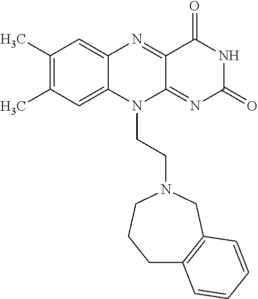 | 416 | 2.62 | C | Prepared using the synthesis of Step 2, Example 29 |
| 33 | 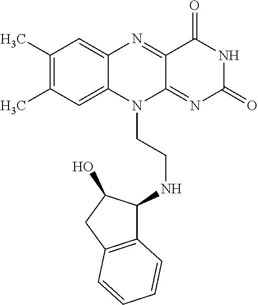 | 418 | 2.43 | C | Prepared using the synthesis of Step 2, Example 29 |
| 34 | 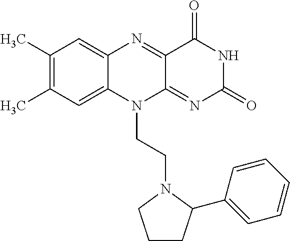 | 416 | 2.57 | C | Prepared using the synthesis of Step 2, Example 29 |
| 35 | 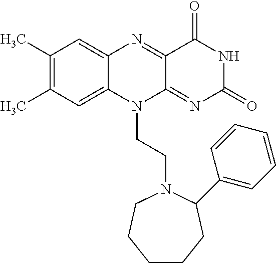 | 444.1 | 2.71 | C | Prepared using the synthesis of Step 2, Example 29 |
| 36 | 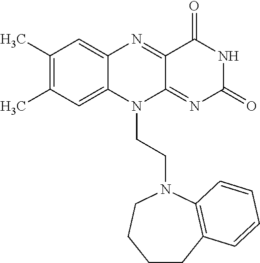 | 416 | 4.13 | C | Prepared using the synthesis of Step 2, Example 29 |
| 37 | 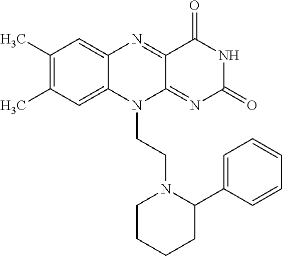 | 430.1 | 2.66 | C | Prepared using the synthesis of Step 2, Example 29 |
| 38 | 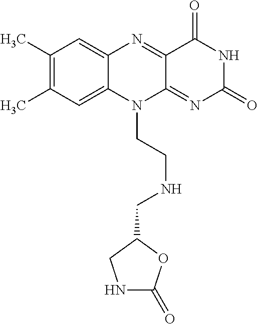 | 385.1 | 3.37 | C | Prepared using the synthesis of Step 2, Example 29 |
| 39 | 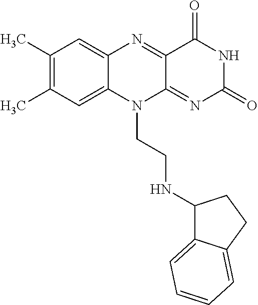 | 401.9 | 2.54 | C | Prepared using the synthesis of Step 2, Example 29 |
| 40 | 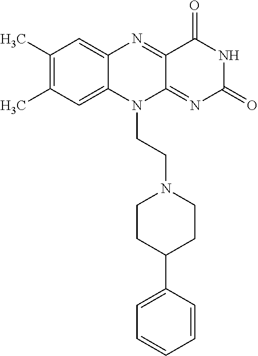 | 430.1 | 2.76 | C | Prepared using the synthesis of Step 2, Example 29 |
| 41 | 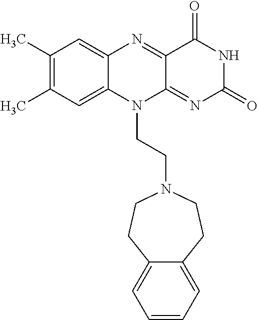 | 416 | 2.63 | C | Prepared using the synthesis of Step 2, Example 29 |
| 42 | 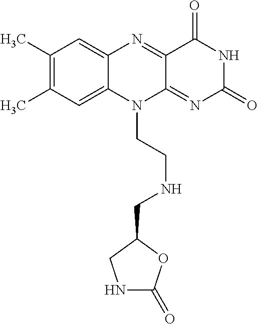 | 385.1 | 3.22 | C | Prepared using the synthesis of Step 2, Example 29 |
| 43 | 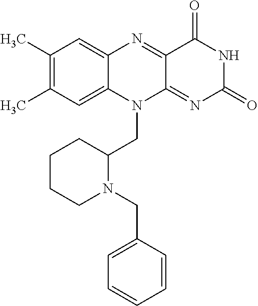 | 430 | 2.67 | C | See Example 34 |
| 44 | 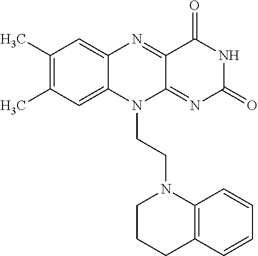 | 402 | 5.54 | C | Prepared using the synthesis of Step 2, Example 29 |
| 45 | 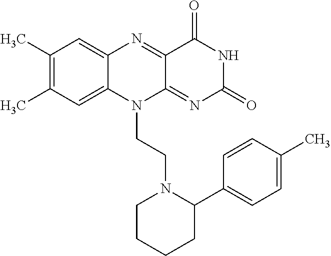 | 444.1 | 4.25 | C | Prepared using the synthesis of Step 2, Example 29 |
| 46 | 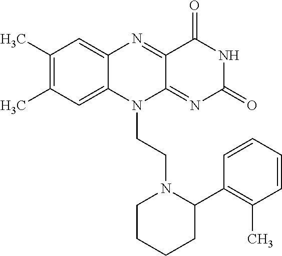 | 444.1 | 4.24 | C | Prepared using the synthesis of Step 2, Example 29 |
| 47 | 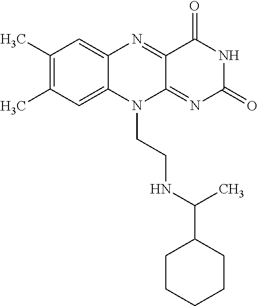 | 396.1 | 2.81 | C | Prepared using the synthesis of Step 2, Example 29 |
| 48 | 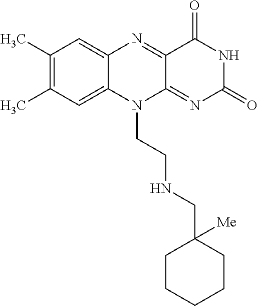 | 396.2 | 2.62 | C | Prepared using the synthesis of Step 2, Example 29 |
| 49 | 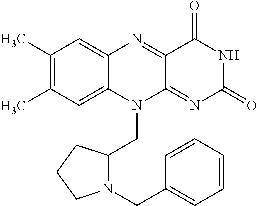 | 416.1 | 2.35 | C | See Example 35 |
| 50 | [symbol] | 367.2 | 4.18 | D | See Example 37 |
| 51 | 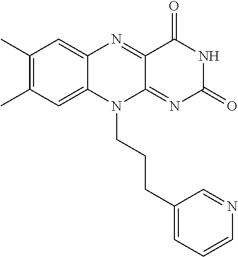 | 361.4 | 2.28 | D | See Example 38 |
| 52 | 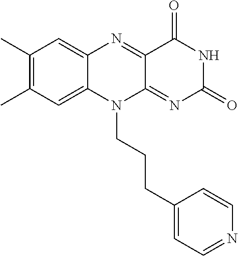 | 361.4 | 3.62 | D | Prepared using synthesis of Example 38 starting from 4,5- dimethyl-2- nitroaniline and 4-(3- bromopropyl) pyridine |
| 53 | 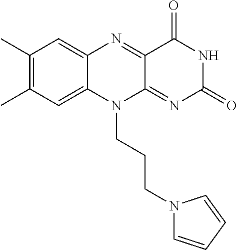 | 350.2 | 3.45 | G | Prepared using synthesis of Example 38 starting from 4,5- dimethyl-2- nitroaniline and 1-(3- bromopropyl)-1H- pyrrole |
| 54 | 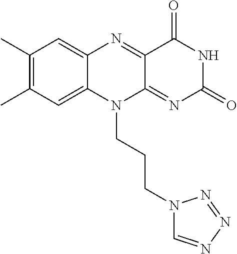 | 353.1 | 2.54 | G | Prepared using synthesis of Example 38 starting from 4,5- dimethyl-2- nitroaniline and 1-(3- bromopropyl)-1H- tetrazole (Bialonska, A., Bronisz, R. Tetrahedron 2008, 64, 9771) |
| 55 | [symbol] | 351.1 | 2.18 | G | Prepared using |
| | | | | | synthesis of |
| | | | | | Example 38 |
| | | | | | starting from 4,5- |
| | | | | | dimethyl-2- |
| | | | | | nitroaniline and |
| | | | | | 1-(3- |
| | | | | | bromopropyl)-1H- |
| | | | | | imidazole |
| 56 | 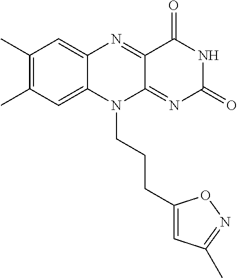 | 365.4 | 3.74 | D | Prepared using synthesis of Example 38 starting from 4,5- dimethyl-2- nitroaniline and 5-(3- bromopropyl)-3- methylisoxazole |
| 57 | 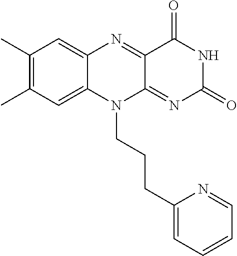 | 361.2 | 2.32 | System E | See Example 40 |
| 58 | 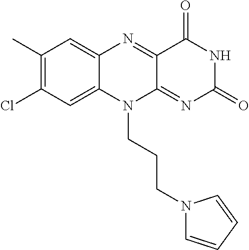 | 370.0 | 3.67 | G | Prepared using synthesis of Example 38 starting from 4- amino-2-chloro-5- nitrotoluene and 1-(3- bromopropyl)-1H- pyrrole |
| 59 | 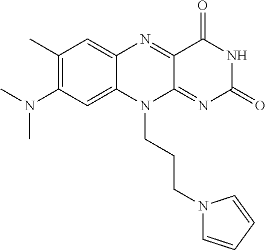 | 379.1 | 3.31 | G | See Example 39 |
| 60 | 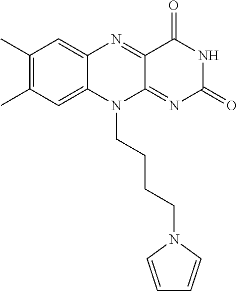 | 364.3 | 3.58 | G | Prepared using synthesis of Example 37 starting from 4,5- dimethyl-o- phenylenediamine and 1-(4-bromo- butyl)-1H-pyrrole |

[symbol]

indicates data missing or illegible when filed

### Synthesis of Compounds of Formula I(B) Through V(B) of the Invention

Synthesis of Compounds of Formula I(B) through V(B) of the Invention are provided below. Temperatures are given in degrees Celsius (° C.); unless otherwise stated, operations are carried out at room or ambient temperature, that is, at a temperature in the range of 18-25° C. Chromatography means flash chromatography on silica gel; thin layer chromatography (TLC) is carried out on silica gel plates. Samples were dissolved in deuterated solvents for NMR spectroscopy. NMR data is in the delta values of major diagnostic protons, given in parts per million (ppm) relative to the appropriate solvent signals. Conventional abbreviations for signal shape are used. For mass spectra (MS), the lowest mass major ion is reported for molecules where isotope splitting results in multiple mass spectral peaks. Solvent mixture compositions are given as volume percentages or volume ratios. In cases where the NMR spectra are complex, only diagnostic signals are reported.

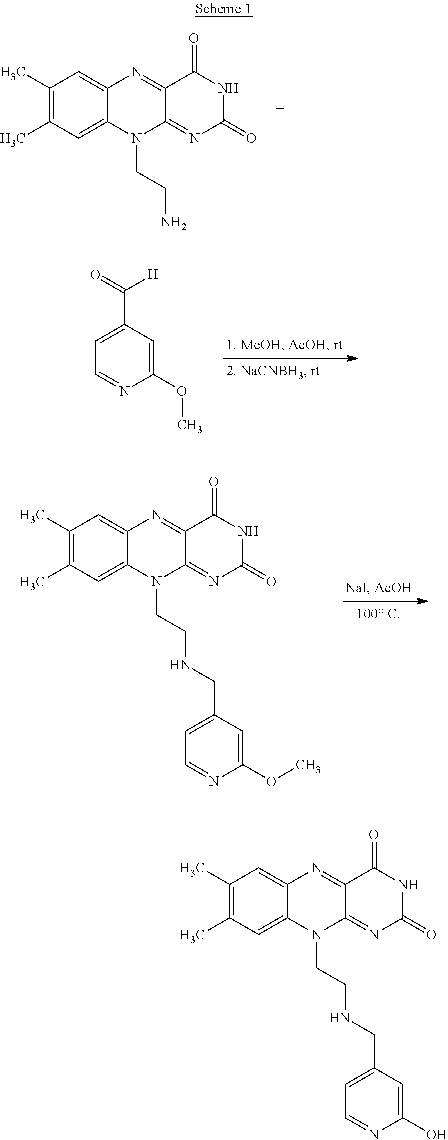

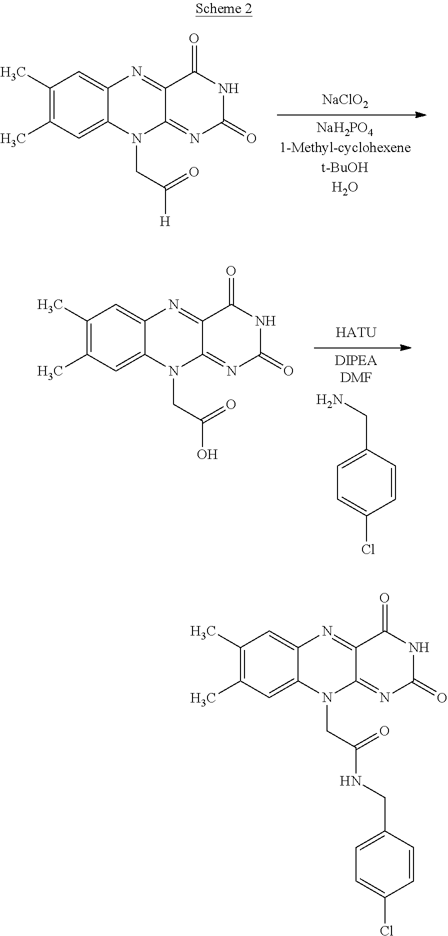

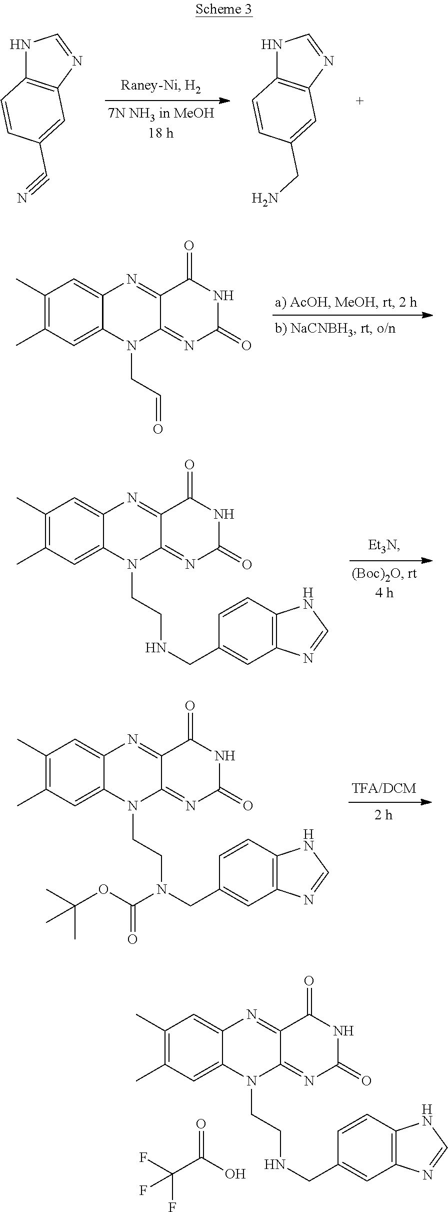

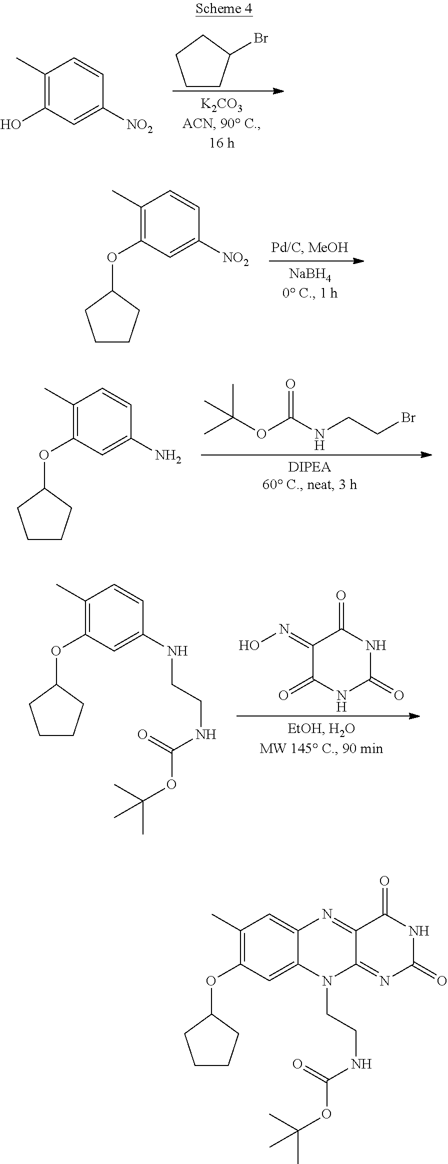

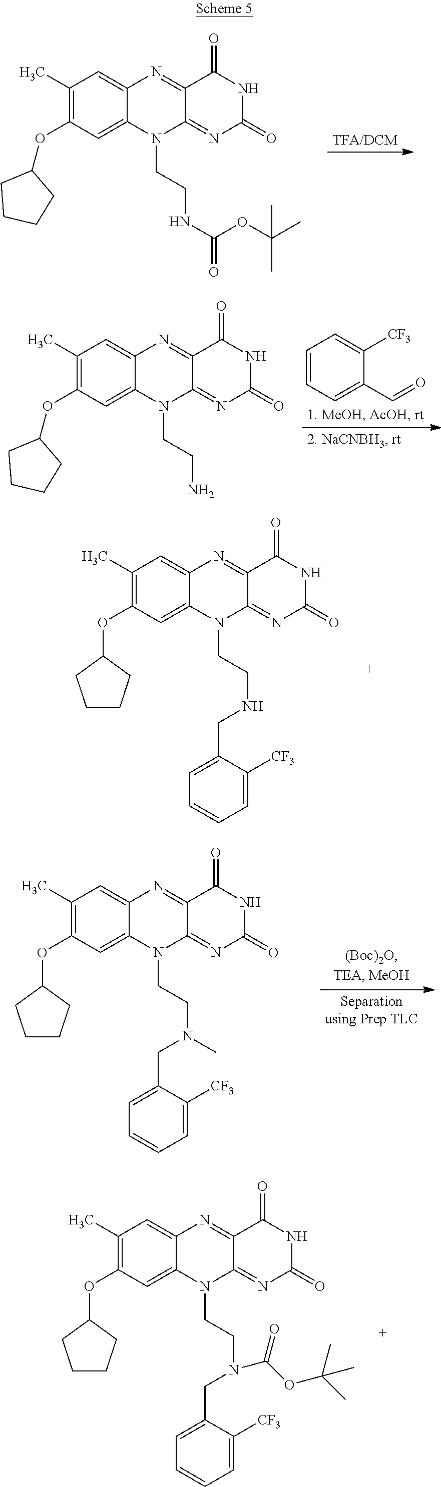

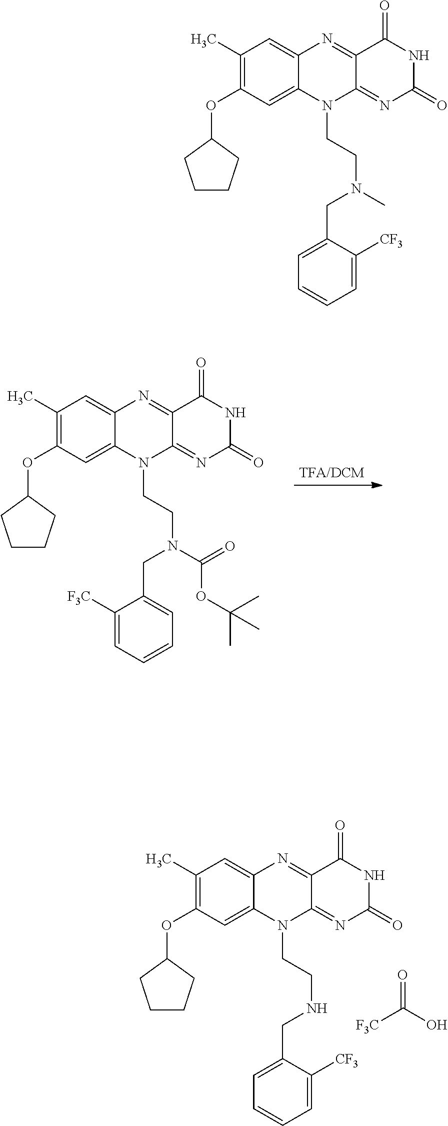

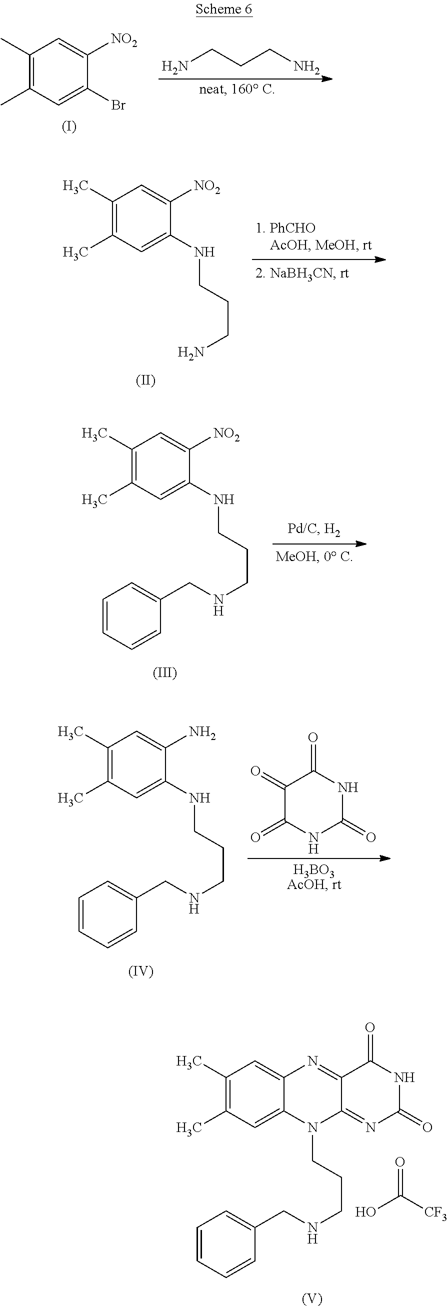

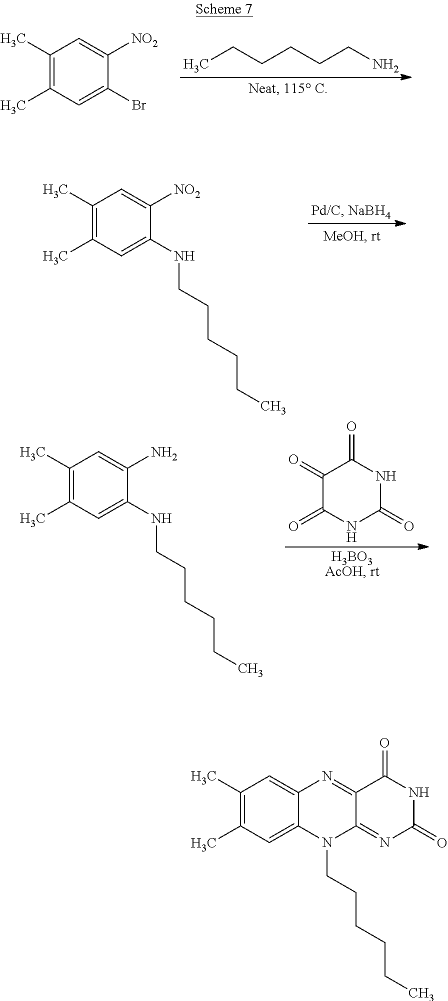

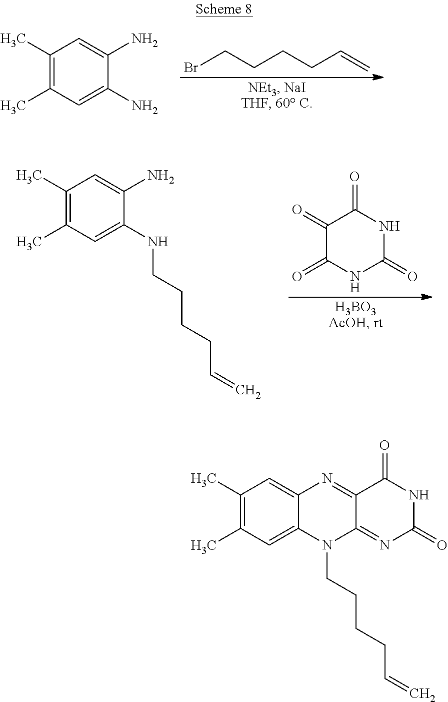

### General Methods for Analytical HPLC Analysis:

Method A′: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: 95% aqueous (0 to 0.5 min); a gradient from 95% aqueous to 98% organic (0.5 to 10.5 min); 98% organic (2 min); a gradient from 98% organic to 95% aqueous (5.5 min); 95% aqueous (1 min).
Method C′: Analytical LCMS is performed using a YMC Combiscreen ODS-AQ, 5 μm, 4.6×50 mm column. The aqueous phase is 1% 2 mM NH₄OAc in 90:10 IPA:H₂O, 0.03% TFA in USP water. The organic phase is 1% 2 mM NH₄OAc in 90:10 IPA:H₂O, 0.03% TFA in acetonitrile. The elution profile is as follows: a gradient from 95% aqueous to 100% organic (0 to 6 min); 100% organic (1 min); a gradient from 100% organic to 95% aqueous (0.1 min); 95% aqueous (2.9 min).
Method D′: Agilent 1100 HPLC, Agilent XDB C18 50×4.6 mm 1.8 micron column, 1.5 mL/min, Solvent A—Water (0.1% TFA), Solvent B—Acetonitrile (0.07% TFA), Gradient—5 min 95% A to 95% B; 1 min hold; then recycle, UV Detection @ 210 and 254 nm.
System G′: Agilent 1100 HPLC, Agilent XDB C18 50×4.6 mm 5 micron column, 1.5 mL/min, Solvent A—Water (0.1% TFA), Solvent B—Acetonitrile (0.07% TFA), Gradient—6 min 95% A to 95% B; 1 min hold; then recycle, UV Detection @ 210 and 250 nm.
Method F′: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: a gradient from 95% aqueous to 60% aqueous (0 to 10 min); a second gradient from 60% aqueous to 2% aqueous (2 min); 2% aqueous (1 min); 2% aqueous to 95% aqueous (4 min).

### General Procedure for Preparative HPLC Conditions.

Method 1′: Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: 100% aqueous (0 to 3 min); a gradient from 100% aqueous to 98% organic (3 to 21 min); 98% organic (1 min); a gradient from 98% organic to 95% aqueous (1 min); 95% aqueous (1 min).
Method 2′: Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: a gradient from 95% aqueous to 25% organic (0 to 10 min); a second gradient from 25% organic to 98% organic (over 2.5 min min); a third gradient to 95% aqueous (over 1 min).
Method 4′: A SunFire™ Prep C18 OBD™ 5 μm, 30×100 mm column. The aqueous phase is 0.1% TFA in USP water. The organic phase is acetonitrile. The elution profile is as follows: a gradient from 100% aqueous to 60% organic (0 to 29 min); then to 98% organic (29 to 31 min); 98% organic (2 min); a gradient from 98% organic to 100% aqueous (2 min); 100% aqueous (2 min).

### TERMS AND ABBREVIATIONS

ACN=acetonitrile,
AcOH=acetic acid
Bn=benzyl,
t-BuOH=tert-butyl alcohol,
Cat.=catalytic,
CAN=ammonium cerium (IV) nitrate,
CBzCl=benzyl chloroformate
Conc.=concentrated,
D-ribose=(2R,3R,4R)-2,3,4,5-tetrahydroxypentane,
DIAD=diisopropyl azodicarboxylate,
DIPEA=diisopropylethylamine,
DMF=N,N-dimethylformamide,
DCM=dichloromethane
DMAP=N,N-dimethylaminopyridine,
DMSO=dimethyl sulfoxide,
Et₂O=diethyl ether,
Et₃N=triethyl amine,
EtOAc=ethyl acetate,
EtOH=ethyl alcohol,
equiv.=equivalent(s),
h=hour(s),
H₂O=water,
HATU=2-(1H-7-Azabenzotriazol-1-yl)-1,1,3,3-tetramethyl uronium hexafluorophosphate methanaminium,
HBTU=2-(1H-Benzotriazole-1-yl)-1,1,3,3-tetramethyluronium hexafluorophosphate,
HCl=hydrochloric acid
HPLC=high performance liquid chromatography,
HOAc=acetic acid,
IPA=isopropyl alcohol,
ISCO=normal phase silica gel cartridges supplied by Teledyne ISCO,
K₂CO₃=potassium carbonate,
Min.=minute(s)
MgCl₂=magnesium chloride
MeOH=methanol,
MW=microwave
NaHCO₃=sodium bicarbonate,
Na₂SO₄=sodium sulfate,
NH₄OH=ammonium hydroxide,
NH₄OAc=ammonium acetate,
NMR=nuclear magnetic resonance,
PMB=p-methoxybenzyl,
POCl₃=phosphorous oxychloride,
POMO=pivaloyloxymethylchloride,
PPh₃=triphenylphosphine,
Prep=preparative
PyBOP=benzotriazol-1-yl-oxytripyrrolidinophosphonium hexafluorophosphate,
rt=room temperature,
RNA=ribonucleic acid,
RNase T1=an endoribonuclease that specifically degrades single-stranded RNA at G residues,
SOCl₂=thionyl chloride,
TBAI=tetrabutylammonium iodide,
TFA=trifluoroacetic acid,
TFAA=trifluoroacetic anhydride,
THF=tetrahydrofuran,
TLC=thin layer chromatography,
TMSBr=trimethylsilyl bromide,
Tris HCl=Tris (hydroxymethyl)aminomethane hydrochloride
USP water=US Pharmacopeia (USP) grade water

### Intermediate 1(B)

### Preparation of 2-(7,8-Dimethyl-2,4-dioxo-3,4-dihydrobenzo[g]pteridin-10(2H)-yl)acetaldehyde

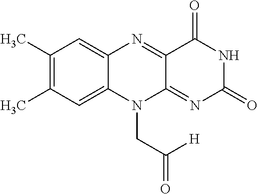

To a suspension of riboflavin (8.5 g, 0.0023 mol) in 2 N aqueous sulfuric acid (225 mL), cooled to 0° C. in a flask covered with tinfoil, is added orthoperiodic acid (18.9 g, 0.0825 mmol) dissolved in water (200 mL). After 30 min., the reaction is allowed to warm to room temperature. Once the reaction mixture becomes clear (a transparent yellow solution), the pH of the reaction solution is adjusted carefully to 3.8-3.9 (using a pH meter) by addition of solid sodium carbonate. [It is extremely important that the pH is adjusted carefully, if not, the product does not precipitate out of solution.] The precipitate is then isolated by filtration and washed liberally with cold water, ethanol, and diethyl ether to yield desired product (6.04 g, 94%) as an orange solid. LC-MS m/z 285.1 [M+H]⁺, retention time 1.63 min.

### General Procedure 1

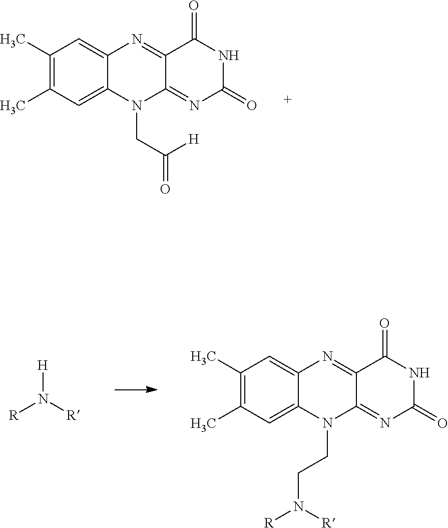

To a suspension of 2-(7,8-dimethyl-2,4-dioxo-3,4-dihydro-2H-benzo[g]pteridin-10-yl)-acetaldehyde (1 mmol) in MeOH (43 mL) are added appropriately substituted amine (3 mmol), and AcOH (0.28 mL) respectively and stirred at room temperature for 2 h, wherein the substituted amine, e.g., (1) H—N(R)(R′) may represent H—N(R₄)-A and R₄ and A are as defined in Formula I(B); or (2) H—N(R)(R′) may represent H—N(R₄)(R₅) and R₄ and R₅ are as defined in Formula III(B); or (3) the H—N(R)(R′) together with the acetaldehyde forms Y wherein Y is as defined in Formula II(B), except Y in this example is not —CH₂C(O)N(H)—C₄H₅—C1 or —CH₂CH₂CH₂N(H)benzyl. NaCNBH₃ (3 mmol) is then added to the reaction mixture and stirred at room temperature for 24 h to yield the product as shown in the structure above wherein —CH₂CH₂N(R)(R′) is -Alk-(X)-A as defined in Formula I(B); (2) —CH₂CH₂N(R₄)(R₅) as defined in Formula III(B); or (3) Y as defined in Formula II(B), except in this instance, Y is not —CH₂C(O)N(H)—C₄H₅—C1 or —CH₂CH₂CH₂N(H)benzyl (when Y is —CH₂C(O)N(H)—C₄H₅—Cl or —CH₂CH₂CH₂N(H)benzyl, the compounds may be prepared using a different procedure described herewith). The solvent is removed under vacuum and the crude is purified by preparative HPLC. Lyophilization of combined fractions affords desired product [NMR, LC-MS].

### General Procedure 2

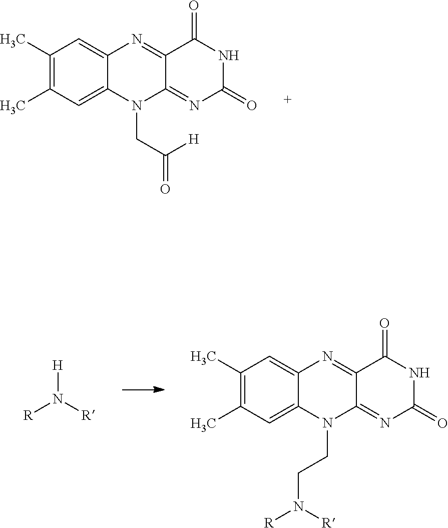

To a suspension of (7,8-dimethyl-2,4-dioxo-3,4-dihydro-2H-benzo[g]pteridin-10-yl)-acetaldehyde (1 mmol) in methanol (28 mL) is added appropriately substituted amine (1 mmol) at room temperature e.g., wherein the substituted amine, e.g., (1) H—N(R)(R′) may represent H—N(R₄)-A and R₄ and A are as defined in Formula I(B); or (2) H—N(R)(R′) may represent H—N(R₄)(R₅) and R₄ and R₅ are as defined in Formula III(B); or (3) the H—N(R)(R′) together with the acetaldehyde forms Y wherein Y is as defined in Formula II(B), except Y in this example is not —CH₂C(O)N(H)—C₄H₅—Cl or —CH₂CH₂CH₂N(H)benzyl. After 30 min., acetic acid (0.57 mL) and sodium cyanoborohydride (4.375 mmol) are added, and the solution is stirred for 6 h to yield the product as shown in the structure above wherein —CH₂CH₂N(R)(R′) is -Alk-(X)-A as defined in Formula I(B); (2) —CH₂CH₂N(R₄)(R₅) as defined in Formula III(B); or (3) Y as defined in Formula II(B), except in this instance, Y is not —CH₂C(O)N(H)—C₄H₅—Cl or —CH₂CH₂CH₂N(H)benzyl (when Y is —CH₂C(O)N(H)—C₄H₅—Cl or —CH₂CH₂CH₂N(H)benzyl, the compounds may be prepared using a different procedure described herewith). The reaction mixture is concentrated, and the residue is dry loaded onto silica, and purified by column chromatography using MeOH in DCM as the eluent (gradient 3-10% MeOH). Desired product [NMR, LC-MS] is isolated following evaporation of the appropriate fractions.

### Example 41

### N-(4-((2-(7,8-Dimethyl-2,4-dioxo-3,4-dihydrobenzo[g]pteridin-10(2H)-yl)ethyl)amino)phenyl)acetamide

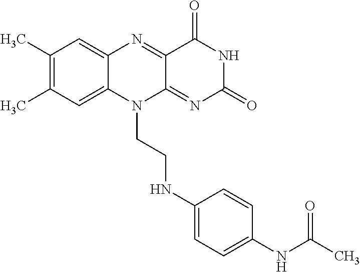

The title compound is prepared using the General Procedure 2 using (7,8-dimethyl-2,4-dioxo-3,4-dihydro-2H-benzo[g]pteridin-10-yl)-acetaldehyde (50 mg, 0.176 mmol) and N-(4-aminophenyl)acetamide (26.4 mg, 0.176 mmol). The desired product is obtained as a dark brown powder (37.7 mg, 51%). ¹H NMR (400 MHz, DMSO-d₆) δ 1.97 (s, 3H), 2.31 (s, 3H), 2.37 (s, 3H), 3.50 (m, 2H), 4.70 (m, 2H), 5.54 (t, 1H), 6.59 (d, 2H), 7.31 (t, 2H), 7.53 (s, 1H), 7.88 (s, 1H), 9.55 (s, 1H), 11.33 (s, 1H). MS m/z 419.2 [M+H]⁺, 441.3 [M+Na]⁺.

### Example 42

### 10-(2-O-(Dimethylamino)ethyl)amino)ethyl)-7,8-dimethylbenzo[g]pteridine-2,4(3H,10H)-dione

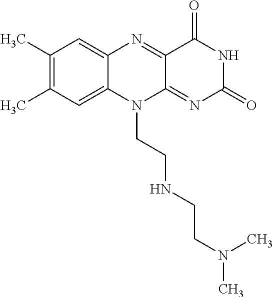

The title compound is prepared using the General Procedure 2 using (7,8-dimethyl-2,4-dioxo-3,4-dihydro-2H-benzo[g]pteridin-10-yl)-acetaldehyde (50 mg, 0.176 mmol) and N¹,N¹-dimethylethane-1,2-diamine (16 mg, 0.176 mmol). The reaction mixture is concentrated, and the residue is dry loaded onto silica gel, and purified by column chromatography using MeOH (20%) in DCM as the eluent with 1% Et₃N. 10-(2-((2-(Dimethylamino)ethyl)amino)ethyl)-7,8-dimethylbenzo[g]pteridine-2,4(3H,10H)-dione (46.5 mg, 74%) is isolated following evaporation of the appropriate fractions. ¹H NMR (400 MHz, DMSO-d₆) δ 2.11 (s, 6H), 2.26 (t, 2H), 2.39 (s, 3H), 2.43 (s, 3H), 2.64 (t, 2H), 2.91 (t, 2H), 3.36 (br s, H₂O), 4.65 (t, 2H), 7.87 (s, 1H), 7.88 (s, 1H). MS m/z 357.3 [M+H]⁺.

### Example 43

### 10-(2-(((2-Hydroxypyridin-4-yl)methyl)amino)ethyl)-7,8-dimethylbenzo[g]pteridine-2,4(3H,10H)-dione

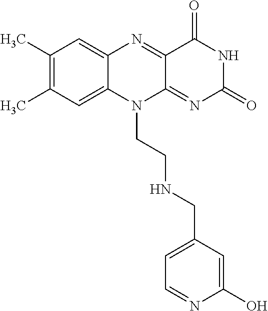

### Step 1 Preparation of 10-(2-(Benzylamino)ethyl)-7,8-dimethylbenzo[g]pteridine-2,4(3H,10H)-dione 2,2,2-trifluoroacetate

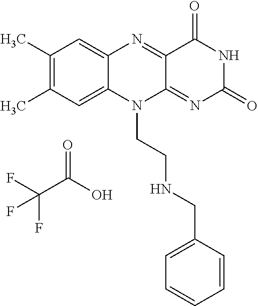

### Step 2 Preparation of 10-(2-(benzylamino)ethyl)-7,8-dimethylbenzo[g]pteridine-2,4(3H,10H)-dione

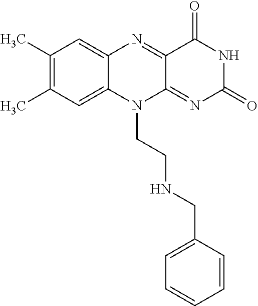

The title compound is prepared using General Procedure 1 except (2.08 g, 7.32 mmol) of 2-(7,8-dimethyl-2,4-dioxo-3,4-dihydrobenzo[g]pteridin-10(2H)-yl)acetaldehyde and (4 mL, 34.3 mmol) of benzylamine are used in place of 1 mmol 2-(7,8-dimethyl-2,4-dioxo-3,4-dihydro-2H-benzo[g]pteridin-10-yl)-acetaldehyde and amine (3 mmol) respectively. The product is contaminated with 10-(2-(benzyl(methyl)amino)ethyl)-7,8-dimethylbenzo[g]pteridine-2,4(3H,10H)-dione. The next two steps are performed to isolate the desired product.

### Step 3 Preparation of tert-butyl benzyl(2-(7,8-dimethyl-2,4-dioxo-3,4-dihydrobenzo[g]pteridin-10(2H)-yl)ethyl) carbamate

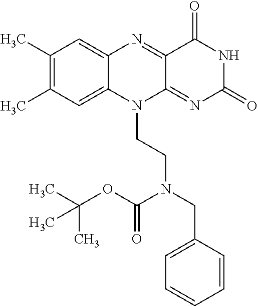

To a solution of crude 10-(2-(benzylamino)ethyl)-7,8-dimethylbenzo[g]pteridine-2,4(3H,10H)-dione (7.53 mmol) in MeOH (200 mL) is added di-tert-butyl dicarbonate (5.2 g, 23.8 mmol) and Et₃N (4 ml). The reaction is concentrated under reduced pressure and purified via silica gel chromatography (ISCO) (100% DCM to 10% MeOH/DCM) to obtain desired product (1.85 g, 54%) as a brown solid.

### Step 4 Preparation of 10-(2-(benzylamino)ethyl)-7,8-dimethylbenzo[g]pteridine-2,4(3H,10H)-dione 2,2,2-trifluoroacetate

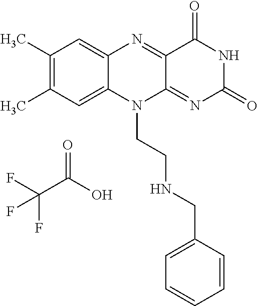

To a solution of tert-butyl benzyl(2-(7,8-dimethyl-2,4-dioxo-3,4-dihydrobenzo[g]pteridin-10(2H)-yl)ethyl)carbamate (50 mg, 0.11 mmol) in DCM (2 mL) is added TFA (2 mL) at rt. After 2 h, the reaction mixture is concentrated and the residual material is dissolved in MeOH (10 ml) and purified by preparative HPLC (Method 2′). Lyophilization of combined fractions (LCMS) affords desired product (33.6 mg, 65%) as a brown solid. ¹H NMR (400 MHz, DMSO-d₆) δ 2.42 (s, 3H), 2.53 (s, 3H), 4.35 (s, 2H), 5.00 (m, 2H), 7.43 (m, 3H), 7.52 (m, 2H), 7.83 (s, 1H), 7.96 (s, 1H), 9.02 (s, 2H), 11.49 (s, 1H).

### Step 5 Preparation of 10-(2-aminoethyl)-7,8-dimethylbenzo[g]pteridine-2,4(3H,10H)-dione-2,2,2-trifluoroacetate salt

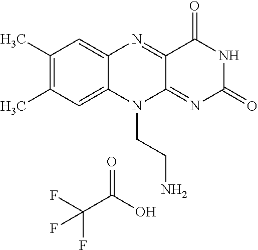

A solution of 10-(2-(benzylamino)ethyl)-7,8-dimethylbenzo[g]pteridine-2,4(3H,10H)-dione (step 4) (395 mg, 1.05 mmol) and Pd/C (75 mg) in absolute EtOH (100 ml) is hydrogenated at 30 psi and 45° C. overnight. The mixture is filtered through a celite pad. The filtrate is concentrated under reduced pressure to dryness to obtain a crude product (230 mg, 77%). Crude product (19.5 mg, 0.07 mmol) is dissolved in MeOH (8 ml) and purified by preparative HPLC (Method 2′). Lyophilization of the combined fractions affords desired product (5.0 mg, 14%) as a brown solid. ¹H NMR (400 MHz, DMSO-d₆) δ 2.42 (s, 3H), 2.50 (s, 3H), 4.20 (m, 2H), 4.87 (m, 2H), 7.81 (s, 1H), 7.88 (m, 2H), 7.97 (s, 1H), 11.45 (s, 1H).

### Step 6 Preparation of 10-(2-((2-methoxypyridin-4-yl)methylamino)ethyl)-7,8-dimethylbenzo[g]pteridine-2,4(3H,10H)-dione

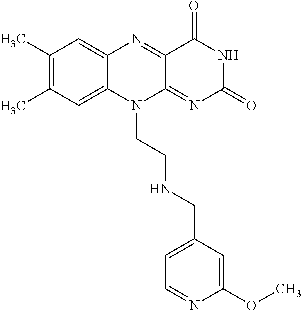

To a suspension of 10-(2-aminoethyl)-7,8-dimethylbenzo[g]pteridine-2,4(3H,10H)-dione (46 mg, 0.16 mmol) in MeOH (5 mL) is added 2-methoxyisonicotinaldehyde (prepared as in C. Subramanyam, M. Noguchi and S. M. Weinreb, J. Org. Chem., 1989, 54, 5580, the contents of which are incorporated by reference in their entirety) (22 mg, 0.16 mmol), followed by acetic acid (0.1 mL) at rt. After 30 min., sodium cyanoborohydride (30 mg, 0.47 mmol) is added, and the solution is stirred for 16 h. The reaction mixture is concentrated, and the residue is dissolved in DMF (4 ml)/water (3 ml), filtered, and purified by preparative HPLC (Method 2′). Lyophilization of the combined fractions affords the desired product (6.5 mg, 10%). ¹H NMR (400 MHz, CD₃OD) δ 2.50 (s, 3H), 2.63 (s, 3H), 3.70 (m, 2H), 3.94 (s, 3H), 4.37 (s, 2H), 5.10 (m, 2H), 6.95 (s, 1H), 7.08 (d, 1H), 7.81 (s, 1H), 7.96 (s, 1H), 8.21 (d, 1H).

### Step 7 Preparation of 10-(2-(((2-hydroxypyridin-4-yl)methyl)amino)ethyl)-7,8-dimethylbenzo[g]pteridine-2,4(3H,10H)-dione

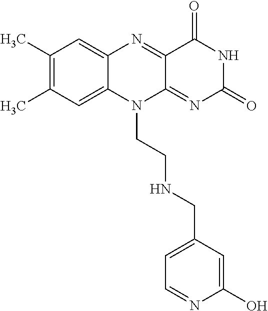

A mixture of 10-(2-(((2-methoxypyridin-4-yl)methyl)amino)ethyl)-7,8-dimethylbenzo[g]pteridine-2,4(3H,10H)-dione (9 mg, 0.02 mmol) and NaI (43 mg, 0.26 mmol) is heated in acetic acid (5 ml) for 2 h. Acetic acid is evaporated and water (10 ml) is added, followed by sodium thiosulfate until the solution turns clear. The solution is concentrated, and the residue is dissolved in water (3 ml) and purified by preparative HPLC (Method 2′). Lyophilization of the combined fractions affords the desired product (3.1 mg, 36%). ¹H NMR (400 MHz, CD₃OD) δ 2.51 (s, 3H), 2.63 (s, 3H), 3.71 (m, 2H), 4.29 (s, 2H), 5.13 (m, 2H), 6.51 (d, 1H), 6.63 (s, 1H), 7.55 (d, 1H), 7.82 (s, 1H), 8.03 (s, 1H). ESI(+) [M+H]⁺=393.1.

### Example 44

### N-(4-Chlorobenzyl)-2-(7,8-dimethyl-2,4-dioxo-3,4-dihydrobenzo pteridin-10(2H)-yl)acetamide

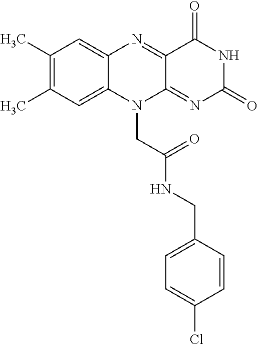

Step 1 Preparation of (7,8-dimethyl-2,4-dioxo-3,4-dihydro-2H-benzo[g]pteridin-10-yl)-acetic acid

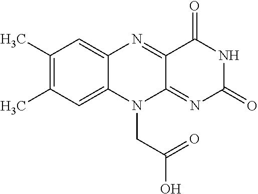

To a suspension of (7,8-dimethyl-2,4-dioxo-3,4-dihydro-2H-benzo[g]pteridin-10-yl)-acetaldehyde (prepared by the method of Example 1, step 1) (50 mg, 0.18 mmol) in acetonitrile (2 ml), tert-butanol (8 mL), and methyl-1-cyclohexene (3 mL) at 0° C., a solution of sodium chlorite (122 mg, 1.35 mmol) and sodium dihydrogen phosphate (148 mg, 1.23 mmol) in 2 mL of water is added dropwise over 5 min. After 2 h, the reaction mixture is diluted with water and the organic layer is removed. The aqueous phase is concentrated under vacuum and the resultant crude mixture is purified via preparative HPLC. (7,8-Dimethyl-2,4-dioxo-3,4-dihydro-2H-benzo[g]pteridin-10-yl)-acetic acid is isolated following lyophilization of the appropriate fractions (36 mg, 68%). LC-MS m/z 301.1 [M+H]⁺, retention time=1.68 min.

### Step 2 Preparation of N-(4-chlorobenzyl)-2-(7,8-dimethyl-2,4-dioxo-3,4-dihydrobenzo[g]pteridin-10(2H)-yl)acetamide

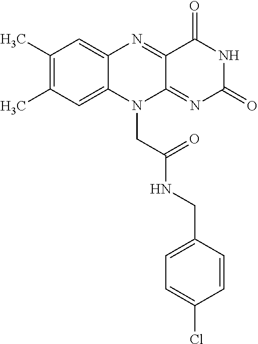

(7,8-Dimethyl-2,4-dioxo-3,4-dihydro-2H-benzo[g]pteridin-10-yl)-acetic acid (100 mg, 0.34 mmol) and (4-chlorophenyl)methanamine (0.084 mL, 0.68 mmol) in DMF (9 mL), is stirred at 0° C. under argon for 10 min. DIPEA (0.18 mL, 0.68 mmol) is added to the reaction mixture, followed by HATU (128 mg, 0.68 mmol) and the mixture is stirred for 18 h and then allowed to warm to rt. Another 2 eq of DIPEA, (4-chlorophenyl)methanamine, and HATU are added and the resulting mixture is stirred another 48 h at rt. The mixture is concentrated to 2 mL and diluted with Et₂O (5 mL) and the solid is removed by filtration. The filtrate is concentrated and MeOH is added to the residue. The precipitate is isolated, washed with MeOH (5 mL), and purified using preparative HPLC (method 1′), followed by preparative TLC using 9/1 DCM/MeOH to obtain the desired product (3 mg, 2%) as a yellow solid. ¹H NMR (400 MHz, MeOH-d₄): δ 2.41 (s, 3H), 2.47 (s, 3H), 4.31 (s, 2H), 5.36 (s, 2H), 7.34 (m, 4H), 7.69 (s, 1H), 7.95 (s, 1H), 8.78 (s, 1H), 11.43 (s, 1H).

### Example 45

### 10-(2-((1H-Benzo[d]imidazol-5-yl)methyl)amino)ethyl)-7,8-dimethylbenzo[g]pteridine-2,4(3H,10H)-dione trifluoro acetic acid salt

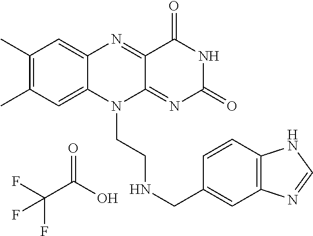

### Step 1 Preparation of (1H-benzo[d]imidazol-5-yl)methanamine

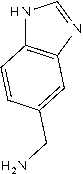

To a solution of 1H-benzo[d]imidazole-5-carbonitrile (0.212 g, 1.48 mmol) in 7N NH₃ in MeOH (15 mL) is added Raney Nickel slurry in water (0.0087 g, 0.148 mmol). The reaction flask is then put under an atmosphere of hydrogen (fitted with a balloon) and let stir at rt for 18 h. After 18 h, an additional catalytic amount (0.0087 g, 0.000148 mol) of Raney Nickel slurry in water is added to the reaction flask. The reaction is allowed to stir for an additional 18 h under an atmosphere of hydrogen. The reaction mixture is filtered through celite and concentrated. The crude reaction mixture is used in the next step. LC-MS m/z 148.0 [M+H]⁺, retention time 0.65 min.

### Step 2 Preparation of 10-(2-(((1H-benzo[d]imidazol-5-yl)methyl)amino)ethyl)-7,8-dimethylbenzo[g]pteridine-2,4(3H,10H)-dione

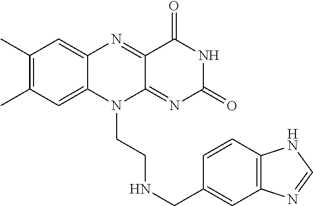

The title compound is prepared using General Procedure 1 and 2-(7,8-dimethyl-2,4-dioxo-3,4-dihydrobenzo[g]pteridin-10(2H)-yl)acetaldehyde (0.05 g, 0.176 mmol) and (1H-benzo[d]imidazol-5-yl)methanamine 0.168 g, 1.14 mmol). This product is contaminated with 10-(2-(((1H-benzo[d]imidazol-5-yl)methyl)(methyl)amino)ethyl)-7,8-dimethylbenzo[g]pteridine-2,4(3H,10H)-dione. The next two steps are performed to isolate the product from the N-methyl side product.

### Step 3 Preparation of tert-butyl ((1H-benzo[d]imidazol-5-yl)methyl)(2-(7,8-dimethyl-2,4-dioxo-3,4-dihydrobenzo[g]pteridin-10(2H)-yl)ethyl)carbamate

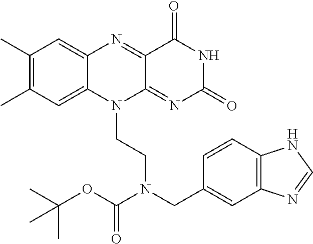

To a solution of crude 10-(2-(((1H-benzo[d]imidazol-5-yl)methyl)amino)ethyl)-7,8-dimethylbenzo[g]pteridine-2,4(3H,10H)-dione (0.176 mmol) in MeOH (200 mL) is added di-tert-butyl dicarbonate (5.2 g, 23.8 mmol) and Et₃N (4 ml). The reaction is stirred for 4 h, at which point the reaction mixture is concentrated. Purification is performed using preparative TLC, using 2% MeOH/DCM as solvent. The appropriate band is collected and the silica gel is filtered off and the filtrate is concentrated. The product is isolated (35.2 mg, 39%) as a yellow solid and used in the next step.

### Step 4 Preparation of 10-(2-(((1H-benzo[d]imidazol-5-yl)methyl)amino)ethyl)-7,8-dimethylbenzo[g]pteridine-2,4(3H,10H)-dione trifluoro acetic acid salt

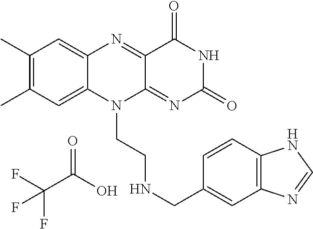

To a solution of tert-butyl ((1H-benzo[d]imidazol-5-yl)methyl)(2-(7,8-dimethyl-2,4-dioxo-3,4-dihydrobenzo[g]pteridin-10(2H)-yl)ethyl)carbamate (35.2 mg, 0.068 mmol) in DCM (2 mL) is added TFA (2 mL) at rt. After 4 h of stirring, the reaction mixture is concentrated and the residual material is dissolved in DMSO (2 mL) and purified by preparative HPLC (Method 4′). Lyophilization of the combined pure fractions affords the desired product (27.3 mg, 37%) as a yellow solid. LC-MS m/z 416.1 [M+H]⁺, retention time 3.14 min. ¹H NMR (400 MHz, DMSO-d₆) δ 2.4 (s, 3H), 3.44 (m, 2H), 4.5 (m, 2H), 4.98 (m, 2H), 7.57 (m, 1H), 7.82 (m, 2H), 7.94 (m, 2H), 9.12 (m, 3H), 11.43 (s, 1H). LC-MS m/z 416.1 [M+H]⁺, retention time 3.14 min.

### Example 46

### 8-(Cyclopentyloxy)-7-methyl-10-(2-((2-(trifluoromethyl)benzyl)amino)ethyl)benzo [g]pteridine-2,4(3H,10H)-dione 2,2,2-trifluoroacetic acid salt

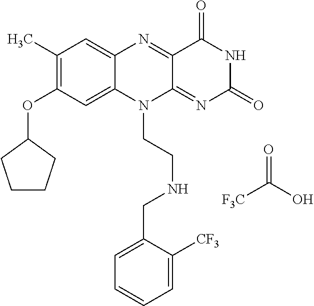

### Step 1 Preparation of 2-(cyclopentyloxy)-1-methyl-4-nitrobenzene

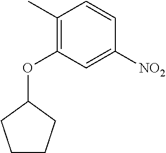

A mixture of 2-methyl-5-nitrophenol (4.5 g, 29 mmol), bromocyclopentane (7.8 g, 52 mmol) and K₂CO₃ is refluxed in ACN (200 mL) for 16 h. The solid is removed by filtration and washed with EtOAc. The filtrate is concentrated and used in the next step.

### Step 2 Preparation of 3-(cyclopentyloxy)-4-methylaniline

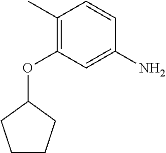

To a solution of 2-(cyclopentyloxy)-1-methyl-4-nitrobenzene (29 mmol) and Pd/C (200 mg, 10% wet) in MeOH (200 mL) at 0° C., is slowly added sodium borohydride (1.25 g, 33 mmol) with vigorous stirring. The resulting mixture is stirred for 1 h at 0° C. The reaction mixture is filtered through a celite pad and the filtrate is concentrated under reduced pressure. The crude is dissolved in DCM and washed with water. The organic layer is dried over Na₂SO₄, filtered, and concentrated under reduced pressure to obtain crude product (5.79 g) as a brown oil. This is used in the next step without further purification. LC-MS ESI(+) [M+H]⁺=191.9, retention time 2.99 min.

### Step 3 Preparation of tert-butyl (2-((3-(cyclopentyloxy)-4-methylphenyl)amino)ethyl)carbamate

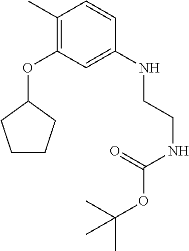

A mixture of 3-(cyclopentyloxy)-4-methylaniline (2.36 g, 12.3 mmol) and tert-butyl (2-bromoethyl)carbamate (2.55 g, 11.3 mmol) is heated in DIPEA (4.2 ml, 24 mmol) for 3 h at 60° C. The reaction is cooled to rt, and EtOAc (20 mL) is added with stirring. The solid is removed by filtration and washed with EtOAc. The filtrate is evaporated and the residue is purified by flash column chromatography using a gradient from 0 to 40% EtOAc in hexane as eluent. The product is isolated as a red oil (1.93 g, 50%). LCMS ESI(+) [M+H]⁺=334.9, retention time 4.29 min.

### Step 4 Preparation of tert-butyl (2-(8-(cyclopentyloxy)-7-methyl-2,4-dioxo-3,4-dihydrobenzo[g]pteridin-10(2H)-yl)ethyl)carbamate

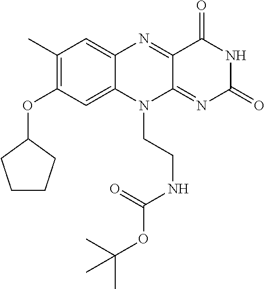

A mixture of tert-butyl (2-((3-(cyclopentyloxy)-4-methylphenyl)amino)ethyl)carbamate (2 g, 5.98 mmol) and violuric acid (1.14 g, 6.5 mmol) is microwaved at 145° C. in EtOH (18 mL) and water (2 mL) for 90 min. The solvent is evaporated and the resulting solid is washed with Et₂O, EtOAc then water (2 mL). The product is isolated as a yellow solid (902 mg, 38%). The product is used in the next step without further purification. LCMS ESI(+) [M+H]⁺=455.9, retention time 4.48 min.

### Step 5 Preparation of 10-(2-aminoethyl)-8-(cyclopentyloxy)-7-methylbenzo[g]pteridine-2,4(3H,10H)-dione

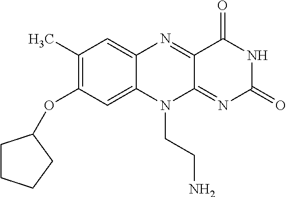

To a solution of tert-butyl (2-(8-(cyclopentyloxy)-7-methyl-2,4-dioxo-3,4-dihydrobenzo[g]pteridin-10(2H)-yl)ethyl)carbamate (57 mg, 0.12 mmol) in DCM (2 mL) is added TFA (2 mL) at rt. After 2 h of stirring, the reaction mixture is concentrated and TEA (2 ml) is added to the residual material, stirred for 20 min, and concentrated to obtain a crude oil. The crude (10-(2-aminoethyl)-8-(cyclopentyloxy)-7-methylbenzo[g]pteridine-2,4(3H,10H)-dione) is used in the next step without further purification. LCMS: ESI(+) [M+H]⁺=356.0

### Step 6 Preparation of 8-(cyclopentyloxy)-7-methyl-10-(2-((2-trifluoromethyl)benzyl)amino)ethyl)benzo[g]pteridine-2,4(3H,10H)-dione

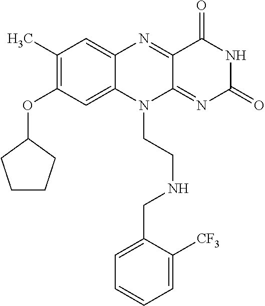

8-(Cyclopentyloxy)-7-methyl-10-(2-((2-(trifluoromethyl)benzyl)amino)ethyl)benzo[g]pteridine-2,4(3H,10H)-dione is prepared using General Procedure 1 and 10-(2-aminoethyl)-8-(cyclopentyloxy)-7-methylbenzo[g]pteridine-2,4(3H,10H)-dione (0.125 mmol, step 2) and 2-(trifluoromethyl)benzaldehyde (17.4 mg, 0.1 mmol). LCMS indicated that the product is contaminated with 8-(cyclopentyloxy)-7-methyl-10-(2-(methyl(2-(trifluoromethyl)benzyl)amino)ethyl)benzo[g]pteridine-2,4(3H,10H)-dione. The next two steps are performed to isolate the product. LCMS: ESI(+) [M+H]⁺=514.0, retention time 3.50 min (desired product) and 528.0, retention time 3.61 min (N-methylated by-product).

### Step 7 Preparation of tert-butyl (2-(8-(cyclopentyloxy)-7-methyl-2,4-dioxo-3,4-dihydrobenzo[g]pteridin-10(2H)-yl)ethyl)(2-(trifluoromethyl)benzyl)carbamate

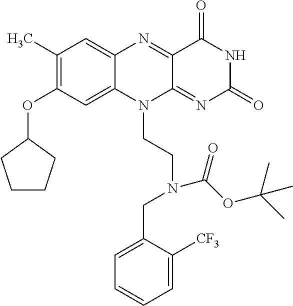

To a solution of crude 8-(cyclopentyloxy)-7-methyl-10-(2-((2-(trifluoromethyl)benzyl)amino)ethyl)benzo[g]pteridine-2,4(3H,10H)-dione in MeOH (200 mL) is added di-tert-butyl dicarbonate (54 mg, 0.25 mmol) and Et₃N (1 ml). The reaction mixture is stirred at rt for 5 h. The reaction is concentrated under reduced pressure and purified via preparatory TLC using 3% MeOH/DCM as eluent. Pure tert-butyl (2-(8-(cyclopentyloxy)-7-methyl-2,4-dioxo-3,4-dihydrobenzo[g]pteridin-10(2H)-yl)ethyl)(2-(trifluoromethyl)benzyl)carbamate (17.0 mg) is obtained as a bright yellow solid.

### Step 8 Preparation of 8-(cyclopentyloxy)-7-methyl-10-(2-((2-(trifluoromethyl)benzyl)amino)ethyl)benzo[g]pteridine-2,4(3H,10H)-dione 2,2,2-trifluoroacetic acid salt

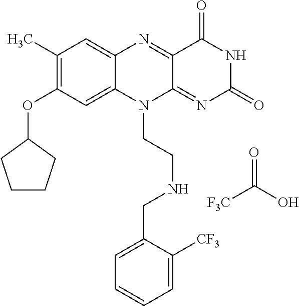

To a solution of tert-butyl (2-(8-(cyclopentyloxy)-7-methyl-2,4-dioxo-3,4-dihydrobenzo[g]pteridin-10(2H)-yl)ethyl)(2-(trifluoromethyl)benzyl)carbamate (17 mg, 0.03 mmol) in DCM (1 mL) is added TFA (1 mL) at rt. After 2 h stirring, the reaction mixture is concentrated and lyophilized. The desired product 8-(cyclopentyloxy)-7-methyl-10-(2-((2-(trifluoromethyl)benzyl)amino)ethyl)benzo[g]pteridine-2,4(3H,10H)-dione 2,2,2-trifluoroacetic acid salt (12.4 mg, 71%) is obtained as a bright yellow solid. ¹H NMR (400 MHz, DMSO-d₆) δ 1.67-1.83 (m, 6H), 2.02 (m, 2H), 2.28 (s, 3H), 3.57 (t, 2H), 4.50 (s, 2H), 5.10 (t, 2H), 5.30 (m, 1H), 7.14 (s, 1H), 7.70 (m, 1H), 7.78 (m, 2H), 7.84 (d, 1H), 7.98 (s, 1H), 9.49 (br s, 2H), 11.41 (s, 1H). ESI(+) [(M−TFA)+H]⁺=514.1

### Example 47

### 10-(3-(Benzylamino)propyl)-7,8-dimethylbenzo[g]pteridine-2,4(3H,10H)-dione 2,2,2-trifluoroacetic acid salt

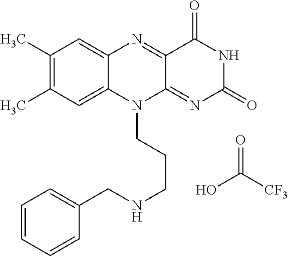

Step 1 Preparation of N1-(4,5-dimethyl-2-nitrophenyl)propane-1,3-diamine

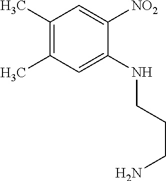

N1-(4,5-Dimethyl-2-nitrophenyl)propane-1,3-diamine is prepared by heating a neat mixture of 1-bromo-4,5-dimethyl-2-nitrobenzene (230 mg, 1.0 mmol) and propane-1,3-diamine (2 mL, excess) at 160° C. for 4 h. The resulting mixture is evaporated to dryness, and then dissolved in DCM (40 ml) and extracted with 2 M HCl (2×30 ml). The aqueous phase is washed with DCM (2×30 ml), and then basified with 2N NaOH to pH 13 (61 mL). The cloudy aqueous phase is then extracted with DCM (3×30 ml) and CHCl₃ (2×30 mL). The organic phase is dried with Na₂SO₄, filtered and evaporated to give N1-(4,5-dimethyl-2-nitrophenyl)propane-1,3-diamine (191 mg) as an orange solid. LC-MS m/z 224.0 [M+H], retention time 4.18 min.

### Step 2 Preparation of N1-benzyl-N3-(4,5-dimethyl-2-nitrophenyl)propane-1,3-diamine

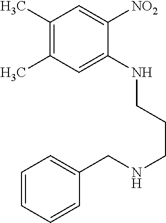

N1-(4,5-Dimethyl-2-nitrophenyl)propane-1,3-diamine (134 mg, 0.60 mmol) is dissolved in MeOH (6 ml) at room temperature, and then benzaldehyde (64 mg, 0.60 mmol) and AcOH (1 drop) are added. This solution is stirred at room temperature for 2 h, and then NaBH₃CN (75 mg, 1.20 mmol) is added in one portion and the resulting solution is stirred at room temperature for 16 h. The reaction is quenched with H₂O (3 drops), and the reaction mixture is evaporated. The crude product is dry loaded onto silica gel (5 g) with DCM, and the product is purified by column chromatography (0-10% MeOH in DCM) to give N1-benzyl-N3-(4,5-dimethyl-2-nitrophenyl)propane-1,3-diamine (147 mg, 78%) as an oily film. LC-MS m/z 314.1 [M+H], retention time 4.96 min.

### Step 3 Preparation of N1-(3-(benzylamino)propyl)-4,5-dimethylbenzene-1,2-diamine

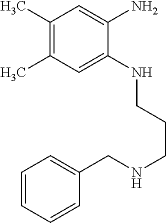

N1-Benzyl-N3-(4,5-dimethyl-2-nitrophenyl)propane-1,3-diamine (147 mg, 0.47 mmol) is dissolved in MeOH (15 ml). The reaction vessel is placed under vacuum and refilled with Ar, and this process is repeated. Pd/C (50 mg, 10% Pd/C, 3% Pd w/w) is added to the solution, and the mixture is cooled to 0° C. under Ar. The vessel is placed under vacuum and then refilled with H₂ (1 atm). The reaction is stirred at 0° C. for 16 h, at which time the reaction mixture is placed under vacuum and refilled with Ar, and then filtered through celite using MeOH (50 ml) to elute the product. The solvent is then evaporated to give N1-(3-(benzylamino)propyl)-4,5-dimethylbenzene-1,2-diamine, (135 mg, quantitative) as an oil which is taken onto the next step without further purification.

### Step 4 Preparation of 10-(3-(benzylamino)propyl)-7,8-dimethylbenzo[g]pteridine-2,4(3H,10H)-dione 2,2,2-trifluoroacetic acid salt

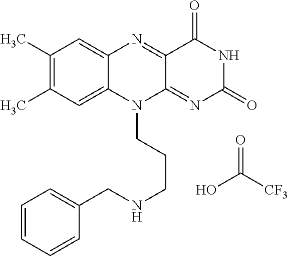

Crude N1-(3-(benzylamino)propyl)-4,5-dimethylbenzene-1,2-diamine (0.47 mmol), alloxan monohydrate (79 mg, 0.49 mmol) and boric acid (58 mg, 0.94 mmol) are dissolved in AcOH (10 ml) at rt, and the mixture is stirred at rt for 3 h. The reaction mixture is then evaporated to dryness, dissolved in ACN (5 ml) and H₂O (5 ml), and purified by preparatory HPLC. 10-(3-(Benzylamino)propyl)-7,8-dimethylbenzo[g]pteridine-2,4(3H,10H)-dione 2,2,2-trifluoroacetate is isolated as 56 mg (0.14 mmol, 30% yield) as a yellow powder. ¹H NMR (400 MHz, DMSO-d₆) δ 2.15 (t, 2H), 2.47 (s, 3H), 2.52 (s, 3H), 3.11 (m, 2H), 4.15 (t, 2H), 4.72 (t, 2H), 7.45 (m, 3H), 7.54 (m, 2H), 7.85 (s, 1H), 7.96 (s, 1H), 8.84 (br s, 2H), 11.43 (s, 1H). LC-MS m/z 390.2 [M+H], retention time 2.46 min.

### Example 48

### 10-Hexyl-7,8-dimethylbenzo[g]pteridine-2,4(3H,10H)-dione

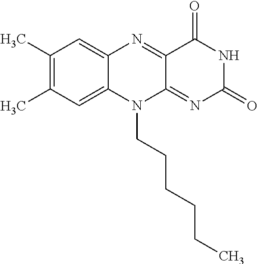

### Step 1 Preparation of N-hexyl-4,5-dimethyl-2-nitroaniline

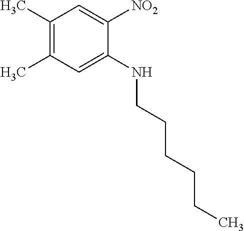

N-Hexyl-4,5-dimethyl-2-nitroaniline is prepared by heating a neat solution of 1-bromo-4,5-dimethyl-2-nitrobenzene (230 mg, 1.0 mmol) in N-hexylamine (300 mg, 3.0 mmol) at 115° C. for 5 h. The resulting mixture is diluted in DCM (40 mL), washed successively with H₂O (40 mL), 1 M HCl (30 mL), and brine (40 mL), and then dried over Na₂SO₄, filtered and evaporated to give 235 mg (0.94 mmol, 94% yield) of product as an orange powder. LC-MS m/z 251.0 [M+H]⁺, retention time 5.33 min.

### Step 2 Preparation of N¹-hexyl-4,5-dimethylbenzene-1,2-diamine

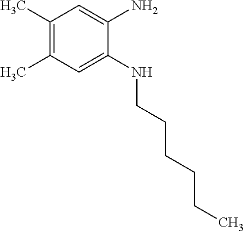

N¹-Hexyl-4,5-dimethylbenzene-1,2-diamine is prepared from N-hexyl-4,5-dimethyl-2-nitroaniline (235 mg, 0.94 mmol) by catalytic reduction with Pd/C (10% Pd/C, 4% Pd w/w) and NaBH₄ (115 mg, 3.0 mmol) in MeOH (10 mL) at room temperature under Ar. The reaction is complete within 40 min, at which time the reaction mixture is filtered through celite using MeOH (30 mL) to elute the product. The solvent is then evaporated to give N¹-hexyl-4,5-dimethylbenzene-1,2-diamine (quantitative) as a mixture of borate salts which is taken onto the next step without further purification.

### Step 3 Preparation of 10-hexyl-7,8-dimethylbenzo[g]pteridine-2,4(3H,10H)-dione

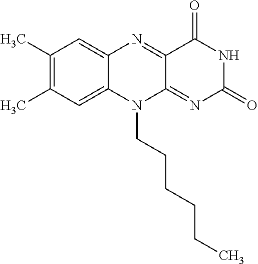

10-Hexyl-7,8-dimethylbenzo[g]pteridine-2,4(3H,10H)-dione is prepared from the crude N¹-hexyl-4,5-dimethylbenzene-1,2-diamine (0.94 mmol), alloxan monohydrate (158 mg, 0.99 mmol) and boric acid (117 mg, 1.9 mmol) in AcOH (10 mL) at rt for 3 h. The reaction mixture is then evaporated to dryness, dissolved in DCM (30 mL) and H₂O (50 mL), and the aqueous phase is extracted with DCM (2×20 mL). The combined organic portions are washed with brine (60 mL), and then dried over Na₂SO₄, filtered and evaporated to give a solid which is purified by preparative TLC (mobile phase 5% MeOH in DCM). The product is isolated as of a bright orange powder (122 mg, 39% yield). ¹H NMR (400 MHz, CDCl₃) δ 0.91 (t, 3H), 1.37 (m, 4H), 1.53 (m, 2H), 1.85 (quint., 2H), 2.45 (s, 3H), 2.57 (s, 3H), 4.69 (s, 2H), 7.39 (s, 1H), 8.06 (s, 1H), 8.59 (s, 1H). LC-MS m/z 327.1 [M+H]⁺, retention time 5.28 min.

### Example 49

### 10-(Hex-5-en-1-yl)-7,8-dimethylbenzo[g]pteridine-2,4(3H,10H)-dione

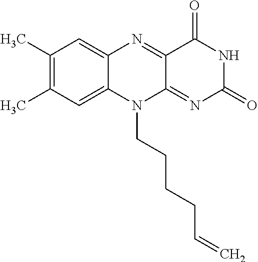

### Step 1 Preparation of N¹-(Hex-5-en-1-yl)-4,5-dimethylbenzene-1,2-diamine

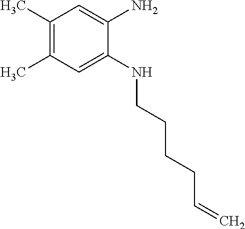

N¹-(Hex-5-en-1-yl)-4,5-dimethylbenzene-1,2-diamine is prepared by heating a solution of 4,5-dimethylbenzene-1,2-diamine (1 g, 7.34 mmol) and 6-bromohex-1-ene (1.197 g, 7.34 mmol), sodium iodide (2.20 g, 14.68 mmol) and triethylamine (1.485 g, 14.68 mmol) in THF (100 mL) at 60° C. for 12 h. The resulting mixture is diluted with EtOAc (100 mL), washed with brine (100 mL), and then dried over Na₂SO₄, filtered and evaporated. The residue is dry loaded on silica gel and purified by column chromatography using EtOAc in hexanes as eluent (gradient 0-50% EtOAc). N¹-(Hex-5-en-1-yl)-4,5-dimethylbenzene-1,2-diamine is isolated following evaporation of the appropriate fractions (870 mg, 54% yield). LC-MS m/z 219.1 [M+H], retention time 2.98 min.

### Step 2 Preparation of 10-(hex-5-en-1-yl)-7,8-dimethylbenzo[g]pteridine-2,4(3H,10H)-dione

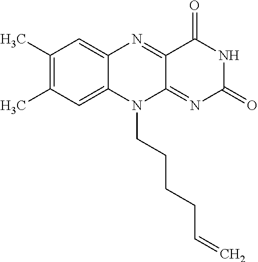

10-(Hex-5-en-1-yl)-7,8-dimethylbenzo[g]pteridine-2,4(3H,10H)-dione is prepared by stirring N¹-(Hex-5-en-1-yl)-4,5-dimethylbenzene-1,2-diamine (830 mg, 3.8 mmol), alloxan monohydrate (608 mg, 3.8 mmol) and boric acid (1.028 g, 3.8 mmol) in AcOH (30 mL) at rt for 3 h. The reaction mixture is then evaporated to dryness, dissolved in EtOAc (100 mL) and H₂O (100 mL), and the organic phase is washed with brine (2×50 mL) and then dried over Na₂SO₄, filtered and evaporated to give a solid which is dry loaded on silica gel and purified by column chromatography using EtOAc in hexanes as eluent (gradient 0-30% EtOAc). The product is isolated as a yellow powder (226 mg, 18% yield). ¹H NMR (400 MHz, CDCl₃) δ 1.66 (m, 2H), 1.90 (m, 2H), 1.20 (t, 2H), 2.48 (s, 3H), 2.59 (s, 3H), 4.73 (br s, 2H), 5.06 (m, 2H), 5.83 (m, 1H), 7.42 (s, 1H), 8.09 (s, 1H), 8.53 (s, 1H). LC-MS m/z 325.0 [M+H]⁺, retention time 4.95 min.

The compounds of the invention particularly those compounds as set forth in Table 3 below which are disclosed and claimed either individually and/or collectively may generally be prepared using similar procedures as set forth in General Procedures 1 and 2 and/or Examples 41-49 above. It is to be understood that the appropriate reagents, solvents and reaction condition for those reactions are used as apparent to one skilled in the art.

TABLE 3

| | | | HPLC | | | |
|---|---|---|---|---|---|---|
| | | LC-MS | retention | | | |
| | | MH⁺ | time | HPLC | | |
| Entry | Compounds of the Invention | (m/z) | (min.) | method | Preparation | Name |
| 1 | 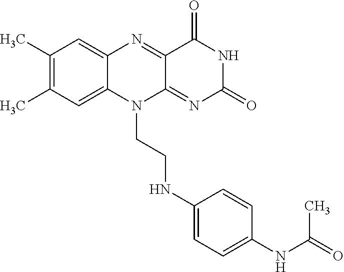 | 419.2 [M + H]+ 441.3 [M + Na]+ | 5.431 | A′ | Example 41 | N-(4-((2-(7,8- Dimethyl-2,4- dioxo-3,4- dihydrobenzo[g] pteridin-10(2H)- yl)ethyl)amino) phenyl)acetamide |
| 2 | 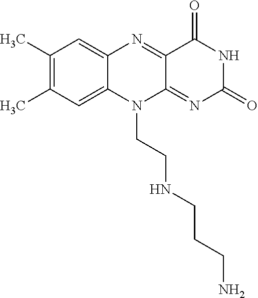 | 343.2 | 4.2 | A′ | Prepared using General Procedure 1 | 10-[2-(3-Amino- propylamino)- ethyl]-7,8- dimethyl-10H- benzo[g]pteridine- 2,4-dione |
| 3 | 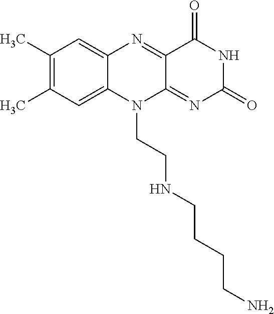 | 357.2 | 4.72 | A′ | Prepared using General Procedure 1 | 10-(2-(4- aminobutylamino) ethyl)-7,8- dimethylbenzo [g]pteridine- 2,4(3H,10H)- dione |
| 4 | 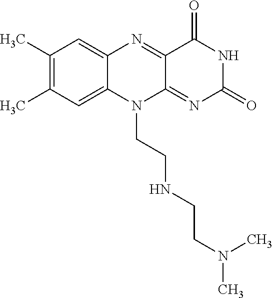 | 357.3 | 4.199 | A′ | Example 42 | 10-(2-((2- (dimethylamino) ethyl)amino) ethyl)-7,8- dimethylbenzo [g]pteridine- 2,4(3H,10H)- dione |
| 5 | 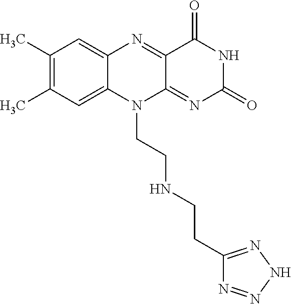 | 382.1 | 1.48 | C′ | Prepared using General Procedure 1 | 10-(2-(2-(2H- tetrazol-5- yl)ethylamino) ethyl)-7,8- dimethylbenzo [g]pteridine- 2,4(3H,10H)- dione |
| 6 | 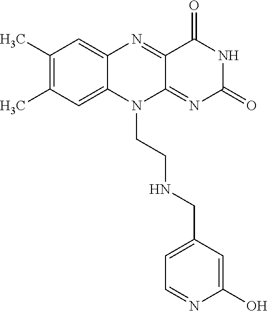 | 393.1 | 3.28 | C′ | Example 43 | 10-(2-((2- hydroxypyridin-4- yl)methylamino) ethyl)-7,8- dimethylbenzo [g]pteridine- 2,4(3H,10H)- dione |
| 7 | 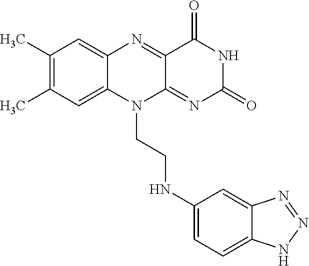 | 403 | 2.31 | C′ | Prepared using General Procedure 1 | 10-(2-(1H- benzo[d][1,2,3] triazol-5- ylamino)ethyl)-7,8- dimethylbenzo [g]pteridine- 2,4(3H,10H)- dione |
| 8 | 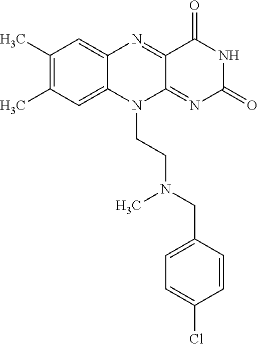 | 424 | 2.65 | C′ | Prepared using General Procedure 1 | 10-(2-((4- chlorobenzyl) (methyl)amino) ethyl)-7,8- dimethylbenzo [g]pteridine- 2,4(3H,10H)- dione |
| 9 | 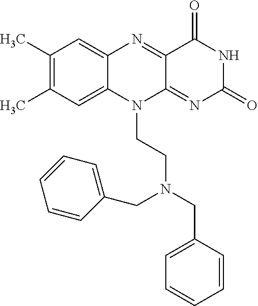 | 465.9 | 4.41 | C′ | Prepared using General Procedure 1 | 10-(2- (dibenzylamino) ethyl)-7,8- dimethylbenzo [g]pteridine- 2,4(3H,10H)- dione |
| 10 | 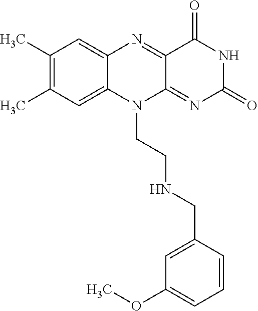 | 405.9 | 3.28 | C′ | Prepared using General Procedure 1 | 10-(2-((3- methoxybenzyl) amino)ethyl)-7,8- dimethylbenzo [g]pteridine- 2,4(3H,10H)- dione |
| 11 | 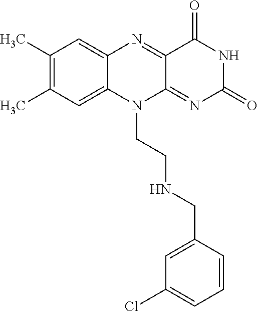 | 409.8 | 3.99 | C′ | Prepared using General Procedure 1 | 10-(2-((3- chlorobenzyl) amino)ethyl)-7,8- dimethylbenzo [g]pteridine- 2,4(3H,10H)- dione |
| 12 | 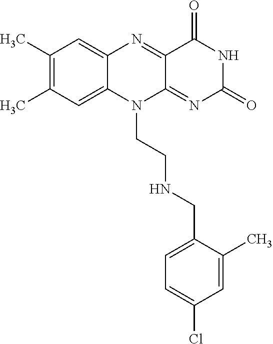 | 423.8 | 4.16 | C′ | Prepared using General Procedure 1 | 10-(2-((4-chloro-2- methylbenzyl) amino)ethyl)-7,8- dimethylbenzo [g]pteridine- 2,4(3H,10H)- dione |
| 13 | 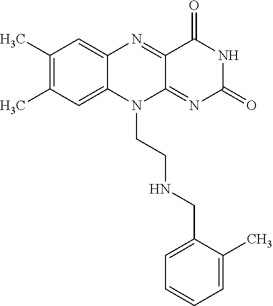 | 390 | 2.56 | C′ | Prepared using General Procedure 1 | 7,8-dimethyl-10- (2-((2- methylbenzyl) amino)ethyl)benzo [g]pteridine- 2,4(3H,10H)- dione |
| 14 | 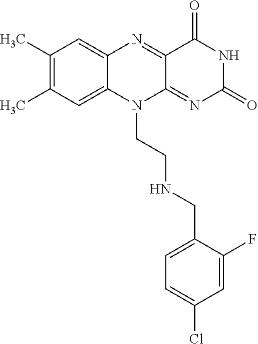 | 427.9 | 3.59 | C′ | Prepared using General Procedure 1 | 10-(2-((4-chloro-2- fluorobenzyl) amino)ethyl)-7,8- dimethylbenzo [g]pteridine- 2,4(3H,10H)- dione |
| 15 | 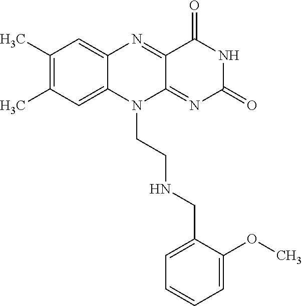 | 405.9 | 3.33 | C′ | Prepared using General Procedure 1 | 10-(2-((2- methoxybenzyl) amino)ethyl)-7,8- dimethylbenzo [g]pteridine- 2,4(3H,10H)- dione |
| 16 | 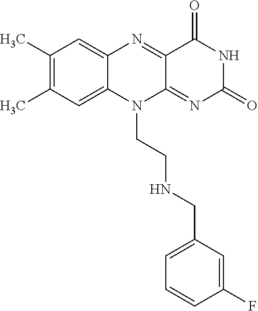 | 394.1 | 3.47 | C′ | Prepared using General Procedure 1 | 10-(2-((3- fluorobenzyl) amino)ethyl)-7,8- dimethylbenzo [g]pteridine- 2,4(3H,10H)- dione |
| 17 | 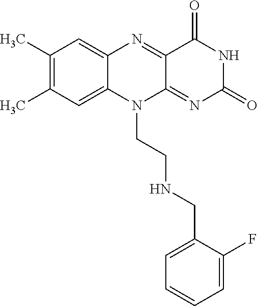 | 394 | 3.36 | C′ | Prepared using General Procedure 1 | 10-(2-((2- fluorobenzyl) amino)ethyl)-7,8- dimethylbenzo [g]pteridine- 2,4(3H,10H)- dione |
| 18 | 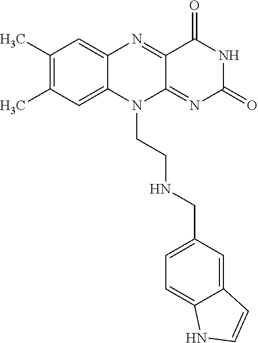 | 414.9 | 2.36 | C′ | Prepared using General Procedure 1 | 10-(2-(((1H- indol-5- yl)methyl)amino) ethyl)-7,8- dimethylbenzo [g]pteridine- 2,4(3H,10H)- dione |
| 19 | 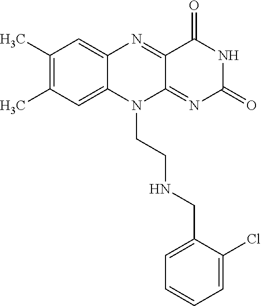 | 410 | 3.52 | C′ | Prepared using General Procedure 1 | 10-(2-((2- chlorobenzyl) amino)ethyl)-7,8- dimethylbenzo [g]pteridine- 2,4(3H,10H)- dione |
| 20 | 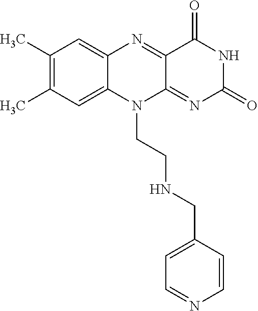 | 377.1 | 6.1 | F′ | Prepared using General Procedure 1 | 7,8-dimethyl-10- (2-((pyridin-4- ylmethyl)amino) ethyl)benzo[g] pteridine- 2,4(3H,10H)- dione |
| 21 | 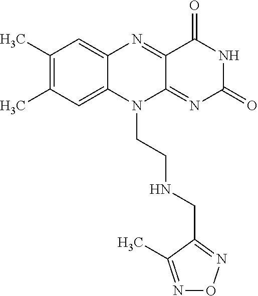 | 381.9 | 2.14 | C′ | Prepared using General Procedure 1 | 7,8-dimethyl-10- (2-(((4-methyl- 1,2,5-oxadiazol-3- yl)methyl)amino) ethyl)benzo[g] pteridine- 2,4(3H,10H)- dione |
| 22 | 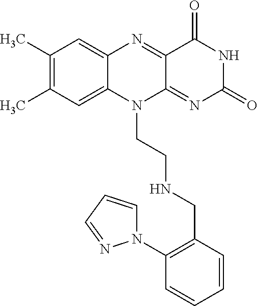 | 442.1 | 3.52 | C′ | Prepared using General Procedure 1 | 10-(2-((2-(1H- pyrazol-1- yl)benzyl)amino) ethyl)-7,8- dimethylbenzo [g]pteridine- 2,4(3H,10H)- dione |
| 23 | 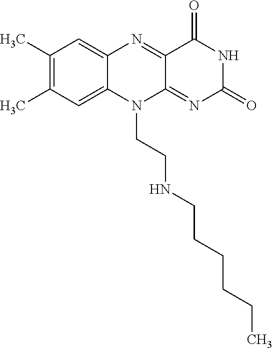 | 370.1 | 3.81 | C′ | Prepared using General Procedure 1 | 10-(2- (hexylamino) ethyl)-7,8- dimethylbenzo [g]pteridine- 2,4(3H,10H)- dione |
| 24 | 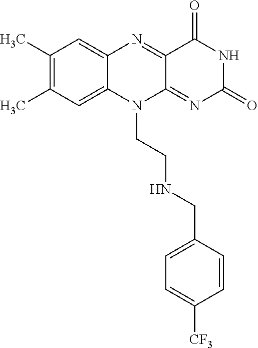 | 444.1 | 3.93 | C′ | Prepared using General Procedure 1 | 7,8-dimethyl-10- (2-((4- (trifluoromethyl) benzyl)amino) ethyl)benzo[g] pteridine- 2,4(3H,10H)- dione |
| 25 | 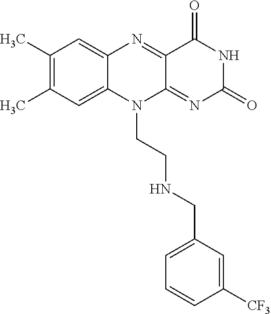 | 444 | 2.82 | C′ | Prepared using General Procedure 1 | 7,8-dimethyl-10- (2-((3- (trifluoromethyl) benzyl)amino) ethyl)benzo[g] pteridine- 2,4(3H,10H)- dione |
| 26 | 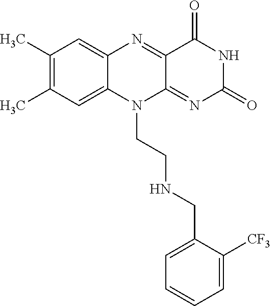 | 444 | 2.72 | C′ | Prepared using General Procedure 1 | 7,8-dimethyl-10- (2-((2- (trifluoromethyl) benzyl)amino) ethyl)benzo[g] pteridine- 2,4(3H,10H)- dione |
| 27 | 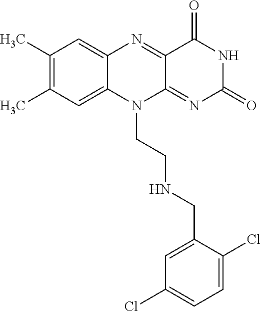 | 443.9 | 2.69 | C′ | Prepared using General Procedure 1 | 10-(2-((2,5- dichlorobenzyl) amino)ethyl)-7,8- dimethylbenzo [g]pteridine- 2,4(3H,10H)- dione |
| 28 | 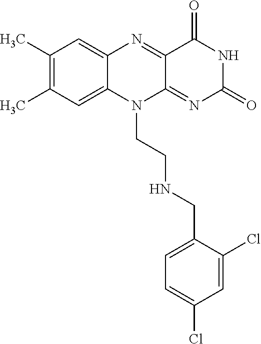 | 444 | 2.79 | C′ | Prepared using General Procedure 1 | 10-(2-((2,4- dichlorobenzyl) amino)ethyl)-7,8- dimethylbenzo [g]pteridine- 2,4(3H,10H)- dione |
| 29 | 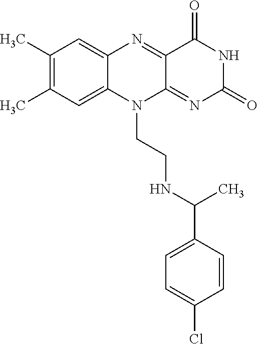 | 423.9 | 2.82 | C′ | Prepared using General Procedure 1 | 10-(2-((1-(4- chlorophenyl) ethyl)amino)ethyl)- 7,8-dimethylbenzo [g]pteridine- 2,4(3H,10H)- dione |
| 30 | 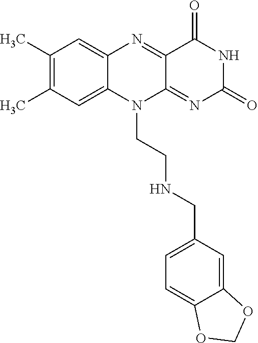 | 419.9 | 2.41 | C′ | Prepared using General Procedure 1 | 10-(2- ((benzo[d][1,3] dioxol-5- ylmethyl)amino) ethyl)-7,8- dimethylbenzo [g]pteridine- 2,4(3H,10H)- dione |
| 31 | 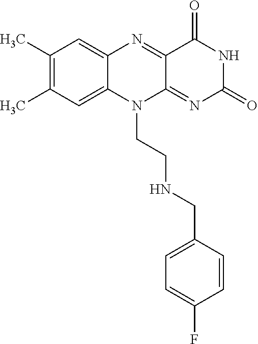 | 394 | 2.46 | C′ | Prepared using General Procedure 1 | 10-(2-((4- fluorobenzyl) amino)ethyl)-7,8- dimethylbenzo [g]pteridine- 2,4(3H,10H)- dione |
| 32 | 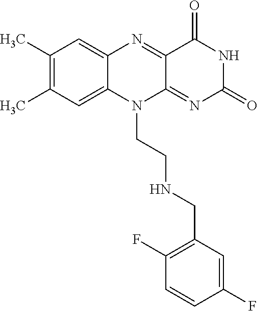 | 411.9 | 3.45 | C′ | Prepared using General Procedure 1 | 10-(2-((2,5- difluorobenzyl) amino)ethyl)-7,8- dimethylbenzo [g]pteridine- 2,4(3H,10H)- dione |
| 33 | 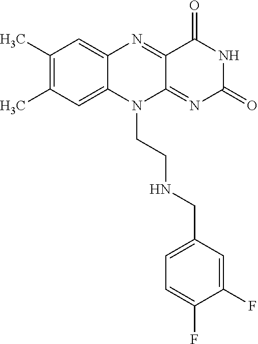 | 411.8 | 3.63 | C′ | Prepared using General Procedure 1 | 10-(2-((3,4- difluorobenzyl) amino)ethyl)-7,8- dimethylbenzo [g]pteridine- 2,4(3H,10H)- dione |
| 34 | 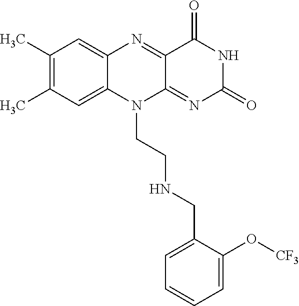 | 460 | 3.88 | C′ | Prepared using General Procedure 1 | 7,8-dimethyl-10- (2-((2- (trifluoromethoxy) benzyl)amino) ethyl)benzo[g] pteridine- 2,4(3H,10H)- dione |
| 35 | 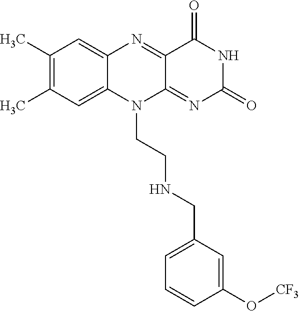 | 460 | 4 | C′ | Prepared using General Procedure 1 | 7,8-dimethyl-10- (2-((3- (trifluoromethoxy) benzyl)amino) ethyl)benzo[g] pteridine- 2,4(3H,10H)- dione |
| 36 | 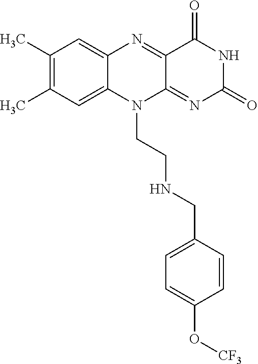 | 459.9 | 4.06 | C′ | Prepared using General Procedure 1 | 7,8-dimethyl-10- (2-((4- (trifluoromethoxy) benzyl)amino) ethyl)benzo[g] pteridine- 2,4(3H,10H)- dione |
| 37 | 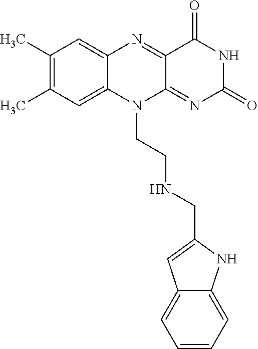 | 414.9 | 2.67 | C′ | Prepared using General Procedure 1 | 10-(2-(((1H- indol-2- yl)methyl)amino) ethyl)-7,8- dimethylbenzo [g]pteridine- 2,4(3H,10H)- dione |
| 38 | 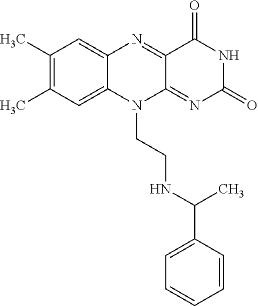 | 389.9 | 2.53 | C′ | Prepared using General Procedure 1 | 7,8-dimethyl-10- (2-((1- phenylethyl) amino)ethyl)benzo [g]pteridine- 2,4(3H,10H)- dione |
| 39 | 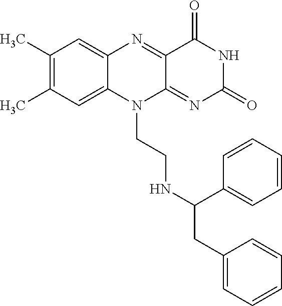 | 466.1 | 5.34 | C′ | Prepared using General Procedure 1 | 10-(2-((1,2- diphenylethyl) amino)ethyl)-7,8- dimethylbenzo [g]pteridine- 2,4(3H,10H)- dione |
| 40 | 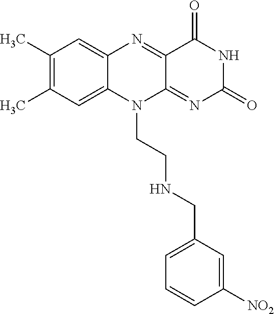 | 421.1 | 3.57 | C′ | Prepared using General Procedure 1 | 7,8-dimethyl-10- (2-((3- nitrobenzyl) amino)ethyl)benzo [g]pteridine- 2,4(3H,10H)- dione |
| 41 | 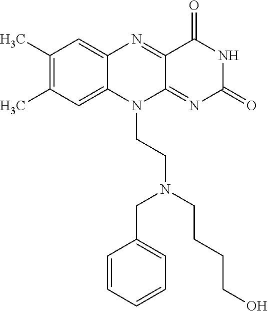 | 448.2 | 4.6 | C′ | Prepared using General Procedure 1 | 10-(2-(benzyl(4- hydroxybutyl) amino)ethyl)-7,8- dimethylbenzo [g]pteridine- 2,4(3H,10H)- dione |
| 42 | 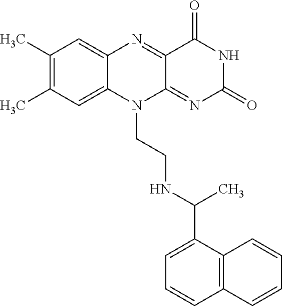 | 439.9 | 5.05 | C′ | Prepared using General Procedure 1 | 7,8-dimethyl-10- (2-((1- (naphthalen-1- yl)ethyl)amino) ethyl)benzo[g] pteridine- 2,4(3H,10H)- dione |
| 43 | 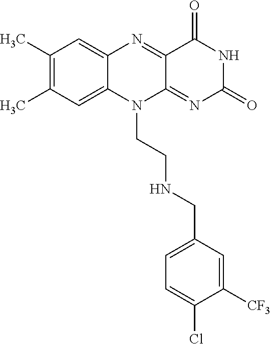 | 477.9 | 3.11 | C′ | Prepared using General Procedure 1 | 10-(2-((4-chloro-3- (trifluoromethyl) benzyl)amino) ethyl)-7,8- dimethylbenzo [g]pteridine- 2,4(3H,10H)- dione |
| 44 | 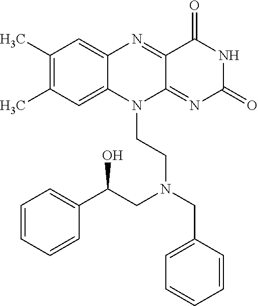 | 496 | 3.07 | C′ | Prepared using General Procedure 1 | (R)-10-(2- (benzyl(2- hydroxy-2- phenylethyl) amino)ethyl)-7,8- dimethylbenzo [g]pteridine- 2,4(3H,10H)- dione |
| 45 | 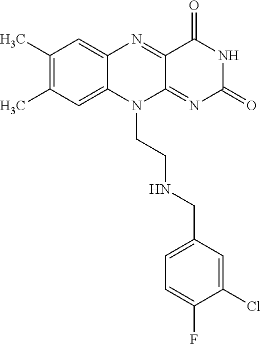 | 428.1 | 4.99 | C′ | Prepared using General Procedure 1 | 10-(2-((3-chloro-4- fluorobenzyl) amino)ethyl)-7,8- dimethylbenzo [g]pteridine- 2,4(3H,10H)- dione |
| 46 | 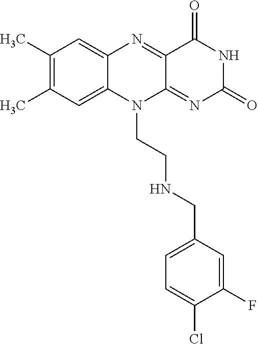 | 427.9 | 4.99 | C′ | Prepared using General Procedure 1 | 10-(2-((4-chloro-3- fluorobenzyl) amino)ethyl)-7,8- dimethylbenzo [g]pteridine- 2,4(3H,10H)- dione |
| 47 | 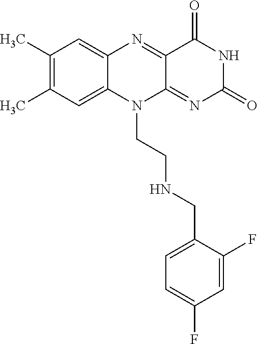 | 412.1 | 4.73 | C′ | Prepared using General Procedure 1 | 10-(2-((2,4- difluorobenzyl) amino)ethyl)-7,8- dimethylbenzo [g]pteridine- 2,4(3H,10H)- dione |
| 48 | 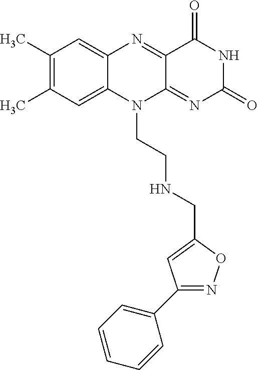 | 443 | 2.81 | C′ | Prepared using General Procedure 1 | 7,8-dimethyl-10- (2-(((3- phenylisoxazol-5- yl)methyl)amino) ethyl)benzo[g] pteridine- 2,4(3H,10H)- dione |
| 49 | 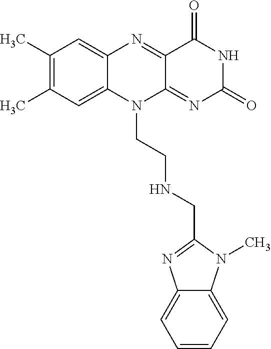 | 430 | 2.35 | C′ | Prepared using General Procedure 1 | 7,8-dimethyl-10- (2-(((1-methyl-1H- benzo[d]imidazol- 2-yl)methyl) amino)ethyl)- benzo[g]pteridine 2,4(3H,10H)- dione |
| 50 | 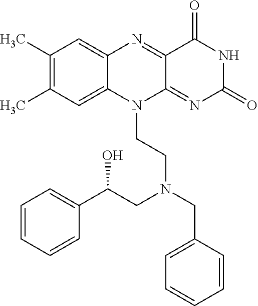 | 496.1 | 5.25 | C′ | Prepared using General Procedure 1 | (S)-10-(2- (benzyl(2- hydroxy-2- phenylethyl) amino)ethyl)-7,8- dimethylbenzo [g]pteridine- 2,4(3H,10H)- dione |
| 51 | 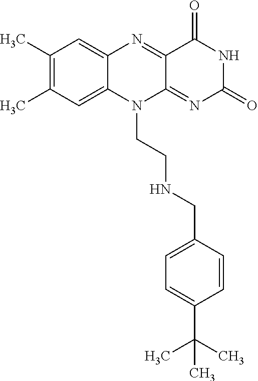 | 432.1 | 3.23 | C′ | Prepared using General Procedure 1 | 10-(2-((4-(tert- butyl)benzyl) amino)ethyl)-7,8- dimethylbenzo [g]pteridine- 2,4(3H,10H)- dione |
| 52 | 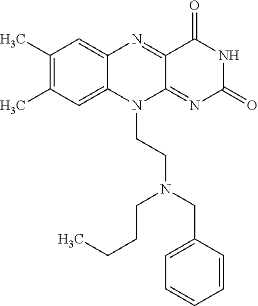 | 432 | 2.83 | C′ | Prepared using General Procedure 1 | 10-(2- (benzyl(butyl) amino)ethyl)-7,8- dimethylbenzo [g]pteridine- 2,4(3H,10H)- dione |
| 53 | 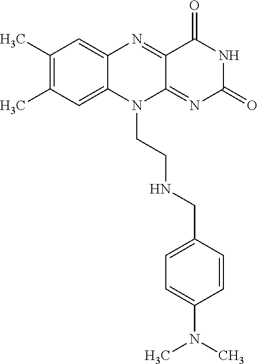 | 418.9 | 2.15 | C′ | Prepared using General Procedure 1 | 10-(2-((4- (dimethylamino) benzyl)amino) ethyl)-7,8- dimethylbenzo [g]pteridine- 2,4(3H,10H)- dione |
| 54 | 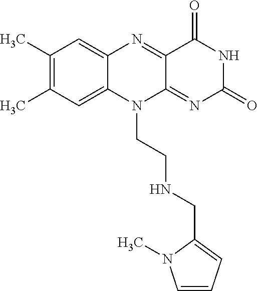 | 380 | 1.56 | C′ | Prepared using General Procedure 1 | 7,8-dimethyl-10- (2-(((1-methyl- 1H-imidazol-5- yl)methyl)amino) ethyl)benzo[g] pteridine- 2,4(3H,10H)- dione |
| 55 | 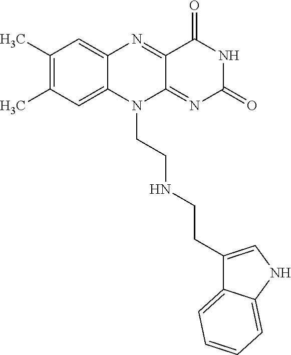 | 429 | 2.62 | C′ | Prepared using General Procedure 1 | 10-(2-((2-(1H- indol-3- yl)ethyl)amino) ethyl)-7,8- dimethylbenzo [g]pteridine- 2,4(3H,10H)- dione |
| 56 | 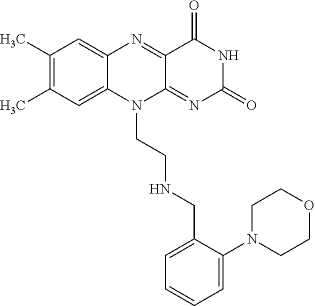 | 461 | 2.54 | C′ | Prepared using General Procedure 1 | 7,8-dimethyl-10- (2-((2- morpholinobenzyl) amino)ethyl) benzo[g]pteridine- 2,4(3H,10H)- dione |
| 57 | 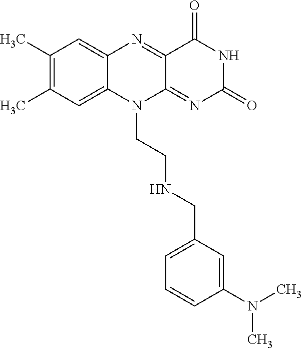 | 419.1 | 2.08 | C′ | Prepared using General Procedure 1 | 10-(2-((3- (dimethylamino) benzyl)amino) ethyl)-7,8- dimethylbenzo [g]pteridine- 2,4(3H,10H)- dione |
| 58 | 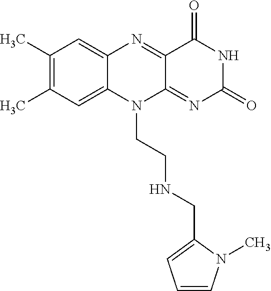 | 378.8 | 2.29 | C′ | Prepared using General Procedure 1 | 7,8-dimethyl-10- (2-(((1-methyl- 1H-pyrrol-2- yl)methyl)amino) ethyl)benzo[g] pteridine- 2,4(3H,10H)- dione |
| 59 | 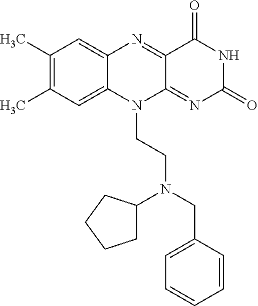 | 444.1 | 2.69 | C′ | Prepared using General Procedure 1 | 10-(2- (benzyl(cyclopentyl) amino)ethyl)-7,8- dimethylbenzo [g]pteridine- 2,4(3H,10H)- dione |
| 60 | 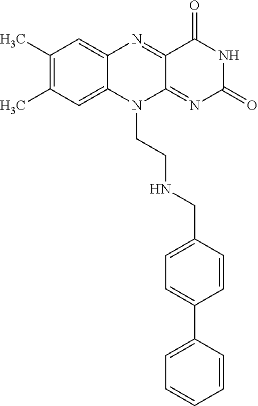 | 452 | 3.15 | C′ | Prepared using General Procedure 1 | 10-(2-(([1,1′- biphenyl]-4- ylmethyl)amino) ethyl)-7,8- dimethylbenzo [g]pteridine- 2,4(3H,10H)- dione |
| 61 | 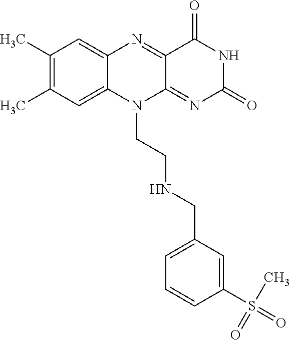 | 454.1 | 3.62 | C′ | Prepared using General Procedure 1 | 7,8-dimethyl-10- (2-((3- (methylsulfonyl) benzyl)amino) ethyl)benzo[g] pteridine- 2,4(3H,10H)- dione |
| 62 | 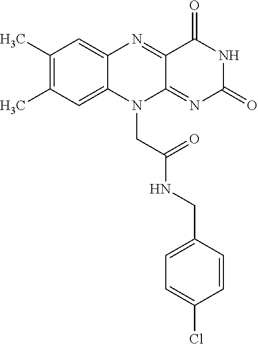 | 423.8 | 3.46 | C′ | Example 44 | N-(4- chlorobenzyl)-2- (7,8-dimethyl- 2,4-dioxo-3,4- dihydrobenzo[g] pteridin-10(2H)- yl)acetamide |
| 63 | 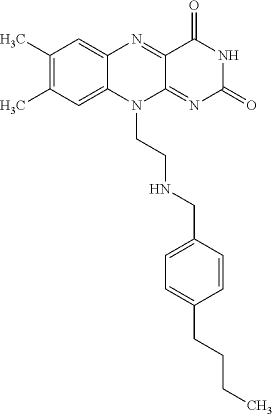 | 432.1 | 4.74 | C′ | Prepared using General Procedure 1 | 10-(2-((4- butylbenzyl) amino)ethyl)-7,8- dimethylbenzo [g]pteridine- 2,4(3H,10H)- dione |
| 64 | 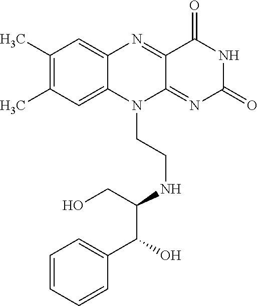 | 435.9 | 2.33 | C′ | Prepared using General Procedure 1 | 10-(2-((1,3- dihydroxy-1- phenylpropan-2- yl)amino)ethyl)- 7,8-dimethylbenzo [g]pteridine- 2,4(3H,10H)- dione |
| 65 | 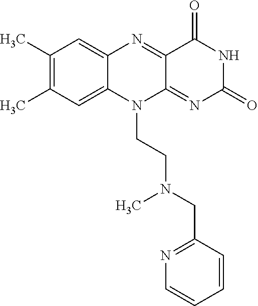 | 391 | 3.51 | C′ | Prepared using General Procedure 1 | 7,8-dimethyl-10-(2- (methyl(pyridin-2- ylmethyl)amino) ethyl)benzo[g] pteridine- 2,4(3H,10H)- dione |
| 66 | 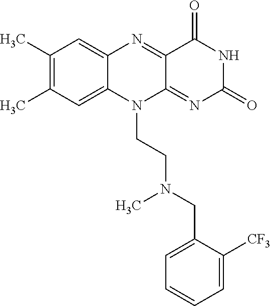 | 458.1 | 4.23 | C′ | Prepared using General Procedure 1 | 7,8-dimethyl-10- (2-(methyl(2- (trifluoromethyl) benzyl)amino) ethyl)benzo[g] pteridine- 2,4(3H,10H)- dione |
| 67 | 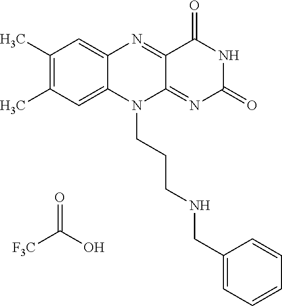 | 390.2 | 2.46 | C′ | Example 47 | 10-(3- (Benzylamino) propyl)-7,8- dimethylbenzo [g]pteridine- 2,4(3H,10H)- dione 2,2,2- trifluoroacetate |
| 68 | 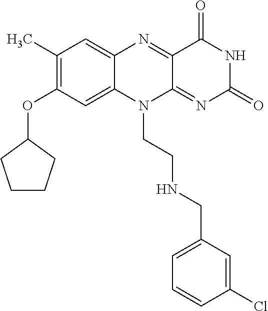 | 480.1 | 6 | C′ | Prepared using a similar synthesis procedure as in Example 46 | 10-(2-((3- chlorobenzyl) amino)ethyl)-8- (cyclopentyloxy)-7- methylbenzo[g] pteridine- 2,4(3H,10H)- dione |
| 69 | 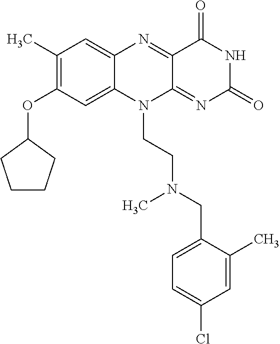 | 508.1 | 3.57 | C′ | Prepared using a similar synthesis procedure as in Example 46 | 10-(2-((4-chloro-2- methylbenzyl) (methyl)amino) ethyl)-8- (cyclopentyloxy)- 7-methylbenzo[g] pteridine- 2,4(3H,10H)- dione |
| 70 | 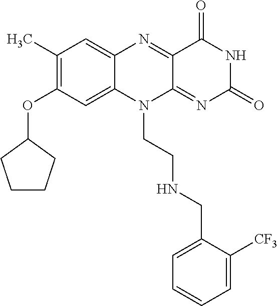 | 514.1 | 6.02 | C′ | Prepared using the synthesis Example 46 | 8-(cyclopentyloxy)- 7-methyl-10-(2-((2- (trifluoromethyl) benzyl)amino) ethyl)benzo[g] pteridine- 2,4(3H,10H)- dione |
| 71 | 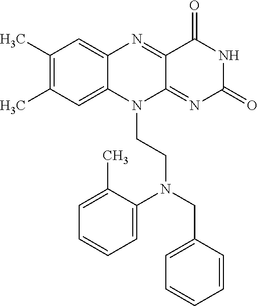 | 465.9 | 5.66 | C′ | Prepared using General Procedure 1 | 7,8-dimethyl-10- (2-((phenyl(o- tolyl)methyl) amino)ethyl)benzo [g]pteridine- 2,4(3H,10H)- dione |
| 72 | 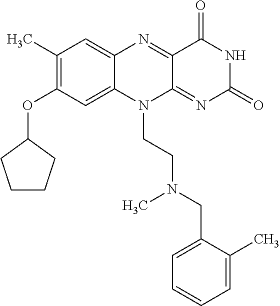 | 474.1 | 3.35 | C′ | Prepared using a similar synthesis procedure as in Example 46 | 8-(cyclopentyloxy)- 7-methyl-10-(2- (methyl(2- methylbenzyl) amino)ethyl)benzo [g]pteridine- 2,4(3H,10H)- dione |
| 73 | 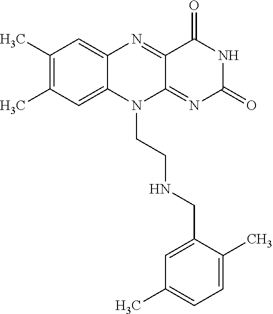 | 404 | 4.16 | C′ | Prepared using General Procedure 1 | 10-(2-((2,5- dimethylbenzyl) amino)ethyl)- 7,8-dimethylbenzo [g]pteridine- 2,4(3H,10H)- dione |
| 74 | 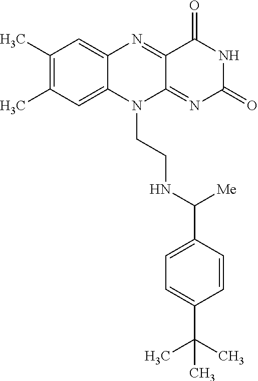 | 446.1 | 3.36 | C′ | Prepared using General Procedure 1 | 10-(2-((1-(4-(tert- butyl)phenyl) ethyl)amino)ethyl)- 7,8-dimethylbenzo [g]pteridine- 2,4(3H,10H)- dione |
| 75 | 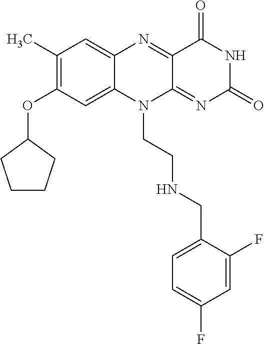 | 482.1 | 3.31 | C′ | Prepared using a similar synthesis procedure as in Example 46 | 8-(cyclopentyloxy)- 10-(2-((2,4- difluorobenzyl) amino)ethyl)-7- methylbenzo[g] pteridine- 2,4(3H,10H)- dione |
| 76 | 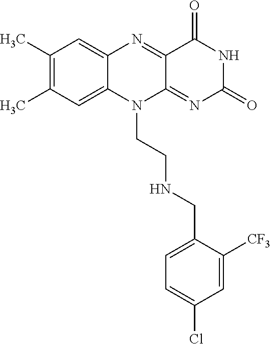 | 478 | 8.82 | F′ | Prepared using General Procedure 1 | 10-(2-((4-chloro-2- (trifluoromethyl) benzyl)amino) ethyl)-7,8- dimethylbenzo [g]pteridine- 2,4(3H,10H)- dione |
| 77 | 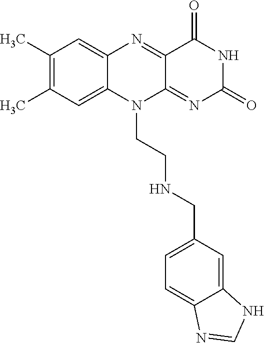 | 416.1 | 3.14 | C′ | Prepared using the synthesis of Example 45 | 10-(2-(((1H- benzo[d]imidazol-6- yl)methyl)amino) ethyl)-7,8- dimethylbenzo [g]pteridine- 2,4(3H,10H)- dione |
| 78 | 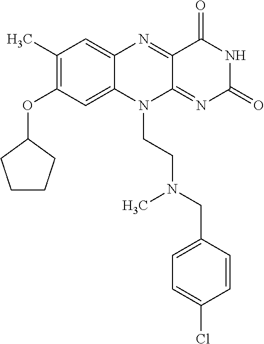 | 494 | 6.17 | C′ | Prepared using the synthesis of Example 45 | 10-(2-((4- chlorobenzyl) (methyl)amino) ethyl)-8- (cyclopentyloxy)-7- methylbenzo[g] pteridine- 2,4(3H,10H)- dione |
| 79 | 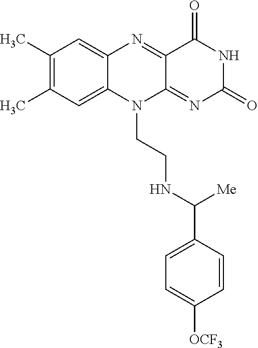 | 474.07 | 2.96 | C′ | Prepared using General Procedure 1 | 7,8-dimethyl-10- (2-((1-(4- (trifluoromethoxy) phenyl)ethyl) amino)ethyl) benzo[g]pteridine- 2,4(3H,10H)- dione |
| 80 | 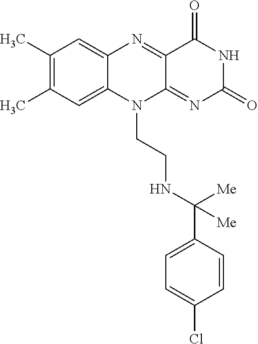 | 438 | 2.78 | C′ | Prepared using General Procedure 1 | 10-(2-((2-(4- chlorophenyl) propan-2- yl)amino)ethyl)- 7,8-dimethylbenzo [g]pteridine- 2,4(3H,10H)- dione |
| 81 | 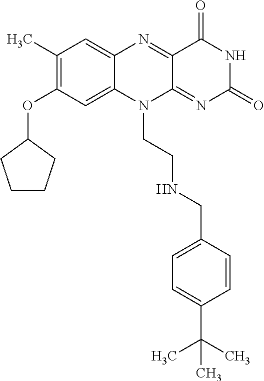 | 502.1 | 3.91 | C′ | Prepared using a similar synthesis procedure as in Example 46 | 10-(2-((4-(tert- butyl)benzyl) amino)ethyl)-8- (cyclopentyloxy)- 7-methylbenzo[g] pteridine- 2,4(3H,10H)- dione |
| 82 | 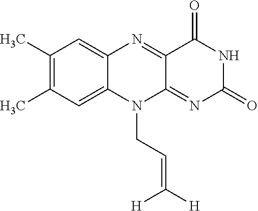 | 283.2 | 2.80 | G′ | Prepared using synthesis of Example 46 starting from 4,5- Dimethyl- o- phenylene diamine and allyl bromide | 10-allyl-7,8- dimethylbenzo [g]pteridine- 2,4(3H,10H)- dione |
| 83 | 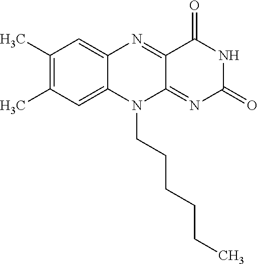 | 327.1 | 5.28 | C′ | Prepared using the synthesis of Example 48 | 10-Hexyl-7,8- dimethylbenzo [g]pteridine- 2,4(3H,10H)- dione |
| 84 | 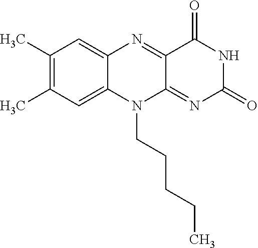 | 313.1 | 4.93 | C′ | Prepared using the synthesis of Example 48 | 7,8-dimethyl-10- pentylbenzo[g] pteridine- 2,4(3H,10H)- dione |
| 85 | 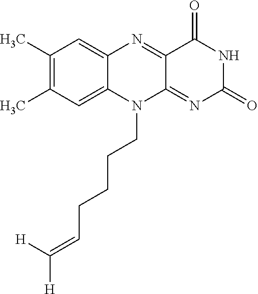 | 325 | 4.95 | C′ | Prepared using the synthesis of Example 49 | 10-(Hex-5-en-1- yl)-7,8- dimethylbenzo [g]pteridine- 2,4(3H,10H)- dione |

1. A compound of Formula Q(i): 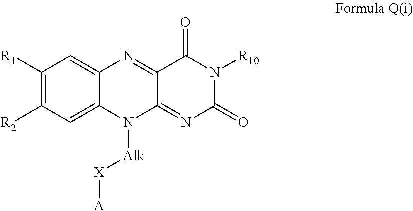
wherein:
(i) Alk is C₁₋₆alkylene;
(ii) X is —N(R₆) and A is:
—C₁₋₄alkyl-N(R₁₁)(R₁₂);
—C₀₋₄alkyl-aryl¹, or —C₀₋₄alkyl-heteroaryl¹, wherein the alkyl group of said -alkylaryl¹ and -alkylheteroaryl¹ is optionally substituted with hydroxy or another aryl¹, and the aryl¹ and heteroaryl¹ group of said -alkylaryl¹ and -alkylheteroaryl¹ are independently substituted with one or more:
—N(Rₐ)—C(O)—C₁₋₄alkyl, wherein Rₐ is H or C₁₋₄alkyl,
—OH,
heteroaryl¹,
heteroC₃₋₈cycloalkyl¹,
aryl¹,
—NO₂,
—N(Rₐ)(R_{b}), wherein Rₐ is H or C₁₋₄alkyl and R_{b} is C₁₋₄alkyl,
—SO₂—C₁₋₄alkyl;
—C₀₋₄alkyl-pyridyl substituted with one or more hydroxy;
—C₀₋₄alkyl-benzotriazolyl;
—C₀₋₄alkyl-indolyl;
—C₀₋₄alkyl-tetrazolyl;
—C₀₋₄alkyl-oxadiazolyl;
—C₀₋₄alkyl-benzodioxolyl;
—C₀₋₄alkyl-benzimidazolyl optionally substituted with —C₀₋₄alkyl;
—C₀₋₄alkyl-imidazolyl optionally substituted with C₁₋₄alkyl;
—C₀₋₄alkyl-pyrrolyl optionally substituted with —C₀₋₄alkyl; or
para-phenylbenzyl;
or
X is a single bond, and A is a monocyclic heteroaryl² wherein said monocyclic heteroaryl² is optionally substituted with C₁₋₄alkyl;
or
X is a single bond, —N(R₆)—, —N(R₆)—CH₂—, —N(R₆)—CH₂CH₂—, —N(R₆)—C(H)(CH₃)—, or —C(O)—; and:
A is a C₃₋₈cycloalkyl² wherein one or more carbon atoms of said cycloalkyl² are optionally and independently replaced with N, O, S, S(O)₂ or —C(O)—,
wherein said cycloalkyl² is optionally substituted with one or more
C₁₋₄alkyl,
—C(O)OR₇,
—CH₂C(O)OR₇,
—N(R₆)C(O)OR₇,
—OH,
hydroxy-C₁₋₄alkyl,
C₁₋₄alkoxy,
—CH₂N(R₆)—C(O)OR₇,
aryl² or aryl²-C₁₋₄alkyl wherein said aryl² group of said aryl² or aryl²-alkyl is optionally substituted with C₁₋₄alkyl,
heteroaryl²,
heteroaryl²-C₁₋₄alkyl,
—C₁₋₄alkyl-N(R₈)(R₉),
C₁₋₄alkoxy,
—C(O)N(R₆)—S(O)₂—C₁₋₄alkyl,
—N(H)—S(O)₂—C₁₋₄alkyl,
—S(O)₂—N(R₈)(R₉),
—C(O)N(H)CN,
—C(O)N(R₈)(R₉), or
—N(R₈)(R₉);
or
A is a 7-11 membered fused cycloalkyl-aryl or spiral compound wherein one or more carbon atoms may be a hetero atom selected from N, O or S and wherein said fused cycloalkyl-aryl or spiral group is optionally substituted with one or more hydroxy, C₁₋₄alkyl or oxo;
(iii) R₁ is H or C₁₋₈ alkyl;
(iv) R₂ is H, halo, C₁₋₄alkyl, —N(R₄)(R₅) or —O—C₃₋₈cycloalkyl;
(v) R₄ and R₅ are independently selected from:
H,
C₃₋₇cycloalkyl²,
—C₁₋₄alkyl, wherein said alkyl is optionally substituted with one or more groups selected from —OH, and —C(O)OR₇,
aryl² optionally substituted with halo,
aryl²-C₁₋₄alkyl wherein said aryl² group is optionally substituted with halo;
(vi) R₆ is H or C₁₋₄alkyl;
(vii) R₇ is H, C₁₋₄alkyl or —CH₂OC(O)CH₃;
(viii) R₈ and R₉ are independently H or C₁₋₄alkyl;
(ix) R₁₀ is H or —C₁₋₄alkyl-OC(O)CH₃;
(x) R₁₁ and R₁₂ are independently H or C₁₋₄alkyl
in free or salt form, provided that:
(a) when R₂ is chloro, Alk is propylene, X is a single bond and A is pyrrolidin-1-yl, then R₁ is C₁₋₈ alkyl or R₁₀ is —C₁₋₄alkyl-OC(O)CH₃;
(b) the compound is not 10-[3-(3,6-dioxo-1,4-cyclohexadien-1-yl)propyl)-3,7,8-trimethyl-benzo[g]pteridine-2,4-(3H,10H)-dione;
(c) the compound is not optionally substituted 1042-(9H-purin-9-yl)ethyl]-, 10-[3-(9H-purin-9-yl)propyl]- or 10-[6-(9H-purin-9-yl)hexyl]-7,8-dimethyl-benzo[g]pteridine-2,4-(3H,10H)-dione;
(d) the compound is not 10-[3-(1H-indol-3-yl)ethyl]- or 10-[3-(1H-indol-3-yl)propyl]-7,8-dimethyl-benzo[g]pteridine-2,4-(3H,10H)-dione;
(e) -Alk-X-A is not 2-(2-oxocylopentylidene)ethyl.

2. The compound according to claim 1, wherein said compound is selected from:
A) a compound of Formula Q-I(i): 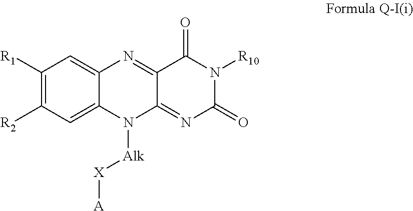
wherein:
(i) Alk is C₁₋₆alkylene;
(ii) X is a single bond, —N(R₆)—, —N(R₆)—CH₂—, —N(R₆)—CH₂CH₂—, —N(R₆)—C(H)(CH₃)—, or —C(O)— and
A is a —C₃₋₈cycloalkyl² wherein one or more carbon atoms of said cycloalkyl² are optionally and independently replaced with N, O, S, S(O)₂ or —C(O)—,
wherein said cycloalkyl² is optionally substituted with one or more:
C₁₋₄alkyl,
—C(O)OR₇,
—CH₂C(O)OR₇,
—N(R₆)C(O)OR₇,
—OH,
hydroxy-C₁₋₄alkyl,
C₁₋₄alkoxy,
—CH₂N(R₆)—C(O)OR₇,
aryl² or aryl²-C₁₋₄alkyl wherein said aryl² group of said aryl² or aryl²-alkyl is optionally substituted with C₁₋₄alkyl,
heteroaryl²,
heteroaryl²-C₁₋₄alkyl,
—C₁₋₄alkyl-N(R₈)(R₉),
C₁₋₄alkoxy,
—C(O)N(R₆)—S(O)₂—C₁₋₄alkyl,
—N(H)—S(O)₂—C₁₋₄alkyl,
—S(O)₂—N(R₈)(R₉),
—C(O)N(H)CN,
—C(O)N(R₈)(R₉), or
—N(R₈)(R₉);
or
A is a 7-11 membered fused cycloalkyl-aryl or spiral compound wherein one or more carbon atoms may be a hetero atom selected from N, O or S and wherein said fused cycloalkyl-aryl or spiral group is optionally substituted with one or more hydroxy, C₁₋₄alkyl or oxo;
(iii) R₁ is H or C₁₋₈alkyl;
(iv) R₂ is H, halo, C₁₋₄alkyl, —N(R₄)(R₅) or —O—C₃₋₈cycloalkyl;
(v) R₄ and R₅ are independently selected from:
H,
C₃₋₇cycloalkyl²,
—C₁₋₄alkyl, wherein said alkyl is optionally substituted with one or more groups selected from —OH, —C(O)OR₇,
aryl² optionally substituted with halo,
aryl²-C₁₋₄alkyl wherein said aryl² group is optionally substituted with halo;
(vi) R₆ is H or C₁₋₄alkyl;
(vii) R₇ is H, C₁₋₄alkyl or —CH₂OC(O)CH₃;
(viii) R₈ and R₉ are independently H or C₁₋₄alkyl;
(ix) R¹⁰ is H or —C₁₋₄alkyl-OC(O)CH₃;
in free or salt form;
B) a compound of Formula Q-II(i): 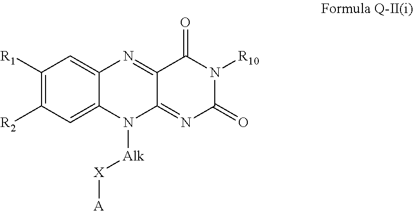
wherein:
(i) Alk is C₁₋₆alkylene;
(ii) X is a single bond and A is:
a monocyclic heteroaryl²,
wherein said heteroaryl² is optionally substituted with one or more C₁₋₄alkyl,
(iii) R₁ is H or C₁₋₈ alkyl;
(iv) R₂ is H, halo, C₁₋₄alkyl, —N(R₄)(R₅) or —O—C₃₋₈cycloalkyl²;
(v) R₄ and R₅ are independently selected from
H,
C₃₋₇cycloalkyl²,
—C₁₋₄alkyl, wherein said alkyl is optionally substituted with one or more groups selected from —OH, —C(O)OR₇,
aryl² optionally substituted with halo,
aryl²-C₁₋₄alkyl wherein said aryl group is optionally substituted with halo;
(vi) R₇ is H, C₁₋₄alkyl or —CH₂OC(O)CH₃;
(vii) R₁₀ is H or —C₁₋₄alkyl-OC(O)CH₃;
in free or salt form;
C) a compound of Formula Q-III(i): 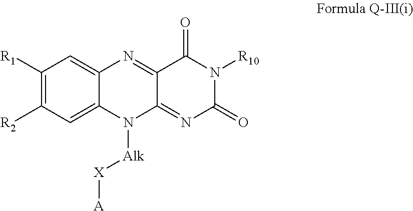
wherein:
(i) Alk is C₁₋₆alkylene;
(ii) X is —N(R₆) and A is:
—C₁₋₄alkyl-N(R₁₁)(R₁₂),
—C₀₋₄alkyl-aryl¹, or —C₀₋₄alkyl-heteroaryl¹, wherein the alkyl group of said -alkylaryl¹ and -alkylheteroaryl¹ is optionally substituted with hydroxy or another aryl¹, and the aryl¹ and heteroaryl¹ group of said -alkylaryl¹ and -alkylheteroaryl¹ are independently substituted with one or more:
—N(Rₐ)—C(O)—C₁₋₄alkyl, wherein Rₐ is H or C₁₋₄alkyl,
—OH,
heteroaryl¹,
heteroC₁₋₈cycloalkyl¹,
aryl¹,
—O-halo-C₁₋₄alkyl
—NO₂,
—N(Rₐ(R_{b}), wherein Rₐ is H or C₁₋₄alkyl and R_{b} is C₁₋₄alkyl,
—SO₂—C₁₋₄alkyl;
—C₀₋₄alkyl-pyridyl substituted with one or more hydroxy;
—C₀₋₄alkyl-benzotriazolyl;
—C₀₋₄alkyl-indolyl;
—C₀₋₄alkyl-tetrazolyl;
—C₀₋₄alkyl-oxadiazolyl;
—C₀₋₄alkyl-benzodioxolyl;
—C₀₋₄alkyl-benzimidazolyl optionally substituted with —C₀₋₄alkyl;
—C₀₋₄alkyl-imidazolyl optionally substituted with C₁₋₄alkyl;
—C₀₋₄alkyl-pyrrolyl optionally substituted with —C₀₋₄alkyl;
para-phenylbenzyl;
(iii) R₁ is H or C₁₋₈ alkyl;
(iv) R₂ is H, halo, C₁₋₄alkyl, —N(R₄(R₅) or —O—C₃₋₈cycloalkyl²;
(v) R₄ and R₆ are independently selected from
H,
C₃₋₇cycloalkyl²,
—C₁₋₄alkyl, wherein said alkyl is optionally substituted with one or more groups selected from —OH, —C(O)OR₇,
aryl² optionally substituted with halo,
aryl²-C₁₋₄alkyl wherein said aryl group is optionally substituted with halo;
(vi) R₆ is H or C₁₋₄alkyl;
(vii) R₇ is H, C₁₋₄alkyl or —CH₂OC(O)CH₃;
(viii) R₁₀ is H or —C₁₋₄alkyl-OC(O)CH₃;
(ix) R₁₁ and R₁₂ are independently H or C₁₋₄alkyl;
in free or salt form;
D) a compound of Formula Q-IV(i): 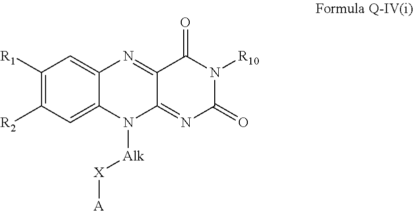
wherein
(i) Alk is C₁₋₆alkylene;
(ii) X is a single bond and A is pyrrolyl or imidazolyl;
or
X is a single bond and A is a pyrrolidinyl or piperidinyl, optionally substituted with another aryl or aryl-C₁₋₄alkyl;
or
X is —N(R₆)— and A is tetralinyl;
(iii) R₁ is H or C₁₋₈alkyl;
(iv) R₂ is H, halo or C₁₋₄alkyl;
(v) R₆ is H or C₁₋₄alkyl;
(vi) R₁₀ is H;
in free or salt form;
E) a compound of Formula Q-V(i): 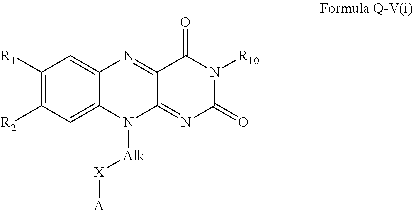
wherein:
(i) Alk is C₁₋₆alkylene;
(ii) X is a single bond and A is pyrrolyl pyrrolidinyl or piperidinyl, optionally substituted with another aryl or aryl-C₁₋₄alkyl;
(iii) R₁ is C₁₋₈ alkyl;
(iv) R₂ is C₁ alkyl;
(v) R₁₀ is H;
in free or salt form;
F) a Compound of Formula I(B): 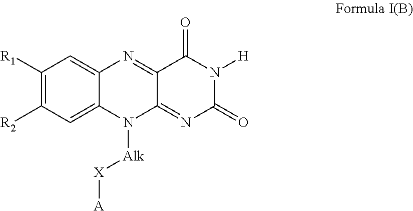
wherein:
(i) Alk is C₁₋₂alkylene;
(ii) X is —N(R₆)—;
(iii) A is selected from a group consisting of:
—C₁₋₄alkyl-N(R₁₁)(R₁₂);
—C₀₋₄alkyl-aryl¹, or —C₀₋₄alkyl-heteroaryl¹, wherein the alkyl group of said -alkylaryl¹ and -alkylheteroaryl¹ is optionally substituted with hydroxy or another aryl, and the aryl¹ and heteroaryl¹ group of said -alkylaryl¹ and -alkylheteroaryl¹ are independently substituted with one or more:
—N(Rₐ)—C(O)—C₁₋₄alkyl, wherein Rₐ is H or C₁₋₄alkyl,
—OH,
Heteroaryl¹,
heteroC₁₋₈cycloalkyl¹,
aryl¹,
—O-halo-C₁₋₄alkyl,
—NO₂
—N(Rₐ(R_{b}), wherein Rₐ is H or C₁₋₄alkyl and R_{b} is C₁₋₄alkyl,
—SO₂—C₁₋₄alkyl;
—C₀₋₄alkyl-pyridyl substituted with one or more hydroxy;
—C₀₋₄alkyl-benzotriazolyl;
—C₀₋₄alkyl-indolyl;
—C₀₋₄alkyl-tetrazolyl;
—C₀₋₄alkyl-oxadiazolyl;
—C₀₋₄alkyl-benzodioxolyl;
—C₀₋₄alkyl-benzimidazolyl optionally substituted with —C₀₋₄alkyl;
—C₀₋₄alkyl-imidazolyl optionally substituted with C₁₋₄alkyl;
—C₀₋₄alkyl-pyrrolyl optionally substituted with —C₀₋₄alkyl;
para-phenyl benzyl;
(iv) R₁ is H or C₁₋₄alkyl;
(v) R₂ is selected from a group consisting of H, C₁₋₄alkyl and —O—C₃₋₈cycloalkyl¹;
(vi) R₆ is H or C₁₋₄alkyl;
(vii) R₁₁ and R₁₂ are independently H or C₁₋₄alkyl;
in free or salt form.

3. (canceled)

4. (canceled)

5. (canceled)

6. (canceled)

7. (canceled)

8. The compound according to claim 1, wherein the compound is selected from any of those described in formulae Q.35, Q.36, Q.37, Q.38, Q.39, Q.40 or Q.41, in free or salt form.

9. A compound selected from:
A) a Compound of Formula I(A)(i): 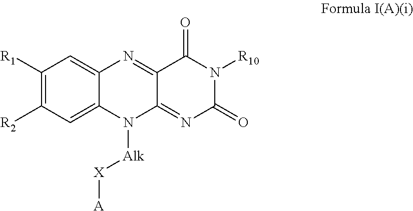
wherein:
(i) Alk is C₁₋₆alkylene;
(ii) X is a single bond, —N(R₆)—, —N(R₆)—CH₂— or —C(O)—;
(iii) A is a monocyclic heteroaryl or C₅₋₆cycloalkyl wherein one or more carbon atoms of said cycloalkyl are optionally and independently replaced with N, O, S, or —C(O)—, wherein said heteroaryl and cycloalkyl are independently optionally substituted with one or more —C(O)OR₇, —CH₂C(O)OR₇, —N(R₆)C(O)OR₇, —OH, hydroxy-C₁₋₄alkyl, —CH₂N(R₆)—C(O)OR₇, heteroaryl, heteroaryl-C₁₋₄alkyl, amineC₁₋₄alkyl, C₁₋₄alkoxy, —C(O)N(R₆)—S(O)₂—C₁₋₄alkyl or —N(R₈)(R₉);
(iv) R₁ is H or C₁₋₈ alkyl;
(v) R₂ is H, halo, C₁₋₄alkyl or —N(R₄)(R₅);
(vi) R₄ and R₅ are independently selected from H, C₃₋₇ cycloalkyl, —C₁₋₄alkyl, wherein said alkyl is optionally substituted with one or more groups selected from —OH, —C(O)OR₇, and aryl optionally substituted with halo;
(vii) R₆ is H or C₁₋₄alkyl;
(viii) R₇ is H, C₁₋₄alkyl or —CH₂OC(O)CH₃;
(ix) R₈ and R₉ are independently H or C₁₋₄alkyl;
(x) R₁₀ is H or —C₁₋₄alkyl-OC(O)CH₃;
in free, salt or prodrug form, with the proviso that when R₂ is chloro, Alk is propylene, X is a single bond and A is pyrrolidin-1-yl, then R₁ is C₁₋₈ alkyl or R₁₀ is —C₁₋₄alkyl-OC(O)CH₃
B) a Compound of Formula II(A): 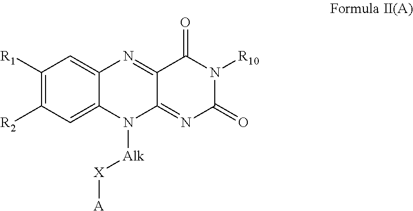
wherein
(i) Alk is C₁₋₆alkylene;
(ii) Y is —N(R₆)—C(O)— or —C(O)—N(R₆)—;
(iii) A is heteroaryl optionally substituted with one or more —C(O)OR₇, —CH₂C(O)OR₇, —N(R₆)C(O)OR₇, —OH, hydroxy-C₁₋₄alkyl, —CH₂N(R₆)—C(O)OR₇, heteroaryl, heteroaryl-C₁₋₄alkyl, amineC₁₋₄alkyl, C₁₋₄alkoxy, —C(O)N(R₆)—S(O)₂—C₁₋₄alkyl or —N(R₈)(R₉);
(iv) R₁ is H or C₁₋₄alkyl;
(v) R₁ is H, halo, C₁₋₄alkyl (e.g., methyl) or —N(R₄)(R₅);
(vi) R₄ and R₅ are independently selected from H, C₃₋₇ cycloalkyl, and —C₁₋₄alkyl, wherein said alkyl is optionally substituted with one or more groups selected from —OH, —C(O)OR₇, and aryl optionally substituted with halo;
(vii) R₆ is H or C₁₋₄alkyl;
(viii) R₂ is H, C₁₋₄alkyl or —CH₂OC(O)CH₃;
(ix) R₈ and R₉ are independently H, or C₁₋₄alkyl;
(x) R₁₀ is H or —C₁₋₄alkyl-OC(O)CH₃,
in free, salt or prodrug form;
C) a Compound of Formula II(B): 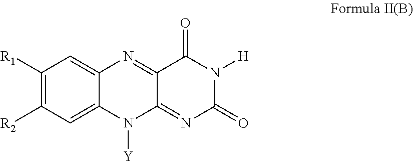
wherein:
(i) R₁ is H or C₁₋₄alkyl;
(ii) R₂ is selected from a group consisting of H, C₁₋₄alkyl and —O—C₃₋₈cycloalkyl¹;
(iii) Y is selected from a group consisting of: 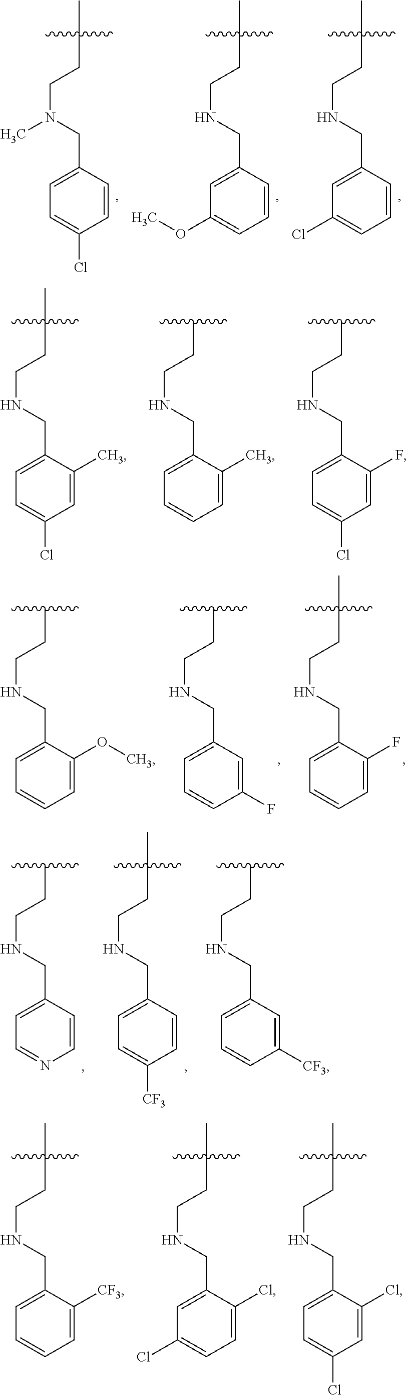 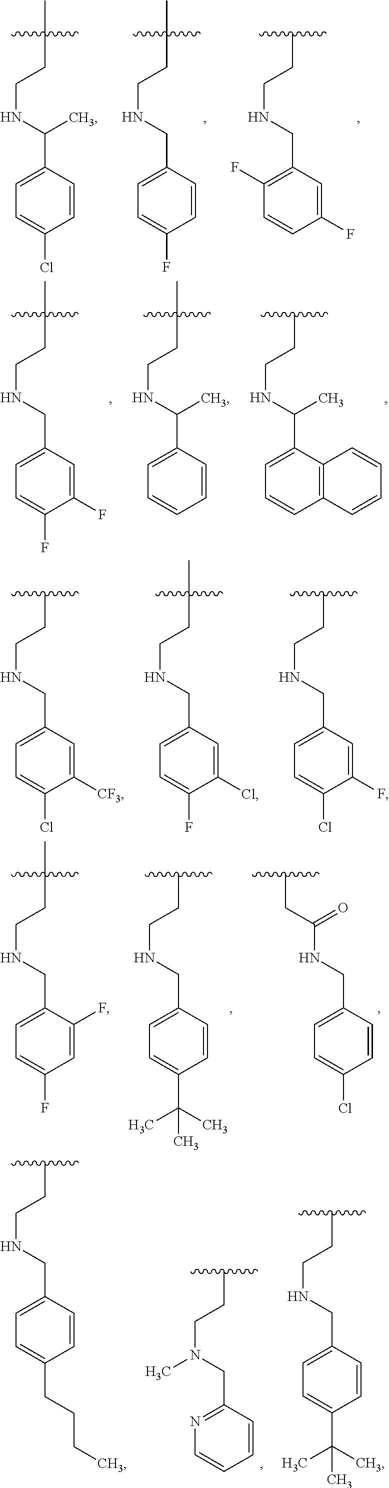 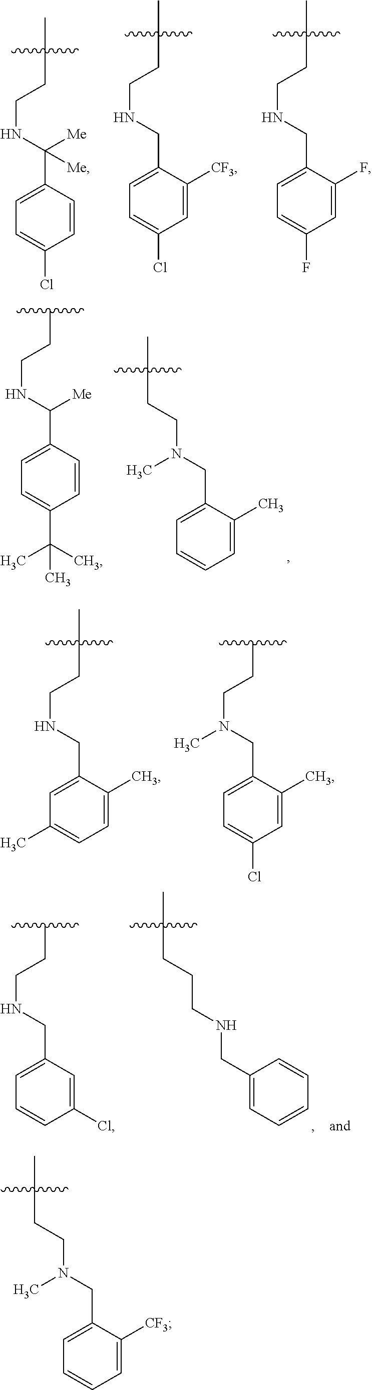
in free or salt form;
D) a Compound of Formula III(B): 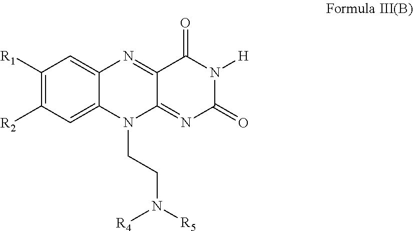
wherein:
(i) R₁ is H or C₁₋₄alkyl;
(ii) R₂ is selected from H, C₁₋₄alkyl and —O—C₃₋₈cycloalkyl;
(iii) R₄ is benzyl;
(iv) R₅ is selected from aryl¹-C₀₋₄alkyl, hydroxyC₁₋₄alkyl, C₁₋₄alkyl, and C₃₋₈cycloalkyl¹, wherein R₅ is optionally substituted with one or more hydroxy or C₁₋₄alkyl (e.g., methyl);
(v) or R₄ is H and R₅ is 1,2-diphenylethyl or 1-hydroxy-2-hydroxymethyl-2-phenyl (—C(H)(CH₂OH)—C(H)(OH)—C₆H₅);
in free or salt form.

10. (canceled)

11. The compound according to claim 9 selected from any one described in formula 2.8, in free, pharmaceutically acceptable salt form.

12. (canceled)

13. The compound according to claim 9, wherein said compound of Formula II(B) is selected from any compounds described in formulae 3.51, 3.52, 3.53 or 3.54 in free or salt form.

14. The compound according to claim 9, wherein said compound of Formula II(B) is selected from any of the following: 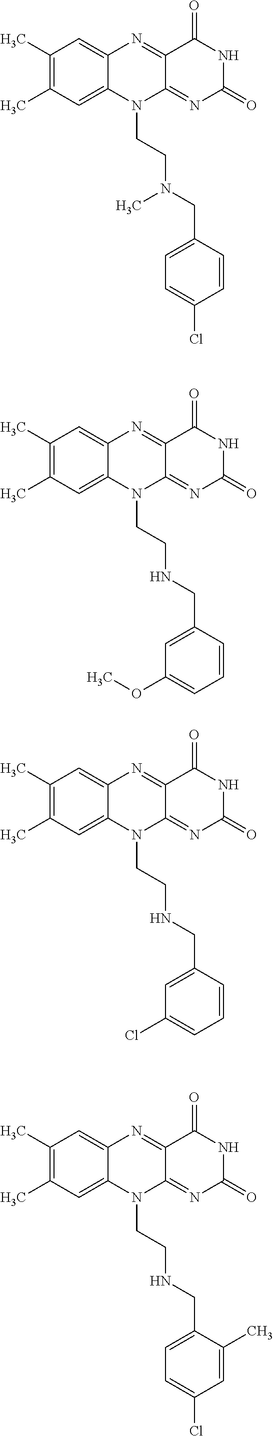 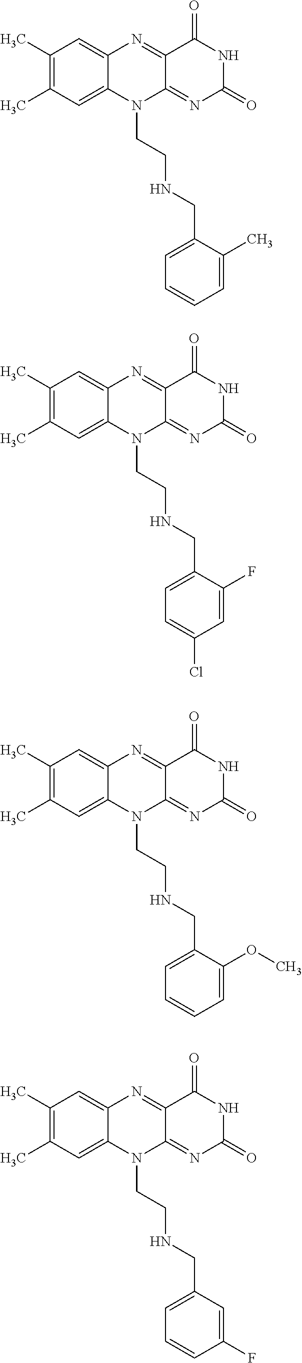 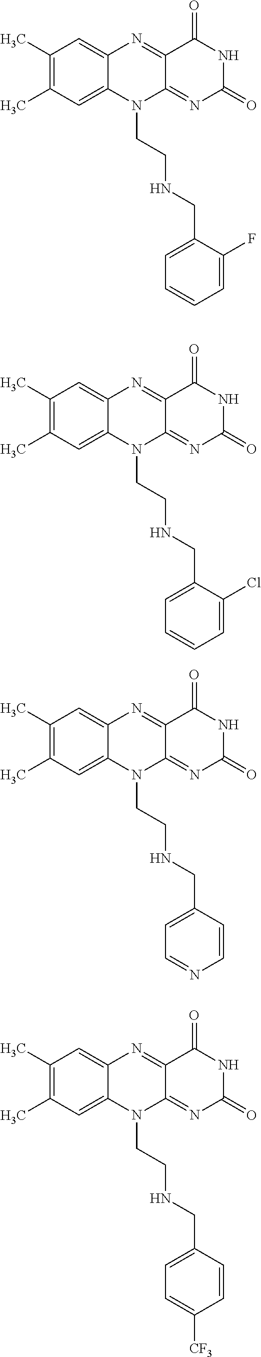 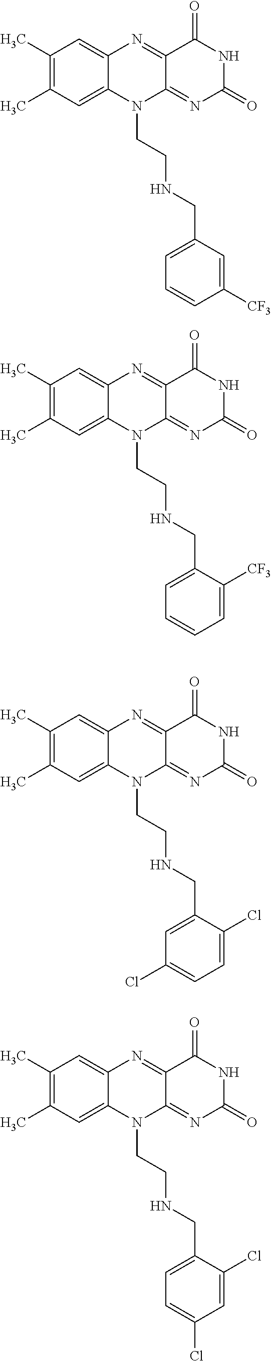 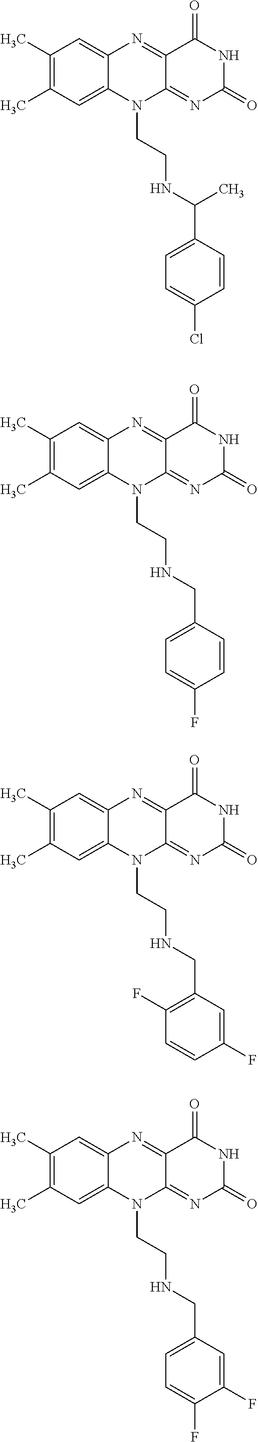 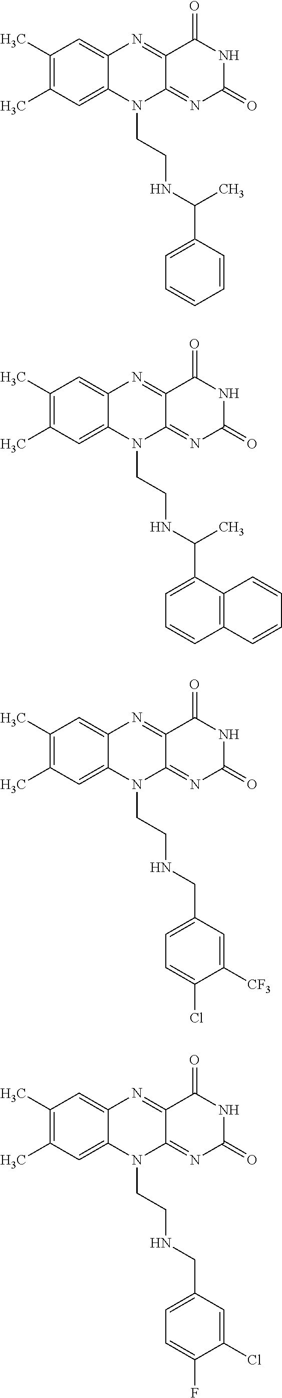 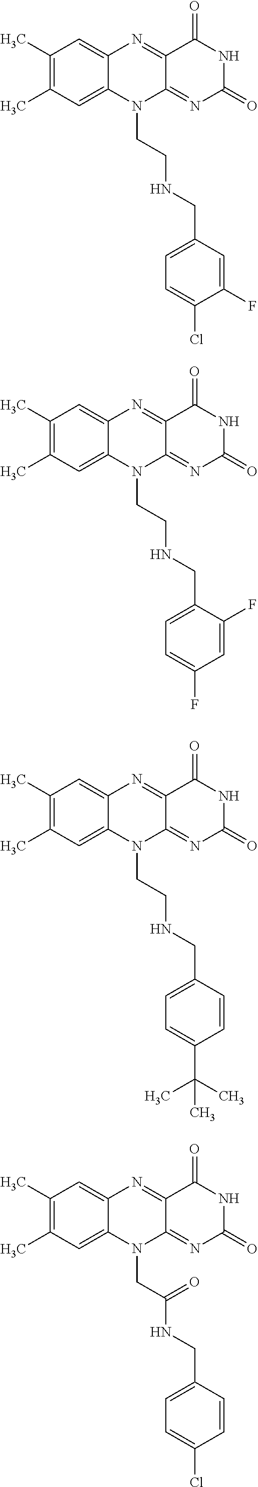 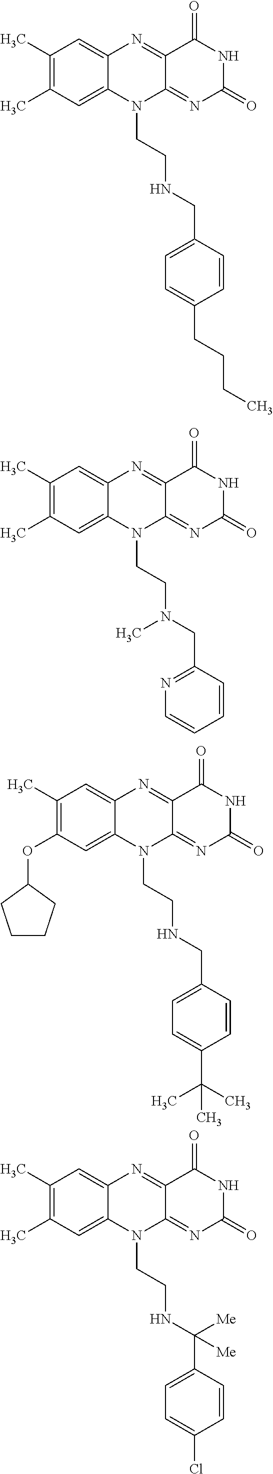 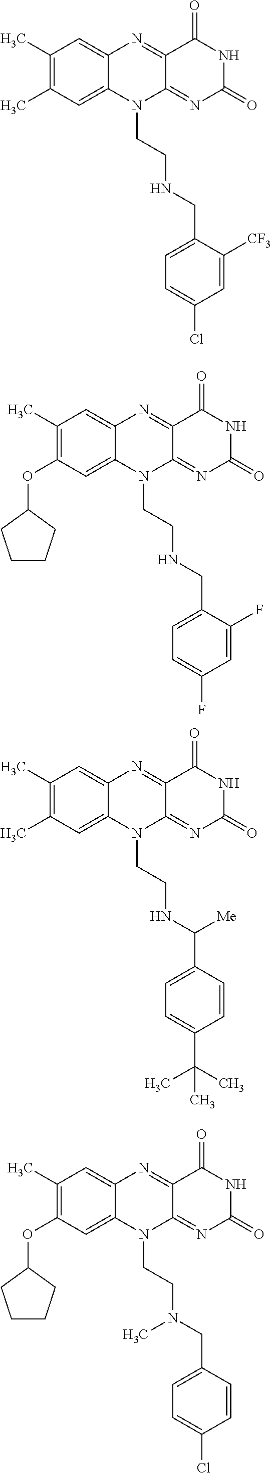 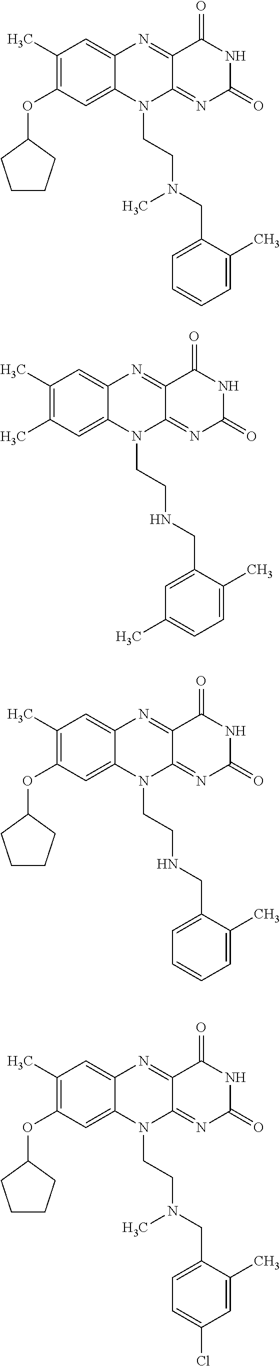 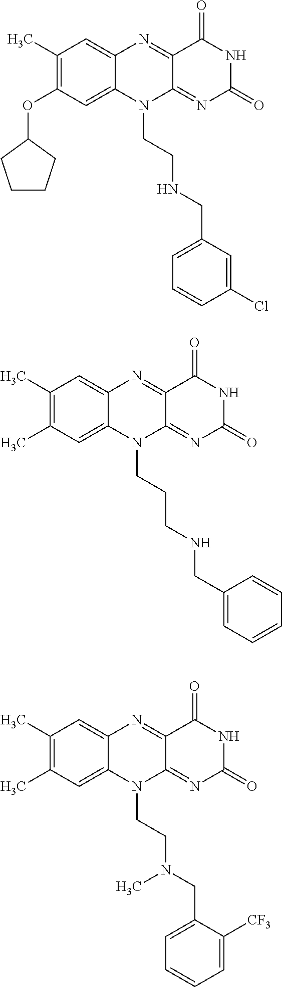
in free or salt form.

15. (canceled)

16. The compound according to claim 9, wherein said compound of Formula III(B) is selected from any of the following: 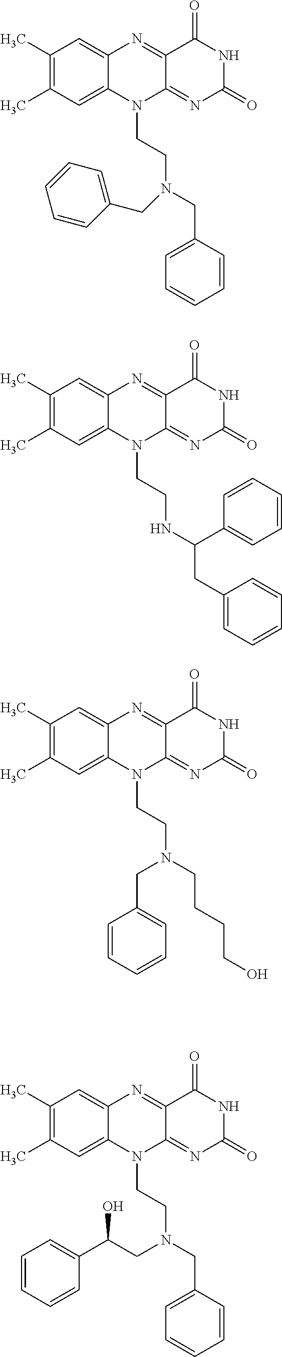 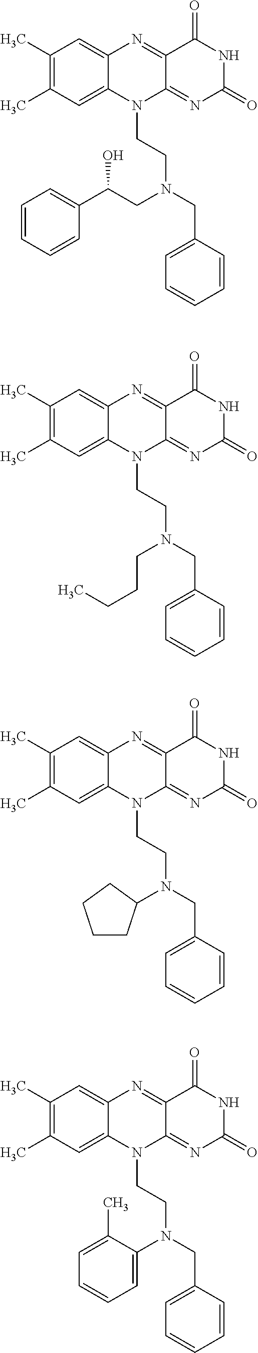 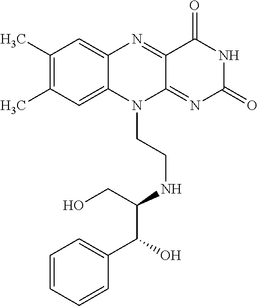
in free or salt form.

17. A compound of Formula IV(B) selected from any of the following: 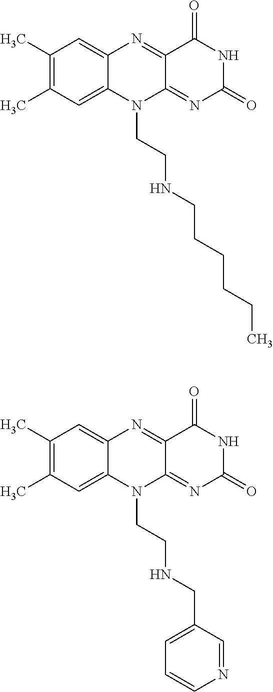
in free or salt form.

18. A compound of Formula V(B) selected from any of the following: 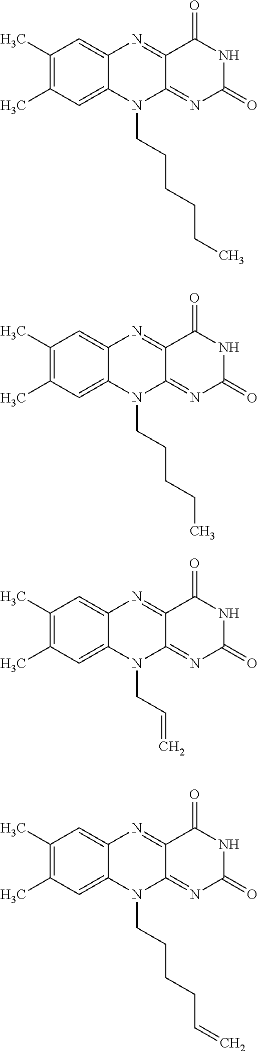
in free or salt form.

19. A method for the treatment or prophylaxis of a bacterial infection comprising administering to a patient in need of such treatment an effective amount of a compound selected from any of the following:
a) a compound of Formula Q: 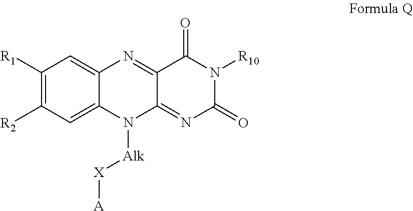
wherein:
(i) Alk is C₁₋₆alkylene;
(ii) X is —N(R₆) and A is:
—C₁₋₄alkyl-N(R₁₁)(R₁₂);
—C₀₋₄alkyl-aryl¹, or —C₀₋₄alkyl-heteroaryl¹, wherein the alkyl group of said -alkylaryl¹ and -alkylheteroaryl¹ is optionally substituted with hydroxy or another aryl¹, and the aryl¹ and heteroaryl¹ group of said -alkylaryl¹ and -alkylheteroaryl¹ are independently substituted with one or more:
—N(R₁)—C(O)—C₁₋₄alkyl (e.g., —NHC(O)CH₃), wherein Rₐ is H or C₁₋₄alkyl,
—OH,
heteroaryl¹,
heteroC₃₋₈cycloalkyl,
aryl¹,
—NO₂,
—N(Rₐ)(R_{b}), wherein Rₐ is H or C₁₋₄alkyl and R_{b} is C₁₋₄alkyl,
—SO₂—C₁₋₄alkyl;
—C₀₋₄alkyl-pyridyl substituted with one or more hydroxy;
—C₀₋₄alkyl-benzotriazolyl;
—C₀₋₄alkyl-indolyl;
—C₀₋₄alkyl-tetrazolyl;
—C₀₋₄alkyl-benzodioxolyl;
—C₀₋₄alkyl-benzimidazolyl optionally substituted with —C₀₋₄alkyl;
—C₀₋₄alkyl-imidazolyl optionally substituted with C₁₋₄alkyl;
—C₀₋₄alkyl-pyrrolyl optionally substituted with —C₀₋₄alkyl; or
para-phenylbenzyl;
or
X is a single bond, and A is a monocyclic heteroaryl², wherein said monocyclic heteroaryl² is optionally substituted with C₁₋₄alkyl;
or
X is a single bond, —N(R₆)—, —N(R₆)—CH₂—, —N(R₆)—CH₂CH₂—, —N(R₆)—C(H)(CH₃)—,
or —C(O)—; and:
A is a C₃₋₈cycloalkyl² wherein one or more carbon atoms of said cycloalkyl² are optionally and independently replaced with N, O, S, S(O)₂ or —C(O)—,
wherein said, cycloalkyl² is optionally substituted with one or more
C₁₋₄-alkyl,
—C(O)OR₇,
—CH₂C(O)OR₇,
—N(R₆)C(O)OR₇,
—OH,
hydroxy-C₁₋₄alkyl,
C₁₋₄alkoxy,
—CH₂N(R₆)—C(O)OR₇,
aryl² or aryl²-C₁₋₄alkyl wherein said aryl² group of said aryl² or aryl²-alkyl is optionally substituted with C₁₋₄alkyl,
heteroaryl²,
heteroaryl²-C₁₋₄alkyl,
—C₁₋₄alkyl-N(Rₛ)(R₉),
C₁₋₄alkoxy,
—C(O)N(R₆)—S(O)₂—C₁₋₄alkyl,
—N(H)—S(O)₂—C₁₋₄alkyl,
—S(O)₂—N(R₈)(R₉),
—C(O)N(H)CN,
—C(O)N(R₈)(R₉), or
—N(R₈)(R₉);
or
A is a 7-11 membered fused cycloalkyl-aryl or spiral compound wherein one or more carbon atoms may be a hetero atom selected from N, O or S and wherein said fused cycloalkyl-aryl or spiral group is optionally substituted with one or more hydroxy, C₁₋₄alkyl or oxo;
(iii) R₁ is H or C₁₋₈ alkyl;
(iv) R₂ is H, halo, C₁₋₄alkyl, —N(R₄)(R₅) or —O—C₃₋₈cycloalkyl;
(v) R₄ and R₅ are independently selected from
H,
C₃₋₇cycloalkyl²,
—C₁₋₄alkyl, wherein said alkyl is optionally substituted with one or more groups selected from —OH, —C(O)OR₇,
aryl² optionally substituted with halo,
aryl²-C₁₋₄alkyl wherein said aryl² group is optionally substituted with halo;
(vi) R₆ is H or C₁₋₄alkyl;
(vii) R₇ is H, C₁₋₄alkyl or —CH₂OC(O)CH₃;
(viii) R₈ and R₉ are independently H or C₁₋₄alkyl;
(ix) R₁₀ is H or —C₁₋₄alkyl-OC(O)CH₃;
(x) R₁₁ and R₁₂ are independently H or C₁₋₄alkyl;
b) a compound of Formula Q-I: 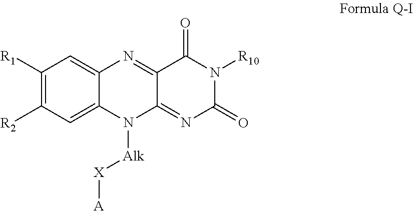
wherein:
(i) Alk is C₁₋₄alkylene;
(ii) X is a single bond, —N(R₆)—, —N(R₆)—CH₂—, —N(R₆)—CH₂CH₂—, —N(R₆)—C(H)(CH₃)—, or —C(O)— and
A is a —C₃₋₈cycloalkyl² wherein one or more carbon atoms of said cycloalkyl² are optionally and independently replaced with N, O, S, S(O)₂ or —C(O)—,
wherein said cycloalkyl² is optionally substituted with one or more
C₁₋₄alkyl
C(O)OR₇,
—CH₂C(O)OR₇,
—N(R₆)C(O)OR₇,
—OH,
hydroxy-C₁₋₄ alkyl,
C₁₋₄alkoxy,
—CH₂N(R₆)—C(O)OR₇,
aryl² or aryl²-C₁₋₄alkyl wherein said aryl² group of said aryl² or aryl²-alkyl is optionally substituted with C₁₋₄alkyl,
heteroaryl²,
heteroaryl²-C₁₋₄alkyl,
—C₁₋₄ alkyl-N(R₈)(R₉),
C₁₋₄alkoxy,
—C(O)N(R₆)—S(O)₂—C₁₋₄alkyl,
—N(H)—S(O)₂—C₁₋₄alkyl,
—S(O)₂—N(R₈)(R₉),
—C(O)N(H)CN,
—C(O)N(R₈)(R₉), or
—N(R₈)(R₉);
or
A is a 7-11 membered fused cycloalkyl-aryl or spiral compound wherein one or more carbon atoms may be a hetero atom selected from N, O or S and wherein said fused cycloalkyl-aryl or spiral group is optionally substituted with one or more hydroxy, C₁₋₄alkyl or oxo;
(iii) R₁ is H or C₁₋₈alkyl;
(iv) R₂ is H, halo, C₁₋₄alkyl, —N(R₄)(R₅) or —O—C₃₋₈cycloalkyl;
(v) R₄ and R₅ are independently selected from
H,
C₃₋₇cycloalkyl²,
—C₁₋₄alkyl, wherein said alkyl is optionally substituted with one or more groups selected from —OH, —C(O)OR₇,
aryl² optionally substituted with halo,
aryl²-C₁₋₄alkyl wherein said aryl² group is optionally substituted with halo;
(vi) R₆ is H or C₁₋₄alkyl;
(vii) R₇ is H, C₁₋₄alkyl or —CH₂OC(O)CH₃;
(viii) R₈ and R₉ are independently H or C₁₋₄alkyl;
(ix) R₁₀ is H or —C₁₋₄alkyl-OC(O)CH₃;
c) a compound of Formula Q-II: 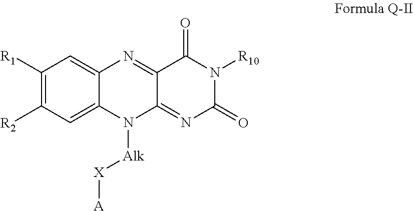
wherein:
(i) Alk is C₁₋₆alkylene;
(ii) X is a single bond and A is:
a monocyclic heteroaryl²,
wherein said heteroaryl² is optionally substituted with one or more C₁₋₄alkyl;
(iii) R₁ is H or C₁₋₈ alkyl;
(iv) R₂ is H, halo, C₁₋₄alkyl, —N(R₄)(R₅) or —O—C₃₋₈cycloalkyl²;
(v) R₄ and R₅ are independently selected from
H,
C₃₋₇cycloalkyl²,
—C₁₋₄alkyl, wherein said alkyl is optionally substituted with one or more groups selected from —OH, —C(O)OR₇,
aryl² optionally substituted with halo,
aryl²-C₁₋₄alkyl wherein said aryl group is optionally substituted with halo;
(vi) R₇ is H, C₁₋₄alkyl or —CH₂OC(O)CH₃;
(vii) R₁₀ is H or —C₁₋₄alkyl-OC(O)CH₃;
d) a compound of Formula Q-III: 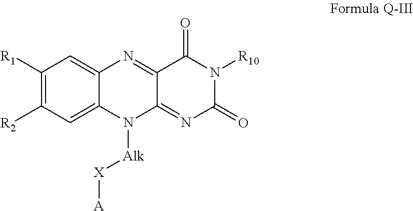
wherein:
(i) Alk is C₁₋₆alkylene;
(ii) X is —N(R₆) and A is:
—C₁₋₄alkyl-N(R₁₁)(R₁₂),
—C₀₋₄alkyl-aryl¹ or —C₀₋₄alkyl-heteroaryl¹, wherein the alkyl group of said -alkylaryl¹ and -alkylheteroaryl¹ is optionally substituted with hydroxy or another aryl¹, and the aryl¹ and heteroaryl¹ group of said -alkylaryl¹ and -alkylheteroaryl¹ are independently substituted with one or more:
—N(Rₐ)—C(O)—C₁₋₄alkyl, wherein Rₐ is H or C₁₋₄alkyl,
—OH,
heteroaryl¹,
heteroC₃₋₈cycloalkyl¹,
aryl¹,
—O-halo-C₁₋₄alkyl,
—NO₂,
—N(Rₐ)(R_{b}), wherein Rₐ is H or C₁₋₄alkyl and R_{b} is C₁₋₄alkyl,
—SO₂—C₁₋₄alkyl;
—C₀₋₄alkyl-pyridyl substituted with one or more hydroxy;
—C₀₋₄alkyl-benzotriazolyl;
—C₀₋₄alkyl-indolyl;
—C₀₋₄alkyl-tetrazolyl;
—C₀₋₄alkyl-oxadiazolyl;
—C₀₋₄alkyl-benzodioxolyl;
—C₀₋₄alkyl-benzimidazolyl optionally substituted with —C₀₋₄alkyl;
—O₀₋₄alkyl-imidazolyl optionally substituted with C₁₋₄alkyl;
—C₀₋₄alkyl-pyrrolyl optionally substituted with —C₀₋₄alkyl;
para-phenylbenzyl;
(iii) R₁ is H or C₁₋₈ alkyl;
(iv) R₂ is H, halo, C₁₋₄alkyl, —N(R₄)(R₅) or —O—C₁₋₈cycloalkyl²;
(v) R₄ and R₅ are independently selected from
H,
C₃₋₇cycloalkyl²,
—C₁₋₄alkyl, wherein said alkyl is optionally substituted with one or more groups selected from —OH, —C(O)OR₇,
aryl² optionally substituted with halo,
aryl²-C₁₋₄alkyl wherein said aryl group is optionally substituted with halo;
(vi) R₆ is H or C₁₋₄alkyl;
(vii) R₇ is H, C₁₋₄alkyl or —CH₂OC(O)CH₃;
(viii) R₁₀ is H or —C₁₋₄alkyl-OC(O)CH₃;
(ix) R₁₁ and R₁₂ are independently H or C₁₋₄alkyl;
e) a compound of Formula Q-IV: 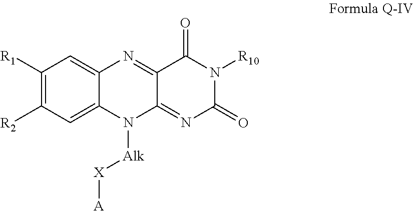
wherein
(i) Alk is C₁₋₆alkylene;
(ii) X is a single bond and A is pyrrolyl or imidazolyl;
or
X is a single bond and A is a pyrrolidinyl or piperidinyl, optionally substituted with another aryl or aryl-C₁₋₄alkyl;
or
X is —N(R₆)— and A is tetralinyl;
(iii) R₁ is H or C₁₋₈alkyl;
(iv) R₂ is H, halo or C₁₋₄alkyl;
(v) R₆ is H or C₁₋₄alkyl;
(vi) R₁₀ is H;
f) a compound of Formula Q-V: 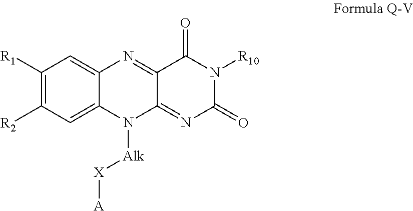
wherein:
(i) Alk is C₁₋₆alkylene;
(ii) X is a single bond and A is pyrrolyl, pyrrolidinyl or piperidinyl optionally substituted with another aryl or aryl-C₁₋₄alkyl;
(iii) R₁ is C₁₋₈ alkyl;
(iv) R₂ is C₁₋₄alkyl;
(v) R₁₀ is H;
g) a compound of Formula I(A): 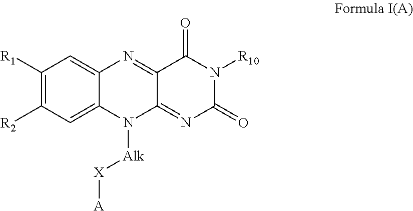
wherein:
(i) Alk is C₁₋₆alkylene;
(ii) X is a single bond, —N(R₆)—, —N(R₆)—CH₂— or —C(O)—;
(iii) A is a monocyclic heteroaryl or C₅₋₆cycloalkyl wherein one or more carbon atoms of said cycloalkyl are optionally and independently replaced with N, O, S, or —C(O)—, wherein said heteroaryl and cycloalkyl are independently optionally substituted with one or more —C(O)OR₂, —CH₂C(O)OR₇, —N(R₆)C(O)OR₂, —OH, hydroxy-C₁₋₄alkyl, —CH₂N(R₆)—C(O)OR₇, heteroaryl, heteroaryl-C₁₋₄alkyl, amineC₁₋₄alkoxy, —C(O)N(R₆)—S(O)₂—C₁₋₄alkyl;
(iv) R₁ is H or C₁₋₈ alkyl;
(v) R₂ is H, halo, C₁₋₄alkyl or —N(R₄)(R₅);
(vi) R₄ and R₅ are independently selected from H, C₃₋₇ cycloalkyl and —C₁₋₄alkyl, wherein said alkyl is optionally substituted with one or more groups selected from —OH, —C(O)OR₇, aryl optionally substituted with halo;
(vii) R₆ is H or C₁₋₄alkyl;
(viii) R₇ is H, C₁₋₄alkyl or —CH₂OC(O)CH₃;
(ix) R₈ and R₉ are independently H or C₁₋₄alkyl;
(x) R₁₀ is H or —C₁₋₄alkyl-OC(O)CH₃;
h) a compound of Formula I(B): 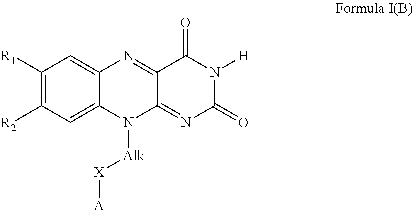
wherein:
(i) Alk is C₁₋₂alkylene;
(ii) X is —N(R₆)—;
(iii) A is selected from a group consisting of:
—C₁₋₄alkyl-N(R₁₁)(R₁₂);
—C₀₋₄alkyl-aryl¹, or —C₀₋₄alkyl-heteroaryl¹, wherein the alkyl group of said -alkylaryl¹ and -alkylheteroaryl¹ is optionally substituted with hydroxy or another aryl, and the aryl¹ and heteroaryl¹ group of said -alkylaryl¹ and -alkylheteroaryl¹ are independently substituted with one or more:
—N(Rₐ)—C(O)—C₁₋₄alkyl (e.g., —NHC(O)CH₃), wherein Rₐ is H or C₁₋₄alkyl,
—OH,
Heteroaryl¹,
heteroC₃₋₈cycloalkyl¹,
aryl¹,
—O-halo-C₁₋₄alkyl,
—NO₂,
—N(Rₐ)(R_{b}), wherein Rₐ is H or C₁₋₄alkyl and R_{b} is C₁₋₄alkyl,
—SO₂—C₁₋₄alkyl;
—C₀₋₄alkyl-pyridyl substituted with one or more hydroxy;
—C₀₋₄alkyl-benzotriazolyl;
—C₀₋₄alkyl-indolyl;
—C₀₋₄alkyl-oxadiazolyl;
—C₀₋₄alkyl-benzodioxolyl;
—C₀₋₄alkyl-benzimidazolyl optionally substituted with —C₀₋₄alkyl;
—C₀₋₄alkyl-imidazolyl optionally substituted with C₁₋₄alkyl;
—C₀₋₄alkyl-pyrrolyl optionally substituted with —C₀₋₄alkyl;
para-phenyl benzyl;
(iv) R₁ is H or C₁₋₄alkyl;
(v) R₂ is selected from a group consisting of H, C₁₋₄alkyl and —O—C₃₋₈cycloalkyl¹;
(vi) R₆ is H or C₁₋₄alkyl;
(vii) R₁₁ and R₁₂ are independently H or C₁₋₄alkyl;
i) a compound of Formula I(A)(i): 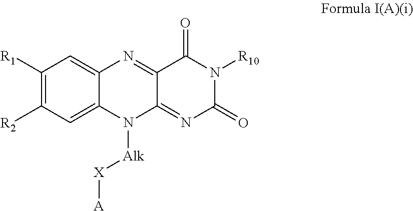
wherein:
(i) Alk is C₁₋₆alkylene;
(ii) X is a single bond, —N(R_{6)—, —N(R}₆)—CH₂— or —C(O)—;
(iii) A is a monocyclic heteroaryl or C₅₋₆cycloalkyl wherein one or more carbon atoms of said cycloalkyl are optionally and independently replaced with N, O, S, or —C(O)—, wherein said heteroaryl and cycloalkyl are independently optionally substituted with one or more —C(O)OR₇—CH₂C(O)OR₇, —N(R)C(O)OR₇, —OH, hydroxy-C₁₋₄alkyl, —CH₂N(R₆)—C(O)OR₇, heteroaryl, heteroaryl-C₁₋₄alkyl, amineC₁₋₄alkyl, C₁₋₄alkoxy, —C(O)N(R₆)—S(O)₂—C₁₋₄alkyl or —N(R₈)(R₉);
(iv) R₁ is H or C₁₋₈alkyl;
(v) R₂ is H, halo, C₁₋₄alkyl or —N(R₄)(R₅);
(vi) R₄ and R₅ are independently selected from H, C₃₋₇cycloalkyl, —C₁₋₄alkyl, wherein said alkyl is optionally substituted with one or more groups selected from —OH, —C(O)OR₇, and aryl optionally substituted with halo;
(vii) R₆ is H or C₁₋₄alkyl;
(viii) R₂ is H, C₁₋₄alkyl or —CH₂OC(O)CH₃;
(ix) R₈ and R₉ are independently H or C₁₋₄alkyl;
(x) R₁₀ is H or —C₁₋₄alkyl-OC(O)CH₃;
with the proviso that when R₂ is chloro, Alk is propylene, X is a single bond and A is pyrrolidin-1, then R₁ is C₁₋₈ alkyl or R₁₀ is —C₁₋₄alkyl-OC(O)CH₃;
i) a Compound of Formula II(A): 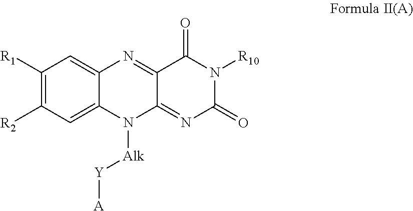
wherein
(i) Alk is C₁₋₆alkylene;
(ii) Y is —N(R₆)—C(O)— or —C(O)—N(R₆)—;
(iii) A is heteroaryl optionally substituted with one or more —C(O)OR₇, —CH₂C(O)OR₇, —N(R₆)C(O)OR₇, —OH, hydroxy-C₁₋₄alkyl, —CH₂N(R₆)—C(O)OR₇, heteroaryl, heteroaryl-C₁₋₄alkyl, amineC₁₋₄alkyl, C₁₋₄alkoxy, —C(O)N(R₆)—S(O)₂—C₁₋₄alkyl or —N(R₈)(R₉);
(iv) R₁ is H or C₁₋₈ alkyl;
(v) R₂ is H, halo, C₁₋₄alkyl (e.g., methyl) or —N(R₄)(R₅);
(vi) R₄ and R₅ are independently selected from H, C₃₋₇ cycloalkyl, and —C₁₋₄alkyl wherein said alkyl is optionally substituted with one or more groups selected from —OH, —C(O)OR₇, and aryl optionally substituted with halo,
(vii) R₆ is H or C₁₋₄alkyl;
(viii) R₇ is H, C₁₋₄alkyl or —CH₂OC(O)CH₃;
(ix) R₈ and R₉ are independently H, or C₁₋₄alkyl;
(x) R₁₀ is H or —C₁₋₄alkyl-OC(O)CH₃;
k) a Compound of Formula II(B): 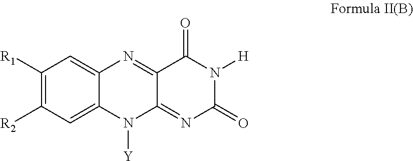
wherein:
(i) R₁ is H or C₁₋₄alkyl;
(ii) R₂ is selected from a group consisting of H, C₁₋₄alkyl and —O—C₃₋₈cycloalkyl¹;
(iii) Y is selected from a group consisting of: 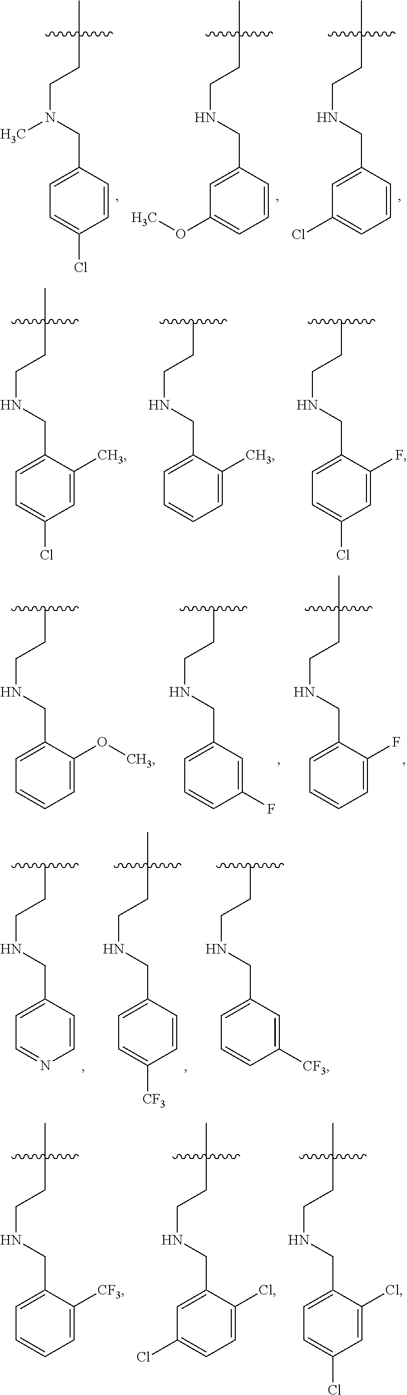 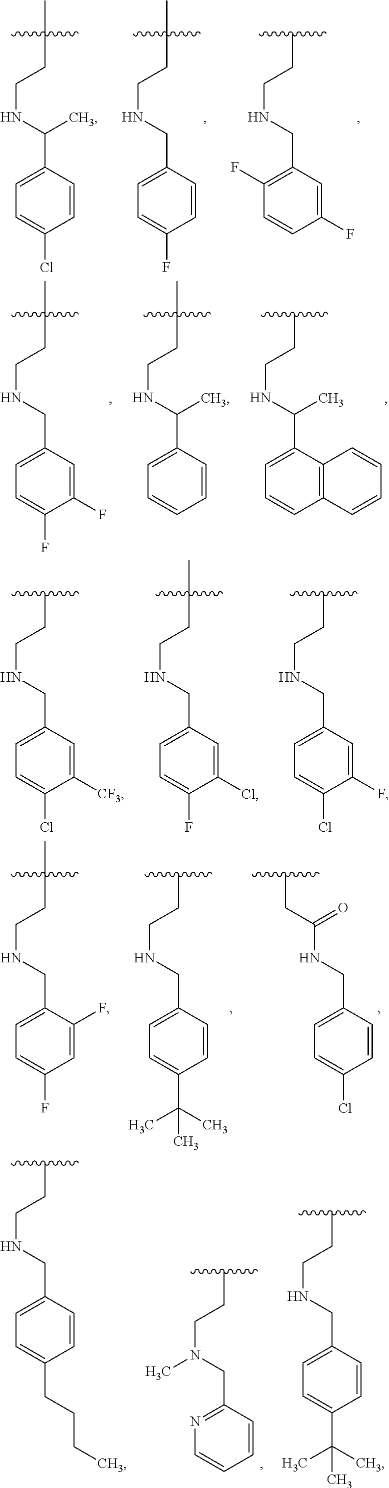 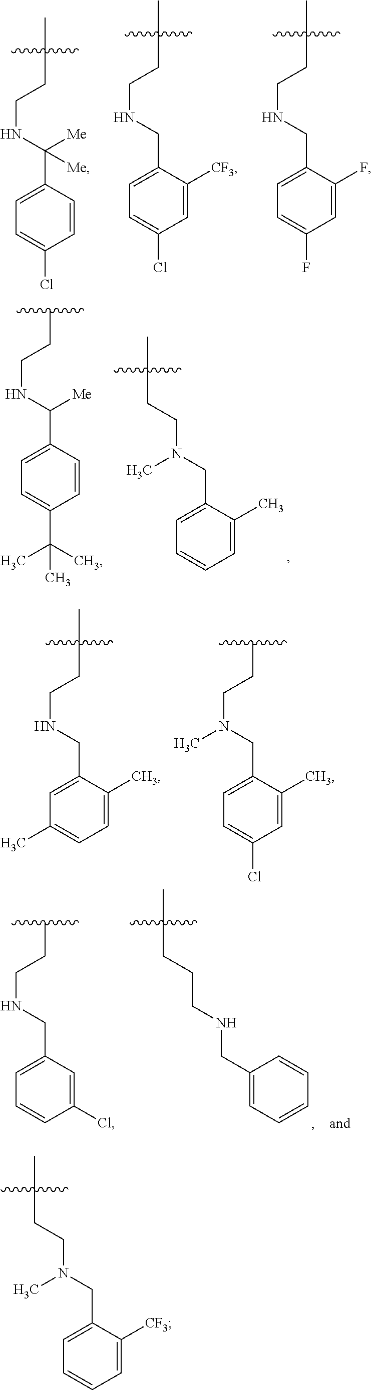
D) a Compound of Formula III(B): 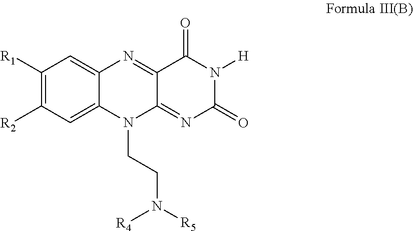
wherein:
(i) R₁ is H or C₁₋₄alkyl;
(ii) R₂ is selected from a group consisting of H, C₁₋₄alkyl and —O—C₃₋₈cycloalkyl;
(iii) R₄ is benzyl;
(iv) R₅ is selected from aryl¹-C₀₋₄alkyl, hydroxyC₁₋₄alkyl, C₁₋₄alkyl, and C₃₋₈cycloalkyl¹, wherein R₅ is optionally substituted with one or more hydroxy or C₁₋₄alkyl (e.g. methyl);
(v) or R₄ is H and R₅ is 1,2-diphenylethyl or 1-hydroxy-2-hydroxymethyl-2-phenyl (—C(H)(CH₂OH)—C(H)(OH)—C₆H₅);
in free or pharmaceutically acceptable salt form.

20. The method according to claim 19, wherein the infection is a Gram-positive or Gram-negative bacterial infection.

21. The method according to claim 19, wherein the bacterial infection is selected from a group consisting of Clostridium difficile, Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and Borrelia burgdorferi.

22. The method according to claim 19, wherein the bacterial infection is a Clostridium difficile infection.

23. The method according claim 19, wherein the bacterial infection is Staphylococcus aureus infection.

24. The method according to claim 19, wherein said infection is by an infectious agent which is resistant to a drug that is not a riboswitch ligand.

25. The method according to claim 19, wherein the infection is an infection which is resistant to one or more drugs selected from a group consisting of a penicillin, vancomycin, cephalosporin and methicillin.

26. The method according to claim 25, wherein the infection is a methicillin-resistant Staphylococcus aureus infection.

27. The method according to claim 19, wherein the infection is a fluoroquinolone-resistant, metronidazole-resistant and/or vancomycin—resistant C. difficile infection.

28. The method according to claim 19, wherein the compound is a compound of Formula Q in free or pharmaceutically acceptable salt form.

29. The method according to claim 19, wherein the compound is selected from a group consisting of those described in any of formulae Q.35, Q.36, Q.37, Q.38, Q.39, Q.40 or Q.41, in free or pharmaceutically acceptable salt form.

30. The method according to claim 19, wherein the compound is selected from a group consisting of those described in formula Q.41, in free or pharmaceutically acceptable salt form.

31. The method according to claim 19, wherein the compound is a compound of Formula II, in free or pharmaceutically acceptable salt form.

32. A pharmaceutical composition comprising the compound according to claim 1, in free or pharmaceutically acceptable salt form, in admixture with a pharmaceutically acceptable diluent or carrier.

33. (canceled)

34. (canceled)

35. (canceled)

36. A method for the treatment or prophylaxis of a bacterial infection in a plant comprising administering to said plant an effective amount of a compound according of any of claims 19, in free or pharmaceutically acceptable salt form.
